(19) United States
(12) Patent Application Publication
(10) Pub. No.: US 2004/0101523 A1
(43) Pub. Date: May 27, 2004

(54) Renal-selective prodrugs for control of renal smpathetic nerve activity in the treatment of hypertension

(75) Inventors: David B. Reitz, Chesterfield, MO (US); John P. Koepke, St. Louis, MO (US); Edward H. Blaine, University City, MO (US); Joseph R. Schuh, St. Louis, MO (US); Robert E. Manning, St. Louis, MO (US); Glenn J. Smits, Ellisville, MO (US)

Correspondence Address: J. Timothy Keane, PHARMACIA CORPORATION of Pfizer Inc.,, Corporate Patent Department, P.O. Box 1027, Chesterfield, MO, 63006, US

(73) Assignee: G.D. Searle & Co., Chicago, IL

(21) Appl. No.: 10/689,919

(22) Filed: Oct. 20, 2003

Related U.S. Application Data
(63) Continuation of application No. 10/151,211, filed on May 20, 2002, now abandoned.
Application No. 10/151,211 is a continuation of application No. 09/678,015, filed on Oct. 2, 2000, now abandoned.
Application No. 09/678,015 is a continuation of application No. 09/444,888, filed on Nov. 22, 1999, now abandoned.
Application No. 09/444,888 is a continuation of application No. 08/639,493, filed on Apr. 29, 1996, now abandoned.
Application No. 08/639,493 is a continuation of application No. 08/280,170, filed on Jul. 25, 1994, now abandoned.
Application No. 08/280,170 is a continuation-in-part of application No. PCT/US90/04168, filed on Jul. 25, 1990.
Application No. PCT/US90/04168 is a continuation-in-part of application No. 07/386,527, filed on Jul. 27, 1989, now abandoned.

(51) Int. Cl. A61K 38/43; C12N 9/99

(52) U.S. Cl. 424/94.1; 435/184

(57) ABSTRACT

Renal-selective prodrugs are described which are preferentially converted in the kidney to compounds capable of inhibiting synthesis of catecholamine-type neurotransmitters involved in renal sympathetic nerve activity. The prodrugs described herein are derived from inhibitor compounds capable of inhibiting one or more of the enzymes involved in catecholamine synthesis, such compounds being classifiable as tyrosine hydroxylase inhibitors, or as dopa-decarboxylase inhibitors, or as dopamine-β-hydroxylase inhibitors. These inhibitor compounds are linked to a chemical moiety, such as a glutamic acid derivative, by a cleavable bond which is recognized selectively by enzymes located predominantly in the kidney. The liberated inhibitor compound is then available in the kidney to inhibit one or more of the enzymes involved in catecholamine synthesis. Inhibition of renal catecholamine synthesis can suppress heightened renal nerve activity associated with sodium-retention related disorders such as hypertension. Conjugates of particular interest are glutamyl derivatives of dopamine-β-hydroxylase inhibitors, of which N-acetyl-γ-glutamyl fusaric acid hydrazide (shown below) is preferred.

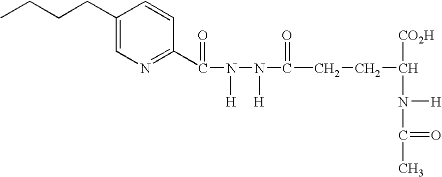

### RELATED APPLICATION

This application is a continuation-in-part of U.S. Application Ser. No. PCT/US90/04168 filed 25 Jul. 1990, which is a continuation-in-part of U.S. application Ser. No. 07/386,527 filed 27 Jul. 1989.

### FIELD OF THE INVENTION

This invention is in the field of cardiovascular therapeutics and relates to a class of compounds useful in control of hypertension. Of particular interest is a class of compounds which prevent or control hypertension by selective action on the renal sympathetic nervous system.

### BACKGROUND OF THE INVENTION

Hypertension has been linked to increased sympathetic nervous system activity stimulated through any of four mechanisms, namely (1) by increased vascular resistance, (2) by increased cardiac rate, stroke volume and output, (3) by vascular muscle defects or (4) by sodium retention and renin release [J. P. Koepke et al, The Kidney in Hypertension, B. M. Brenner and J. H. Laragh (Editors), Vol. 1, p. 53 (1987)]. As to this fourth mechanism in particular, stimulation of the renal sympathetic nervous system can affect renal function and maintenance of homeostasis. For example, an increase in efferent renal sympathetic nerve activity may cause increased renal vascular resistance, renin release and sodium retention [A. Zanchetti et al, Handbook of Hypertension, Vol. 8, Ch. 8, vasoconstriction has been identified as an element in the pathogenesis of early essential hypertension in man. [R. E. Katholi, Amer. J. Physiol., 245, F1-F14 (1983)].

Proper renal function is essential to maintenance of homeostasis so as to avoid hypertensive conditions. Excretion of sodium is key to maintaining extracellular fluid volume, blood volume and ultimately the effects of these volumes on arterial pressure. Under steady-state conditions, arterial pressure rises to that pressure level which will cause balance between urinary output and water/salt intake. If a perturbation in normal kidney function occurs causing renal sodium and water retention, as with sympathetic stimulation of the kidneys, arterial pressure will increase to a level to maintain sodium output equal to intake. In hypertensive patients, the balance between sodium intake and output is achieved at the expense of an elevated arterial pressure.

During the early stages of genetically spontaneous or deoxycorticosterone acetate-sodium chloride (DOCA-NaCl) induced hypertension in rats, a positive sodium balance has been observed to precede hypertension. Also, surgical sympathectomy of the kidneys has been shown to reverse the positive sodium balance and delay the onset of hypertension [R. E. Katholi, Amer. J. Physiol., 245, F1-F14 (1983)]. Other chronic sodium retaining disorders are linked to heightened sympathetic nervous system stimulation of the kidneys. Congestive heart failure, cirrhosis and nephrosis are characterized by abnormal chronic sodium retention leading to edema and ascites. These studies support the concept that renal selective pharmacological inhibition of heightened sympathetic nervous system activity to the kidneys may be an effective therapeutic treatment for chronic sodium-retaining disorders, such as hypertension, congestive heart failure, cirrhosis, and nephrosis.

One approach to reduce sympathetic nervous system effects on renal function is to inhibit the synthesis of one or more compounds involved as intermediates in the “catecholamine cascade”, that is, the pathway involved in synthesis of the neurotransmitter norepinephrine. Stepwise, these catecholamines are synthesized in the following manner: (1) tyrosine is converted to dopa by the enzyme tyrosine hydroxylase; (2) dopa is converted to dopamine by the enzyme dopa decarboxylase; and (3) dopamine is converted to norepinephrine by the enzyme dopamine-β-hydroxylase. Inhibition of dopamine-β-hydroxylase activity, in particular, would increase the renal vasodilatory, diuretic and natriuretic effects due to dopamine. Inhibition of the action of any of these enzymes would decrease the renal vasoconstrictive, antidiuretic and antinatriuretic effects of norepinephrine. Therapeutically, these effects oppose chronic sodium retention.

Many compounds are known to inhibit the action of the catecholamine-cascade-converting enzymes. For example, the compound α-methyltyrosine inhibits the action of the enzyme tyrosine hydroxylase. The compound α-methyldopa inhibits the action of the enzyme dopa-decarboxylase, and the compound fusaric acid inhibits the action of dopamine-β-hydroxylase. Such inhibitor compounds often cannot be administered systemically because of the adverse side effects induced by such compounds. For example, the desired therapeutic effects of dopamine-β-hydroxylase inhibitors, such as fusaric acid, may be offset by hypotension-induced compensatory stimulation of the renin-angiotensin system and sympathetic nervous system, which promote sodium and water retention.

To avoid such systemic side effects, drugs may be targetted to the kidney by creating a conjugate compound that would be a renal-specific prodrug containing the targetted drug modified with a chemical carrier moiety. Cleavage of the drug from the carrier moiety by enzymes predominantly localized in the kidney releases the drug in the kidney. Gamma glutamyl transpeptidase and acylase are examples of such cleaving enzymes found in the kidney which have been used to cleave a targetted drug from its prodrug carrier within the kidney.

Renal targetted prodrugs are known for delivery of a drug selectively to the kidney. For example, the compound L-γ-glutamyl amide of dopamine when administered to dogs was reported to generate dopamine in vivo by specific enzymatic cleavage by γ-glutamyl transpeptidase [J. J. Kyncl et al, Adv. Biosc., 20, 369-380 (1979)]. In another study, γ-glutamyl and N-acyl-γ-glutamyl derivatives of the anti-bacterial compound sulfamethoxazole were shown to deliver relatively high concentrations of sulfamethoxazole to the kidney which involved enzymatic cleavage of the prodrug by acylamino acid deacylase and γ-glutamyl transpeptidase [M. Orlowski et al, J. Pharmacol. Exp. Ther., 212, 167-172 (1980)]. The N-γ-glutamyl derivatives of 2-, 3-, or 4-aminophenol and p-fluoro-L-phenylalanine have been found to be readily solvolyzed in vitro by γ-glutamyl transpeptidase [S. D. J. Magnan et al, J. Med. Chem., 25, 1018-1021 (1982)]. The hydralazine-like vasodilator 2-hydrazino-5-g-butylpyridine (which stimulates guanylate cyclase activity) when substituted with the N-acetyl-γ-glutamyl residue resulted in a prodrug which provided selective renal vasodilation [K. G. Hofbauer et al, J. Pharmacol. Exp. Ther., 212, 838-844 (1985)]. The dopamine prodrug γ-L-glutamyl-L-dopa (“gludopa”) has been shown to be relatively specific for the kidney and to increase renal blood flow, glomerular filtration and urinary sodium excretion in normal subjects [D. P. Worth et al, Clin. Sci. 69, 207-214 (1985)]. In another study, gludopa was reported to an effective renal dopamine prodrug whose activity can be blocked by the dopa-decarboxylase inhibitor carbidopa [R. F. Jeffrey et al, Br. J. Clin. Pharmac., 25, 195-201 (1988)].

### BRIEF DESCRIPTION OF THE DRAWING FIGURES

FIG. 1 shows the acute effects of i.v. injection of vehicle and Example #3 conjugate on mean arterial pressure in rats.

FIG. 2 shows the acute effects of i.v. injection of vehicle and Example #3 conjugate on renal blood flow in rats.

FIG. 3 shows the chronic effects of i.v. infusion of vehicle and Example #464 conjugate on mean arterial pressure in spontaneously hypertensive rats.

FIG. 4 shows time-dependent formation of the dopamine-β-hydroxylase inhibitor fusaric acid from the Example #859 conjugate incubated with rat kidney homogenate.

FIG. 5 shows time-dependent formation of fusaric acid from the Example #859 conjugate incubated with a mixture of purified acylase I and gamma-glutamyl transpeptidase at pH 7.4 and 8.1.

FIG. 6 shows the concentration-dependent effect of fusaric acid and the Example #859 conjugate on norepinephrine production by dopamine-β-hydroxylase in vitro.

FIG. 7 shows dopamine-β-hydroxylase inhibition in vitro by fusaric acid, the Example #859 conjugate and possible metabolites at a concentration of 20 μM.

FIG. 8 shows the acute effects of i.v. injection of fusaric acid and Example #859 conjugate on mean arterial pressure in spontaneously hypertensive rats.

FIG. 9 shows the acute effects of i.v. injection of fusaric acid and Example #859 conjugate on renal blood flow in spontaneously hypertensive rats.

FIG. 10 shows the effects of chronic i.v. infusion of vehicle, fusaric acid, and Example #859 conjugate for 5 days on mean arterial pressure in spontaneously hypertensive rats.

FIG. 11 shows the effects of chronic i.v. infusion of vehicle and Example #863 conjugate for 4 days on mean arterial pressure in spontaneously hypertensive rats.

FIG. 12 shows the heart tissue concentrations of norepinephrine following the 5 day infusion experiment described in FIG. 10.

FIG. 13 shows the kidney tissue concentrations of norepinephrine following the 5 day infusion experiment described in FIG. 10.

FIG. 14 shows the effects of Example #859 conjugate on mean arterial pressure in anesthetized dogs after i.v. injection at three doses, plus vehicle.

FIG. 15 shows the effects of Example #859 conjugate on renal blood flow in anesthetized dogs after i.v. injection at three doses, plus vehicle.

FIG. 16 shows the effects of Example #858 conjugate on mean arterial pressure in conscious DOCA hypertensive micropigs after i.v. infusion for three days.

### DESCRIPTION OF THE INVENTION

Treatment of chronic hypertension or sodium-retaining disorders such as congestive heart failure, cirrhosis and nephrosis, may be accomplished by administering to a susceptible or afflicted subject a therapeutically-effective amount of a renal-selective prodrug capable of causing selective blockage of heightened sympathetic nervous system effects on the kidney. An advantage of such renal-selective prodrug therapy resides in reduction or avoidance of adverse side effects associated with systemically-acting drugs.

A renal-selective prodrug capable of providing renal sympathetic nerve blocking action may be provided by a conjugate comprising a first residue and a second residue connected together by a cleavable bond. The first residue is derived from an inhibitor compound capable of inhibiting formation of a benzylhydroxyamine intermediate in the biosynthesis of an adrenergic neurotransmitter, and wherein said second residue is capable of being cleaved from the first residue by an enzyme located predominantly in the kidney.

The first and second residues are provided by precursor compounds having suitable chemical moieties which react together to form a cleavable bond between the first and second residues. For example, the precursor compound of one of the residues will have a reactable carboxylic acid moiety and the precursor of the other residue will have a reactable amino moiety or a moiety convertible to a reactable amino moiety, so that a cleavable bond may be formed between the carboxylic acid moiety and the amino moiety. An inhibitor compound which provides the first residue may be selected from tyrosine hydroxylase inhibitor compounds, dopa-decarboxylase inhibitor compounds, dopamine-β-hydroxylase inhibitor compounds, and mimics of any of these inhibitor compounds.

The inhibitor compounds described herein have been classified as tyrosine hydroxylase inhibitors, or as dopa-decarboxylase inhibitors, or as dopamine-β-hydroxylase inhibitors, for convenience of description. Some of the inhibitor compounds may be classifiable in more than one of these classes. For example, 2-vinyl-3-phenyl-2-aminopropionic acid derivatives are classified herein as tyrosine hydroxylase inhibitors, but such derivatives may also act as dopa-decarboxylase inhibitors. The term “inhibitor compound” means a compound of any of the three foregoing classes and which has the capability to inhibit formation of a benzylhydroxyamine intermediate involved in biosynthesis of an adrenergic neurotransmitter. Thus, a compound which does not inhibit formation of such benzylhydroxyamine intermediate is not embraced by the definition of “inhibitor compound” as used herein. For example, compounds which do not inhibit a benzylhydroxyamine intermediate are the compounds L-dopa and dopamine.

A class of compounds from which a suitable tyrosine hydroxylase inhibitor compound may be selected to provide the conjugate first residue is represented by Formula I:

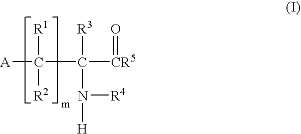

wherein each of R¹ through R³ is independently selected from hydrido, hydroxy, alkyl, cycloalkyl, cycloalkylalkyl, aralkyl, aryl, alkoxy, aryloxy, aralkoxy, alkoxyalkyl, haloalkyl, hydroxyalkyl, halo, cyano, amino, monoalkylamino, dialkylamino, carboxyl, carboxyalkyl, alkanoyl, alkenyl, cycloalkenyl and alkynyl; wherein R⁴ selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein R⁵ is selected from —OR⁶ and

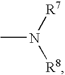

wherein R⁶ is selected from hydrido, alkyl, cycloalkyl, cycloalkylalkyl, aralkyl and aryl, and wherein each of R⁷ and R⁸ is independently selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein m is a number selected from zero through six; wherein A is a phenyl ring of the formula

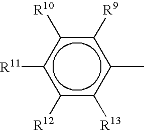

wherein each of R⁹ through R¹³ is independently selected from hydrido, hydroxy, alkyl, cycloalkyl, cycloalkylalkyl, aralkyl, aryl, alkoxy, aralkoxy, alkoxyalkyl, haloalkyl, hydroxyalkyl, halo, cyano, amino, monoalkylamino, dialkylamino, carboxyl, carboxyalkyl, alkanoyl, alkenyl, cycloalkenyl, alkynyl, cyanoamino, carboxyl, cyano, thiocarbamoyl, aminomethyl, alkylsulfanamido, nitro, alkylsulfonyloxy, carboxyalkoxy, formyl and a substituted or unsubstituted 5- or 6-membered heterocyclic ring selected from the group consisting of pyrrol-1-yl, 2-carboxypyrrol-1-yl, imidazol-2-ylamino, indol-1-yl, carbozol9-yl, 4,5-dihydro-4-hydroxy-4-trifluoromethylthiazol3-yl, 4-trifluoromethylthiazol-2-yl, imidazol-2-yl and 4,5-dihydroimidazol-2-yl; wherein any two of the R⁹ through R¹³ groups may be taken together to form a benzoheterocylic ring selected from the group consisting of indolin-5-yl, 1-(N-benzoylcarbamimidoyl)indolin5-yl, 1-carbamimidoylindolin-5-yl, 1H-2-oxindol-5-yl, insol-5-yl, 2-mercaptobenzimidazol-5(6)-yl, 2-aminobenzimidazol-5-(6)-yl, 2-methanesulfonamidobenzimidazol-5(6)-yl, 1H-benzoxanol-2-on-6-yl, 2-aminobenzothiazol-6-yl, 2-amino-4-mercaptobenzothiazol6-yl, 2,1,3-benzothiadiazol-5-yl, 1,3-dihydro-2,2-dioxo-2,1,3-benzothiadiazol-5-yl, 1,3-dihydro-1,3-dimethyl2,2-dioxo-2,1,3-benzothiadiazol-5-yl, 4-methyl-2(H) oxoquinolin-6-yl, quinoxalin-6-yl, 2-hydroxyquinoxalin-6-yl, 2-hydroxquinoxalin-7-yl, 2,3-dihydroxyquinoxalin6-yl and 2,3-didydro-3(4H)-oxo-1,4-benzoxazin-7-yl; 5-hydroxy-4H-pyran-4-on-2-yl, 2-hydroxypyrid-4-yl, 2-aminopyrid-4-yl, 2-carboxypyrid-4-yl and tetrazolo-[1,5-a]pyrid-7-yl; and wherein A may be selected from

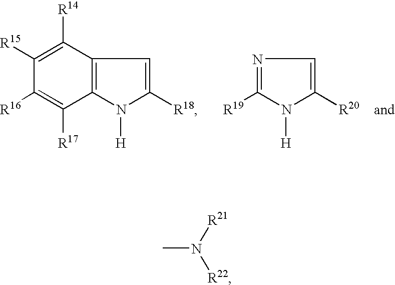

wherein each of R¹⁴ through R²⁰ is independently selected from hydrido, alkyl, hydroxy, hydroxyalkyl, alkoxy, cycloalkyl, cycloalkylalkyl, halo, haloalkyl, aryloxy, alkoxycarboxyl, aryl, aralkyl, cyano, cyanoalkyl, amino, monoalkylamino and dialkylamino, wherein each of R²¹ and R²² is independently selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; or a pharmaceutically-acceptable salt thereof.

A preferred class of tyrosine hydroxylase inhibitor compounds within Formula I is provided by compounds of Formula II:

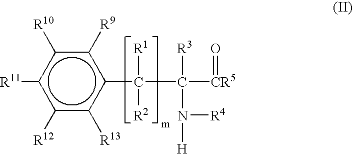

wherein each of R¹ and R² is hydrido; wherein m is one or two; wherein R³ is selected from alkyl, alkenyl and alkynyl; wherein R⁴ is selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein R⁵ is selected from —OR⁶ and

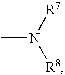

wherein R⁶ is selected from hydrido, alkyl, cycloalkyl, cycloalkylalkyl, phenalkyl and phenyl, and wherein each of R⁷ and R⁸ is independently selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein each of R⁹ through R¹³ is independently selected from hydrido, hydroxy, alkyl, cycloalkyl, cycloalkylalkyl, aralkyl, aryl, alkoxycarbonyl, alkoxycarbonyl, alkoxy, arykoxy, aralkoxy, alkoxyalkyl, haloalkyl, hydroxyalkyl, halo, cyano, amino, monoalkylamino, dialkylamino, dialkylamino, carboxyl, carboxyalkyl, alkanoyl, alkenyl, cycloalkenyl, alkynyl, pyrrol-1-yl 2-carboxypyrrol-1-yl, imidazol-2-ylamino, indol-1-yl, carbazol-9-yl, 4,5-dihydro-4-trifluoromethylthiazol-3-yl, 4-trifluoromethylthiazol-2-yl, imidazol-2-yl and 4,5-dihydroimidazol-2-yl, and wherein any two of the R⁹ through R¹³ groups may be taken together to form a benzoheterocyclic ring selected from the group consisting of indolin-5-yl, 1-(N-benzoylcarbamimidoyl)indolin-5-yl, 1-carbamimidoylindolin-5-yl, 1H-2-oxindol-5-yl, indol-5-yl, 2-mercaptobenzimidazol-5(6)-yl, 2-aminobenzimidazol-5-(6)-yl, 2-methanesulfonamidobenzimidazol-5(6)-yl, 1H-benzoxanol-2-on-6-yl, 2-amino-benzothiazol-6-yl, 2-amino-4-mercaptobenzothiazol-6-yl, 2,1,3-benzothiadiazol-5-yl, 1,3-dihydro-2,2-dioxo-2,1, 3-benzothiadiazol-5-yl, 1,3-dihydro-1,3-dimethyl-2,2-dioxo-2,1,3-benzothiadiazol-5-yl, 4-methyl-2(H)-oxoquinolin-6-yl, quinoxalin-6-yl, 2-hydroxyquinoxalin-6-yl, 2-hydroxquinoxalin-7-yl, 2,3-dihydroxyquinoxalin-6-yl and 2,3-didydro-3(4H)-oxo-1,4-benzoxazin-7-yl; wherein R³ is —CH═CH₂ or —C═CH; wherein R⁵ is selected from —OR⁶ and

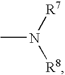

wherein R⁶ is selected from hydrido, alkyl, hydroxy, hydroxyalkyl, alkoxy, halo, haloalkyl, cycloalkyl, cycloalkylalkyl, aryl, aralkyl, amino, monoalkylamino, dialkylamino; and wherein each of R⁷ and R⁸ independently is selected from hydrido, alkyl, hydroxyalkyl, cycloalkyl, cycloalkylalkyl, aryl and aralkyl; or a pharmaceutically-acceptable salt thereof.

A first sub-class of preferred tyrosine hydroxylase inhibitor compounds consists of the following specific compounds within Formula II:

4-cyanoamino-α-methylphenyalanine;

3-carboxy-α-methylphenylalanine;

3-cyano-α-methylphenylalanine methyl ester;

α-methyl-4-thiocarbamoylphenylalanine methyl ester;

4-(aminomethyl)-α-methylphenylalanine;

4-guanidino-α-methylphenylalanine;

3-hydroxy-4-methanesulfonamido-α-methylphenylalanine;

3-hydroxy-4-nitro-α-methylphenylalanine;

4-amino-3-methanesulfonyloxy-α-methylphenylalanine;

3-carboxymethoxy-4-nitro-α-methylphenylalanine;

α-methyl-4-amino-3-nitrophenylalanine;

3,4-diamino-α-methylphenylalanine;

α-methyl-4-(pyrrol-1-yl)phenylalanine;

4-(2-aminoimidazol-1-yl)-α-methylphenylalanine;

4-(imidazol-2-ylamino)-α-methylphenylalanine;

4-(4,5-dihydro-4-hydroxy-4-trifluoromethyl-thiazol-2-yl)-a-methylphenylalanine methyl ester;

α-methyl-4-(4-trifluoromethylthiazol-2-yl)phenylalanine;

α-methyl-3-(4-trifluoromethylthiazol-2-yl)-phenylalanine;

4-(imidazol-2-yl)-α-methylphenylalanine;

4-(4,5-dihydroimidazol-2-yl)-α-methylphenylalanine;

3-(imidazol-2-yl)-α-methylphenylalanine;

3-(4,5-dihydroimidazol-2-yl)-a-methylphenylalanine;

4-(imidazol-2-yl)phenylalanine;

4,5-dihydroimidazol-2-yl)phenylalanine;

3-(imidazol-2-yl)phenylalanine;

3-(2,3-dihydro-1H-indol-4-yl)-α-methylalanine;

α-methyl-3-(1H-2-oxindol-5-yl)alanine;

3-1-(N-benzoylcarbamimidoyl)-2,3-dihydro-1H-indol-5-yl)-α-methylalanine;

3-(1-carbamimidoyl-2,3-dihydro-1H-indol-5-yl-α-methylalanine;

3-(1H-indol-5-yl-α-methylalanine;

3-(benzimidazol-2-thione-5-yl)-α-methylalanine;

3-(2-aminobenzimidazol-5-yl-2-methylalanine;

2-methyl-3-(benzoxazol-2-on-6-yl)alanine;

3-(2-aminobenzothiazol-6-yl)-2-methylalanine;

3-(2-amino-4-mercaptobenzothiazol-6-yl)-2methylalanine;

3-(2-aminobenzothiazol-6-yl)alanine;

2-methyl-3-(2,1,3-benzothiadiazol-5-yl)alanine;

3-(1,3-dihydrobenzo-2,1,3-thiadiazol-5-yl)-2-methylalanine-2,2-dioxide;

3-(1,3-dihydrobenzo-2,1,3-thiadiazol-5-yl)-2-methylalanine-2,2-dioxide methyl ester;

3-(1,3-dihydrobenzo-2,1,3-thiadiaxol-5-yl)alanine 2,2-dioxide;

3-(1,3-dihydro-1,3-dimethylbenzo-2,1,3-thiadiazol-5-yl-)-2-methylalanine 2,2-dioxide;

α-methyl-3-[4-methyl-2(1H)-oxoquinolin-6-yl]alanine;

3-4-methyl-2(1H)-oxoquinolin-6-yl]alanine;

2-methyl-3-(quinoxalin-6-yl)alanine;

2-methyl-3-(2-hydroxyquinoxalin-6-yl)alanine;

2-methyl-3-(2-hydroxyquinoxalin-7-yl)alanine;

3-(2,3-dihydroxyquinoxalin-6-yl)-2-methylalanine;

3-(quinoxalin-6-yl)alanine;

3-(2,3-dihydroxyquinoxalin-6-yl)alanine;

3-(1,4-benzoxazin-3-one-6-yl)-2-methylalanine;

3-(1,4-benzoxazin-3-one-7-yl)alanine;

3-(5-hydroxy-4H-pyran-4-on-2-yl)-2-methylalanine;

3-(2-hydroxy-4-pyridyl)-2-methylalanine;

3-(2-carboxy-4-pyridyl)-2-methylamine;

α-methyl-4-(pyrrol-1-yl)phenylalanine;

α-ethyl-4-(pyrrol-1-yl)phenylalanine;

α-propyl-4-(pyrrol-1-yl)phenylalanine;

4-[2-(carboxy)pyrrol-1-yl)phenylalanine;

α-methyl-4-(pyrrol-1-yl)phenylalanine;

3-hydroxy-α-4-(pyrrol-1-yl)phenylalanine;

3-methoxy-α-4-(pyrrol-1-yl)phenylalanine;

4-methoxy-α-3-(pyrrol-1-yl)phenylalanine;

4-(indol-1-yl)-α-methylphenylalanine;

4-(carbazol-9-yl)-α-methylphenylalanine;

2-methyl-3-(2-methanesulfonylamidobenzimidazol-5-yl)alanine;

2-methyl-3-(2-amino-4-pyridyl)alanine;

2-methyl-3[tetrazolo-(1,5)-α-pyrid-7-yl]alanine;

D,L-α-β-(4-hydroxy-3-methyl)phenylalanine;

D,L-α-β-(4-hydroxy-3-phenyl)phenylalanine;

D,L-O— (4-hydroxy-3-benzyl) phenylalanine;

D,L-α-β-(4-methoxy-3-cyclohexyl)phenylalanine;

α, β, β trimethyl-β-(3,4-dihydroxyphenyl)alanine;

α, β, β trimethyl-β-(4-hydroxyphenyl)alanine;

N-methyl α, β, β trimethyl-β-(3,4-dihydroxphenyl) alanine;

D,L α, β, β trimethyl-β-(3,4-dihyroxyphenyl)alanine;

trimethyl-β-(3,4-dimethoxyphenyl)alanine;

L-α-methyl-β-3,4-dihydroxyphenylalanine;

L-α-ethyl-β-3,4-dihydroxyphenylalanine;

L-α-propyl-β-3,4-dihydroxyphenylalanine;

L-α-butyl-β-3,4-dihydroxyphenylalanine;

L-α-methyl-β-2,3-dihydroxphenylalanine;

L-α-ethyl-β-2,3-dihydroxphenylalanine;

L-α-propyl-β-2,3-dihydroxphenylalanine;

L-α-butyl-β-2,3-dihydroxphenylalanine;

L-α-methyl-4-chloro-2,3-dihydroxyphenylalanine;

L-α-ethyl-4-chloro-2,3-dihydroxyphenylalanine;

L-α-propyl-4-chloro-2,3-dihydroxyphenylalanine;

L-α-butyl-4-chloro-2,3-dihydroxyphenylalanine;

L-α-ethyl-β-4-methyl-2,3-dihydroxyphenylalanine;

L-α-methyl-β-4-methyl-2,3-dihydroxyphenylalanine;

L-α-propyl-β-4-methyl-2,3-dihydroxyphenylalanine;

L-α-butyl-β-4-methyl-2,3-dihydroxyphenylalanine;

L-α-methyl-β-4-fluoro-2,3-dihydroxyphenylalanine;

L-α-ethyl-β-4-fluoro-2,3-dihydroxyphenylalanine;

L-α-propyl-β-4-fluoro-2,3-dihydroxyphenylalanine;

L-α-butyl-β-4-fluoro-2,3-dihydroxyphenylalanine;

L-α-methyll-b-4-trifluoromethyl-2,3-dihydroxyphenyl alanine

L-α-ethyl-β-4-trifluoromethyl-2,3-dihydroxyphenyl alanine

L-α-propyl-β-4-trifluoromethyl-2,3-dihydroxyphenyl alanine

L-α-butyl-β-4-trifluoromethyl-2,3-dihydroxyphenyl alanine

L-α-methyl-β-3,5-dihydroxyphenylalanine;

L-α-ethyl-β-3,5-dihydroxyphenylalanine;

L-α-propyl-β-3,5-dihydroxyphenylalanine;

L-α-butyl-β-3,5-dihydroxyphenylalanine;

L-α-methyl-β-4-chloro-3,5-dihydroxphenylalanine;

L-α-ethyl-β-4-chloro-3,5-dihydroxphenylalanine;

L-α-propyl-β-4-chloro-3,5-dihydroxphenylalanine;

L-α-butyl-β-4-chloro-3,5-dihydroxphenylalanine;

L-α-methyl-β-4-fluoro-3,5-dihydroxyphenylalanine;

L-α-ethyl-β-4-fluoro-3,5-dihydroxyphenylalanine;

L-α-propyl-β-4-fluoro-3,5-dihydroxyphenylalanine;

L-α-butyl-β-4-fluoro-3,5-dihydroxyphenylalanine;

L-methyl-β-4-trifluoromethyl-3,5-dihydroxyphenyl alanine;

L-α-ethyl-β-4-trifluoromethyl-3,5-dihydroxyphenyl alanine;

L-α-propyl-β-4-trifluoromethyl-3,5-dihydroxyphenyl alanine;

L-α-butyl-β-4-trifluoromethyl-3,5-dihydroxyphenyl alanine;

L-α-methyl-2,5-dihydroxphenylalanine;

L-α-ethyl-2,5-dihydroxphenylalanine;

L-α-propyl-2,5-dihydroxphenylalanine;

L-α-butyl-2,5-dihydroxphenylalanine;

L-α-methyl-β-4-chloro-2,5-dihydroxyphenylalanine;

L-α-ethyl-β-4-chloro-2,5-dihydroxyphenylalanine;

L-α-propyl-β-4-chloro-2,5-dihydroxyphenylalanine;

L-α-butyl-β-4-chloro-2,5-dihydroxyphenylalanine;

L-α-methyl-β-4-chloro-2,5-dihydroxyphenylalanine;

L-α-ethyl-β-4-chloro-2,5-dihydroxyphenylalanine;

L-α-propyl-β-4-chloro-2,5-dihydroxyphenylalanine;

L-α-butyl-O-4-chloro-2,5-dihydroxyphenylalanine;

L-α-methyl-β-methyl-2,5-dihydroxyphenylalanine;

L-α-ethyl-β-methyl-2,5-dihydroxyphenylalanine;

L-α-propyl-β-methyl-2,5-dihydroxyphenylalanine;

L-α-butyl-β-methyl-2,5-dihydroxyphenylalanine;

L-α-methyl-β-4-trifluoromethyl-2,5-dihydroxyphenyl alanine;

L-α-ethyl-β-4-trifluoromethyl-2,5-dihydroxyphenyl alanine;

L-α-propyl-β-4-trifluoromethyl-2,5-dihydroxyphenyl alanine;

L-α-butyl-β-4-trifluoromethyl-2,5-dihydroxyphenyl alanine;

L-α-methyl-β-3,4,5-trihydroxyphenylalanine;

L-α-ethyl-β-3,4,5-trihydroxyphenylalanine;

L-α-propyl-β-3,4,5-trihydroxyphenylalanine;

L-α-butyl-β-3,4,5-trihydroxyphenylalanine;

L-α-methyl-β-2,3,4-trihydroxyphenylalanine;

L-α-ethyl-β-2,3,4-trihydroxyphenylalanine;

L-α-propyl-β-2,3,4-trihydroxyphenylalanine;

L-α-butyl-β-2,3,4-trihydroxyphenylalanine;

L-α-methyl-β-2,4,5-trihydroxyphenylalanine;

L-α-ethyl-β-2,4,5-trihydroxyphenylalanine;

L-α-propyl-β-2,4,5-trihydroxyphenylalanine;

L-α-butyl-β-2,4,5-trihydroxyphenylalanine;

L-phenylalanine;

D,L-α-methylphenylalanine;

D,L-3-iodophenylalanine;

D,L-3-iodo-α-methylphenylalanine;

3-iodotyrosine;

3,5-diiodotyrosine;

L-α-methylphenylalanine;

D,L-α-β-(4-hydroxy-3-methylphenyl) alanine;

D,L-α-β-(4-methoxy-3-benzylphenyl) alanine;

D,L-α-β-(4-hydroxy-3-benzylphenyl) alanine;

D,L-α-β-(4-methoxy-3-cyclohexylphenyl) alanine;

D,L-α-β-(4-hydroxy-3-cyclohexylphenyl) alanine;

D,L-α-β-(4-methoxy-3-methylphenyl) alanine;

D,L-α-β-(4-hydroxy-3-methylphenyl)alanine;

N,O-dibenzyloxycarbonyl-D,L-α-β-(4-hydroxy-3-methylphenyl)alanine;

N,O-dibenzyloxycarbonyl-D,L-α-β-(4-hydroxy-3-methylphenyl)alanine amide;

D,L-α-β-(4-hydroxy-3-methylphenyl) alanine amide;

N,O-diacetyl-D,L-α-β-(4-hydroxy-3-methylphenyl)alanine;

D,L-N-acetyl-α-β-(4-hydroxy-3-methylphenyl)alanine;

L-3,4-dihydroxy-α-methylphenylalanine;

L-4-hydroxy-3-methoxy-α-methylphenylalanine;

L-3,4-methylene-dioxy-α-methylphenylalanine;

2-vinyl-2-amino-3-(2-methoxyphenyl)propionic acid;

2-vinyl-2-amino-3-(2,5-dimethoxyphenyl)propionic acid;

2-vinyl-2-amino-3-(2-imidazolyl)propionic acid;

2-vinyl-2-amino-3-(2-methoxyphenyl)propionic acid ethyl ester;

α-methyl-β-(2,5-dimethoxyphenyl) alanine;

α-methyl-β-(2,5-dihydroxyphenyl) alanine;

α-ethyl-β-(2,5-dimethoxyphenyl)alanine;

α-ethyl-β-(2,5-dihydroxyphenyl)alanine;

α-methyl-β-(2,4-dimethoxyphenyl) alanine;

α-methyl-β-(2,4-dihydroxyphenyl) alanine;

α-ethyl-β-(2,4-dimethoxyphenyl)alanine;

α-ethyl-β-(2,4-dihydroxyphenyl) alanine;

α-methyl-β-(2,5-dimethoxyphenyl)alanine ethyl ester;

2-ethynyl-2-amino-3-(3-indolyl)propionic acid;

2-ethynyl-2,3-(2-methoxyphenyl)propionic acid;

2-ethynyl-2,3-(5-hydroxyindol-3-yl)propionic acid;

2-ethynyl-2-amino-3-(2,5-dimethoxyphenyl)propionic acid;

2-ethynyl-2-amino-3-(2-imidazolyl)propionic acid;

2-ethynyl-2-amino-3-(2-methoxyphenyl)propionic acid ethyl ester;

3-carbomethoxy-3-(4-benzyloxybenzyl)-3-aminoprop-1-yne;

α-ethynyltyrosine hydrochloride;

α-ethynyltyrosine;

α-ethynyl-m-tyrosine;

α-ethynyl-β-(2-methoxyphenyl)alanine;

α-ethynyl-β-(2,5-dimethoxyphenyl)alanine; and

α-ethynylhistidine.

A second sub-class of preferred tyrosine hydroxylase inhibitor compounds consists of compounds wherein at least one of R¹⁰, R¹¹ and R¹² is selected from hydroxy, alkoxy, aryloxy, aralkoxy and alkoxycarbonyl. More preferred compounds of this second sub-class are

α-methyl-3-(pyrrol-1-yl)tyrosine;

α-methyl-3-(4-trifluoromethylthiazol-2-yl)tyrosine;

3-(imidazol-2-yl)-α-methyltyrosine;

Lα-m-tyrosine;

L-α-ethyl-m-tyrosine;

L-α-propyl-m-tyrosine;

L-α-butyl-m-tyrosine;

Lα-p-chloro-m-tyrosine;

L-α-ethyl-β-chloro-m-tyrosine;

L-α-butyl-β-chloro-m-tyrosine;

Lα-p-bromo-m-tyrosine;

L-α-ethyl-p-bromo-m-tyrosine;

L-α-butyl-p-bromo-m-tyrosine;

Lα-p-fluoro-m-tyrosine;

Lα-p-iodo-m-tyrosine;

L-α-ethyl-p-iodo-m-tyrosine;

Lα-p-methyl-m-tyrosine;

Lα-p-ethyl-m-tyrosine;

L-α-ethyl-p-ethyl-m-tyrosine;

L-α-ethyl-p-methyl-m-tyrosine;

Lα-p-butyl-m-tyrosine;

Lα-p-trifluoromethyl-m-tyrosine;

L-3-iodotyrosine;

L-3-chlorotyrosine;

L-3,5-diiodotyrosine;

L-α-methyltyrosine;

D,L-α-methyltyrosine;

D,L-3-iodo-α-methyltyrosine;

L-3-bromo-α-methyltyrosine;

D,L-3-bromo-α-methyltyrosine;

L-3-chloro-α-methyltyrosine;

D,L-3-chloro-α-methyltyrosine; and

2-vinyl-2-amino-3-(4-hydroxyphenyl)propionic acid.

Another preferred class of tyrosine hydroxylase inhibitor compounds within Formula I consists of compounds

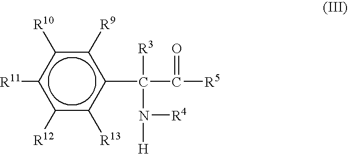

wherein R³ is selected from alkyl, alkenyl and alkynyl; wherein R⁴ is selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein m is a number selected from zero through five, inclusive; wherein R⁵ is selected from OR⁶ and

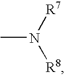

wherein R⁶ is selected from hydrido, alkyl, cycloalkyl, cycloalkylalkyl, phenalkyl and phenyl, and wherein each of R⁷ and R⁸ is independently selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein each of R⁹ through R¹³ is independently selected from hydrido, hydroxy, alkyl, cycloalkyl, cycloalkylalkyl, aralkyl, aryl, alkoxycarbonyl, alkoxy, aryloxy, aralkoxy, alkoxyalkyl, haloalkyl, alkoxycarbonyl, hydroxyalkyl, halo, cyano, amino, monoalkylamino, dialkylamino, carboxyl, carboxyalkyl, alkanoyl, alkenyl, cycloalkenyl and alkynyl.

A preferred sub-class of compounds within Formula III consists of compounds wherein at least one of R¹⁰, R¹¹ and R¹² is selected from hydroxy, alkoxy, aryloxy, aralkoxy and alkoxycarbonyl. More preferred compounds of this sub-class are methyl (+)-2-(4-hydroxyphenyl)glycinate; isopropyl and 3-methyl butyl esters of (+)-2-(4-hydroxyphenyl) glycine; (+)-2-(4-hydroxyphenyl)glycine; (−)-2-(4-hydroxyphenyl) glycine; (+)-2-(4-methoxyphenyl-glycine; and (+)-2-(4-hydroxyphenyl)glycinamide.

Still another preferred class of tyrosine hydroxylase inhibitor compounds within Formula I is provided by compounds of Formula Iv:

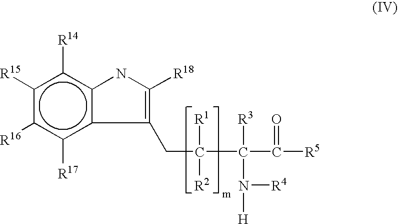

wherein each of R¹ and R² is hydrido; wherein m is a number selected from zero through five, inclusive; wherein R³ is selected from alkyl, alkenyl and alkynyl; wherein R⁴ is selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein each of R¹⁴ through R¹⁷ is independently selected from hydrido, hydroxy, alkyl, cycloalkyl, cycloalkylalkyl, aralkyl, aryl, alkoxy, aralkoxy, alkoxyalkyl, haloalkyl, hydroxyalkyl, halo, cyano, amino, monoalkylamino, dialkylamino, carboxyl, carboxyalkyl, alkanoyl, alkenyl, cycloalkenyl, alkynyl, cyanoamino, carboxyl, cyano, thiocarbamoyl, aminomethyl, alkylsulfanamido, nitro, alkylsulfonyloxy, carboxyalkoxy and formyl.

A preferred sub-class of compounds within Formula IV consists of L-α-methyltryptophan; D,L-5-methyltryptophan; D,L-5-chlorotryptophan; D,L-5-bromotryptophan; D,L-5-iodotryptophan; L-5-hydroxytryptophan; D,L-5-hydroxy-α-methyltryptophan; α-ethynyltryptophan; 5-methoxymethoxy-α-ethynyltryptophan; and 5-hydroxy-α-ethynyltryptophan.

Still another preferred class of tyrosine hydroxylase inhibitor compounds within Formula I is provided by compounds wherein A is

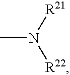

wherein R⁶ is selected from three, inclusive. More preferred compounds in this class are 2-vinyl-2-amino-5-aminopentanoic acid and 2-ethynyl-2-amino-5-aminopentanoic acid.

Still another preferred class of tyrosine hydroxylase inhibitor compounds within Formula I is provided by compounds of Formula V:

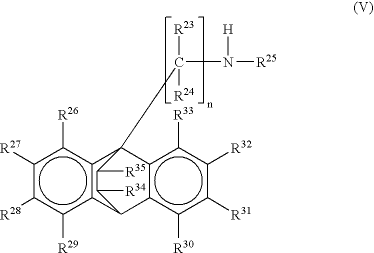

wherein each of R²³ and R²⁴ is independently selected from hydrido, hydroxy, alkyl, cycloakyl, cycloalkylalkyl, aralkyl, aryl, alkoxy, aralkoxy, aryloxy, alkoxyalkyl, haloalkyl, hydroxyalkyl, halo, cyano, amino, monoalkylamino, dialkylamino, carboxy, carboxyalkyl, alkanoyl, alkenyl, cycloalkenyl and alkynyl; wherein R²⁵ is selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein each of R²⁶ through R³⁵ is independently selected from hydrido, hydroxy, alkyl, cycloalkyl, cycloalkylalkyl, aralkyl, aryl, alkoxy, aralkoxy, alkoxyalkyl, haloalkyl, hydroxyalkyl, halo, cyano, amino, monoalkylamino, dialkylamino, carboxyl, carboxyalkyl, alkanoyl, alkenyl, cycloalkenyl, alkynyl, cyanoamino, carboxyl, cyano, thiocarbamoyl, aminomethyl, alkylsulfanamido, nitro, alkylsulfonyloxy, alkoxy and formyl; wherein n is a number selected from zero through five, inclusive; or a pharmaceutically-acceptable salt thereof. A more preferred compound of this class is benzoctamine.

A class of compounds from which a suitable dopa-decarboxylase inhibitor compound may be selected to provide the conjugate first residue is represented by Formula VI:

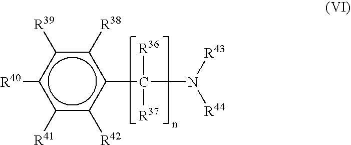

wherein each of R³⁶ through R⁴² is independently selected from hydrido, hydroxy, alkyl, cycloalkyl, cycloalkylalkyl, aralkyl, aryl, alkoxy, aralkoxy, alkoxyalkyl, haloalkyl, hydroxyalkyl, halo, cyano, amino, monoalkylamino, dialkylamino, carboxyl, carboxyalkyl, alkanoyl, alkenyl, cycloalkenyl, alkynyl, cyanoamino, cyano, thiocarbamoyl, aminomethyl, alkylsulfanamido, nitro, alkylsulfonyloxy, carboxyalkoxy and formyl; wherein n is a number from zero through four; wherein each of R⁴³ and R⁴⁴ is independently selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, monoalkylcarbonylamino, alkylsulfinyl, alkylsulfonyl, arylsulfinyl, arylsulfonyl, alkenyl, cycloalkenyl and alkynyl; wherein any R⁴³ and R⁴⁴ substituent having a substitutable position may be further substituted with one or more groups selected from hydroxyalkyl, halo, haloalkyl, carboxyl, alkoxyalkyl, alkoxycarbonyl; with the proviso that R⁴³ and R⁴⁴ cannot both be carboxyl at the same time, with the further proviso that when R³⁶ is hydrido then R³⁷ cannot be carboxyl, and with the further proviso that at least one of R⁴³ through R⁴⁴ is a primary or secondary amino group; or a pharmaceutically-acceptable salt thereof.

A preferred class of compounds within Formula VI consists of compounds wherein each of R³⁶ through R⁴² is independently selected from hydrido, hydroxy, alkyl, cycloalkyl, cycloalkylalkyl, aralkyl, aryl, alkoxy, aralkoxy, alkoxyalkyl, haloalkyl, hydroxyalkyl, halo, amino, monoalkylamino, dialkylamino, carboxyl, carboxyalkyl, alkanoyl, alkenyl, cycloalkenyl, alkynyl, cyanoamino, cyano, aminomethyl, carboxyalkoxy and formyl; wherein n is a number from one through three; wherein each of R⁴³ and R⁴⁴ is independently selected from hydrido, alkyl, cycloalkyl, cycloalkylalkyl, aralkyl, aryl, alkoxyalkyl, haloalkyl, hydroxyalkyl, amino, mono-alkylamino, dialkylamino, carboxyl, carboxyalkyl and alkanoyl; and wherein any R⁴³ and R⁴⁴ substituent having a substitutable position may be further substituted with one or more groups selected from hydroxyalkyl, halo, haloalkyl, carboxyl, alkoxyalkyl, alkoxycarbonyl.

A more preferred class of compounds within Formula VI consists of those compounds wherein each of R³⁶ through R⁴² is independently selected from hydrido, hydroxy, alkyl, benzyl, phenyl, alkoxy, benzyloxy, alkoxyalkyl, haloalkyl, hydroxyalkyl, amino, monoalkylamino, dialkylamino, carboxyl, carboxyalkyl, alkanoyl, cyanoamino, cyano, aminomethyl, carboxyl, carboxyalkoxy and formyl; wherein n is one or two; wherein each of R⁴³ and R⁴⁴ is independently selected from hydrido, alkyl, benzyl, phenyl, alkoxyalkyl, haloalkyl, hydroxyalkyl, cyano, amino, monoalkylamino, dialkylamino, carboxyl, carboxyalkyl and alkanoyl; and wherein any R⁴³ and R⁴⁴ substituent having a substitutable position may be further substituted with one or more groups selected from hydroxyalkyl, halo, haloalkyl, carboxyl, alkoxyalkyl, alkoxycarbonyl.

An even more preferred class of compounds within Formula VI consists of those compounds wherein each of R³⁶ through R⁴² is independently selected from hydrido, hydroxy, alkyl, alkoxy, haloalkyl, hydroxyalkyl, amino, monoalkylamino, carboxyl, carboxyalkyl, aminomethyl, carboxyalkoxy and formyl; wherein n is one or two; wherein each of R⁴³ and R⁴⁴ is independently selected from hydrido, alkyl, haloalkyl, hydroxyalkyl, amino, monoalkylamino, carboxyl and carboxyalkyl; and wherein any R⁴³ and R⁴⁴ substituent having a substitutable position may be further substituted with one or more groups selected from hydroxyalkyl, halo, haloalkyl, carboxyl, alkoxyalkyl, alkoxycarbonyl.

A more highly preferred class of compounds within Formula VI consists of those compounds wherein each of R³⁶ and R³⁷ is hydrido and n is one; wherein each of R³⁸ through R⁴² is independently selected from hydroxy, alkyl, alkoxy, haloalkyl, hydroxyalkyl, amino, monoalkylamino, carboxyl, carboxyalkyl, aminomethyl, carboxyalkoxy and formyl; wherein each of R⁴³ and R⁴⁴ is independently selected from hydrido, alkyl, haloalkyl, hydroxyalkyl, amino, monoalkylamino, carboxyl and carboxyalkyl; and wherein any R⁴³ and R⁴⁴ substituent having a substitutable position may be further substituted with one or more groups selected from hydroxyalkyl, halo, haloalkyl, carboxyl, alkoxyalkyl, alkoxycarbonyl. Compounds of specific interest are (2,3,4-trihydroxy)-benzylhydrazine, 1-(D,L-seryl-2(2,3,4-trihydroxybenzyl)hydrazine (Benserazide) and 1-(3-hydroxylbenzyl)-1-methylhydrazine.

Another more highly preferred class of compounds consists of those compounds wherein each of R³⁶ and R³⁷ is independently selected from hydrido, alkyl and amino and n is two; wherein each of R³⁸ through R⁴² is independently selected from hydroxy, alkyl, alkoxy, haloalkyl, hydroxyalkyl, amino, monoalkylamino, carboxyl, carboxyalkyl, aminomethyl, carboxyalkoxy and formyl; wherein each of R⁴³ and R⁴⁴ is independently selected from hydrido, alkyl, haloalkyl, hydroxyalkyl, amino, monoalkylamino, carboxyl and carboxyalkyl. Compounds of specific interest are 2-hydrazino-2-methyl-3-(3,4-dihydroxyphenyl)propionic acid (Carbidopa), α-(monofluoromethyl)dopa, α-(difluoromethyl)dopa and α-methyldopa.

Another class of compounds from which a suitable dopa-decarboxylase inhibitor compound may be selected to provide the conjugate first residue is represented by Formula VII

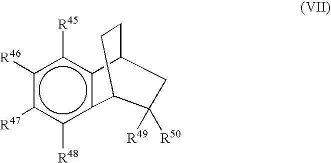

wherein each of R⁴⁵ through R⁴⁸ is independently selected from hydrido, hydroxy, alkyl, cycloalkyl, cycloalkylalkyl, aralkyl, aryl, alkoxy, aralkoxy, alkoxyalkyl, haloalkyl, hydroxyalkyl, halo, amino, monoalkylamino, dialkylamino, carboxyl, carboxyalkyl, alkanoyl, alkenyl, cycloalkenyl, alkynyl, cyanoamino, cyano, thiocarbamoyl, aminomethyl, alkylsulfanamido, nitro, alkylsulfonyloxy, carboxyalkoxy and formyl; wherein each of R⁴⁹ and R⁵⁰ is independently selected from hydrido, alkyl, cycloalkyl, cycloalkylalkyl, aralkyl, aryl, alkoxyalkyl, haloalkyl, hydroxyalkyl, cyano, amino, monoalkylamino, dialkylamino, carboxyalkyl, alkanoyl, alkenyl, cycloalkenyl, alkynyl and

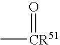

wherein R⁵¹ is selected from hydroxy, alkoxy, aryloxy, aralkoxy, amino, monoalkylamino and dialkylamino with the proviso that R⁴⁹ and R⁵⁰ cannot both be carboxyl at the same time, and with the further proviso that at least one of R⁴⁵ through R⁴⁸ is a primary or secondary amino group or a carboxyl group; or a pharmaceutically-acceptable salt thereof.

A preferred class of compounds within Formula VII consists of those compounds wherein each of R⁴⁵ through R⁴⁸ is independently selected from hydrido, hydroxy, alkyl, cycloalkyl, cycloalkylalkyl, aralkyl, aryl, alkoxy, aralkoxy, alkoxyalkyl, haloalkyl, hydroxyalkyl, halo, cyano, amino, monoalkylamino, dialkylamino, carboxyl, carboxyalkyl, alkanoyl, alkenyl, cycloalkenyl, alkynyl, cyanoamino, cyano, aminomethyl, carboxyalkoxy and formyl; wherein each of R⁴⁹ and R⁵⁰ is independently selected from hydrido, alkyl, cycloalkyl, cycloalkylalkyl, aralkyl, aryl, alkoxyalkyl, haloalkyl, hydroxyalkyl, cyano, amino, monoalkylamino, dialkylamino, carboxyalkyl and alkanoyl and

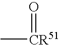

wherein R⁵¹ is selected from hydroxy, alkoxy, phenoxy, benzyloxy, amino, monoalkylamino and dialkylamino.

A more preferred class of compounds within Formula VII consists of those compounds wherein each of R⁴⁵ through R⁴⁸ is independently selected from hydrido, hydroxy, alkyl, benzyl, phenyl, alkoxy, benzyloxy, alkoxyalkyl, haloalkyl, hydroxyalkyl, cyano, amino, monoalkylamino, dialkylamino, carboxyl, carboxyalkyl, alkanoyl, cyanoamino, cyano, aminomethyl, carboxyalkoxy and formyl; wherein each of R⁴⁹ and R⁵⁰ s independently selected from hydrido, alkyl, benzyl, phenyl, alkoxyalkyl, haloalkyl, hydroxyalkyl, cyano, amino, monoalkylamino, dialkylamino, carboxyalkyl and alkanoyl and

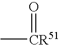

wherein R⁵¹ is selected from hydroxy, alkoxy, amino and monoalkylamino.

An even more preferred class of compounds of Formula VII consists of those compounds wherein each of R⁴⁵ through R⁴⁸ is independently selected from hydrido, hydroxy, alkyl, alkoxy, haloalkyl, hydroxyalkyl, amino, monoalkylamino, carboxyl, carboxyalkyl aminomethyl, carboxyalkoxy and formyl; wherein each of R⁴⁹ and R⁵⁰ is independently selected from hydrido, alkyl, amino, monoalkylamino, carboxyalkyl and

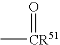

wherein R⁵¹ is selected from hydroxy, alkoxy, amino and monoalkylamino.

A highly preferred class of compounds within Formula VII consists of those compounds wherein each of R⁴⁵ through R⁴⁸ is independently selected from hydrido, hydroxy, alkyl, alkoxy and hydroxyalkyl; wherein each of R⁴⁹ and R⁵⁰ is independently selected from alkyl, amino, monoalkylamino, and

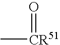

wherein R⁵¹ is selected from hydroxy, methoxy, ethoxy, propoxy, butoxy, amino, methylamino and ethylamino.

A more highly preferred class of compounds within Formula VII consists of those compounds wherein said inhibitor compound is selected from endo-2-amino1,2,3,4-tetrahydro-1,2-ethanonaphthalene-2-carboxylic acid; ethyl-endo-2-amino-1,2,3,4-tetra-hydro-1,4-ethano-naphthalene-2-carboxylate hydrochloride; exo-2-amino1,2,3,4-tetrahydro-1,4-ethanonaphthalene-2-carboxylic acid; and ethyl-exo-2-amino-1,2,3,4-tetrahydro-1,4-ethano-naphthalene-2-carboxylate hydrochloride.

Another family of specific dopa-decarboxylase inhibitor compounds consists of

2,3-dibromo-4,4-bis(4-ethylphenyl)-2-butenoic acid;

3-bromo-4-(4-methoxyphenyl)-4-oxo-2-butenoic acid;

N-(5′-phosphopyridoxyl)-L-3,4-dihydroxyphenylalanine;

N-(5′-phosphopyridoxyl)-L-m-aminotyrosine;

D,L-β-(3,4-dihydroxyphenyl)lactate;

D,L-β-(5-hydroxyindolyl-3)lactate;

2,4-dihydroxy-5-(1-oxo-2-propenyl)benzoic acid;

2,4-dimethoxy-5-[1-oxo-3-(2,3,4-trimethoxyphenyl-2-propenyl]benzoic acid;

2,4-dihydroxy-5-[1-oxo-3-(2-thienyl)-2-propenyl]benzoic acid;

2,4-dihydroxy-5-[3-(4-hydroxyphenyl)-1-oxo-2-propenyl]benzoic acid;

5-[3-(4-chlorophenyl)-1-oxo-2-propenyl]-2,4-dihydroxy benzoic acid;

2,4-dihydroxy-5-(1-oxo-3-phenyl-2-propenyl)benzoic acid;

2,4-dimethoxy-5-[1-oxo-3-(4-pyridinyl)-2-propenyl]benzoic acid;

5-[3-(3,4-dimethoxyphenyl)-1-oxo-2-propenyl]-2,4 dimethoxy benzoic acid;

2,4-dimethoxy-5-(1-oxo-3-phenyl-2-propenyl)benzoic acid;

5-[3-(2-furanyl)-1-oxo-2-propenyl]-2,4-dimethoxy benzoic acid;

2,4-dimethoxy-5-[1-oxo-3-(2-thienyl)-2-propenyl]benzoic acid;

2,4-dimethoxy-5-[3-(4-methoxyphenyl)-1-oxo-2-propenyl]benzoic acid;

5-[3-(4-chlorophenyl)-1-oxo-2-propenyl]-2,4-dimethoxy benzoic acid; and

5-[3-[4-(dimethylamino)phenyl]-1-oxo-2-propenyl]-2,4 dimethoxy benzoic acid.

Another class of compounds from which a suitable dopa-decarboxylase inhibitor may be selected to provide the conjugate first residue is represented by Formula VIII:

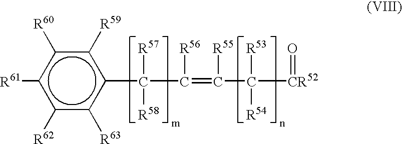

wherein R⁵² is selected from hydrido, OR⁶⁴ and

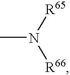

wherein R⁶⁴ is selected from hydrido, alkyl, cycloalkyl, cycloalkylalkyl, phenalkyl and phenyl, and wherein each of R⁶⁵ and R⁶⁶ is independently selected from hydrido, alkyl, alkanoyl, amino, monoalkylamino, dialkylamino, phenyl and phenalkyl; wherein each of R⁵³, R⁵⁴ and R⁵⁷ through R⁶³ is independently selected from hydrido, hydroxy, alkyl, cycloalkyl, cycloalkylalkyl, aralkyl, aryl, alkoxycarbonyl, hydroxyalkyl, halo, cyano, amino, monoalkylamino, dialkylamino, carboxyl, carboxyalkyl, alkanoyl, alkenyl, cycloalkenyl and alkynyl; wherein each of R⁵⁵ and R⁵⁶ is independently selected from hydrido, alkyl, cycloalkyl, cycloalkylalkyl, aralkyl, aryl, alkoxyalkyl, halo, haloalkyl, hydroxyalkyl and carboxyalkyl; wherein each of m and n is a number independently selected from zero through six, inclusive; or a pharmaceutically-acceptable salt thereof.

A preferred class of compounds of Formula VIII consists of those compounds wherein R⁵² is OR⁶⁴ wherein R⁶⁴ is selected from hydrido, alkyl, cycloalkyl, cycloalkylalkyl, benzyl and phenyl; wherein each of R⁵³, R⁵⁴ and R⁵⁷ through R⁶³ is independently selected from hydrido, alkyl, cycloalkyl, hydroxy, alkoxy, benzyl and phenyl; wherein each of R⁵⁵ and R⁵⁶ is independently selected from hydrido, alkyl, cycloalkyl, benzyl and phenyl; wherein each of m and n is a number independently selected from zero through three, inclusive.

A more preferred class of compounds of Formula VIII consists of those compounds wherein R⁵² is OR⁶⁴ wherein R⁶⁴ is selected from hydrido and lower alkyl; wherein each of R⁵³ through R⁵⁸ is hydrido; wherein each of R⁵⁹ through R⁶³ is independently selected from hydrido, alkyl, hydroxy and alkoxy, with the proviso that two of the R⁵⁹ through R⁶³ substituents are hydroxy; wherein each of m and n is a number independently selected from zero through two, inclusive.

A preferred compound within Formula IX is 3-(3,4-dihydroxyphenyl)-2-propenoic acid, also known as caffeic acid.

Another class of compounds from which a suitable dopa-decarboxylase inhibitor compound may be selected to provide the conjugate first residue is a class of aromatic amino acid compounds comprising the following subclasses of compounds:

amino-haloalkyl-hydroxyphenyl propionic acids, such as 2-amino-2-fluoromethyl-3-hydroxy-phenylpropionic acid;

alpha-halomethyl-phenylalanine derivatives such as alpha-fluoroethylphenethylamine; and

indole-substituted halomethylamino acids.

Still other classes of compounds from which a suitable dopa-decarboxylase inhibitor compound may be selected to provide the conjugate first residue are as follows:

isoflavone extracts from fungi and streptomyces, such as 3′,5,7-trihydroxy-4′,6-dimethoxyisoflavone, 3′,5,7-trihydroxy-4′,8-dimethoxyisoflavone and 3′,8-dihydroxy-4′, 6,7-trimethoxy isoflavone;

sulfinyl substituted dopa and tyrosine derivatives such as shown in U.S. Pat. No. 4,400,395 the content of which is incorporated herein by reference;

hydroxycoumarin derivatives such as shown in U.S. Pat. No. 3,567,832, the content of which is incorporated herein by reference;

l-benzylcyclobutenyl alkyl carbamate derivatives such as shown in U.S. Pat. No. 3,359,300, the content of which is incorporated herein by reference;

arylthienyl-hydroxylamine derivatives such as shown in U.S. Pat. No. 3,192,110, the content of which is incorporated herein by reference; and

β-2-substituted-cyclohepta-pyrrol-8-1H-on-7-yl alanine derivatives.

Suitable dopamine-β-hydroxylase inhibitors may be generally classified mechanistically as chelating-type inhibitors, time-dependent inhibitors and competitive inhibitors.

A class of compounds from which a suitable dopamine-β-hydroxylase inhibitor may be selected to provide the conjugate first residue consists of time-dependent inhibitors represented by Formula IX:

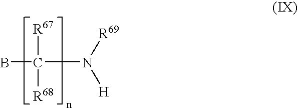

wherein B is selected from aryl, an ethylenic moiety, an acetylenic moiety and an ethylenic or acetylenic moiety substituted with one or more radicals selected from substituted or unsubstituted alkyl, aryl and heteroaryl; wherein each of R⁶⁷ and R⁶⁸ is independently selected from hydrido, alkyl, alkenyl and alkynyl; wherein R⁶⁹ is selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; and wherein n is a number selected from zero through five.

A preferred class of compounds of Formula IX consists of those compounds wherein B is phenyl or hydroxyphenyl; wherein R⁶⁷ is ethenyl or ethynyl; or an acetylenic moiety substituted with an aryl or heteroaryl radical; and wherein n is a number from zero through three.

Another preferred class of compounds of Formula IX consists of those compounds wherein B is an ethylenic or acetylenic moiety incorporating carbon atoms in the beta- and gamma-positions relative to the nitrogen atom; and wherein n is zero or one. More preferred are compounds wherein the ethylenic or acetylenic moiety is substituted at the gamma carbon with an aryl or heteroaryl radical. Even more preferred are compounds wherein said aryl radical is selected from phenyl, 2-thiophene, 3-thiophene, 2-furanyl, 3-furanyl, oxazolyl, thiazolyl and isoxazolyl, any one of which radicals may be substituted with one or more groups selected from halo, hydroxyl, alkyl, haloalkyl, cyano, alkoxy, alkoxyalkyl and cycloalkyl. More highly preferred are compounds wherein said aryl radical is selected from phenyl, hydroxyphenyl, 2-thiophene and 2-furanyl; and wherein each of R⁶⁷, R⁶⁸ and R⁶⁹ is hydrido.

A family of specifically-preferred compounds within Formula IX consists of the compounds 3-amino-2-(2′-thienyl)propene; 3-amino-2-(2′-thienyl)butene; 3-(N-methylamino)-2-(2′-thienyl)propene; 3-amino-2-(3′-thienyl)propene; 3-amino-2-(2′furanyl)propene; 3-amino-2-(3′-furanyl)propene; 1-phenyl-3aminopropyne; and 3-amino-2-phenylpropene. Another family of specifically-preferred compounds of Formula VIII consists of the compounds (±)4-amino-3-phenyl-1-butyne; (±)4-amino-3-(3′-hydroxyphenyl)-1-butyne; (±)4-amino-3-(4′-hydroxyphenyl)-1-butyne; (±)4-amino3-phenyl-1-butene; (±)4-amino-3-(3′-hydroxyphenyl)-1-butene; and (±)4-amino-3-(4′-hydroxyphenyl)-1-butene.

Another class of compounds from which a suitable dopamine-β-hydroxylase inhibitor may be selected to provide the conjugate first residue is represented by Formula X:

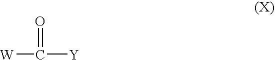

wherein W is selected from alkyl, cycloalkyl, alkenyl, alkynyl, cycloalkenyl, cycloalkynyl, cycloalkylalkyl, aryl, aralkyl, heterocycloalkyl and heteroaryl; wherein Y is selected from

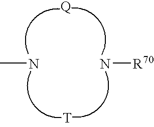

wherein R⁷⁰ is selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein each of Q and T is one or more groups independently selected from

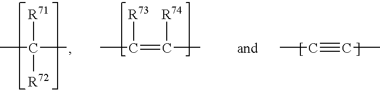

wherein each of R⁷¹ through R⁷⁴ is independently selected from hydrido, hydroxy, alkyl, cycloalkyl, cycloalkylalkyl, aralkyl, aryl, alkoxy, aralkoxy, aryloxy, alkoxyalkyl, haloalkyl, hydroxyalkyl, halo, cyano, amino, monoalkylamino, dialkylamino, carboxy, carboxyalkyl, alkanoyl, alkenyl, cycloalkenyl and alkynyl; or a pharmaceutically-acceptable salt thereof.

A preferred class of compounds within Formula X consists of compounds wherein W is heteroaryl and Y is

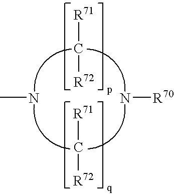

wherein R⁷⁰ is selected from hydrido, alkyl, amino, monoalkylamino, dialkylamino, phenyl and phenalkyl; wherein each of R⁷¹ and R⁷² is independently selected from hydrido, hydroxy, alkyl, phenalkyl, phenyl, alkoxy, benzyloxy, phenoxy, alkoxyalkyl, hydroxyalkyl, halo, amino, monoalkylamino, dialkylamino, carboxy, carboxyalkyl and alkanoyl; and wherein each of p and q is a number independently selected from one through six, inclusive.

A more preferred class of compounds of Formula X consists of wherein R⁷⁰ is selected from hydrido, alkyl, amino and monoalkylamino; wherein each of R⁷¹ and R⁷² is independently selected from hydrido, hydroxy, alkyl, alkoxy, amino, monoalkylamino, carboxy, carboxyalkyl and alkanoyl; and wherein each of p and q is a number indpendently selected from two through four, inclusive. Even more preferred are compounds wherein R⁷⁰ is selected from hydrido, alkyl and amino; wherein each of R⁷¹ and R⁷² is independently selected from hydrido, amino, monoalkylamino and carboxyl; and wherein each of p and q is independently selected from the numbers two and three. Most preferred are compounds wherein R⁷⁰ is hydrido; wherein each of R⁷¹ and R⁷² is hydrido; and wherein each of p and q is two.

Another class of compounds from which a suitable dopamine-β-hydroxylase inhibitor may be selected to provide the conjugate first residue is represented by Formula XI:

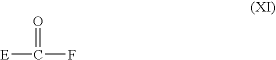

wherein E is selected from alkyl, cycloalkyl, alkenyl, alkynyl, cycloalkenyl, cycloalkynyl, cycloalkylalkyl, aryl, aralkyl, heterocycloalkyl and heteroaryl; wherein F is selected from

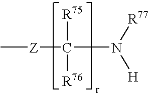

wherein Z is selected from O, S and N—R⁷⁸; wherein each of R⁷⁵ and R⁷⁶ is independently selected from hydrido, hydroxy, alkyl, cycloalkyl, cycloalkylalkyl, aralkyl, aryl, alkoxy, aralkoxy, aryloxy, alkoxyalkyl, haloalkyl, hydroxyalkyl, halo, cyano, amino, minoalkylamino, dialkylamino, carboxy, carboxyalkyl, alkanoyl, alkenyl, cycloalkenyl and alkynyl; wherein R⁷⁵ and R⁷⁶ may form oxo or thio; wherein r is a number selected from zero through six, inclusive; wherein each of R⁷⁷ and R⁷⁸ is independently selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; or a pharmaceutically acceptable salt thereof.

Another class of compounds from which a suitable dopamine-β-hydroxylase inhibitor may be selected to provide the conjugate first residue is represented by Formula XII:

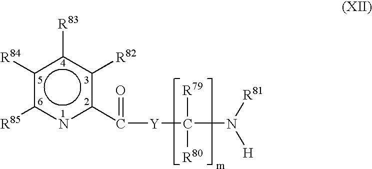

wherein each of R⁸² through R⁸⁵ is independently selected from hydrido, alkyl, haloalkyl, mercapto, alkylthio, cyano, alkoxy, alkoxyalkyl and cycloalkyl; wherein Y is selected from oxygen atom and sulfur atom; wherein each of R⁷⁹ and R⁸⁰ is independently selected from hydrido and alkyl; wherein R⁸¹ is selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; and wherein m is a number from one through six; or a pharmaceutically acceptable salt thereof.

A preferred family of compounds of Formula XII consists of those compounds wherein each of R⁸² through R⁸⁵ is independently selected from hydrido, alkyl and haloalkyl; wherein Y is selected from oxygen atom or sulfur atom; wherein each of R⁷⁹, R⁸⁰ and R⁸¹ is independently hydrido and alkyl; and wherein m is a number selected from one through four, inclusive.

A family of preferred specific compounds within Formula XII consists of the following compounds:

aminomethyl-5-n-butylthiopicolinate;

aminomethyl-5-n-butylpicolinate;

2′-aminoethyl-5-n-butylthiopicolinate;

2′-aminoethyl-5-n-butylpicolinate;

(2′-amino-1′, 1′-dimethyl)ethyl-5-n-butylthiopicolinate;

(2′-amino-1′,1′-dimethyl)ethyl-5-n-butylpicolinate;

(2′-amino-1′-methyl)ethyl-5-n-butylthiopicolinate;

(2′-amino-1′-methyl)ethyl-5-n-butylpicolinate;

3′-aminopropyl-5-n-butylthiopicolinate;

3′-aminopropyl-5-n-butylpicolinate;

(2′-amino-2′-methyl)propyl-5-n-butylthiopicolinate;

(2′-amino-2′-methyl)propyl-5-n-butylpicolinate;

(3′-amino-1′, 1′-dimethyl)propyl-5-n-butylthiopicolinate;

(3′-amino-1′, 1′-dimethyl)propyl-5-n-butylpicolinate;

(3′-amino-2′, 2′-dimethyl)propyl-5-n-butylthiopicolinate;

(3′-amino-2′, 2′-dimethyl)propyl-5-n-butylpicolinate;

2′-aminopropyl-5-n-butylthiopicolinate;

2′-aminopropyl-5-n-butylpicolinate;

4′-aminobutyl-5-n-butylthiopicolinate;

4′-amino-3′-methyl)butyl-5-n-butylthiopicolinate;

(3′-amino-3′-methyl)butyl-5-n-butylthiopicolinate;

and (3′-amino-3′-methyl)butyl-5-n-butylpicolinate.

Another preferred class of compounds within Formula XII consists of those compounds of Formula XIII:

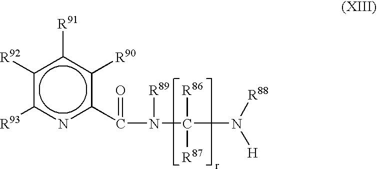

wherein each of R⁸⁶, R⁸⁷ and R⁹⁰ through R⁹³ is independently selected from hydrido, hydroxy, alkyl, cycloalkyl, cycloalkylalkyl, aralkyl, aryl, alkoxy, aralkoxy, aryloxy, alkoxyalkyl, haloalkyl, hydroxyalkyl, halo, cyano, amino, monoalkylamino, dialkylamino, carboxy, carboxyalkyl, alkanoyl, alkenyl, cycloalkenyl and alkynyl; wherein R⁸⁶ and R⁸⁷ together may form oxo or thio; wherein r is a number selected from zero through six, inclusive; wherein each of R⁸⁸ and R⁸⁹ is independently selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl.

A more preferred class of compounds within Formula XIII consists of those compounds wherein each of R⁸⁶, R⁸⁷ and R⁹⁰ through R⁹³ is independently selected from hydrido, hydroxy, alkyl, phenalkyl, phenyl, alkoxy, benzyloxy, phenoxy, alkoxyalkyl, hydroxyalkyl, halo, amino, monoalkylamino, dialkylamino, carboxy, carboxyalkyl and alkanoyl; wherein r is a number selected from zero through four, inclusive; wherein each of R⁸⁸ and R⁸⁹ is independently selected from hydrido, alkyl, amino, monoalkylamino, dialkylamino, phenyl and phenalkyl.

An even more preferred class of compounds within Formula XIII consists of those compounds wherein each of R⁸⁶, R⁸⁷ and R⁹⁰ through R⁹³ is independently selected from hydrido, hydroxy, alkyl, alkoxy, amino, monoalkylamino, carboxy, carboxyalkyl and alkanoyl; and wherein r is a number selected from zero through three, inclusive; and wherein each of R⁸⁸ and R⁸⁹ is selected from hydrido, alkyl, amino and monoalkylamino. Most preferred are compounds wherein each of R⁹⁰ through R⁹³ is independently selected from hydrido and alkyl; wherein each of R⁸⁶ and R⁸⁷ is hydrido; wherein r is selected from zero, one and two; wherein R⁸⁸ is selected from hydrido, alkyl and amino; and wherein R⁸⁹ is selected from hydrido and alkyl. Especially preferred within this class is the compound 5-n-butylpicolinic acid hydrazide (fusaric acid hydrazide) shown below:

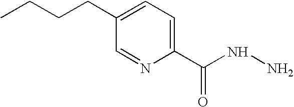

Another class of compounds from which a suitable dopamine-β-hydroxylase inhibitor compound may be selected to provide the conjugate first residue is represented by Formula XIV:

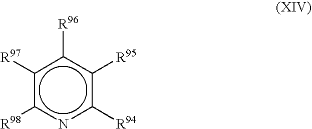

wherein each of R⁹⁴ through R⁹⁸ is independently selected from hydrido, hydroxy, alkyl, cycloalkyl, cycloalkylalkyl, aralkyl, aryl, aryloxy, alkoxy, alkylthio, aralkoxy, alkoxyalkyl, haloalkyl, hydroxyalkyl, halo, cyano, amino, monoalkylamino, dialkylamino, amido, alkylamido, hydroxyamino, carboxyl, carboxyalkyl, alkanoyl, alkenyl, cycloalkenyl, alkynyl, cyanoamino, carboxyl, tetrazolyl, thiocarbamoyl, aminomethyl, alkylsulfanamido, nitro, alkylsulfonyloxy, formoyl and alkoxycarbonyl; with the proviso that at least one of R⁹⁴ through R⁹⁸ is

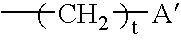

wherein A′ is

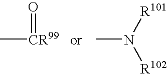

wherein R⁹⁹ is selected from hydrido, alkyl, hydroxy, alkoxy, alkylthio, phenyl, phenoxy, benzyl, benzyloxy,

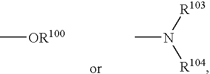

wherein R¹⁰⁰ is selected from hydrido, alkyl, cycloalkyl, cycloalkylalkyl, phenyl and benzyl; wherein each of R¹⁰¹, R¹⁰², R¹⁰³ and R¹⁰⁴ is independently selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein t is a number selected from zero through four, inclusive; or a pharmaceutically-acceptable salt thereof.

A preferred family of compounds within Formula XIV consists of those compounds characterized as chelating-type inhibitors of Formula XV:

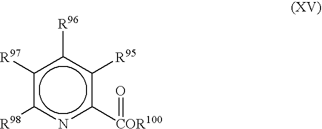

wherein each of R⁹⁵ through R⁹⁸ is independently selected from hydrido, hydroxy, alkyl, cycloalkyl, phenyl, benzyl, alkoxy, phenoxy, benzyloxy, alkoxyalkyl, hydroxyalkyl, halo, cyano, amino, monoalkylamino, dialkylamino, amido, alkylamido, hydroxyamino, carboxyl, carboxyalkyl, alkanoyl, cyanoamino, carboxyl, thiocarbamoyl, aminomethyl, nitro, formoyl, formyl and alkoxycarbonyl; and wherein R¹⁰⁰ is selected from hydrido, alkyl, phenyl and benzyl.

A class of specifically-preferred compounds of Formula XV consists of

5-n-butylpicolinic acid (fusaric acid);

5-ethylpicolinic acid;

picolinic acid;

5-nitropicolinic acid;

5-aminopicolinic acid;

5-N-acetylaminopicolinic acid;

5-N-propionylaminopicolinic acid;

5-N-hydroxyaminopicolinic acid;

5-iodopicolinic acid;

5-bromopicolinic acid;

5-chloropicolinic acid;

5-hydroxypicolinic acid

5-methoxypicolinic acid;

5-N-propoxypicolinic acid;

5-N-butoxypicolinic acid;

5-cyanopicolinic acid;

5-carboxylpicolinic acid;

5-n-butyl-4-nitropicolinic acid;

5-n-butyl-4-methoxypicolinic acid;

5-n-butyl-4-ethoxypicolinic acid;

5-n-butyl-4-aminopicolinic acid;

5-n-butyl-4-hydroxyaminopicolinic acid; and

5-n-butyl-4-methylpicolinic acid.

Especially preferred of the foregoing class of compounds of Formula XV is the compound 5-n-butylpicolinic acid (fusaric acid) shown below:

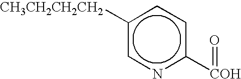

Another class of compounds from which a suitable dopamine-β-hydroxylase inhibitor may be selected to provide the conjugate first residue consists of azetidine-2-carboxylic acid derivatives represented by Formula XVI:

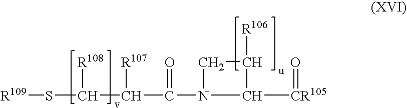

wherein R¹⁰⁵ is hydrido, hydroxy, alkyl, amino and alkoxy; wherein R¹⁰⁶ is selected from hydrido, hydroxy and alkyl; wherein each of R¹⁰⁷ and R¹⁰⁸ is independently selected from hydrido, alkyl and phenalkyl; wherein R¹⁰⁹ is selected from hydrido and

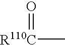

with R¹¹⁰ selected from alkyl, phenyl and phenalkyl; wherein u is a number from one to three, inclusive; and wherein v is a number from zero to two, inclusive; or a pharmaceutically-acceptable salt thereof.

A preferred class of compounds within Formula XVI consists of those compounds wherein R¹⁰⁵ is selected from hydroxy and lower alkoxy; wherein R¹⁰⁶ is hydrido; wherein R¹⁰⁷ is selected from hydrido and lower alkyl; wherein R¹⁰⁸ is hydrido; wherein R¹⁰⁹ is selected from hydrido and

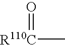

with R¹¹⁰ selected from lower alkyl and phenyl; wherein u is two; and wherein v is a number from zero to two, inclusive.

A more preferred class of compounds within Formula XVI consists of those compounds of Formula XVII:

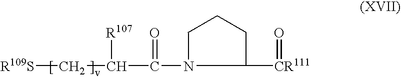

wherein R¹¹¹ is selected from hydroxy and lower alkyl; wherein R¹⁰⁷ is selected from hydrido and lower alkyl; wherein R¹⁰⁹ is selected from hydrido and

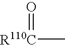

with R¹¹⁰ selected from lower alkyl and phenyl and v is a number from zero to two, inclusive.

A more preferred class of compounds within Formula XVII consists of those compounds wherein R¹¹¹ is hydroxy; wherein R¹⁰⁷ is hydrido or methyl; wherein R¹⁰⁹ is hydrido or acetyl; and wherein n is a number from zero to two, inclusive.

Most preferred within the class of compounds of Formula XVII are the compounds 1-(3-mercapto-2-methyl-1-oxopropyl)-L-proline and 1-(2-mercaptoacetyl)-L-proline (also known as captopril).

Another class of compounds from which a suitable dopamine-β-hydroxylase inhibitor compound may be selected to provide the conjugate first residue is represented by Formula XVIII:

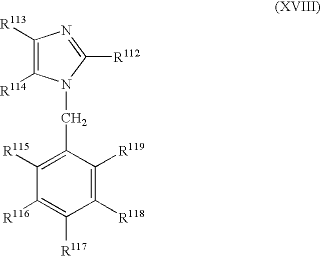

wherein each of R¹¹² through R¹¹⁹ is independently selected from hydrido, hydroxy, alkyl, cycloalkyl, cycloalkylalkyl, alkoxy, alkoxyalkyl, aralkyl, aryl, alkoxycarbonyl, hydroxyalkyl, halo, haloalkyl, cyano, amino, aminoalkyl, monoalkylamino, dialkylamino, carboxyl, carboxyalkyl, alkanoyl, alkenyl, cycloalkenyl, alkynyl, mercapto and alkylthio; or a pharmaceutically-acceptable salt thereof.

A first preferred class of compounds within Formula XVIII consists of those compounds wherein R¹¹² is selected from mercapto and alkylthio; wherein each of R¹¹³ and R¹¹⁴ is independently selected from hydrido, amino, aminoalkyl, monoalkylamino, monoalkylaminoalkyl, carboxyl and carboxyalkyl; wherein each of R¹¹⁵ and R¹¹⁹ is hydrido; and wherein each of R¹¹⁶, R¹¹⁷ and R¹¹⁸ is independently selected from hydrido, hydroxy, alkyl, halo and haloalkyl; or a pharmaceutically-acceptable salt thereof.

A second preferred class of compounds within Formula XVIII consists of those compounds wherein R¹¹² is selected from amino, aminoalkyl, monoalkylamino, monoalkylaminoalkyl, carboxy and carboxyalkyl; wherein each of R¹¹³, R¹¹⁴, R¹¹⁵ and R¹¹⁹ is hydrido; and wherein each of R¹¹⁶, R¹¹⁷ and R¹¹⁸ is independently selected from hydrido, hydroxy, alkyl, halo and haloalkyl; or a pharmaceutically-acceptable salt thereof.

Compounds which fall within any of the aforementioned inhibitor compounds, but which lack a reactive acid or amino moiety to form a cleavable bond, may be modified or derivatized to contain such acid of amino moiety. Examples of classes of such compounds lacking an amino on acidic moiety are the following: 1-(3,5-dihaloaryl)imidazol-2-thione derivatives such as 1-(3,5-difluorobenzyl)imidazol-2-thione; and hydroxyphenolic derivatives such as resorcinol.

The second component of a conjugate of the invention is provided by a residue which forms a kidney-enzyme-cleavable bond with the residue of the first-component AII antagonist compound. Such residue is preferably selected from a class of compounds of Formula XIX:

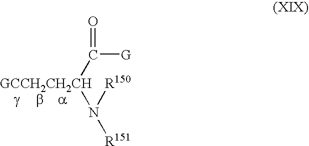

wherein each of R¹⁵⁰ and R¹⁵¹ may be independently selected from hydrido, alkylcarbonyl, alkoxycarbonyl, alkoxyalkyl, hydroxyalkyl and haloalkyl; and wherein G is selected from hydroxyl, halo, mercapto, —OR¹⁵², —SR¹⁵³ and

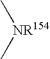

with each R¹⁵², R¹⁵³ and R¹⁵⁴ is independently selected from hydrido and alkyl; with the proviso that said Formula XIX compound is selected such that formation of the cleavable bond occurs at carbonyl moiety attached at the gamma-position carbon of said Formula XIX compound.

More preferred are compounds of Formula XIX wherein each G is hydroxy.

A more highly preferred class of compounds within Formula XIX consists of those compounds wherein each G is hydroxy; wherein R¹⁵⁰ is hydrido; and wherein R¹⁵¹ is selected from

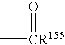

wherein R¹⁵⁵ is selected from methyl, ethyl, n-propyl, isopropyl, n-butyl, sec-butyl, iso-butyl, tert-butyl, n-pentyl, neopentyl, n-hexyl and chloromethyl.

A most highly preferred compound of Formula XIX is N-acetyl-γ-glutamic acid which provides a residue for the second component of a conjugate of the invention as shown below:

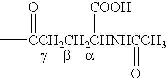

The phrase “terminal primary or secondary amino moiety or a moiety convertible to a primary or secondary amino terminal moiety” characterizes a structural requirement for selection of a suitable angiotensin II antagonist compound as the “active” first residue of a conjugate of the invention. Such terminal amino moiety must be available to react with a terminal carboxylic moiety of the cleavable second residue to form a kidney-enzyme-specific hydrolyzable bond.

The first component used to form the conjugate of the invention provides a first residue derived from an inhibitor compound capable of inhibiting formation of a benzylhydroxylamine intermediate involved in the biosynthesis of an adrenergic neurotransmitter, hereinafter generally referred to as an “inhibitor compound”. In one embodiment of the invention, the first component used to form a conjugate of the invention provides a first residue containing a terminal primary or secondary amino moiety. Examples of such terminal amino moiety are amino and linear or branched aminoalkyl moieties containing linear or branched alkyl groups such as aminomethyl, aminoethyl, aminopropyl, aminoisopropyl, aminobutyl, aminosecbutyl, aminoisobutyl, aminotertbutyl, aminopentyl, aminoisopentyl and aminoneopentyl.

In another embodiment of the invention, the first component used to form the conjugate of the invention provides a first residue derived from an inhibitor compound containing a moiety convertible to a primary or secondary amino terminal moiety. An example of a moiety convertible to an amino terminal moiety is a carboxylic acid group reacted with hydrazine so as to convert the acid moiety to carboxylic acid hydrazide. The hydrazide moiety thus contains the terminal amino moiety which may then be further reacted with the carboxylic acid containing residue of the second component to form a hydrolyzable amide bond. Such hydrazide moiety thus constitutes a “linker” group between the first and second components of a conjugate of the invention.

Suitable linker groups may be provided by a class of diamino-terminated linker groups based on hydrazine as defined by Formula XX:

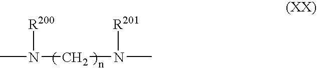

wherein each of R²⁰⁰ and R²⁰¹ may be independently selected from hydrido, alkyl, cycloalkyl, cycloalkylalkyl, alkoxyalkyl, hydroxyalkyl, aralkyl, aryl, haloalkyl, amino, monoalkylamino, dialkylamino, cyanoamino, carboxyalkyl, alkylsulfino, alkylsulfonyl, arylsulfinyl and arylsulfonyl; and wherein n is zero or a number selected from three through seven, inclusive. In Table I there is shown a class of specific examples of diamino-terminated linker groups within Formula XX, identified as Linker Nos. 1-73. These linker groups would be suitable to form a conjugate between a carbonyl moiety of an inhibitor compound residue (designated as “I”) and a carbonyl moiety of a carbonyl terminated second residue such as the carbonyl moiety attached to the gamma carbon of a glutamyl residue (designated as “T”).

TABLE I

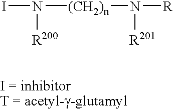

| LINKER | | | |
|---|---|---|---|
| NO. | n | R²⁰⁰ | R²⁰¹ |
| 1 | 0 | H | H |
| 2 | 0 | CH₃ | H |
| 3 | 0 | C₂H₅ | H |
| 4 | 0 | C₃H₇ | H |
| 5 | 0 | CH(CH₃)₂ | H |
| 6 | 0 | C₄H₉ | H |
| 7 | 0 | CH(CH₃)CH₂CH₃ | H |
| 8 | 0 | C(CH₃)3 | H |
| 9 | 0 | C₅H₉ | H |
| 10 | 0 | C₆H₁₁(cyclo) | H |
| 11 | 0 | C₆H₅ | H |
| 12 | 0 | CH₂C₆H₅ | H |
| 13 | 0 | H | CH₃ |
| 14 | 0 | H | C₂H₅ |
| 15 | 0 | H | C₃H₇ |
| 16 | 0 | H | CH(CH₃)₂ |
| 17 | 0 | H | C₄H₉ |
| 18 | 0 | H | CH(CH₃)CH₂CH₃ |
| 19 | 0 | H | C(CH₃)₃ |
| 20 | 0 | H | C₅H₉ |
| 21 | 0 | H | C₆H₁₃ |
| 22 | 0 | H | C₆H₅ |
| 23 | 0 | H | CH₂C₆H₅ |
| 24 | 0 | H | C₆H₁₁(cyclo) |
| 25 | 0 | C₆H₁₃ | H |
| 26 | 0 | CH₃ | CH₃ |
| 27 | 0 | C₂H₅ | C₂H₅ |
| 28 | 0 | C₃H₇ | C₃H₇ |
| 29 | 0 | CH(CH₃)₂ | CH(CH₃)₂ |
| 30 | 0 | C₄H₉ | C₄H₉ |
| 31 | 0 | CH(CH₃)cK2CH₃ | CH(CH₃)Qi2CH₃ |
| 32 | 0 | C(CH₃)₃ | C(CH₃)₃ |
| 33 | 0 | C₅H₉ | C₅H₉ |
| 34 | 0 | C₆H₁₃ | C₆H₁₃ |
| 35 | 0 | C₆H₁₁(cyclo) | C₆H₁₁(cyclo) |
| 36 | 0 | C₆H₅ | C₆H₅ |
| 37 | 0 | CH₂C₆H₅ | CH₂C₆H₅ |
| 38 | 3 | H | H |
| 39 | 3 | CH₃ | H |
| 40 | 3 | H | CH₃ |
| 41 | 3 | C₆H₅ | H |
| 42 | 3 | H | C₆H₅ |
| 43 | 3 | CH₃ | C₆H₅ |
| 44 | 3 | C₆H₅ | CH₃ |
| 45 | 3 | CH₂C₆H₅ | H |
| 46 | 3 | H | CH₂C₆H₅ |
| 47 | 4 | H | H |
| 48 | 4 | CH₃ | H |
| 49 | 4 | H | CH₃ |
| 50 | 4 | C₆H₅ | H |
| 51 | 4 | H | C₆H₅ |
| 52 | 4 | CH₃ | C₆H₅ |
| 53 | 4 | C₆H₅ | CH₃ |
| 54 | 4 | CH₂C₆H₅ | H |
| 55 | 4 | H | CH₂C₆H₅ |
| 56 | 5 | H | H |
| 57 | 5 | CH₃ | H |
| 58 | 5 | H | |
| 59 | 5 | C₆H₅ | H |
| 60 | 5 | H | C₆H₅ |
| 61 | 5 | CH₃ | C₆H₅ |
| 62 | 5 | C₆H₅ | CH₃ |
| 63 | 5 | CH₂C₆H₅ | H |
| 64 | 5 | H | CH₂C₆H₅ |
| 65 | 6 | H | H |
| 66 | 6 | CH₃ | H |
| 67 | 6 | H | CH₃ |
| 68 | 6 | C₆H₅ | H |
| 69 | 6 | H | C₆H₅ |
| 70 | 6 | CH₃ | C₆H₅ |
| 71 | 6 | C₆H₅ | CH₃ |
| 72 | 6 | CH₂C₆H₅ | H |
| 73 | 6 | H | CH₂C₆H₅ |

Another class of suitable diamino terminal linker groups is defined by Formula XXI:

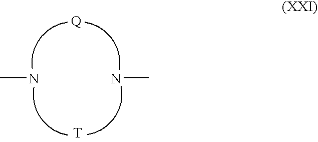

wherein each of Q and T is one or more groups independently selected from

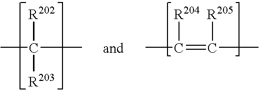

wherein each of R²⁰² through R²⁰⁵ is independently selected from hydrido, hydroxy, alkyl, cycloalkyl, cycloalkylalkyl, aralkyl, aryl, alkoxy, aralkoxy, aryloxy, alkoxyalkyl, haloalkyl, hydroxyalkyl, halo, cyano, amino, monoalkylamino, dialkylamino, carboxy, carboxyalkyl, alkanoyl, alkenyl, cycloalkenyl and alkynyl.

A preferred class of linker groups within Formula XX is defined by Formula XXII:

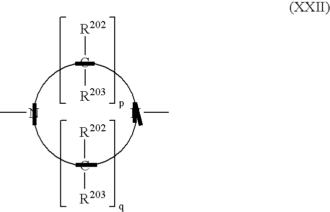

wherein each of R²⁰² and R²⁰³ is independently selected from hydrido, hydroxy, alkyl, phenalkyl, phenyl, alkoxy, benzyloxy, phenoxy, alkoxyalkyl, hydroxyalkyl, halo, amino, monoalkylamino, dialkylamino, carboxy, carboxyalkyl and alkanoyl; and wherein each of p and q is a number independently selected from one through six, inclusive; with the proviso that when each of R²⁰² and R²⁰³ is selected from halo, hydroxy, amino, monoalkylamino and dialkylamino, then the carbon to which R²⁰² or R²⁰³ is attached in Formula XXII is not adjacent to a nitrogen atom of Formula XXII.

A more preferred class of linker groups of Formula XXII consists of divalent radicals wherein each of R²⁰² and R²⁰³ is independently selected from hydrido, hydroxy, alkyl, alkoxy, amino, monoalkylamino, carboxy, carboxyalkyl and alkanoyl; and wherein each of p and q is a number independently selected from two through four, inclusive. Even more preferred are linker groups wherein each of R²⁰² and R²⁰³ is independently selected from hydrido, amino, monoalkylamino and carboxyl; and wherein each of p and q is independently selected from the numbers two and three. Most preferred is a linker group wherein each of R²⁰² and R²⁰³ is hydrido; and wherein each of p and q is two; such most preferred linker group is derived from a piperazinyl group and has the structure

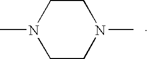

In Table II there is shown a class of specific examples of cyclized, diamino-terminated linker groups within Formula XXII. These linker groups, identified as Linker Nos. 74-95, would be suitable to form a conjugate between a carbonyl moiety of an inhibitor compound residue (designated as “I”) and a carbonyl moiety of carbonyl terminated second residue such as the carbonyl moiety attached to the gamma carbon of a glutamyl residue (designated as “T”).

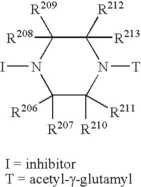

| LINKER | | | | | | | | |
|---|---|---|---|---|---|---|---|---|
| No. | R²⁰⁶ | R²⁰⁷ | R²⁰⁸ | R²⁰⁹ | R²¹⁰ | R²¹¹ | ²¹² | R²¹³ |
| 74 | H | H | H | H | H | H | H | H |
| 75 | CH₃ | H | H | H | H | H | H | H |
| 76 | H | H | H | H | CH₃ | H | H | H |
| 77 | CH₃ | H | H | H | CH₃ | H | H | H |
| 78 | CH₃ | H | CH₃ | H | H | H | H | H |
| 79 | CH₃ | H | H | H | H | H | CH₃H | |
| 80 | CH₃ | CH₃ | H | H | H | H | H | H |
| 81 | H | H | H | H | CH₃ | CH₃ | H | H |
| 82 | CH₃ | CH₃ | H | H | CH₃ | CH₃ | H | H |
| 83 | CH₃ | CH₃ | CH₃ | CH₃ | H | H | H | H |
| 84 | CH₃ | CH₃ | H | H | H | H | CH₃ | CH₃ |
| 85 | H | H | H | H | CH₃ | CH₃ | CH₃ | CH₃ |
| 86 | C₆H₅ | H | H | H | H | H | H | H |
| 87 | H | H | H | H | C₆H₅ | H | H | H |
| 88 | C₆H₅ | H | H | H | C₆H₅ | H | H | H |
| 89 | C₆H₅ | H | H | H | H | H | C₆H5H | |
| 90 | C₆H₅ | H | C₆H₅ | H | H | H H | H | |
| 91 | CH₂C₆H₅ | H | H | H | H | H H | H | |
| 92 | H | H | H | H | CH₂C₆H₅ | H H | H | |
| 93 | CH₂C₆H₅ | H | H | H | CH₂C₆H₅ | H | H | H |
| 94 | CH₂C₆H₅ | H | H | H | H | H | CH₂C₆H₅H | |
| 95 | CH₂C₆H₅ | H | CH₂C₆H₅ | H | H | H | H | H |

Another class of suitable diamino terminal linker groups is defined by Formula XXIII:

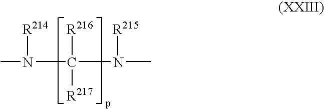

wherein each of R²¹⁴ through R²¹⁷ is independently selected from hydrido, alkyl, cycloalkyl, cycloalkylalkyl, hydroxyalkyl, alkoxyalkyl, aralkyl, aryl, haloalkyl, amino, monoalkylamino, dialkylamino, cyanoamino, carboxyalkyl, alkylsulfino, alkylsulfonyl, arylsulfinyl and arylsulfonyl; and wherein p is a number selected from one through six inclusive.

A preferred class of linker groups within Formula XXIII consists of divalent radicals wherein each of R²¹⁴ and R²¹⁵ is hydrido; wherein each of R²¹⁶ and R²¹⁷ is independently selected from hydrido, alkyl, phenalkyl, phenyl, alkoxyalkyl, hydroxyalkyl, haloalkyl and carboxyalkyl; and wherein p is two or three. A more preferred class of linker groups within Formula XXIII consists of divalent radicals wherein each of R²¹⁴ and R²¹⁵ is hydrido; wherein each of R²¹⁶ and R²¹⁷ is independently selected from hydrido and alkyl; and wherein p is two. A specific example of a more preferred linker within Formula XXIII is the divalent radical ethylenediamino. In Table III there is shown a class of specific examples of diamino-terminated linker gorups within Formula XXIII. These linker groups, identified as Linker Nos. 96-134, would be suitable to form a conjugate between a carbonyl moiety of an inhibitor compound residue (designated as “I”) and a carbonyl moiety of carbonyl terminated second residue such as the carbonyl moiety attached to the gamma carbon of a glutamyl residue (designated as “T”).

TABLE III

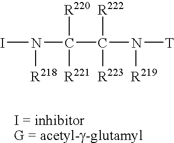

| LINKER | | | | | | |
|---|---|---|---|---|---|---|
| NO. | R²¹⁸ | R²¹⁹ | R²²⁰ | R²²¹ | R²²² | R²²³ |
| 96 | H | H | H | H | H | H |
| 97 | H | H | H | H | H | CH₃ |
| 98 | H | H | H | CH₃ | H | H |
| 99 | H | H | H | CH₃ | H | CH₃ |
| 100 | CH₃ | H | H | H | H | H |
| 101 | H | CH₃ | H | H | H | H |
| 102 | H | H | H | H | CH₃ | CH₃ |
| 103 | H | H | CH₃ | CH₃ | H | H |
| 104 | CH₃ | CH₃ | H | H | H | H |
| 105 | H | H | H | H | H | C₆H₅ |
| 106 | H | H | H | C₆H₅ | H | H |
| 107 | H | H | H | C₆H₅ | H | C₆H₅ |
| 108 | C₆H₅ | H | H | H | H | H |
| 109 | H | C₆H₅ | H | H | H | H |
| 110 | H | H | H | H | C₆H₅ | C₆H₅ |
| 111 | H | H | C₅H₅ | C₆H₅ | H | H |
| 112 | C₆H₅ | C₆H₅ | H | H | H | H |
| 113 | H | H | H | H | H | C₂H₅ |
| 114 | H | H | H | C₂H₅ | H | H |
| 115 | H | H | H | C₂H₅ | H | C₂H₅ |
| 116 | C₂H₅ | H | H | H | H | H |
| 117 | H | C₂H₅ | H | H | H | H |
| 118 | H | H | H | H | C₂H₅ | C₂H₅ |
| 119 | H | H | C₂H₅ | C₂H₅ | H | H |
| 120 | C₂H₅ | C₂H₅ | H | H | H | H |
| 121 | CH₃ | H | C₆H₅ | H | H | H |
| 122 | CH₃ | H | H | H | C₆H₅ | H |
| 123 | H | CH₃ | C₆H₅ | H | H | H |
| 124 | H | CH₃ | H | H | C₆H₅ | H |
| 125 | CH₃ | CH₃ | H | C₆H₅ | H | H |
| 126 | CH₃ | CH₃ | H | H | H | C₆H₅ |
| 127 | H | H | H | H | H | CH₂C₆H₅ |
| 128 | H | H | H | CH₂C₆H₅ | H | H |
| 129 | CH₂C₆H₅ | H | H | H | H | H |
| 130 | H | CH₂C₆H₅ | H | H | H | H |
| 131 | CH₃ | H | CH₂C₆H₅ | H | H | H |
| 132 | CH₃ | H | H | H | CH₂C₆H₅ | H |
| 133 | H | CH₃ | CH₂C₆H₅ | H | H | H |
| 134 | H | CH₃ | H | H | CH₂C₆H₅ | H |

The term “hydrido” denotes a single hydrogen atom (H). This hydrido group may be attached, for example, to an oxygen atom to form a hydroxyl group; or as another example, two hydrido groups may be attached to a carbon atom to form a divalent —CH₂— group, that is, a “methylene” group; or as another example, one hydrido group may be attached to a carbon atom to form a trivalent

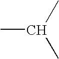

group. Where the term “alkyl” is used, either alone or within other terms such as “haloalkyl”, “aralkyl” and “hydroxyalkyl”, the term “alkyl” embraces linear or branched radicals having one to about ten carbon atoms unless otherwise specifically described. Preferred alkyl radicals are “lower alkyl” radicals having one to about five carbon atoms. The term “cycloalkyl” embraces radicals having three to ten carbon atoms, such as cyclopropyl, cyclobutyl, cyclohexyl and cycloheptyl. The term “haloalkyl” embraces radicals wherein any one or more of the carbon atoms is substituted with one or more halo groups, preferably selected from bromo, chloro and fluoro. Specifically embraced by the term “haloalkyl” are monohaloalkyl, dihaloalkyl and polyhaloalkyl groups. A monohaloalkyl group, for example, may have either a bromo, a chloro, or a fluoro atom within the group. Dihaloalkyl and polyhaloalkyl groups may be substituted with two or more of the same halo groups, or may have a combination of different halo groups. Examples of a dihaloalkyl group are dibromomethyl, dichloromethyl and bromochloromethyl. Examples of a polyhaloalkyl are trifluoromethyl, 2,2,2-trifluoroethyl, perfluoroethyl and 2,2,3,3-tetrafluoropropyl groups. The term “alkoxy”, embraces linear or branched oxy-containing radicals having an alkyl portion of one to about ten carbon atoms, such as methoxy, ethoxy, isopropoxy and butoxy. The term “alkylthio” embraces radicals containing a linear or branched alkyl group, of one to about ten carbon atoms attached to a divalent sulfur atom, such as a methythio group. The term “aryl” embraces aromatic radicals such as phenyl, naphthyl and biphenyl. The term “aralkyl” embraces aryl-substituted alkyl radicals such as benzyl, diphenylmethyl, triphenylmethyl, phenylethyl, phenylbutyl and diphenylethyl. The terms “benzyl” and “phenylmethyl” are interchangeable. The terms “aryloxy” and “arylthio” denote radical respectively, aryl groups having an oxygen or sulfur atom through which the radical is attached to a nucleus, examples of which are phenoxy and phenylthio. The terms “sulfinyl” and “sulfonyl”, whether used alone or linked to other terms, denotes respectively divalent radicals

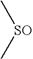

and

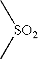

The term “acyl” whether used alone, or within a term such as acyloxy, denotes a radical provided by the residue after removal of hydroxyl from an organic acid, examples of such radical being acetyl and benzoyl. “Lower alkanoyl” is an example of a more preferred sub-class of acyl.

Within the classes of conjugates of the invention described herein are the pharmaceutically-acceptable salts of such conjugates including acid addition salts and base addition salts. The term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts of conjugates of the invention may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, p-hydroxybenzoic, salicyclic, phenylacetic, mandelic, embonic (pamoic), methansulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactaric and galacturonic acid. Suitable pharmaceutically-acceptable base addition salts of the conjugates include metallic salts made from aluminium, calcium, lithium, magnesium, potassium, sodium and zinc or organic salts made from N,N′dibenzylethylenediamine, chloroprocaine, choline, diethanolamine, ethylenediamine, meglumine (N-methylglucamine) and procaine. All of these salts may be prepared by conventional means from the corresponding conjugates described herein by reacting, for example, the appropriate acid or base with the conjugate.

Conjugates of the invention can possess one or more asymmetric carbon atoms and are thus capable of existing in the form of optical isomers as well as in the form of racemic or non-racemic mixtures thereof. The optical isomers can be obtained by resolution of the racemic mixtures according to conventional processes, for example by formation of diastereoisomeric salts by treatment with an optically active acid or base. Examples of appropriate acids are tartaric, diacetyltartaric, dibenzoyltartaric, ditoluoyltartaric and camphorsulfonic acid and then separation of the mixture of diastereoisomers by crystallization followed by liberation of the optically active bases from these salts. A different process for separation of optical isomers involves the use of a chiral chromatography column optimally chosen to maximize the separation of the enantiomers. Still another available method involves synthesis of covalent diastereoisomeric molecules by reacting conjugates with an optically pure acid in an activated form or an optically pure isocyanate. The synthesized diastereoisomers can be separated by conventional means such as chromatography, distillation, crystallization or sublimation, and then hydrolyzed to deliver the enantiomerically pure compound. The optically active conjugates can likewise be obtained by utilizing optically active starting materials. These isomers may be in the form of a free acid, a free base, an ester or a salt.

### Synthetic Procedures

Conjugates of the invention are synthesized by reaction between precursors of the first and second residues. One of such precursors must contain a reactive acid moiety, and the other precursor must contain a reactive amino moiety, so that a conjugate is formed having a cleavable bond. Either precursor of the first and second residues may contain such reactive acid or amino moieties. Preferably, the precursors of the first residue are inhibitors of benzylhydroxyamine biosynthesis and will contain a reactive amino moiety or a moiety convertible to a reactive amino moiety. Many of the tyrosine hydroxylase inhibitors and dopa-decarboxylase inhibitors are characterized in having a reactive amino moiety. Inhibitor compounds lacking a reactive amino moiety, such as the dopamine-β-hydroxylase inhibitor fusaric acid, may be chemically modified to provide such reactive amino moiety. Chemical modification of these inhibitor compounds lacking a reactive amino group may be accomplished by reacting an acid or an ester group on the inhibitor compound with an amino compound, that is, a compound having at least one reactive amino moiety and another reactive hetero atom selected from O, S and N. A suitable amino compound would be a diamino compound such as hydrazine or urea. Hydrazine, for example, may be reacted with the acid or ester moiety of the inhibitor compound to form a hydrazide derivative of such inhibitor compound.

The dopamine-β-hydroxylase inhibitor compound 5-butyl-n-butylpicolinic acid (fusaric acid) may be used as a model compound to illustrate the chemical modification of an acid-containing inhibitor compound to make a reactive amino-containing precursor for synthesizing a conjugate of the invention. In the following General Synthetic Procedures, the substituents and reagents are defined as follows: each of R⁷⁹, R⁸⁰, R⁸¹, R⁸⁶, R⁸⁷, R⁸⁸, R⁸⁹ and R¹¹⁵ is as defined above; W is selected from alkyl, cycloalkyl, alkenyl, alkynyl, cycloalkenyl, cycloalkynyl, cycloalkylalkyl, aryl, aralkyl, heterocycloalkyl and heteroaryl; and Z is selected from oxygen and sulfur. DCC is an abbreviation for dicyclohexylcarbodiimide.

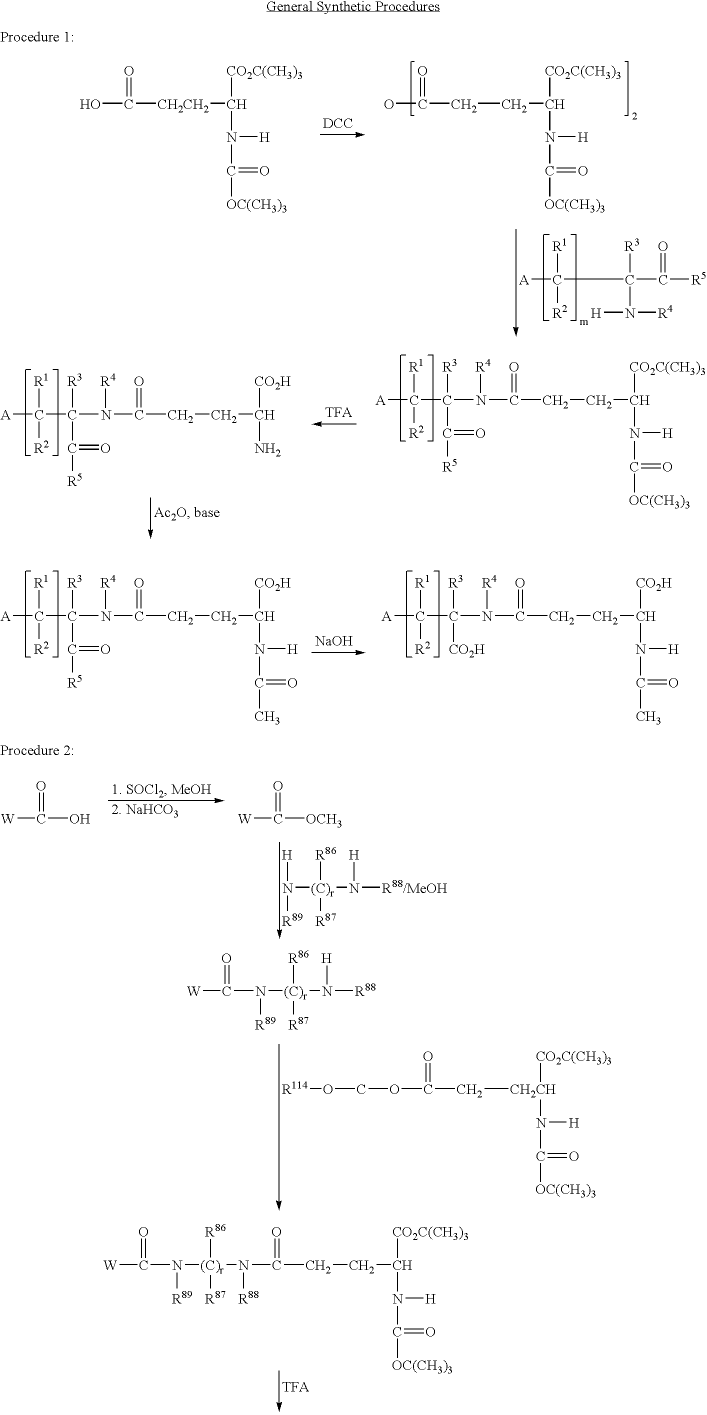

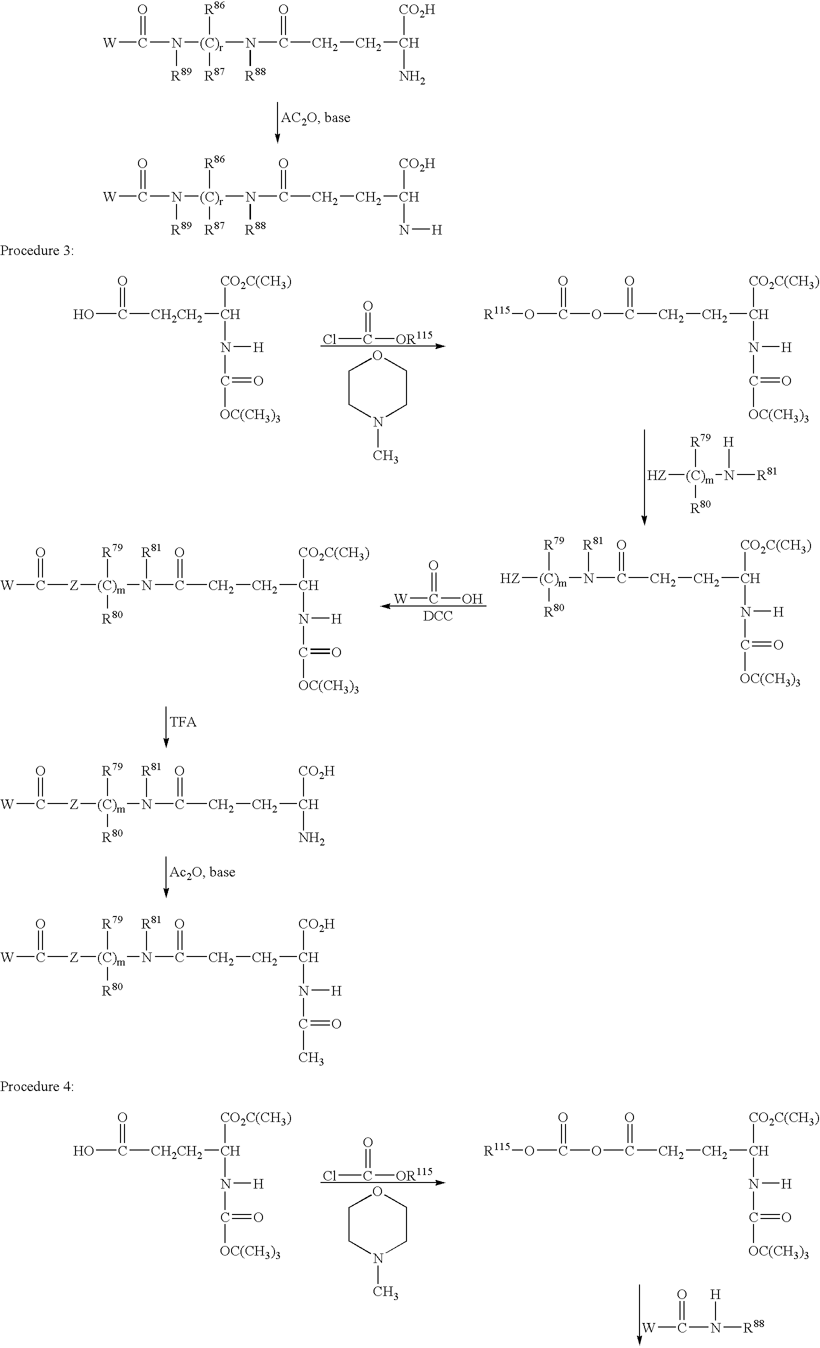

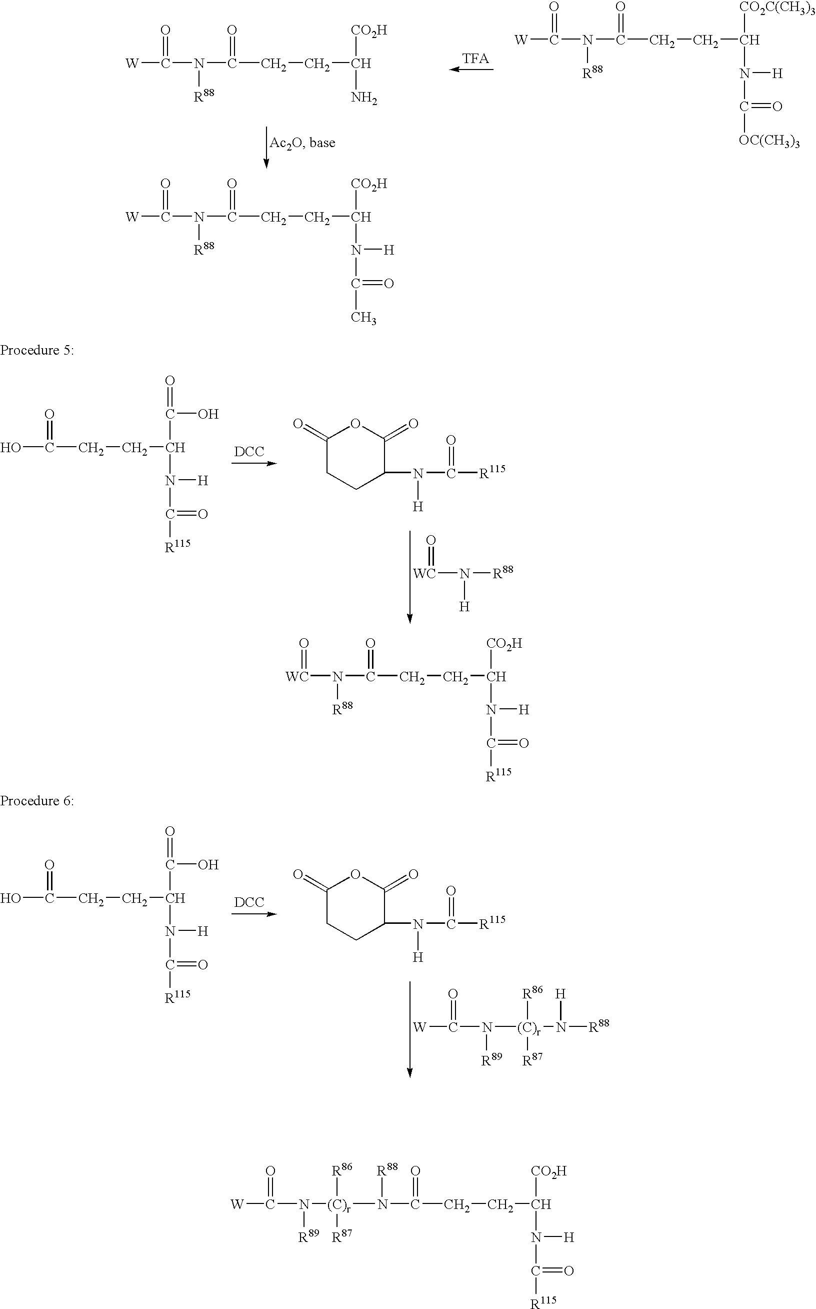

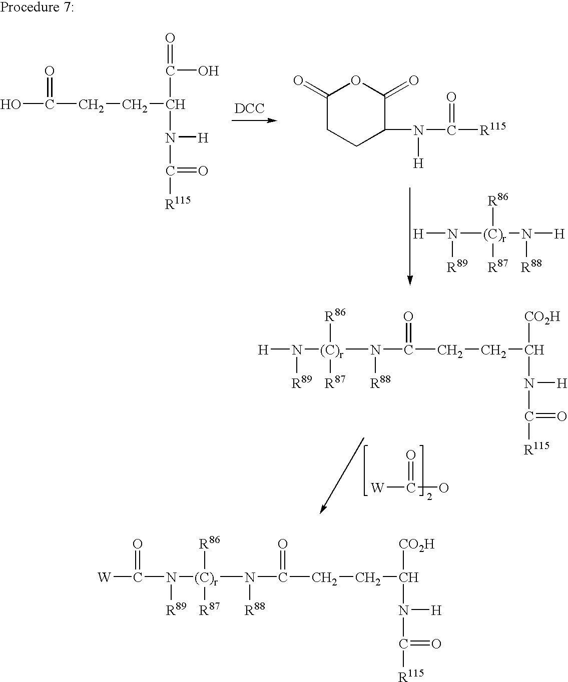

The following Examples 1 through 1857 shown in Tables IV-XVII are highly preferred conjugates of the invention. These conjugates fall within three classes, namely, conjugates of tyrosine hydroxylase inhibitors of Tables IV-VI, conjugates of dopa-decarboxylase inhibitors of Tables VII-XI, and conjugates of dopamine-β-hydroxylase inhibitors of Tables XII-XVII. These conjugates may be prepared generally by the procedures outlined above in Schemes 1-7. Also, specific procedures for preparation of Examples 1-1857 are found in the conjugate preparations described in the examples appearing with the tables of conjugates.

The following Examples #1-#461 comprise three classes of highly preferred conjugates formed from tyrosine hydroxylase inhibitor compounds and glutamic acid derivatives. Examples #1-#3 are descriptions of specific preparations of such conjugates. Examples #4-#461, as shown in Tables IV-VI, may be prepared by procedures shown in these specific examples and in the foregoing general synthetic procedures of Schemes 1-7.

### EXAMPLE 1

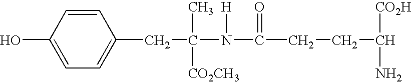

4-amino-4-carboxy-1-oxobutyl-α-methyl-L-tyrosine, Methyl Ester

Step. 1. Preparation of Methyl α-methyl-L-tyrosinate, Hydrochloride.

A solution of 11.0 g (56.4 mmol) of α-methyl-L-tyrosine in 100 mL of absolute methanol was cooled to 0° C. and treated with 20.1 g (169 mmol) of thionyl chloride under a nitrogen atmosphere. The reaction was allowed to warm to ambient temperature and stir at reflux for 2 days. Concentration followed by trituration with 150 mL of ether gave 13.3 g (96%) of colorless product: NMR (DMSO-d₆) δ 1.49 (s, 3H), 3.02 (s, 2H), 3.73 (s, 3H), 6.73 (d, J=11 Hz, 2H), 6.97 (d, J=11 Hz, 2H), 8.50-8.70 (br s, 3H), 9.50 (s, 1H).

Step. 2. Preparation of 4-amino-4-carboxy-1-oxobutyl-α-methyl-L-tyrosine, Methyl Ester

Under nitrogen, a solution of 35.1 g (116 mmol) of N-Boc-L-γ-glutanic acid-α-t-butyl ester (BACHEM) in 200 mL of methylene chloride was treated with 11.95 g (58 mmol) of solid dicyclohexylcarbodiimide (DCC). The reaction was allowed to stir for 2 hr prior to filtration under a nitrogen atmosphere. The methylene chloride was removed in vacuo and the residue dissolved in 100 mL of anhydrous dimethylformamide (DMF). The anhydride solution was slowly added to a solution of 7.0 g (29 mmol) of the α-methyl tyrosine ester from step 1 and 18.73 g (145 mmol) of diisopropylethylamine (DIEA) in 100 mL of anhydrous DMF. The reaction was allowed to stir overnight and was concentrated in vacuo. The residue was dissolved in ethyl acetate, washed with cold 1 M K₂CO₃ followed by water, dried (MgSO₄), and concentrated in vacuo to give the protected coupled product; a solution of this material in 150 mL of methylene chloride was cooled to 0° C. and treated with 150 mL of trifluoracetic acid (TFA) under nitrogen. The reaction was allowed to warm to ambient temperatures and stir overnight. Concentration in vacuo gave 4-amino-4-carboxy-1-oxobutyl-α-methyl-L-tyrosine, methyl ester: NMR (DMSO-d₆) δ 1.20 (s, 3H), 1.90-2.20 (m, 2H), 2.23-2.38 (m, 0.2H), 2.95 (d, J=13 Hz, 1H), 3.26 (d, J=13 Hz), 3.57 (s, 3H), 3.92-4.06 (m, 1H), 7.06 (d, J=9 Hz, 2H), 7.12 (d, J=9 Hz, 2H).

### EXAMPLE 2

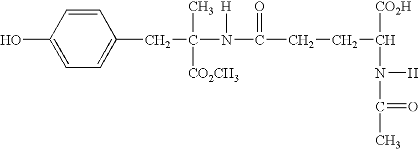

N-[4-(acetylamino)-4-carboxy-1-oxobutyl]-α-methyl-L-tyrosine, Methyl Ester.

The compound of Example 1 was dissolved in 100 mL of water and the pH adjusted to 9 with 1 M K₂CO₃. The solution was cooled to 0° C. and 3.30 mL (35 mmol) of acetic anhydride and 35 mL (35 mmol) of 1 M K₂CO₃ was added every 30 min. for 5 h; the pH was maintained at 9 and the reaction temperature kept below 5° C. After the last addition, the reaction was allowed to warm to ambient temperature overnight. The pH was adjusted to 4 with 6 M HCl and concentrated to 100 mL. Purification by reverse phase chromatography (Waters Deltaprep-3000) using isocratic 25% acetonitrile/water (0.05% TFA) gave 9.0 g (82%) of colorless product: NMR (DMSO-d₆) δ 1.18 (s, 3H), 1.72-2.03 (m, 2H), 1.85 (s, 3H), 2.15 (t, J=8 Hz, 2H), 2.93 (d, J=13 Hz, 1H), 3.38 (d, J=13 Hz, 1H), 3.57 (s, 3H), 4.12-4.23 (m, 1H), 7.02 (d, J=9 Hz, 2H), 7.09 (d, J=9 Hz, 2H), 8.06 (s, 1H), 8.12 (d, J=8 Hz, 1H).

### EXAMPLE 3

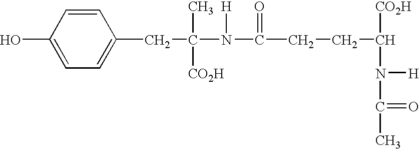

N-[4-(acetylamino)-4-carboxy-1-oxobutyl]-α-methyl-L-tyrosine.

A solution of 9.0 g (23.7 mmol) of the compound of Example 2 in 225 mL of water was cooled to 0° C. and treated with 3.3 g (82.5 mmol) of solid NaOH in portions over 15 min. The reaction was stirred at 0-5° C. overnight, the pH adjusted to pH 5 with 6N HCl, and concentrated to 100 mL. Purification by reverse phase chromatography (Waters Deltaprep-3000) using isocratic 15% acetonitrite/water (0.05% TFA) gave 5.50 g (63%) of colorless product: NMR (DMSO-d₆) δ 1.17 (s, 3H), 1.70-2.00 (m, 2H), 1.85 (s, 3H), 2.14 (t, J=8 Hz, 2H), 2.83 (d, J=13 Hz, 1H), 3.14 (d, J=13 Hz, 1H), 4.12-4.23 (m, 1H), 6.56 (d, J=9 Hz, 2H), 6.85 (d, J=9 Hz, 2H), 7.69 (s, 1H), 8.12 (d, J=8 Hz, 1H); MS (FAB) m/e (rel intensity) 367 (70), 196 (52), 179 (58) 150 (100), 130 (80); HRMS. Calcd for M+H: 367.1505. Found: 367.1547. Anal. Calcd for C₁₇H₂₂N₂O₇.H₂O.0.125 TFA: C, 52.00; H, 6.03; N, 7.03; F, 1.60. Found: C, 51.96; H, 6.25; N, 7.12; F, 1.60.

The following Examples #4-#109 of Table IV are highly preferred conjugates formed from tyrosine hydroxylase inhibitor compounds and glutamic acid derivatives. These tyrosine hydroxylase inhibitors utilized to make these conjugates are embraced by generic Formula I and II, above.

TABLE IV

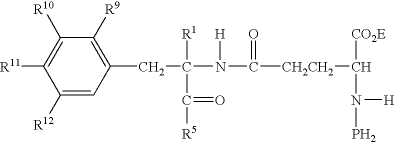

| EXAMPLE | | | | | | | | |
|---|---|---|---|---|---|---|---|---|
| NO. | R¹ | R⁹ | R¹⁰ | R¹¹ | R¹² | R⁵ | E | P |
| 4 | CH₃ | H | H | OH | H | OCH₃ | CH₃ | COCH₃ |
| 5 | CH₃ | H | H | OH | H | OH | H | H |
| 6 | CH₃ | H | H | OH | H | OCH₃ | CH₃ | H |
| 7 | CH₃ | H | H | OH | H | OH | CH₃ | H |
| 8 | CH₃ | H | H | OH | H | OH | CH₃ | COCH₃ |
| 9 | CH₂F | H | H | OH | H | OCH₃ | H | H |
| 10 | CH₂F | H | H | OH | H | OCH₃ | H | COCH₃ |
| 11 | CH₂F | H | H | OH | H | OCH₃ | CH₃ | H |
| 12 | CH₂F | H | H | OH | H | OCH₃ | OCH₃ | COCH₃ |
| 13 | CH₂F | H | H | OH | H | OH | H | H |
| 14 | CH₂F | H | H | OH | H | OH | H | COCH₃ |
| 15 | CH₂F | H | H | OH | H | OH | CH₃ | H |
| 16 | CH₂F | H | H | OH | H | OH | CH₃ | COCH₃ |
| 17 | CHF₂ | H | H | OH | H | OCH₃ | H | H |
| 18 | CHF₂ | H | H | OH | H | H | COCH₃ | |
| 19 | CHF₂ | H | H | OH | H | OCH₃ | CH₃ | H |
| 20 | CHF₂ | H | H | OH | H | OCH₃ | CH₃ | COCH₃ |
| 21 | CHF₂ | H | H | OH | H | OH | H | H |
| 22 | CHF₂ | H | H | OH | H | OH | H | COCH₃ |
| 23 | CHF₂ | H | H | OH | H | OH | CH₃ | H |
| 24 | CHF₂ | H | H | OH | H | OH | CH₃ | COCH₃ |
| 25 | CF₃ | H | H | OH | H | OCH₃ | H | H |
| 26 | CF₃ | H | H | OH | H | OCH₃ | H | COCH₃ |
| 27 | CF₃ | H | H | OH | H | OCH₃ | CH₃ | H |
| 28 | CF₃ | H | H | OH | H | OCH₃ | CH₃ | COCH₃ |
| 29 | CF₃ | H | H | OH | H | OH | H | H |
| 30 | CF₃ | H | H | OH | H | OH | H | COCH₃ |
| 31 | CF₃ | H | H | OH | H | OH | CH₃ | H |
| 32 | CF₃ | H | H | OH | H | OH | CH₃ | COCH₃ |
| 33 | C₂H₅ | H | H | OH | H | OCH₃ | H | H |
| 34 | C₂H₅ | H | H | OH | H | OCH₃ | H | COCH₃ |
| 35 | C₂H₅ | H | H | OH | H | OCH₃ | CH₃ | H |
| 36 | C₂H₅ | H | H | OH | H | OCH₃ | CH₃ | COCH₃ |
| 37 | C₂H₅ | H | H | OH | H | OH | H | H |
| 38 | C₂H₅ | H | H | OH | H | OH | H | COCH₃ |
| 39 | C₂H₅ | H | H | OH | H | OH | CH₃ | H |
| 40 | C₂H₅ | H | H | OH | H | OH | CH₃ | COCH₃ |
| 41 | C₃H₇ | H | H | OH | H | OCH₃ | H | H |
| 42 | C₃H₇ | H | H | OH | H | OCH₃ | H | COCH₃ |
| 43 | C₃H₇ | H | H | OH | H | OCH₃ | CH₃ | H |
| 44 | C₃H₇ | H | H | OH | H | OCH₃ | CH₃ | COCH₃ |
| 45 | C₃H₇ | H | H | OH | H | OH | H | H |
| 46 | C₃H₇ | H | H | OH | H | OH | H | COCH₃ |
| 47 | C₃H₇ | H | H | OH | H | OH | CH₃ | H |
| 48 | C₃H₇ | H | H | OH | H | OH | CH₃ | COCH₃ |
| 49 | CH₃ | H | H | NHCN | H | OH | H | COCH₃ |
| 50 | CH₃ | H | CO₂H | H | H | H | OH | COCH₃ |
| 51 | CH₃ | H | H | H | H | OH | H | COCH₃ |
| 52 | CH₃ | H | H | CH₂NH₂ | H | OH | H | COCH₃ |
| 53 | CH₃ | H | H | CH₂CH₂CN | H | OH | H | COCH₃ |
| 54 | CH₃ | H | OH | CH₃SO₂NH | H | OH | H | COCH₃ |
| 55 | CH₃ | H | OH | NO₂ | H | OH | H | COCH₃ |
| 56 | CH₃ | H | CH₃SO₃ | NH₂ | H | OH | H | COCH₃ |
| 57 | CH₃ | H | CO₂CH₃ | NO₂ | H | OH | H | COCH₃ |
| 58 | CH₃ | H | NO₂ | NH₂ | H | OH | H | COCH₃ |
| 59 | CH₃ | H | NH₂ | NH₂ | H | OH | H | COCH₃ |
| 60 | CH₃ | H | CH₃ | OH | H | OH | H | COCH₃ |
| 61 | CH₃ | H | C₆H₅ | OH | H | OH | H | COCH₃ |
| 62 | CH₃ | H | CH₂C₆H₅ | OH | H | OH | H | COCH₃ |
| 63 | CH₃ | H | C₆H₁₁(cyclo) | CH₃O | H | OH | H | COCH₃ |
| 64 | CH₃ | OH | OH | H | H | OH | H | COCH₃ |
| 65 | CH₃ | OH | OH | Cl | H | OH | H | COCH₃ |
| 66 | CH₃ | OH | OH | CH₃ | H | OH | H | COCH₃ |
| 67 | CH₃ | OH | OH | F | H | OH | H | COCH₃ |
| 68 | CH₃ | OH | OH | CF₃ | H | OH | H | COCH₃ |
| 69 | CH₃ | H | OH | H | OH | OH | H | COCH₃ |
| 70 | CH₃ | H | OH | Cl | OH | OH | H | COCH₃ |
| 71 | CH₃ | H | OH | F | OH | OH | H | COCH₃ |
| 72 | CH₃ | H | OH | CF₃ | OH | OH | H | COCH₃ |
| 73 | CH₃ | OH | H | H | OH | OH | H | COCH₃ |
| 74 | CH₃ | OH | H | Cl | OH | OH | H | COCH₃ |
| 75 | CH₃ | OH | H | CH₃ | OH | OH | H | COCH₃ |
| 76 | CH₃ | OH | H | CF₃ | OH | OH | H | COCH₃ |
| 77 | CH₃ | H | OH | OH | OH | OH | H | COCH₃ |
| 78 | CH₃ | OH | OH | OH | H | OH | H | COCH₃ |
| 79 | CH₃ | OH | H | OH | OH | OH | H | COCH₃ |
| 80 | CH₃ | H | H | H | H | OH | H | COCH₃ |
| 81 | H | H | H | H | H | OH | H | COCH₃ |
| 82 | H | H | I | H | H | H | H | COCH₃ |
| 83 | CH₃ | H | I | H | H | H | H | COCH₃ |
| 84 | H | H | I | OH | H | H | H | COCH₃ |
| 85 | H | H | I | H | I | H | H | COCH₃ |
| 86 | CH₃ | H | C₆CH₃ | OH | H | H | H | COCH₃ |
| 87 | CH₃ | H | C₆H₅CH₂ | CH₃O | H | H | H | COCH₃ |
| 88 | CH₃ | H | C₆H₅CH₂ | OH | H | H | H | COCH₃ |
| 89 | CH₃ | H | C₆H₁₁(cyclo) | CH₃O | H | H | H | COCH₃ |
| 90 | CH₃ | H | C₆H₁₁(cyclo) | OH | H | H | H | COCH₃ |
| 91 | CH₃ | H | CH₃ | CH₃O | H | H | H | COCH₃ |
| 92 | CH₃ | H | CH₃ | OH | H | H | H | COCH₃ |
| 93 | CH₃ | H | CH₃ | C₆H₅CH₂CO₂ | H | H | H | COCH₃ |
| 94 | CH₃ | H | CH₃ | H | H | H | COCH₃ | |
| 95 | CH₃ | H | CH₃ | C₆H₅CH₂CO₂ | H H | H | COCH₃ | |
| 96 | CH₃ | H | CH₃ | CH₃CO₂ | H | H | H | COCH₃ |
| 97 | CH₃ | H | CH₃O | OH | H | H | H | COCH₃ |
| 98 | CH₃ | H | —OCH₂O— | | H | H | H | COCH₃ |
| 99 | CH₃ | CH₃O | H | H | CH₃O | H | H | COCH₃ |
| 100 | CH₃ | OH | H | H | OH | H | H | COCH₃ |
| 101 | CH₃ | CH₃ | H | CH₃ | H | H | H | COCH₃ |
| 102 | CH₃ | OH | H | OH | H | H | H | COCH₃ |
| 103 | CH₃ | CH₃O | H | H | CH₃O | OC₂H₅ | H | COCH₃ |
| 104 | C≡CH | CH₃O | H | H | H | H | H | COCH₃ |
| 105 | C≡CH | CH₃O | H | H | CH₃O | H | H | COCH₃ |
| 106 | C≡CH | CH₃ | H | OH | H | H | H | COCH₃ |
| 107 | C≡CH | H | OH | H | H | H | H | COCH₃ |
| 108 | CH═CH₂ | CH₃O | H | H | H | H | H | COCH₃ |
| 109 | CH═CH₂ | CH₃O | H | H | CH₃O | H | H | COCH₃ |

The following Examples #110-#413 of Table V are highly preferred conjugates formed from tyrosine hydroxylase inhibitor compounds and glutamic acid derivatives. These tyrosine hydroxylase inhibitors utilized to make these conjugates are embraced by generic Formula I, above.

TABLE V

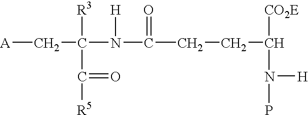

| EXAMPLE | | | | | |
|---|---|---|---|---|---|
| NO. | A | R³ | R⁵ | E | P |
| 110 | 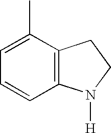 | CH₃ | OCH₃ | H | H |
| 111 | 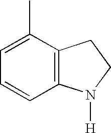 | CH₃ | OCH₃ | H | COCH₃ |
| 112 | 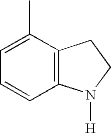 | CH₃ | OCH₃ | CH₃ | H |
| 113 | 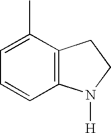 | CH₃ | OCH₃ | CH₃ | COCH₃ |
| 114 | 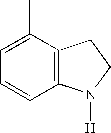 | CH₃ | OH | H | H |
| 115 | 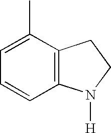 | CH₃ | OH | H | COCH₃ |
| 116 | 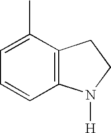 | CH₃ | OH | CH₃ | H |
| 117 | 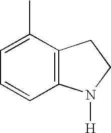 | CH₃ | OH | CH₃ | COCH₃ |
| 118 | 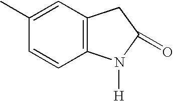 | CH₃ | OCH₃ | H | H |
| 119 | 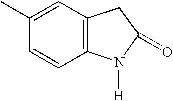 | CH₃ | OCH₃ | H | COCH₃ |
| 120 | 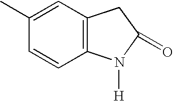 | CH₃ | OCH₃ | CH₃ | H |
| 121 | 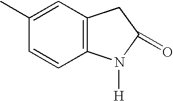 | CH₃ | OCH₃ | CH₃ | COCH₃ |
| 122 | 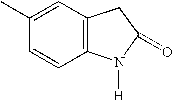 | CH₃ | OH | H | H |
| 123 | 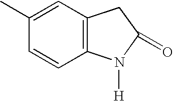 | CH₃ | OH | H | COCH₃ |
| 124 | 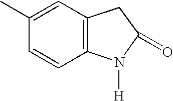 | CH₃ | OH | CH₃ | H |
| 125 | 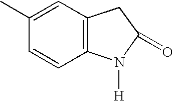 | CH₃ | OH | CH₃ | COCH₃ |
| 126 | 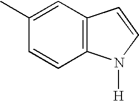 | CH₃ | OCH₃ | H | H |
| 127 | 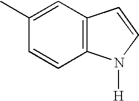 | CH₃ | OCH₃ | H | COCH₃ |
| 128 | 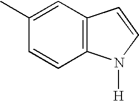 | CH₃ | OCH₃ | CH₃ | H |
| 129 | 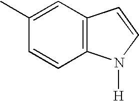 | CH₃ | OCH₃ | CH₃ | COCH₃ |
| 130 | 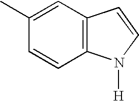 | CH₃ | OH | H | H |
| 131 | 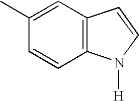 | CH₃ | OH | H | COCH₃ |
| 132 | 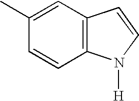 | CH₃ | OH | CH₃ | H |
| 133 | 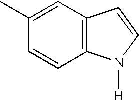 | CH₃ | OH | CH₃ | COCH₃ |
| 134 | 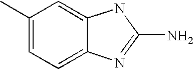 | CH₃ | OCH₃ | H | H |
| 135 | 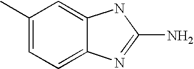 | CH₃ | OCH₃ | H | COCH₃ |
| 136 | 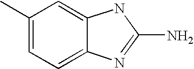 | CH₃ | OCH₃ | CH₃ | H |
| 137 | 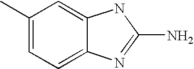 | CH₃ | OCH₃ | CH₃ | COCH₃ |
| 138 | 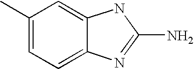 | CH₃ | OH | H | H |
| 139 | 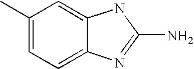 | CH₃ | OH | H | COCH₃ |
| 140 | 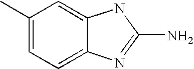 | CH₃ | OH | CH₃ | H |
| 141 | 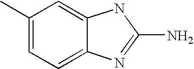 | CH₃ | OH | CH₃ | COCH₃ |
| 142 | 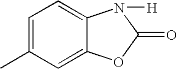 | CH₃ | OCH₃ | H | H |
| 143 | 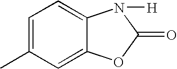 | CH₃ | OCH₃ | H | COCH₃ |
| 144 | 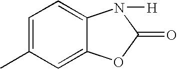 | CH₃ | OCH₃ | CH₃ | H |
| 145 | 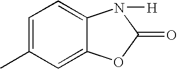 | CH₃ | OCH₃ | CH₃ | COCH₃ |
| 146 | 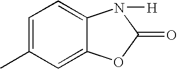 | CH₃ | OH | H | H |
| 147 | 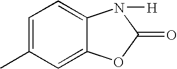 | CH₃ | OH | H | COCH₃ |
| 148 | 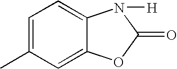 | CH₃ | OH | CH₃ | H |
| 149 | 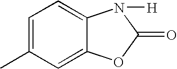 | CH₃ | OH | CH₃ | COCH₃ |
| 150 | 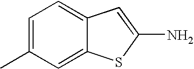 | CH₃ | OCH₃ | H | H |
| 151 | 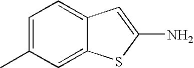 | CH₃ | OCH₃ | H | COCH₃ |
| 152 | 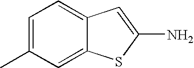 | CH₃ | OCH₃ | CH₃ | H |
| 153 | 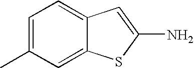 | CH₃ | OCH₃ | CH₃ | COCH₃ |
| 154 | 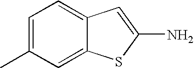 | CH₃ | OH₃ | H | H |
| 155 | 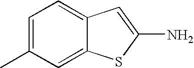 | CH₃ | OH₃ | H | COCH₃ |
| 156 | 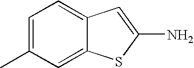 | CH₃ | OH | CH₃ | H |
| 157 | 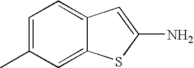 | CH₃ | OH | CH₃ | COCH₃ |
| 158 | 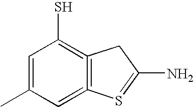 | CH₃ | OCH₃ | H | H |
| 159 | 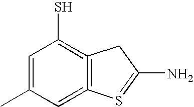 | CH₃ | OCH₃ | H | COCH₃ |
| 160 | 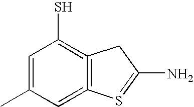 | CH₃ | OCH₃ | CH₃ | H |
| 161 | 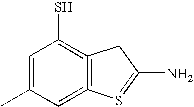 | CH₃ | OCH₃ | CH₃ | COCH₃ |
| 162 | 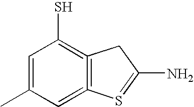 | CH₃ | OH | H | H |
| 163 | 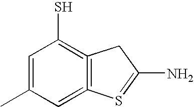 | CH₃ | OH | H | COCH₃ |
| 164 | 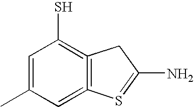 | CH₃ | OH | CH₃ | H |
| 165 | 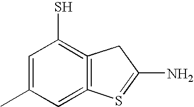 | CH₃ | OH | CH₃ | COCH₃ |
| 166 | 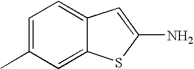 | CH₃ | OCH₃ | H | H |
| 167 | 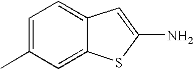 | CH₃ | OCH₃ | H | COCH₃ |
| 168 | 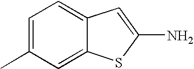 | CH₃ | OCH₃ | CH₃ | H |
| 169 | 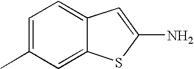 | CH₃ | OCH₃ | CH₃ | COCH₃ |
| 170 | 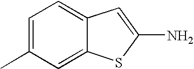 | CH₃ | OH | H | H |
| 171 | 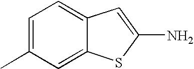 | CH₃ | OH | H | COCH₃ |
| 172 | 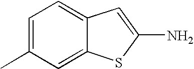 | CH₃ | OH | CH₃ | H |
| 173 | 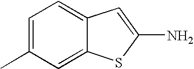 | CH₃ | OH | CH₃ | COCH₃ |
| 174 | 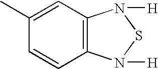 | CH₃ | OCH₃ | H | H |
| 175 | 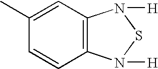 | CH₃ | OCH₃ | H | COCH₃ |
| 176 | 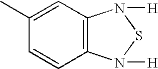 | CH₃ | OCH₃ | CH₃ | H |
| 177 | 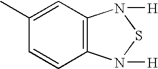 | CH₃ | OCH₃ | CH₃ | COCH₃ |
| 178 | 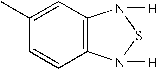 | CH₃ | OH | H | H |
| 179 | 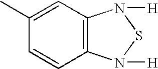 | CH₃ | OH | H | COCH₃ |
| 180 | 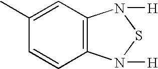 | CH₃ | OH | CH₃ | H |
| 181 | 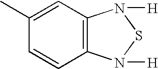 | CH₃ | OH | CH₃ | COCH₃ |
| 182 | 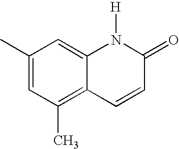 | CH₃ | OCH₃ | H | H |
| 183 | 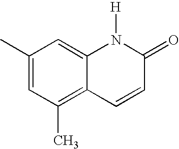 | CH₃ | OCH₃ | H | COCH₃ |
| 184 | 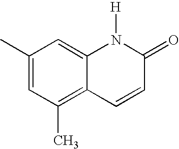 | CH₃ | OCH₃ | CH₃ | H |
| 185 | 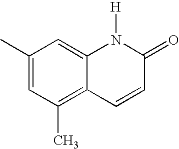 | CH₃ | OCH₃ | CH₃ | COCH₃ |
| 186 | 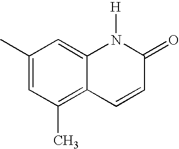 | CH₃ | OH | H | H |
| 187 | 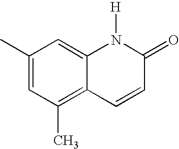 | CH₃ | OH | H | COCH₃ |
| 188 | 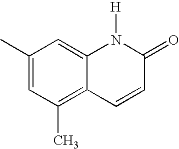 | CH₃ | OH | CH₃ | H |
| 189 | 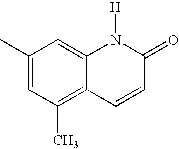 | CH₃ | OH | CH₃ | COCH₃ |
| 190 | 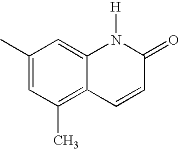 | H | OCH₃ | H | H |
| 191 | 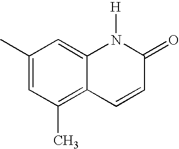 | H | OCH₃ | H | COCH₃ |
| 192 | 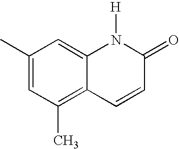 | H | OCH₃ | CH₃ | H |
| 193 | 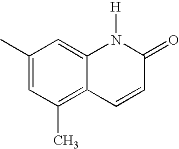 | H | OCH₃ | CH₃ | COCH₃ |
| 194 | 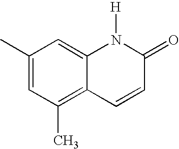 | H | OH | H | H |
| 195 | 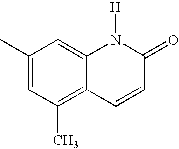 | H | OH | H | COCH₃ |
| 196 | 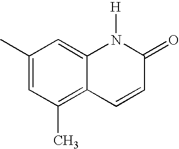 | H | OH | CH₃ | H |
| 197 | 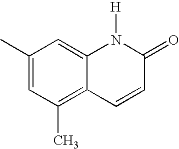 | H | OH | CH₃ | COCH₃ |
| 198 | 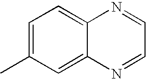 | CH₃ | OCH₃ | H | H |
| 199 | 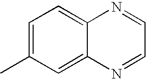 | CH₃ | OCH₃ | H | COCH₃ |
| 200 | 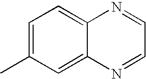 | CH₃ | OCH₃ | CH₃ | H |
| 201 | 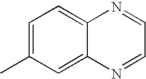 | CH₃ | OCH₃ | CH₃ | COCH₃ |
| 202 | 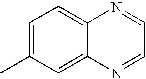 | CH₃ | OH | H | H |
| 203 | 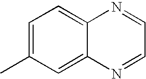 | CH₃ | OH | H | COCH₃ |
| 204 | 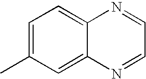 | CH₃ | OH | CH₃ | H |
| 205 | 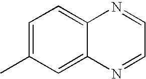 | CH₃ | OH | CH₃ | COCH₃ |
| 206 | 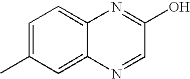 | CH₃ | OCH₃ | H | H |
| 207 | 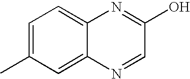 | CH₃ | OCH₃ | H | COCH₃ |
| 208 | 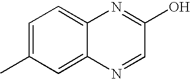 | CH₃ | OCH | CH₃ | H |
| 209 | 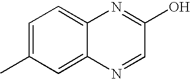 | CH₃ | OCH | CH₃ | COCH₃ |
| 210 | 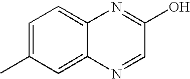 | CH₃ | OH | H | H |
| 211 | 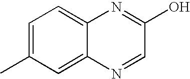 | CH₃ | OH | H | COCH₃ |
| 212 | 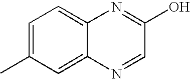 | CH₃ | OH | CH₃ | H |
| 213 | 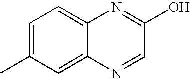 | CH₃ | OH | CH₃ | COCH₃ |
| 214 | 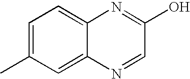 | CH₃ | OCH₃ | H | H |
| 215 | 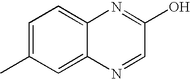 | CH₃ | OCH₃ | H | COCH₃ |
| 216 | 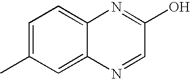 | CH₃ | OCH₃ | CH₃ | H |
| 217 | 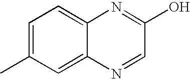 | CH₃ | OCH₃ | CH₃ | COCH₃ |
| 218 | 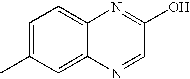 | CH₃ | OH | H | H |
| 219 | 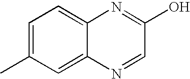 | CH₃ | OH | H | COCH₃ |
| 220 | 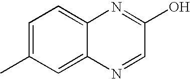 | CH₃ | OH | CH₃ | H |
| 221 | 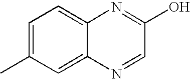 | CH₃ | OH | CH₃ | COCH₃ |
| 222 | 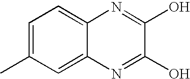 | CH₃ | OCH₃ | H | H |
| 223 | 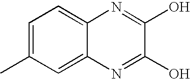 | CH₃ | OCH₃ | H | COCH₃ |
| 224 | 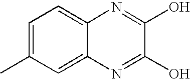 | CH₃ | OCH₃ | CH₃ | H |
| 225 | 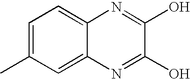 | CH₃ | OCH₃ | CH₃ | COCH₃ |
| 226 | 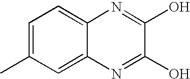 | CH₃ | OH | H | H |
| 227 | 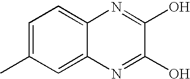 | CH₃ | OH | H | COCH₃ |
| 228 | 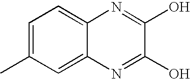 | CH₃ | OH | CH₃ | H |
| 229 | 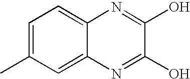 | CH₃ | OH | CH₃ | COCH₃ |
| 230 | 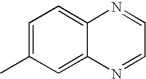 | H | OCH₃ | H | H |
| 231 | 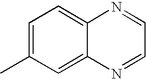 | H | OCH₃ | H | COCH₃ |
| 232 | 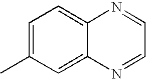 | H | OCH₃ | CH₃ | H |
| 233 | 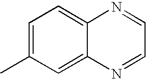 | H | OCH₃ | CH₃ | COCH₃ |
| 234 | 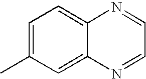 | H | OH | H | H |
| 235 | 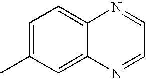 | H | OH | H | COCH₃ |
| 236 | 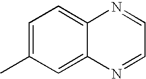 | H | OH | CH₃ | H |
| 237 | 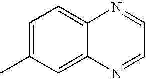 | H | OH | CH₃ | COCH₃ |
| 238 | 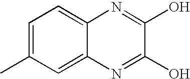 | H | OCH₃ | H | H |
| 239 | 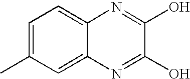 | H | OCH₃ | H | COCH₃ |
| 240 | 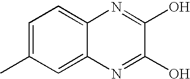 | H | OCH₃ | CH₃ | H |
| 241 | 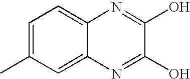 | H | OCH₃ | CH₃ | COCH₃ |
| 242 | 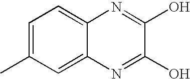 | H | OH | H | H |
| 243 | 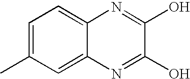 | H | OH | H | COCH₃ |
| 244 | 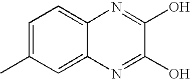 | H | OH | CH₃ | H |
| 245 | 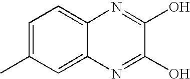 | H | OH | CH₃ | COCH₃ |
| 246 | 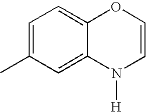 | CH₃ | OCH₃ | H | H |
| 247 | 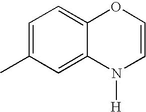 | CH₃ | OCH₃ | H | COCH₃ |
| 248 | 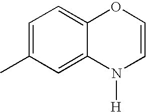 | CH₃ | OCH₃ | CH₃ | H |
| 249 | 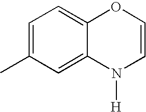 | CH₃ | OCH₃ | CH₃ | COCH₃ |
| 250 | 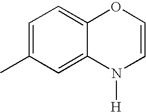 | CH₃ | OH | H | H |
| 251 | 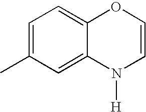 | CH₃ | OH | H | COCH₃ |
| 252 | 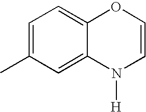 | CH₃ | OH | CH₃ | H |
| 253 | 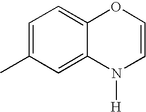 | CH₃ | OH | CH₃ | COCH₃ |
| 254 | 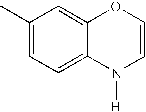 | H | OCH₃ | H | H |
| 255 | 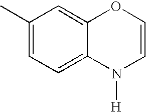 | H | OCH₃ | H | COCH₃ |
| 256 | 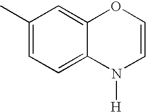 | H | OCH₃ | CH₃ | H |
| 257 | 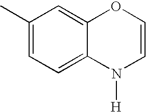 | H | OCH₃ | CH₃ | COCH₃ |
| 258 | 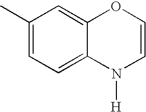 | H | OH | H | H |
| 259 | 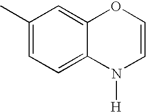 | H | OH | H | COCH₃ |
| 260 | 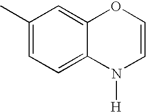 | H | OH | CH₃ | H |
| 261 | 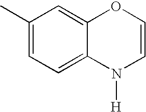 | H | OH | CH₃ | COCH₃ |
| 262 | 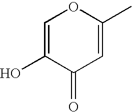 | CH₃ | OCH₃ | H | H |
| 263 | 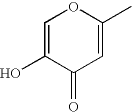 | CH₃ | OCH₃ | H | COCH₃ |
| 264 | 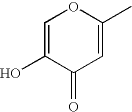 | CH₃ | OCH₃ | CH₃ | H |
| 265 | 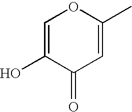 | CH₃ | OCH₃ | CH₃ | COCH₃ |
| 266 | 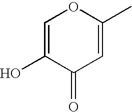 | CH₃ | OH | H | H |
| 267 | 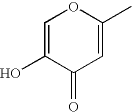 | CH₃ | OH | H | COCH₃ |
| 268 | 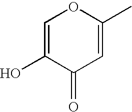 | CH₃ | OH | CH₃ | H |
| 269 | 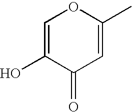 | CH₃ | OH | CH₃ | COCH₃ |
| 270 | 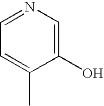 | CH₃ | OCH₃ | H | H |
| 271 | 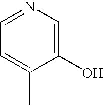 | CH₃ | OCH₃ | H | COCH₃ |
| 272 | 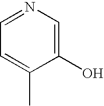 | CH₃ | OCH₃ | CH₃ | H |
| 273 | 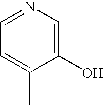 | CH₃ | OCH₃ | CH₃ | COCH₃ |
| 274 | 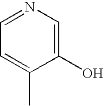 | CH₃ | OH | H | H |
| 275 | 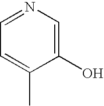 | CH₃ | OH | H | COCH₃ |
| 276 | 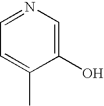 | CH₃ | OH | CH₃ | H |
| 277 | 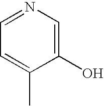 | CH₃ | OH | CH₃ | COCH₃ |
| 278 | 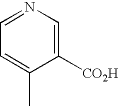 | CH₃ | OCH₃ | H | H |
| 279 | 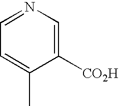 | CH₃ | OCH₃ | H | COCH₃ |
| 280 | 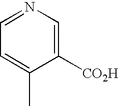 | CH₃ | OCH₃ | CH₃ | H |
| 281 | 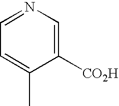 | CH₃ | OCH₃ | CH₃ | COCH₃ |
| 282 | 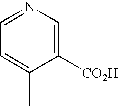 | CH₃ | OH | H | H |
| 283 | 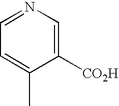 | CH₃ | OH | H | COCH₃ |
| 284 | 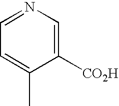 | CH₃ | OH | CH₃ | H |
| 285 | 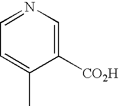 | CH₃ | OH | CH₃ | COCH₃ |
| 286 | 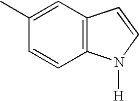 | CH₃ | OCH₃ | H | H |
| 287 | 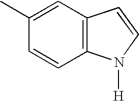 | CH₃ | OCH₃ | H | COCH₃ |
| 288 | 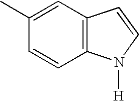 | CH₃ | OCH₃ | CH₃ | H |
| 289 | 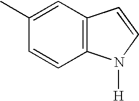 | CH₃ | OCH₃ | CH₃ | COCH₃ |
| 290 | 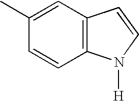 | CH₃ | OH | H | H |
| 291 | 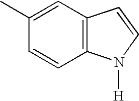 | CH₃ | OH | H | COCH₃ |
| 292 | 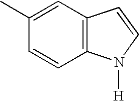 | CH₃ | OH | CH₃ | H |
| 293 | 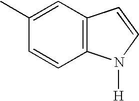 | CH₃ | OH | CH₃ | COCH₃ |
| 294 | 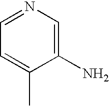 | CH₃ | OCH₃ | H | H |
| 295 | 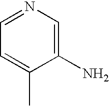 | CH₃ | OCH₃ | H | COCH₃ |
| 296 | 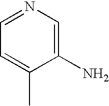 | CH₃ | OCH₃ | CH₃ | H |
| 297 | 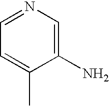 | CH₃ | OCH₃ | CH₃ | COCH₃ |
| 298 | 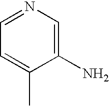 | CH₃ | OH | H | H |
| 299 | 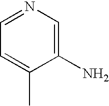 | CH₃ | OH | H | COCH₃ |
| 300 | 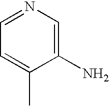 | CH₃ | OH | CH₃ | H |
| 301 | 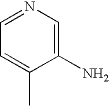 | CH₃ | OH | CH₃ | COCH₃ |
| 302 | 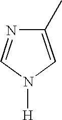 | CH≡CH | OCH₃ | H | H |
| 303 | 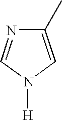 | CH≡CH | OCH₃ | H | COCH₃ |
| 304 | 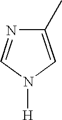 | CH≡CH | OCH₃ | CH₃ | H |
| 305 | 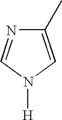 | CH≡CH | OCH₃ | CH₃ | COCH₃ |
| 306 | 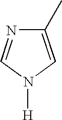 | CH≡CH | OH | H | H |
| 307 | 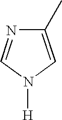 | CH≡CH | OH | H | COCH₃ |
| 308 | 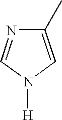 | CH≡CH | OH | CH₃ | H |
| 309 | 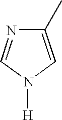 | CH≡CH | OH | CH₃ | COCH₃ |
| 310 | 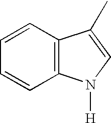 | CH≡CH | OCH₃ | H | H |
| 311 | 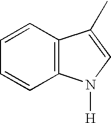 | CH≡CH | OCH₃ | H | COCH₃ |
| 312 | 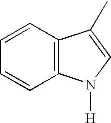 | CH≡CH | OCH₃ | CH₃ | H |
| 313 | 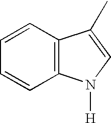 | CH≡CH | OCH₃ | CH₃ | COCH₃ |
| 314 | 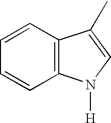 | CH≡CH | OH | H | H |
| 315 | 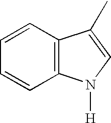 | CH≡CH | OH | H | COCH₃ |
| 316 | 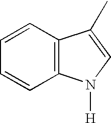 | CH≡CH | OH | CH₃ | H |
| 317 | 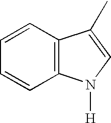 | CH≡CH | OH | CH₃ | COCH₃ |
| 318 | 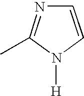 | CH≡CH₂ | OCH₃ | H | H |
| 319 | 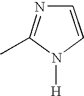 | CH≡CH₂ | OCH₃ | H | COCH₃ |
| 320 | 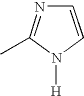 | CH≡CH₂ | OCH₃ | CH₃ | H |
| 321 | 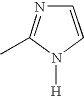 | CH≡CH₂ | OCH₃ | CH₃ | COCH₃ |
| 322 | 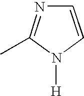 | CH≡CH₂ | OH | H | H |
| 323 | 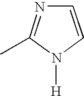 | CH≡CH₂ | OH | H | COCH₃ |
| 324 | 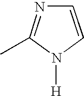 | CH≡CH₂ | OH | CH₃ | H |
| 325 | 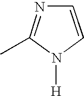 | CH≡CH₂ | OH | CH₃ | COCH₃ |
| 326 | 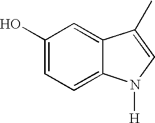 | C≡CH | OCH₃ | H | H |
| 327 | 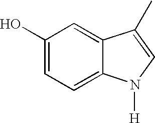 | C≡CH | OCH₃ | H | COCH₃ |
| 328 | 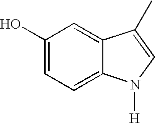 | C≡CH | OCH₃ | CH₃ | H |
| 329 | 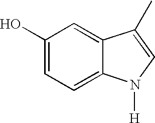 | C≡CH | OCH₃ | CH₃ | COCH₃ |
| 330 | 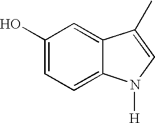 | C≡CH | OH | H | H |
| 331 | 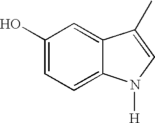 | C≡CH | OH | H | COCH₃ |
| 332 | 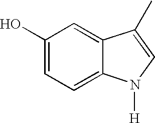 | C≡CH | OH | CH₃ | H |
| 333 | 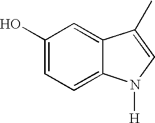 | C≡CH | OH | CH₃ | COCH₃ |
| 334 | 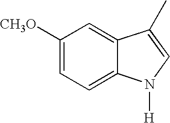 | C≡CH | OCH₃ | H | H |
| 335 | 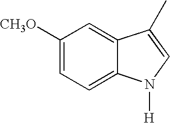 | C≡CH | OCH₃ | H | COCH₃ |
| 336 | 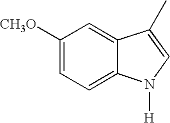 | C≡CH | OCH₃ | CH₃ | H |
| 337 | 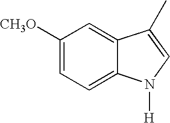 | C≡CH | OCH₃ | CH₃ | COCH₃ |
| 338 | 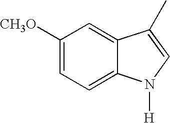 | C≡CH | OH | H | H |
| 339 | 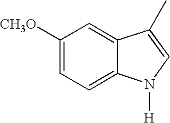 | C≡CH | OH | H | COCH₃ |
| 340 | 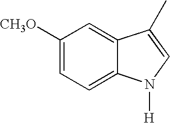 | C≡CH | OH | CH₃ | H |
| 341 | 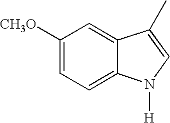 | C≡CH | OH | CH₃ | COCH₃ |
| 342 | 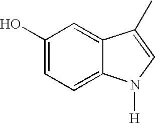 | CH₃ | OCH₃ | H | H |
| 343 | 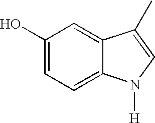 | CH₃ | OCH₃ | H | COCH₃ |
| 344 | 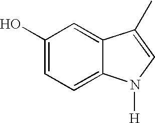 | CH₃ | OCH₃ | CH₃ | H |
| 345 | 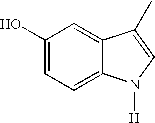 | CH₃ | OCH₃ | CH₃ | COCH₃ |
| 346 | 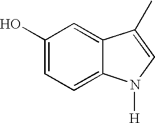 | CH₃ | OH | H | H |
| 347 | 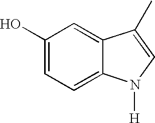 | CH₃ | OH | H | COCH₃ |
| 348 | 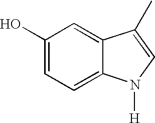 | CH₃ | OH | CH₃ | H |
| 349 | 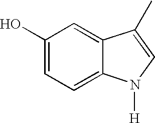 | CH₃ | OH | CH₃ | COCH₃ |
| 350 | 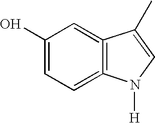 | H | OCH₃ | H | H |
| 351 | 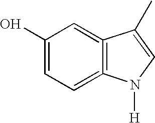 | H | OCH₃ | H | COCH₃ |
| 352 | 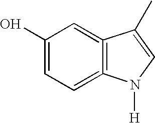 | H | OCH₃ | CH₃ | H |
| 353 | 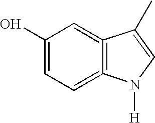 | H | OCH₃ | CH₃ | COCH₃ |
| 354 | 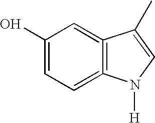 | H | OH | H | H |
| 355 | 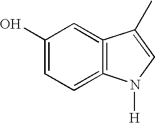 | H | OH | H | COCH₃ |
| 356 | 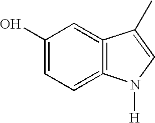 | H | OH | CH₃ | H |
| 357 | 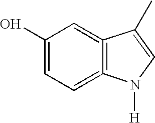 | H | OH | CH₃ | COCH₃ |
| 358 | 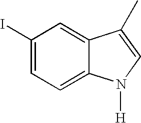 | H | OCH₃ | H | H |
| 359 | 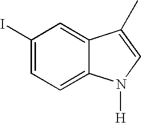 | H | OCH₃ | H | COCH₃ |
| 360 | 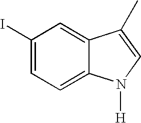 | H | OCH₃ | CH₃ | H |
| 361 | 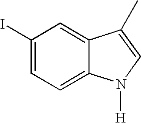 | H | OCH₃ | CH₃ | COCH₃ |
| 362 | 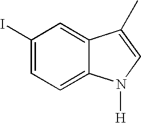 | H | OH | H | H |
| 363 | 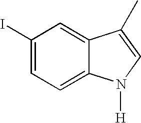 | H | OH | H | COCH₃ |
| 364 | 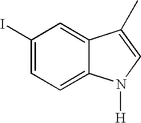 | H | OH | CH₃ | H |
| 365 | 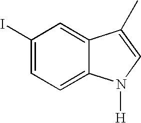 | H | OH | CH₃ | COCH₃ |
| 366 | 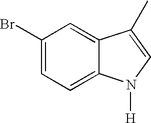 | H | OCH₃ | H | H |
| 367 | 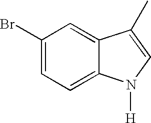 | H | OCH₃ | H | COCH₃ |
| 368 | 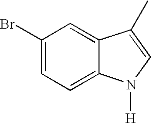 | H | OCH₃ | CH₃ | H |
| 369 | 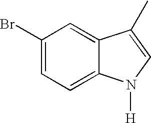 | H | OCH₃ | CH₃ | COCH₃ |
| 370 | 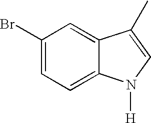 | H | OH | H | H |
| 371 | 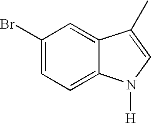 | H | OH | H | COCH₃ |
| 372 | 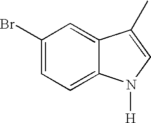 | H | OH | CH₃ | H |
| 373 | 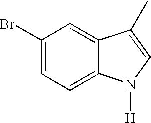 | H | OH | CH₃ | COCH₃ |
| 374 | 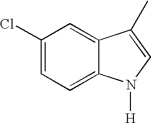 | H | OCH₃ | H | H |
| 375 | 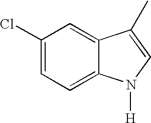 | H | OCH₃ | H | COCH₃ |
| 376 | 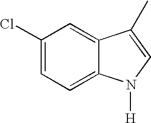 | H | OCH₃ | CH₃ | H |
| 377 | 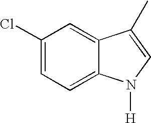 | H | OCH₃ | CH₃ | COCH₃ |
| 378 | 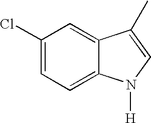 | H | OH | H | H |
| 379 | 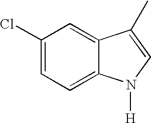 | H | OH | H | COCH₃ |
| 380 | 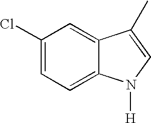 | H | OH | CH₃ | H |
| 381 | 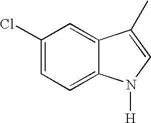 | H | OH | CH₃ | COCH₃ |
| 382 | 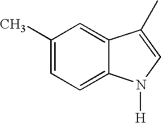 | H | OCH₃ | H | H |
| 387 | 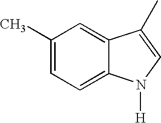 | H | OCH₃ | H | COCH₃ |
| 384 | 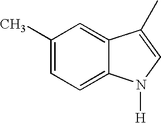 | H | OCH₃ | CH₃ | H |
| 385 | 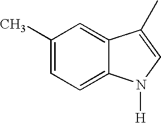 | H | OCH₃ | CH₃ | COCH₃ |
| 386 | 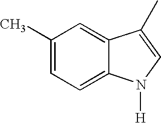 | H | OH | H | H |
| 387 | 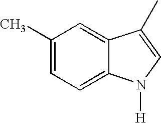 | H | OH | H | COCH₃ |
| 388 | 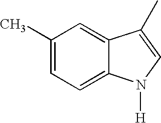 | H | OH | CH₃ | H |
| 389 | 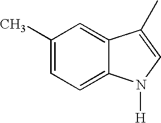 | H | OH | CH₃ | COCH₃ |
| 390 | 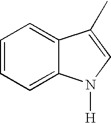 | CH₃ | OCH₃ | H | H |
| 391 | 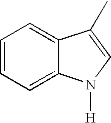 | CH₃ | OCH₃ | H | COCH₃ |
| 392 | 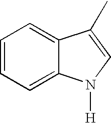 | CH₃ | OCH₃ | CH₃ | H |
| 393 | 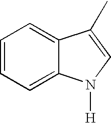 | CH₃ | OCH₃ | CH₃ | COCH₃ |
| 394 | 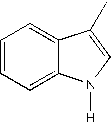 | CH₃ | OH | H | H |
| 395 | 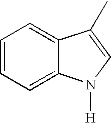 | CH₃ | OH | H | COCH₃ |
| 396 | 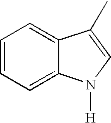 | CH₃ | OH | CH₃ | H |
| 397 | 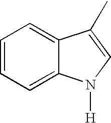 | CH₃ | OH | CH₃ | COCH₃ |
| 398 | C₂H | CH═CH2 | CH₃ | H | H |
| 399 | C₂H₅ | CH═CH2 | OCH₃ | H | COCH₃ |
| 400 | C₂H₅ | CH═CH2 | OCH₃ | CH₃ | H |
| 401 | C₂H₅ | CH═CH2 | OCH₃ | CH₃ | COCH₃ |
| 402 | C₂H₅ | CH═CH2 | OH | H | H |
| 403 | C₂H₅ | CH═CH2 | OH | H | COCH₃ |
| 404 | C₂H₅ | CH═CH2 | OH | H | COCH₃ |
| 405 | C₂H₅ | CH═CH2 | OH | CH₃ | COCH₃ |
| 406 | C₂H₅ | C≡CH | OCH₃ | H | H |
| 407 | C₂H₅ | C≡CH | OCH₃ | H | COCH₃ |
| 408 | C₂H₅ | C≡CH | OCH₃ | CH₃ | H |
| 409 | C₂H₅ | C≡CH | OCH₃ | CH₃ | COCH₃ |
| 410 | C₂H₅ | C≡CH | OH | H | H |
| 411 | C₂H₅ | C≡CH | OH | H | COCH₃ |
| 412 | C₂H₅ | C≡CH | OH | H | COCH₃ |
| 413 | C₂H₅ | C≡CH | OH | CH₃ | COCH₃ |

The following Examples #414-#461 of Table VI are highly preferred conjugates formed from tyrosine hydroxylase inhibitor compounds and glutamic acid derivatives. These tyrosine hydroxylase inhibitors utilized to make these conjugates are embraced by generic Formula III, above.

TABLE VI

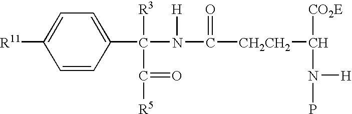

| EXAMPLE | | | | | |
|---|---|---|---|---|---|
| NO. | R¹¹ | R³ | R⁵ | E | P |
| 414 | OH | H | OH | H | H |
| 415 | OH | H | OH | H | COCH₃ |
| 416 | OH | H | OH | CH₃ | H |
| 417 | OH | H | OH | CH₃ | COCH₃ |
| 418 | OH | H | OCH₃ | H | H |
| 419 | OH | H | OCH₃ | H | COCH₃ |
| 420 | OH | H | OCH₃ | CH₃ | H |
| 421 | OH | H | OCH₃ | CH₃ | COCH₃ |
| 422 | OH | CH₃ | OH | H | H |
| 423 | OH | CH₃ | OH | H | COCH₃ |
| 424 | OH | CH₃ | OH | CH₃ | H |
| 425 | OH | CH₃ | OH | CH₃ | COCH₃ |
| 426 | OH | CH₃ | OCH₃ | H | H |
| 427 | OH | CH₃ | OCH₃ | H | COCH₃ |
| 428 | OH | CH₃ | OCH₃ | CH₃ | H |
| 429 | OH | CH₃ | OCH₃ | CH₃ | COCH₃ |
| 430 | OH | H | NH₂ | H | H |
| 431 | OH | H | NH₂ | H | COCH₃ |
| 432 | OH | H | NH₂ | CH₃ | H |
| 433 | OH | H | NH₂ | CH₃ | COCH₃ |
| 434 | OH | CH₃ | NH₂ | H | H |
| 435 | OH | CH₃ | NH₂ | H | COCH₃ |
| 436 | OH | CH₃ | NH₂ | CH₃ | H |
| 437 | OH | CH₃ | NH₂ | CH₃ | COCH₃ |
| 438 | OCH₃ | H | OH | H | H |
| 439 | OCH₃ | H | OH | H | COCH₃ |
| 440 | OCH₃ | H | OH | CH₃ | H |
| 441 | OCH₃ | H | OH | CH₃ | COCH₃ |
| 442 | OCH₃ | H | OCH₃ | H | H |
| 443 | OCH₃ | H | OCH₃ | H | COCH₃ |
| 444 | OCH₃ | H | OCH₃ | CH₃ | H |
| 445 | OCH₃ | H | OCH₃ | CH₃ | COCH₃ |
| 446 | OCH₃ | CH₃ | OH | H | H |
| 447 | OCH₃ | CH₃ | OH | H | COCH₃ |
| 448 | OCH₃ | CH₃ | OH | CH₃ | H |
| 449 | OCH₃ | CH₃ | OH | CH₃ | COCH₃ |
| 450 | OCH₃ | CH₃ | OCH₃ | H | H |
| 451 | OCH₃ | CH₃ | OCH₃ | H | COCH₃ |
| 452 | OCH₃ | CH₃ | OCH₃ | CH₃ | H |
| 453 | OCH₃ | CH₃ | OCH₃ | CH₃ | COCH₃ |
| 454 | OCH₃ | H | NH₂ | H | H |
| 455 | OCH₃ | H | NH₂ | H | COCH₃ |
| 456 | OCH₃ | H | NH₂ | CH₃ | H |
| 457 | OCH₃ | H | NH₂ | CH₃ | COCH₃ |
| 458 | OCH₃ | CH₃ | NH₂ | H | H |
| 459 | OCH₃ | CH₃ | NH₂ | H | COCH₃ |
| 460 | OCH₃ | CH₃ | NH₂ | CH₃ | H |
| 461 | OCH₃ | CH₃ | NH₂ | CH₃ | COCH₃ |

The following Examples #462-#857 comprise five classes of highly preferred conjugates composed of dopa-decarboxylase inhibitor compounds and glutamic acid derivatives. Examples #462-#464 are descriptions of specific preparations of such conjugates. Examples #465-#857, as shown in Tables VII-XI, may be prepared by procedures shown in these specific examples and in the foregoing general synthetic procedures of Schemes 1-7

### EXAMPLE 462

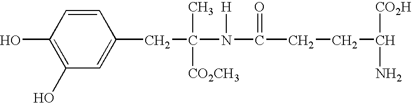

4-amino-4-carboxy-1-oxobutyl-3-hydroxy-α-methyl-L-tyrosine, Methyl Ester.

Step. 1: Preparation of α-methyl-L-DOPA, Methyl Ester, Hydrochloride.

A suspension of 29.7 g (141 mmol) of α-methyl-L-DOPA in 300 mL of absolute methanol was cooled to −15° C. and treated with 125.8 g (1.06 mol) thionyl chloride under a nitrogen atmosphere. The reaction was allowed to warm to ambient temperature and stir at reflux for 3 days. Concentration followed by trituration with ether gave 31.7 g (97%) as an off-white solid: NMR (DMSO-d₆) δ 1.47 (s, 3H), 2.92 (d, J=12 Hz, 1H), 2.98 (d, J=12 Hz, 1H), 3.74 (s, 3H), 6.41 (d of d, J=9 Hz AND 2 Hz, 1H), 6.54 (d, J=2 Hz, 1H), 6.68 (d, J=9 Hz, 1H), 8.46-8.90 (br s, 3H), 8.93 (s, 1H), 8.96 (s, 1H).

Step 2: Prepraration of 4-amino-4-carboxy-1-oxobutyl-3-hydroxy-α-methyl-L-tyrosine, Methyl Ester.

Under nitrogen, a solution of 32.7 g (108 mmol) of N-Boc-L-γ-glutamic acid-α-t-butyl ester (BACHEM) in 150 mL of methylene chloride was treated with 11.14 g (54 mmol) of solid dicyclohexylcarbodiimide (DCC). The reaction was allowed to stir for 2 hr prior to filtration under a nitrogen atmosphere. The methylene chloride was removed in vacuo and the residue dissolved in 110 mL of dimethylformamide (DMF). The anhydride solution was slowly added to a solution of 12.9 g (49 mmol) of the α-methyl-DOPA ester from step 1 and 12.6 g (98 mmol) of diisopropylethylamine (DIEA) in 50 mL of anhydrous DMF. The reaction was allowed to stir overnight and was concentrated in vacuo. The residue was dissolved in ethyl acetate, washed with 1N citric acid, 1N NaHCO₃, water, and brine, dried (Na₂SO₄), and concentrated in vacuo to give the protected coupled product; a solution of this material in 100 mL of methylene chloride was cooled to 0° C. and treated with 400 mL of trifluoroacetic acid (TFA) under nitrogen. The reaction was allowed to warm to ambient temperature and stir for 72 hr. Concentration in vacuo gave 4-amino-4-carboxy-1-oxobutyl-3-hydroxy-α-methyl-tyrosine, methyl ester: NMR (DMSO-d₆) δ 1.40 (s, 3H), 1.85-2.30 (m, 2H), 2.30-2.50 (m, 2H), 2.77 (d, J=12 Hz, 1H), 3.00 (d, J=12 Hz, 1H), 3.58 (s, 3H), 3.85-4.10 (m, 1H), 6.29 (d of d, J=9 Hz and 2 Hz, 1H), 6.45 (d, J=2 Hz, 1H), 6.62 (d, J=9 Hz, 1H); MS (FAB) m/e (rel intensity) 355 (92), 225 (51), 148 (35).

### EXAMPLE 463

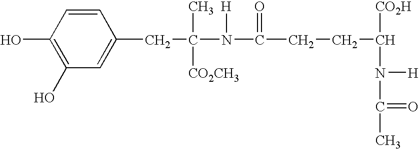

N-[4-(acetylamino)-4-carboxy-1-oxobutyl]-3-hydroxy-α-methyl-L-tyrosine, Methyl Ester.

The compound of Example 462 was dissolved in 100 mL of degassed water and under nitrogen the pH adjusted to 9 with 1 M K₂CO₃. The solution was cooled to 0° C. and 12 mL (127 mmol) of acetic anhydride and 180 mL (180 mmol) of 1 M K₂CO₃ was added every 30 min. for 5 h; the pH was maintained at 9 and the reaction temperature kept below 5° C. After the last addition, the reaction was allowed to warm to ambient temperature overnight. The pH was adjusted to 3 with 3 M HCl and concentrated to 100 mL. Purification by reverse phase chromatography (Waters Deltaprep-3000) using a 5-15% gradient of acetonitrile/water (0.05% TFA) gave 14.0 g (49%) of colorless product: NMR (DMSO-d₆) δ 1.15 (s, 3H), 1.70-1.83 (m, 2H), 1.85 (s, 3H), 1.87-2.00 (m, 2H), 2.15 (t, J=7 Hz, 2H), 2.75 (d, J=12 Hz, 1H), 3.00 (d, J=12 Hz, 1H), 3.55 (s, 3H), 4.10-4.22 (m, 1H), 6.29 (d of d, J=9 Hz and 2 Hz, 1H), 6.43 (d, J=2 Hz, 1H), 6.60 (d, J=9 Hz, 1H), 7.96 (s, 1H), 8.12 (d, J=8 Hz, 1H); MS (FAB) m/e (rel intensity) 397 (100), 365 (10), 226 (70), 166 (90), 153 (22), 130 (72), 102 (28).

### EXAMPLE 464

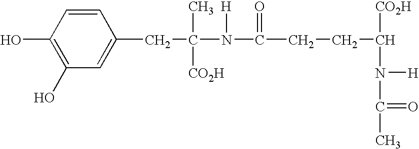

N-[4-(acetylamino)-4-carboxy-1-oxobutyl]-3-hydroxy-α-methyl-L-tyrosine.

A solution of 13.5 g (102 mmol) of the compound of Example 463 in 34 mL of water was cooled to 0° C. and treated with 102 mL (102 mmol) of 1N NaOH (all solutions were degassed in vacuo and flushed with nitrogen prior to use). The reaction was stirred at ambient temperature for 5 hr and the pH adjusted to pH 1 with 6N HCl. Purification by reverse phase chromatography (Waters Deltaprep-3000) using a 2-10% gradient of acetonitrile/water (0.05% TFA) gave 8.9 g (68%) of colorless product: NMR (DMSO-d₆) δ 1.18 (s, 3H), 1.70-1.83 (m, 2H), 1.85 (s, 3H), 1.87-2.00 (m, 2H), 2.15 (t, J=7 Hz, 2H), 2.75 (d, J=12 Hz, 1H), 3.05 (d, J=12 Hz, 1H), 4.10-4.23 (m, 0.1H), 6.31 (d of d, J=9 Hz and 2 Hz, 1H), 6.47 (d, J=2 Hz, 1H), 6.60 (d, J=9 Hz, 1H), 7.71 (s, 1H), 8.15 (d, J=8 Hz, 1H); MS (FAB) m/e (rel intensity) 383 (23), 212 (10), 166 (18), 130 (21), 115 (23); HRMS. Calcd for M+H: 383.1454. Found: 383.1450. Anal: Calcd for C₁₇H₂₂N₂O₈.1.06 H₂O.0.85 TFA: C, 48.67; H, 5.59; N, 6.46; F, 3.73. Found: C, 49.02; H, 5.73; N, 6.40; F, 3.70.

The following Examples #465-#541 of Table VII are highly preferred conjugates composed of dopa-decarboxylase inhibitor compounds and glutamic acid derivatives. These dopa-decarboxylase inhibitors utilized to make these conjugates are embraced by generic Formula IV, above.

TABLE VII

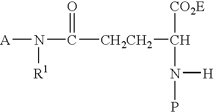

| EXAMPLE | | | | |
|---|---|---|---|---|
| NO. | A | R¹ | E | P |
| 465 | 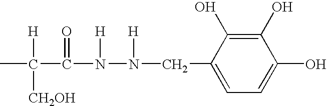 | H | CH₃ | COCH₃ |
| 466 | 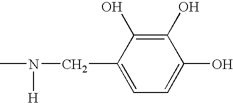 | H | H | H |
| 467 | 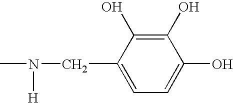 | H | H | COCH₃ |
| 468 | 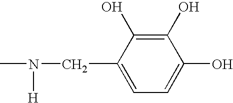 | H | CH₃ | H |
| 469 | 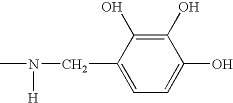 | H | CH₃ | COCH₃ |
| 470 | 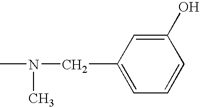 | H | H | H |
| 471 | 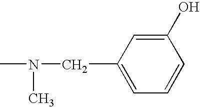 | H | H | COCH₃ |
| 472 | 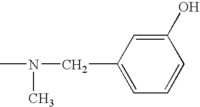 | H | CH₃ | H |
| 473 | 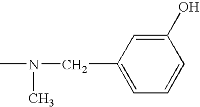 | H | CH₃ | COCH₃ |
| 474 | 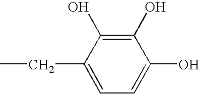 | NH₂ | H | H |
| 475 | 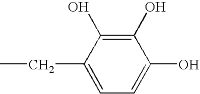 | NH₂ | H | COCH₃ |
| 476 | 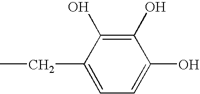 | NH₂ | CH₃ | H |
| 477 | 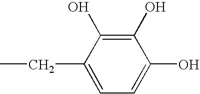 | NH₂ | CH₃ | COCH₃ |
| 478 | 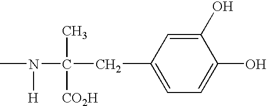 | H | H | H |
| 479 | 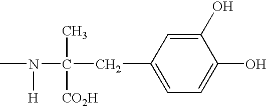 | H | H | COCH₃ |
| 480 | 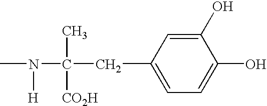 | H | CH₃ | H |
| 481 | 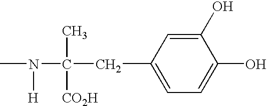 | H | CH₃ | COCH₃ |
| 482 | 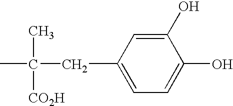 | NH₂ | H | H |
| 483 | 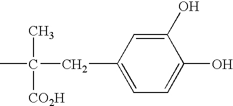 | NH₂ | H | COCH₃ |
| 484 | 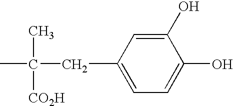 | NH₂ | CH₃ | H |
| 485 | 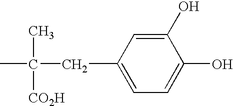 | NH₂ | CH₃ | COCH₃ |
| 486 | 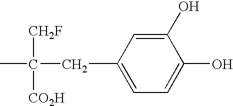 | H | H | H |
| 487 | 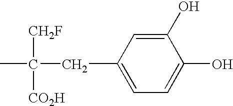 | H | H | COCH₃ |
| 488 | 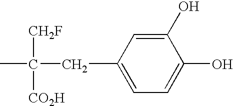 | H | CH₃ | H |
| 489 | 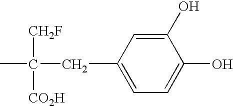 | H | CH₃ | COCH₃ |
| 490 | 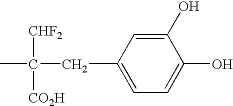 | H | H | H |
| 491 | 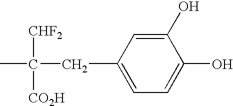 | H | H | COCH₃ |
| 492 | 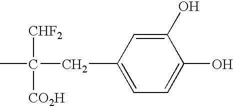 | H | CH₃ | H |
| 493 | 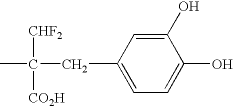 | H | CH₃ | COCH₃ |
| 494 | 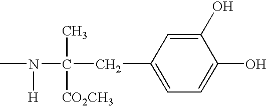 | H | H | H |
| 495 | 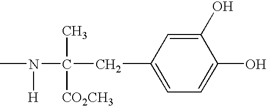 | H | H | COCH₃ |
| 496 | 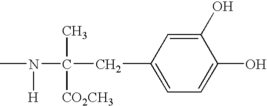 | H | CH₃ | H |
| 497 | 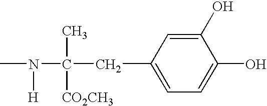 | H | CH₃ | COCH₃ |
| 498 | 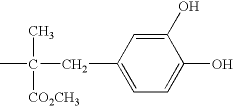 | NH₂ | H | H |
| 499 | 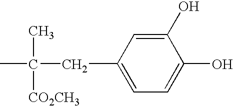 | NH₂ | H | COCH₃ |
| 500 | 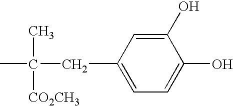 | NH₂ | CH₃ | H |
| 501 | 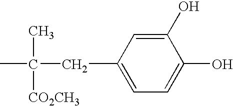 | NH₂ | CH₃ | COCH₃ |
| 502 | 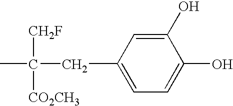 | H | H | H |
| 503 | 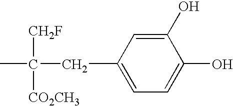 | H | H | COCH₃ |
| 504 | 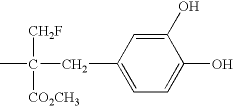 | H | CH₃ | H |
| 505 | 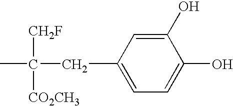 | H | CH₃ | COCH₃ |
| 506 | 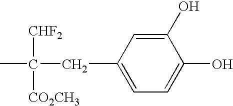 | H | H | H |
| 507 | 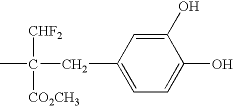 | H | H | COCH₃ |
| 508 | 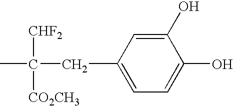 | H | CH₃ | H |
| 509 | 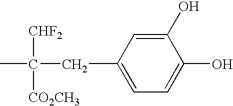 | H | CH₃ | COCH₃ |
| 510 | 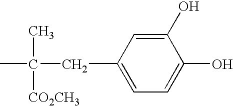 | H | H | H |
| 511 | 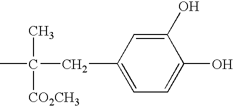 | H | H | COCH₃ |
| 512 | 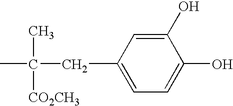 | H | CH₃ | H |
| 513 | 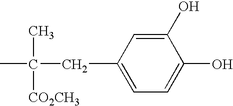 | H | CH₃ | COCH₃ |
| 514 | 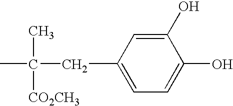 | H | H | H |
| 515 | 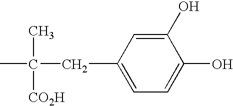 | H | H | COCH₃ |
| 516 | 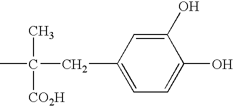 | H | CH₃ | H |
| 517 | 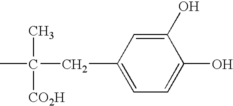 | H | CH₃ | COCH₃ |
| 518 | 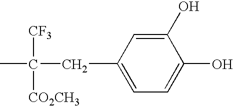 | H | H | H |
| 519 | 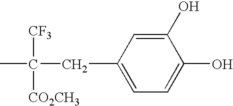 | H | H | COCH₃ |
| 520 | 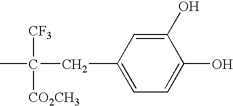 | H | CH₃ | H |
| 521 | 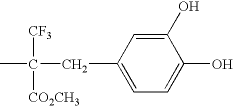 | H | CH₃ | COCH₃ |
| 522 | 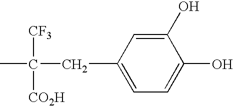 | H | H | H |
| 523 | 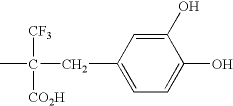 | H | H | COCH₃ |
| 524 | 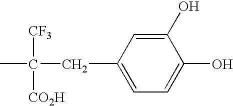 | H | CH₃ | H |
| 525 | 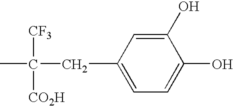 | H | CH₃ | COCH₃ |
| 526 | 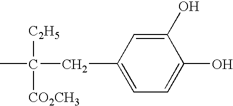 | H | H | H |
| 527 | 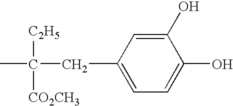 | H | H | COCH₃ |
| 528 | 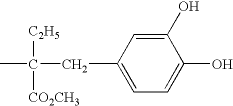 | H | CH₃ | H |
| 529 | 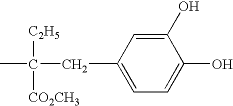 | H | CH₃ | COCH₃ |
| 530 | 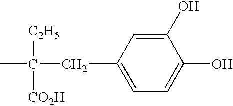 | H | H | H |
| 531 | 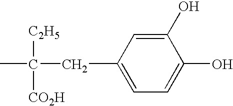 | H | H | COCH₃ |
| 532 | 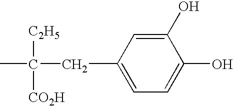 | H | CH₃ | H |
| 533 | 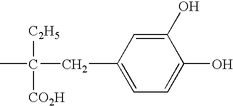 | H | CH₃ | COCH₃ |
| 534 | 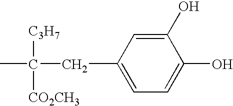 | H | H | H |
| 535 | 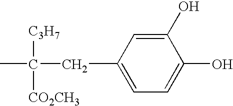 | H | H | COCH₃ |
| 536 | 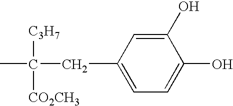 | H | H | H |
| 537 | 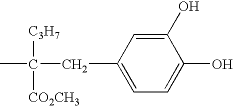 | H | H | COCH₃ |
| 538 | 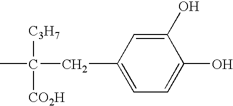 | H | H | H |
| 539 | 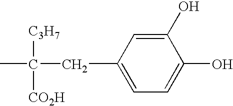 | H | H | COCH₃ |
| 540 | 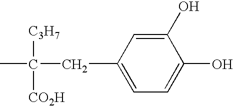 | H | CH₃ | H |
| 541 | 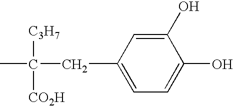 | H | CH₃ | COCH₃ |

The following Examples #542-#577 of Table VIII are highly preferred conjugates composed of dopa-decarboxylase inhibitor compounds and glutamic acid derivatives. These dopa-decarboxylase inhibitors utilized to make these conjugates are embraced by generic Formula VIII, above.

TABLE VIII

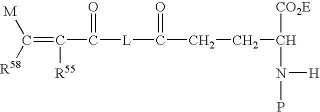

| EXAMPLE | | | | | | |
|---|---|---|---|---|---|---|
| NO. | L | M | R⁵⁶ | R⁵⁵ | E | P |
| 542 | NHNH | 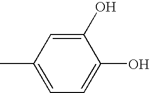 | H | H | H | H |
| 543 | NHNH | 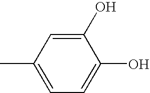 | H | H | H | COCH₃ |
| 544 | NHNH | 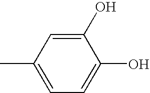 | H | H | CH₃ | H |
| 545 | NHNH | 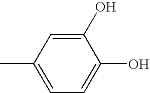 | H | H | CH₃ | COCH₃ |
| 546 | NHNH | 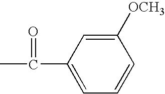 | Br | H | H | H |
| 547 | NHNH | 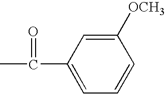 | Br | H | H | COCH₃ |
| 548 | NHNH | 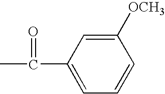 | Br | H | CH₃ | H |
| 549 | NHNH | 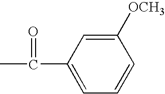 | Br | H | CH₃ | COCH₃ |
| 550 | NHNH | 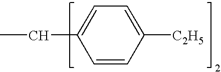 | Br | Br | H | H |
| 551 | NHNH | 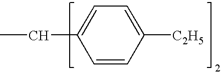 | Br | Br | H | COCH₃ |
| 552 | NHNH | 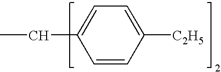 | Br | Br | CH₃ | H |
| 553 | NHNH | 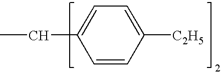 | Br | Br | CH₃ | |
| 554 | NHCH₂CH₂NH | 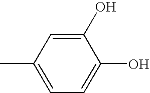 | H | H | H | H |
| 555 | NHCH₂CH₂NH | 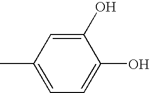 | H | H | H | COCH₃ |
| 556 | NHCH₂CH₂NH | 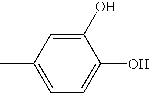 | H | H | CH₃ | H |
| 557 | NHCH₂CH₂NH | 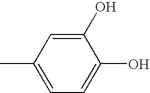 | H | H | CH₃ | COCH₃ |
| 558 | NHCH₂CH₂NH | 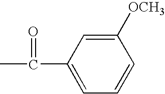 | Br | H | H | H |
| 559 | NHCH₂CH₂NH | 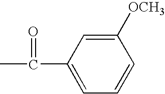 | Br | H | H | COCH₃ |
| 560 | NHCH₂CH₂NH | 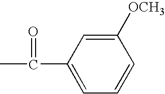 | Br | H | CH₃ | H |
| 561 | NHCH₂CH₂NH | 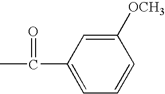 | Br | H | CH₃ | COCH₃ |
| 562 | NHCH₂CH₂NH | 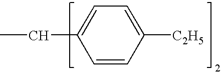 | Br | Br | H | H |
| 563 | NHCH₂CH₂NH | 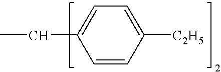 | Br | Br | H | COCH₃ |
| 564 | NHCH₂CH₂NH | 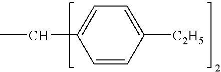 | Br | Br | CH₃ | H |
| 565 | NHCH₂CH₂NH | 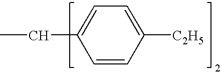 | Br | Br | CH₃ | COCH₃ |
| 566 | piperazinyl | 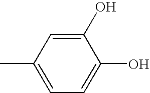 | H | H | H | H |
| 567 | piperazinyl | 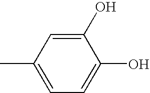 | H | H | H | COCH₃ |
| 568 | piperazinyl | 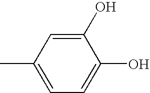 | H | H | CH₃ | H |
| 569 | piperazinyl | 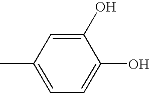 | H | H | CH₃ | COCH₃ |
| 570 | piperazinyl | 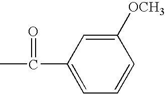 | Br | H | H | H |
| 571 | piperazinyl | 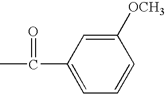 | Br | H | H | COCH₃ |
| 572 | piperazinyl | 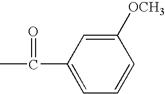 | Br | H | CH₃ | H |
| 573 | piperazinyl | 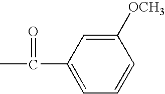 | Br | H | CH₃ | COCH₃ |
| 574 | piperazinyl | 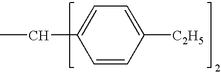 | Br | Br | H | H |
| 575 | piperazinyl | 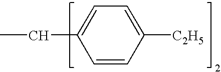 | Br | Br | H | COCH₃ |
| 576 | piperazinyl | 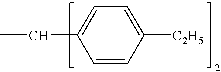 | Br | Br | CH₃ | H |
| 577 | piperazinyl | 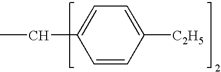 | Br | Br | CH₃ | COCH₃ |

The following Examples #578-#757 of Table IX are highly preferred conjugates composed of dopa-decarboxylase inhibitor compounds and glutamic acid derivatives. These dopa-decarboxylase inhibitors utilized to make these conjugates are benzoic acid type derivatives based on the list of similar compounds described earlier.

TABLE IX

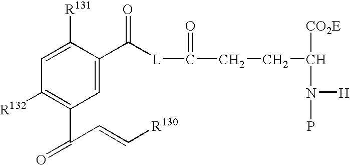

| EXAMPLE | | | | | | |
|---|---|---|---|---|---|---|
| NO. | L | R¹³⁰ | R¹³¹ | R¹³² | E | P |
| 578 | NHNH | H | OH | OH | H | H |
| 579 | NHNH | H | OH | OH | H | COCH₃ |
| 580 | NHNH | H | OH | OH | CH₃ | H |
| 581 | NHNH | H | OH | OH | CH₃ | COCH₃ |
| 582 | NHNH | 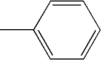 | OH | OH | H | H |
| 583 | NHNH | 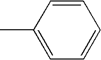 | OH | OH | H | COCH₃ |
| 584 | NHNH | 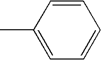 | OH | OH | CH₃ | H |
| 585 | NHNH | 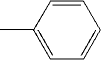 | OH | OH | CH₃ | COCH₃ |
| 586 | NHNH | 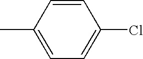 | OH | OH | H | H |
| 587 | NHNH | 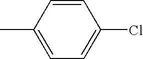 | OH | OH | H | COCH₃ |
| 588 | NHNH | 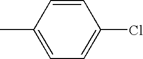 | OH | OH | CH₃ | H |
| 589 | NHNH | 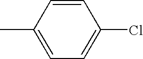 | OH | OH | CH₃ | COCH₃ |
| 590 | NHNH | 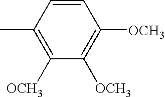 | OCH₃ | OCH₃ | H | H |
| 591 | NHNH | 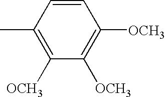 | OCH₃ | OCH₃ | H | COCH₃ |
| 592 | NHNH | 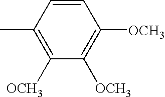 | OCH₃ | OCH₃ | CH₃ | H |
| 593 | NHNH | 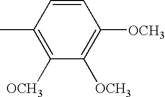 | OCH₃ | OCH₃ | CH₃ | COCH₃ |
| 594 | NHNH | 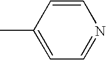 | OCH₃ | OCH₃ | H | H |
| 595 | NHNH | 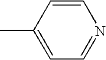 | OCH₃ | OCH₃ | H | COCH₃ |
| 596 | NHNH | 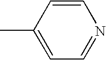 | OCH₃ | OCH₃ | CH₃ | H |
| 597 | NHNH | 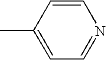 | OCH₃ | OCH₃ | CH₃ | COCH₃ |
| 598 | NHNH | 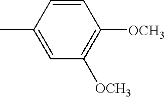 | OCH₃ | OCH₃ | H | H |
| 599 | NHNH | 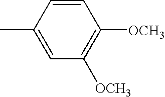 | OCH₃ | OCH₃ | H | COCH₃ |
| 600 | NHNH | 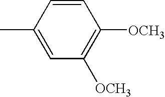 | OCH₃ | OCH₃ | CH₃ | H |
| 601 | NHNH | 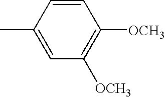 | OCH₃ | OCH₃ | CH₃ | COCH₃ |
| 602 | NHNH | 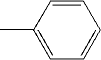 | OCH₃ | OCH₃ | H | H |
| 603 | NHNH | 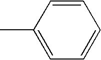 | OCH₃ | OCH₃ | H | COCH₃ |
| 604 | NHNH | 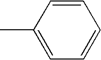 | OCH₃ | OCH₃ | CH₃ | H |
| 605 | NHNH | 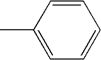 | OCH₃ | OCH₃ | CH₃ | COCH₃ |
| 606 | NHNH | 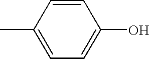 | OH | OH | H | H |
| 607 | NHNH | 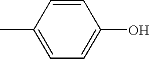 | OH | OH | H | COCH₃ |
| 608 | NHNH | 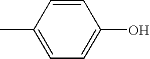 | OH | OH | CH₃ | H |
| 609 | NHNH | 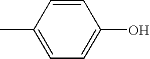 | OH | OH | CH₃ | COCH₃ |
| 610 | NHNH | 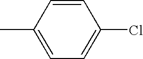 | OCH₃ | OCH₃ | H | H |
| 611 | NHNH | 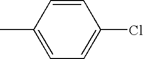 | OCH₃ | OCH₃ | H | COCH₃ |
| 612 | NHNH | 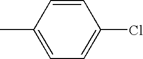 | OCH₃ | OCH₃ | CH₃ | H |
| 613 | NHNH | 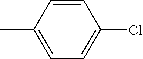 | OCH₃ | OCH₃ | CH₃ | COCH₃ |
| 614 | NHNH | 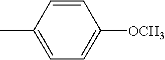 | OCH₃ | OCH₃ | H | H |
| 615 | NHNH | 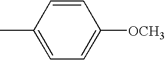 | OCH₃ | OCH₃ | H | COCH₃ |
| 616 | NHNH | 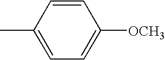 | OCH₃ | OCH₃ | CH₃ | H |
| 617 | NHNH | 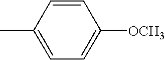 | OCH₃ | OCH₃ | CH₃ | COCH₃ |
| 618 | NHNH | 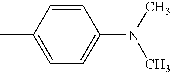 | OCH₃ | OCH₃ | H | H |
| 619 | NHNH | 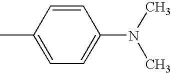 | OCH₃ | OCH₃ | H | COCH₃ |
| 620 | NHNH | 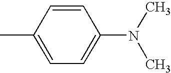 | OCH₃ | OCH₃ | CH₃ | H |
| 621 | NHNH | 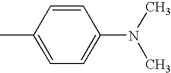 | OCH₃ | OCH₃ | CH₃ | COCH₃ |
| 622 | NHNH | 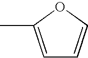 | OH | OH | H | H |
| 623 | NHNH | 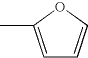 | OH | OH | H | COCH₃ |
| 624 | NHNH | 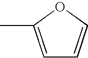 | OH | OH | CH₃ | H |
| 625 | NHNH | 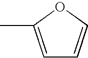 | OH | OH | CH₃ | COCH₃ |
| 626 | NHNH | 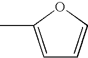 | OCH₃ | OCH₃ | H | H |
| 627 | NHNH | 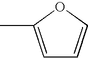 | OCH₃ | OCH₃ | H | COCH₃ |
| 628 | NHNH | 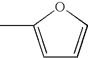 | OCH₃ | OCH₃ | CH₃ | H |
| 629 | NHNH | 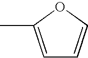 | OCH₃ | OCH₃ | CH₃ | COCH₃ |
| 630 | NHNH | 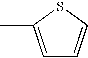 | OCH₃ | OCH₃ | H | H |
| 631 | NHNH | 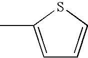 | OCH₃ | OCH₃ | H | COCH₃ |
| 632 | NHNH | 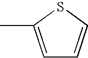 | OCH₃ | OCH₃ | CH₃ | H |
| 633 | NHNH | 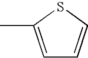 | OCH₃ | OCH₃ | CH₃ | COCH₃ |
| 634 | NHNH | 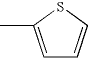 | OH | OH | H | H |
| 635 | NHNH | 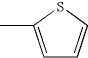 | OH | OH | H | COCH₃ |
| 636 | NHNH | 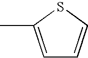 | OH | OH | CH₃ | H |
| 637 | NHNH | 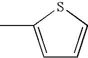 | OH | OH | CH₃ | COCH₃ |
| 638 | NHCH₂CH₂NH | H | OH | OH | H | H |
| 639 | NHCH₂CH₂NH | H | OH | OH | H | COCH₃ |
| 640 | NHCH₂CH₂NH | H | OH | OH | CH₃ | H |
| 641 | NHCH₂CH₂NH | H | OH | OH | CH₃ | COCH₃ |
| 642 | NHCH₂CH₂NH | 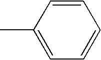 | OH | OH | H | H |
| 643 | NHCH₂CH₂NH | 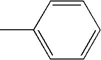 | OH | OH | H | COCH₃ |
| 644 | NHCH₂CH₂NH | 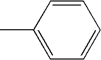 | OH | OH | CH₃ | H |
| 645 | NHCH₂CH₂NH | 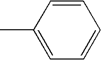 | OH | OH | CH₃ | COCH₃ |
| 646 | NHCH₂CH₂NH | 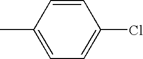 | OH | OH | H | H |
| 647 | NHCH₂CH₂NH | 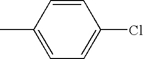 | OH | OH | H | COCH₃ |
| 648 | NHCH₂CH₂NH | 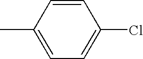 | OH | OH | CH₃ | H |
| 649 | NHCH₂CH₂NH | 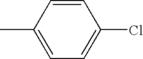 | OH | OH | CH₃ | COCH₃ |
| 650 | NHCH₂CH₂NH | 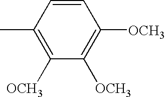 | OCH₃ | OCH₃ | H | H |
| 651 | NHCH₂CH₂NH | 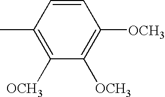 | OCH₃ | OCH₃ | H | COCH₃ |
| 652 | NHCH₂CH₂NH | 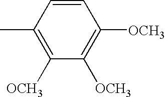 | OCH₃ | OCH₃ | CH₃ | H |
| 653 | NHCH₂CH₂NH | 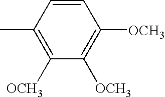 | OCH₃ | OCH₃ | CH₃ | COCH₃ |
| 654 | NHCH₂CH₂NH | 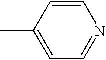 | OCH₃ | OCH₃ | H | H |
| 655 | NHCH₂CH₂NH | 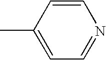 | OCH₃ | OCH₃ | H | COCH₃ |
| 656 | NHCH₂CH₂NH | 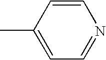 | OCH₃ | OCH₃ | CH₃ | H |
| 657 | NHCH₂CH₂NH | 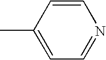 | OCH₃ | OCH₃ | CH₃ | COCH₃ |
| 658 | NHCH₂CH₂NH | 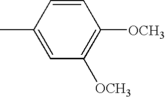 | OCH₃ | OCH₃ | H | H |
| 659 | NHCH₂CH₂NH | 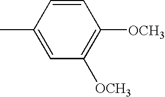 | OCH₃ | OCH₃ | H | COCH₃ |
| 660 | NHCH₂CH₂NH | 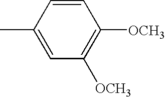 | OCH₃ | OCH₃ | CH₃ | H |
| 661 | NHCH₂CH₂NH | 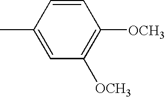 | OCH₃ | OCH₃ | CH₃ | COCH₃ |
| 662 | NHCH₂CH₂NH | 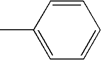 | OCH₃ | OCH₃ | H | H |
| 663 | NHCH₂CH₂NH | 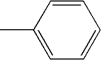 | OCH₃ | OCH₃ | H | COCH₃ |
| 664 | NHCH₂CH₂NH | 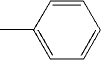 | OCH₃ | OCH₃ | CH₃ | H |
| 665 | NHCH₂CH₂NH | 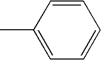 | OCH₃ | OCH₃ | CH₃ | COCH₃ |
| 666 | NHCH₂CH₂NH | 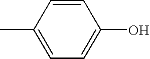 | OH | OH | H | H |
| 667 | NHCH₂CH₂NH | 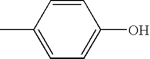 | OH | OH | H | COCH₃ |
| 668 | NHCH₂CH₂NH | 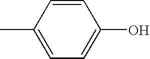 | OH | OH | CH₃ | H |
| 669 | NHCH₂CH₂NH | 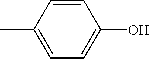 | OH | OH | CH₃ | COCH₃ |
| 670 | NHCH₂CH₂NH | 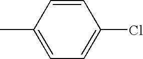 | OCH₃ | OCH₃ | H | H |
| 671 | NHCH₂CH₂NH | 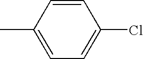 | OCH₃ | OCH₃ | H | COCH₃ |
| 672 | NHCH₂CH₂NH | 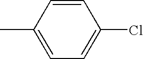 | OCH₃ | OCH₃ | CH₃ | H |
| 673 | NHCH₂CH₂NH | 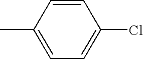 | OCH₃ | OCH₃ | CH₃ | COCH₃ |
| 674 | NHCH₂CH₂NH | 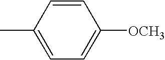 | OCH₃ | OCH₃ | H | H |
| 675 | NHCH₂CH₂NH | 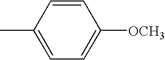 | OCH₃ | OCH₃ | H | COCH₃ |
| 676 | NHCH₂CH₂NH | 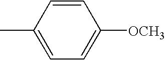 | OCH₃ | OCH₃ | CH₃ | H |
| 677 | NHCH₂CH₂NH | 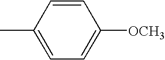 | OCH₃ | OCH₃ | CH₃ | COCH₃ |
| 678 | NHCH₂CH₂NH | 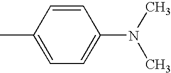 | OCH₃ | OCH₃ | H | H |
| 679 | NHCH₂CH₂NH | 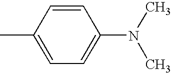 | OCH₃ | OCH₃ | H | COCH₃ |
| 680 | NHCH₂CH₂NH | 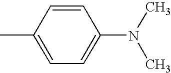 | OCH₃ | OCH₃ | CH₃ | H |
| 681 | NHCH₂CH₂NH | 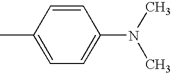 | OCH₃ | OCH₃ | CH₃ | COCH₃ |
| 682 | NHCH₂CH₂NH | 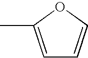 | OH | OH | H | H |
| 683 | NHCH₂CH₂NH | 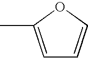 | OH | OH | H | COCH₃ |
| 684 | NHCH₂CH₂NH | 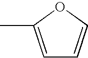 | OH | OH | CH₃ | H |
| 685 | NHCH₂CH₂NH | 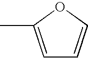 | OH | OH | CH₃ | COCH₃ |
| 686 | NHCH₂CH₂NH | 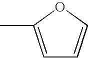 | OCH₃ | OCH₃ | H | H |
| 687 | NHCH₂CH₂NH | 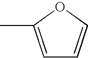 | OCH₃ | OCH₃ | H | COCH₃ |
| 688 | NHCH₂CH₂NH | 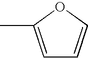 | OCH₃ | OCH₃ | CH₃ | H |
| 689 | NHCH₂CH₂NH | 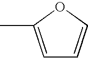 | OCH₃ | OCH₃ | CH₃ | COCH₃ |
| 690 | NHCH₂CH₂NH | 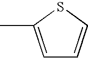 | OCH₃ | OCH₃ | H | H |
| 691 | NHCH₂CH₂NH | 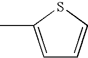 | OCH₃ | OCH₃ | H | COCH₃ |
| 692 | NHCH₂CH₂NH | 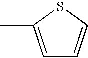 | OCH₃ | OCH₃ | CH₃ | H |
| 693 | NHCH₂CH₂NH | 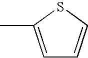 | OCH₃ | OCH₃ | CH₃ | COCH₃ |
| 694 | NHCH₂CH₂NH | 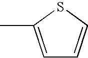 | OH | OH | H | H |
| 695 | NHCH₂CH₂NH | 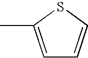 | OH | OH | H | COCH₃ |
| 696 | NHCH₂CH₂NH | 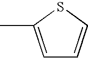 | OH | OH | CH₃ | H |
| 697 | NHCH₂CH₂NH | 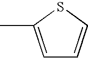 | OH | OH | CH₃ | COCH₃ |
| 698 | piperazinyl | H | OH | OH | H | H |
| 699 | piperazinyl | H | OH | OH | H | COCH₃ |
| 700 | piperazinyl | H | OH | OH | CH₃ | H |
| 701 | piperazinyl | H | OH | OH | CH₃ | COCH₃ |
| 702 | piperazinyl | 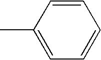 | OH | OH | H | H |
| 703 | piperazinyl | 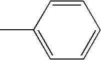 | OH | OH | H | COCH₃ |
| 704 | piperazinyl | 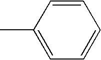 | OH | OH | CH₃ | H |
| 705 | piperazinyl | 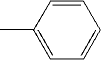 | OH | OH | CH₃ | COCH₃ |
| 706 | piperazinyl | 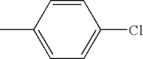 | OH | OH | H | H |
| 707 | piperazinyl | 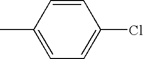 | OH | OH | H | COCH₃ |
| 708 | piperazinyl | 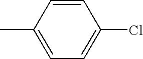 | OH | OH | CH₃ | H |
| 709 | piperazinyl | 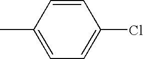 | OH | OH | CH₃ | COCH₃ |
| 710 | piperazinyl | 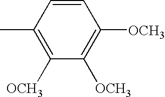 | OCH₃ | OCH₃ | H | H |
| 711 | piperazinyl | 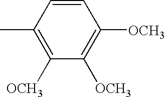 | OCH₃ | OCH₃ | H | COCH₃ |
| 712 | piperazinyl | 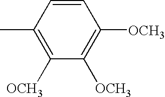 | OCH₃ | OCH₃ | CH₃ | H |
| 713 | piperazinyl | 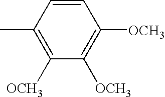 | OCH₃ | OCH₃ | CH₃ | COCH₃ |
| 714 | piperazinyl | 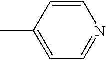 | OCH₃ | OCH₃ | H | H |
| 715 | piperazinyl | 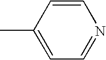 | OCH₃ | OCH₃ | H | COCH₃ |
| 716 | piperazinyl | 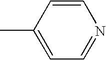 | OCH₃ | OCH₃ | CH₃ | H |
| 717 | piperazinyl | 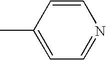 | OCH₃ | OCH₃ | CH₃ | COCH₃ |
| 718 | piperazinyl | 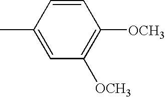 | OCH₃ | OCH₃ | H | H |
| 719 | piperazinyl | 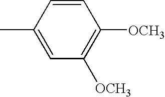 | OCH₃ | OCH₃ | H | COCH₃ |
| 720 | piperazinyl | 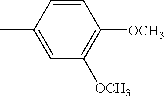 | OCH₃ | OCH₃ | CH₃ | H |
| 721 | piperazinyl | 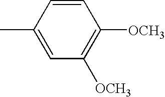 | OCH₃ | OCH₃ | CH₃ | COCH₃ |
| 722 | piperazinyl | 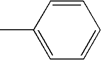 | OCH₃ | OCH₃ | H | H |
| 723 | piperazinyl | 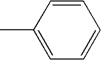 | OCH₃ | OCH₃ | H | COCH₃ |
| 724 | piperazinyl | 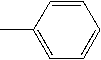 | OCH₃ | OCH₃ | CH₃ | H |
| 725 | piperazinyl | 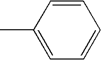 | OCH₃ | OCH₃ | CH₃ | COCH₃ |
| 726 | piperazinyl | 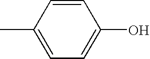 | OH | OH | H | H |
| 727 | piperazinyl | 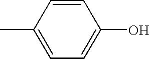 | OH | OH | H | COCH₃ |
| 728 | piperazinyl | 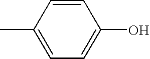 | OH | OH | CH₃ | H |
| 729 | piperazinyl | 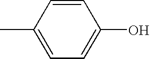 | OH | OH | CH₃ | COCH₃ |
| 730 | piperazinyl | 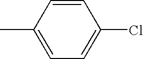 | OCH₃ | OCH₃ | H | H |
| 731 | piperazinyl | 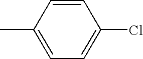 | OCH₃ | OCH₃ | H | COCH₃ |
| 732 | piperazinyl | 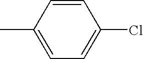 | OCH₃ | OCH₃ | CH₃ | H |
| 733 | piperazinyl | 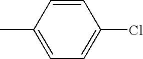 | OCH₃ | OCH₃ | CH₃ | COCH₃ |
| 734 | piperazinyl | 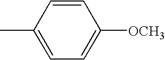 | OCH₃ | OCH₃ | H | H |
| 735 | piperazinyl | 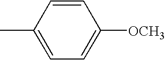 | OCH₃ | OCH₃ | H | COCH₃ |
| 736 | piperazinyl | 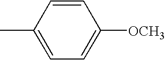 | OCH₃ | OCH₃ | CH₃ | H |
| 737 | piperazinyl | 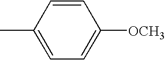 | OCH₃ | OCH₃ | CH₃ | COCH₃ |
| 738 | piperazinyl | 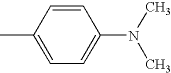 | OCH₃ | OCH₃ | H | H |
| 739 | piperazinyl | 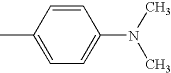 | OCH₃ | OCH₃ | H | COCH₃ |
| 740 | piperazinyl | 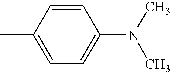 | OCH₃ | OCH₃ | CH₃ | H |
| 741 | piperazinyl | 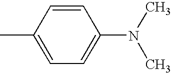 | OCH₃ | OCH₃ | CH₃ | COCH₃ |
| 742 | piperazinyl | 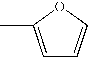 | OH | OH | H | H |
| 743 | piperazinyl | 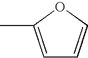 | OH | OH | H | COCH₃ |
| 744 | piperazinyl | 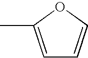 | OH | OH | CH₃ | H |
| 745 | piperazinyl | 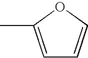 | OH | OH | CH₃ | COCH₃ |
| 746 | piperazinyl | 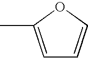 | OCH₃ | OCH₃ | H | H |
| 747 | piperazinyl | 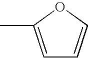 | OCH₃ | OCH₃ | H | COCH₃ |
| 748 | piperazinyl | 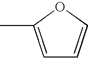 | OCH₃ | OCH₃ | CH₃ | H |
| 749 | piperazinyl | 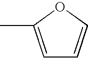 | OCH₃ | OCH₃ | CH₃ | COCH₃ |
| 750 | piperazinyl | 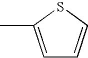 | OCH₃ | OCH₃ | H | H |
| 751 | piperazinyl | 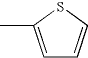 | OCH₃ | OCH₃ | H | COCH₃ |
| 752 | piperazinyl | 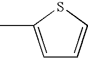 | OCH₃ | OCH₃ | CH₃ | H |
| 753 | piperazinyl | 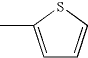 | OCH₃ | OCH₃ | CH₃ | COCH₃ |
| 754 | piperazinyl | 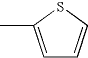 | OH | OH | H | H |
| 755 | piperazinyl | 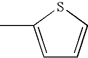 | OH | OH | H | COCH₃ |
| 756 | piperazinyl | 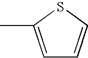 | OH | OH | CH₃ | H |
| 757 | piperazinyl | 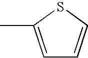 | OH | OH | CH₃ | COCH₃ |

The following Examples #758-#809 of Table X are highly preferred conjugates composed of dopa-decarboxylase inhibitor compounds and glutamic acid derivatives. These dopa-decarboxylase inhibitors utilized to-make these conjugates are propenoic acid derivatives based on the list of similar compounds described earlier.

TABLE X

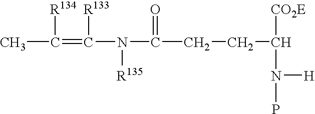

| EXAMPLE | | | | | |
|---|---|---|---|---|---|
| NO. | R¹³³ | R¹³⁴ | R¹³⁵ | E | P |
| 758 | H | 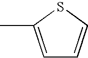 | H | H | H |
| 759 | H | 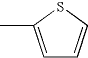 | H | H | COCH₃ |
| 760 | H | 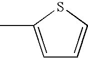 | H | CH₃ | H |
| 761 | H | 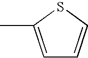 | H | CH₃ | COCH₃ |
| 762 | CH₃ | 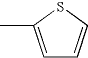 | H | H | H |
| 763 | CH₃ | 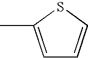 | H | H | COCH₃ |
| 764 | CH₃ | 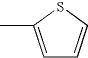 | H | CH₃ | H |
| 765 | CH₃ | 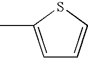 | H | CH₃ | COCH₃ |
| 766 | H | 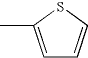 | CH₃ | H | H |
| 767 | H | 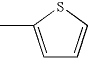 | CH₃ | H | COCH₃ |
| 768 | H | 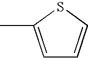 | CH₃ | CH₃ | H |
| 769 | H | 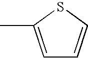 | CH₃ | CH₃ | COCH₃ |
| 770 | H | 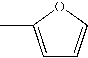 | H | H | H |
| 771 | H | 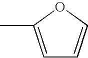 | H | H | COCH₃ |
| 772 | H | 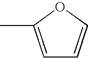 | H | CH₃ | H |
| 773 | H | 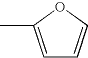 | H | CH₃ | COCH₃ |
| 774 | CH₃ | 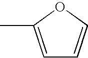 | H | H | H |
| 775 | CH₃ | 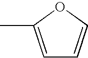 | H | H | COCH₃ |
| 776 | CH₃ | 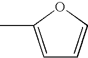 | H | CH₃ | H |
| 777 | CH₃ | 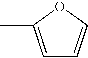 | H | CH₃ | COCH₃ |
| 778 | H | 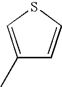 | H | H | H |
| 779 | H | 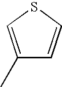 | H | H | COCH₃ |
| 780 | H | 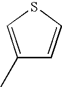 | H | CH₃ | H |
| 781 | H | 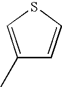 | H | CH₃ | COCH₃ |
| 782 | CH₃ | 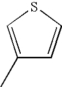 | H | H | H |
| 783 | CH₃ | 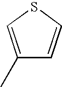 | H | H | COCH₃ |
| 784 | CH₃ | 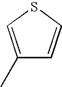 | H | CH₃ | H |
| 785 | CH₃ | 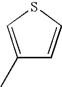 | H | CH₃ | COCH₃ |
| 786 | H | 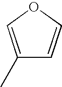 | H | H | H |
| 787 | H | 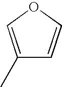 | H | H | COCH₃ |
| 788 | H | 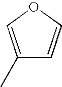 | H | CH₃ | H |
| 789 | H | 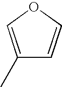 | H | CH₃ | COCH₃ |
| 790 | CH₃ | 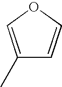 | H | H | H |
| 791 | CH₃ | 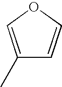 | H | H | COCH₃ |
| 792 | CH₃ | 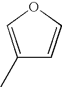 | H | CH₃ | H |
| 793 | CH₃ | 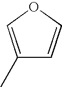 | H | CH₃ | COCH₃ |
| 794 | H | 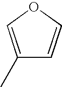 | CH₃ | H | H |
| 795 | H | 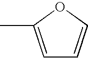 | CH₃ | H | COCH₃ |
| 796 | H | 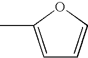 | CH₃ | CH₃ | H |
| 797 | H | 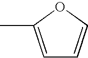 | CH₃ | CH₃ | COCH₃ |
| 798 | H | 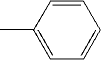 | H | H | H |
| 799 | H | 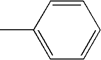 | H | H | COCH₃ |
| 800 | H | 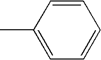 | H | CH₃ | H |
| 801 | H | 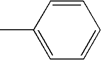 | H | CH₃ | COCH₃ |
| 802 | CH₃ | 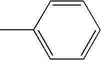 | H | H | H |
| 803 | CH₃ | 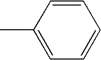 | H | H | COCH₃ |
| 804 | CH₃ | 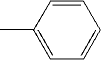 | H | CH₃ | H |
| 805 | CH₃ | 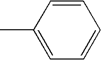 | H | CH₃ | COCH₃ |
| 806 | H | 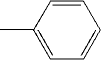 | CH₃ | H | H |
| 807 | H | 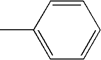 | CH₃ | H | COCH₃ |
| 808 | H | 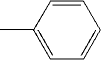 | CH₃ | CH₃ | H |
| 809 | H | 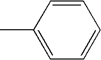 | CH₃ | CH₃ | COCH₃ |

The following Examples #810-#833 of Table XI are highly preferred conjugates composed of dopa-decarboxylase inhibitor compounds and glutamic acid derivatives. These dopa-decarboxylase inhibitors utilized to make these conjugates are embraced by generic Formula IX, above.

TABLE XI

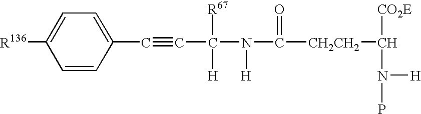

| EXAMPLE | | | | |
|---|---|---|---|---|
| NO. | R⁶⁷ | R¹³⁶ | E | P |
| 810 | H | H | H | H |
| 811 | H | H | H | COCH₃ |
| 812 | H | H | CH₃ | H |
| 813 | H | H | CH₃ | COCH₃ |
| 814 | H | OH | H | H |
| 815 | H | OH | H | COCH₃ |
| 816 | H | OH | CH₃ | H |
| 817 | H | OH | CH₃ | COCH₃ |
| 818 | H | OCH₃ | H | H |
| 819 | H | OCH₃ | H | COCH₃ |
| 820 | H | OCH₃ | CH₃ | H |
| 821 | H | OCH₃ | CH₃ | COCH₃ |
| 822 | CH₃ | H | H | H |
| 823 | CH₃ | H | H | COCH₃ |
| 824 | CH₃ | H | CH₃ | H |
| 825 | CH₃ | H | CH₃ | COCH₃ |
| 826 | CH₃ | OH | H | H |
| 827 | CH₃ | OH | H | COCH₃ |
| 828 | CH₃ | OH | CH₃ | H |
| 829 | CH₃ | OH | CH₃ | COCH₃ |
| 830 | CH₃ | OCH₃ | H | H |
| 831 | CH₃ | OCH₃ | H | COCH₃ |
| 832 | CH₃ | OCH₃ | CH₃ | H |
| 833 | CH₃ | OCH₃ | CH₃ | COCH₃ |

The following Examples #834-#857 of Table XII are highly preferred conjugates composed of dopa-decarboxylase inhibitor compounds and glutamic acid derivatives. These dopa-decarboxylase inhibitors utilized to make these conjugates are embraced by generic Formula IX, above.

TABLE XII

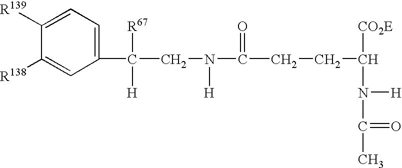

| EXAMPLE | | | | | |
|---|---|---|---|---|---|
| NO. | R¹³⁸ | R¹³⁹ | R⁶⁷ | E | P |
| 834 | H | H | C≡CH | H | H |
| 835 | H | H | C≡CH | H | COCH₃ |
| 836 | H | H | C≡CH | CH₃ | H |
| 837 | H | H | C≡CH | CH₃ | COCH₃ |
| 838 | OH | H | C≡CH | H | H |
| 839 | OH | H | C≡CH | H | COCH₃ |
| 840 | OH | H | C≡CH | CH₃ | H |
| 841 | OH | H | C≡CH | CH₃ | COCH₃ |
| 842 | H | OH | C≡CH | H | H |
| 843 | H | OH | C≡CH | H | COCH₃ |
| 844 | H | OH | C≡CH | CH₃ | H |
| 845 | H | OH | C≡CH | CH₃ | COCH₃ |
| 846 | H | H | CH═CH₂ | H | H |
| 847 | H | H | CH═CH₂ | H | COCH₃ |
| 848 | H | H | CH═CH₂ | CH₃ | H |
| 849 | H | H | CH═CH₂ | CH₃ | COCH₃ |
| 850 | OH | H | CH═CH₂ | H | H |
| 851 | OH | H | CH═CH₂ | H | COCH₃ |
| 852 | OH | H | CH═CH₂ | CH₃ | H |
| 853 | OH | H | CH═CH₂ | CH₃ | COCH₃ |
| 854 | H | OH | CH═CH₂ | H | H |
| 855 | H | OH | CH═CH₂ | H | COCH₃ |
| 856 | H | OH | CH═CH₂ | CH₃ | H |
| 857 | H | OH | CH═CH₂ | CH₃ | COCH₃ |

The following Examples #858-#1857 comprise five classes of highly preferred conjugates composed of dopamine-β-hydroxylase inhibitor compounds and glutamic acid derivatives. Examples #858-#863 are descriptions of specific preparations of such conjugates. Examples #864-#1857, as shown in Tables XIII-XVII, may be prepared by procedures shown in these specific examples and in the foregoing general synthetic procedures of Schemes 1-7.

### EXAMPLE 858

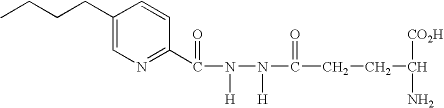

L-glutamic acid, 5-{[(5-butyl-2-pyridinyl)carbonyl]hydrazide}

Step. 1: Preparation of 5-n-Butylpicolinic (Fusaric) Acid Hydrazide

A solution of 36.0 g (0.20 mol) of fusaric acid (Sigma) in 800 ml of absolute methanol was cooled to −10° C. by means of an ice/methanol bath and 120 ml (199 g, 1.67 mol) of SOCl₂ was added dropwise over a 1 hr period. The reaction was allowed to slowly warm to ambient temperature and then stirred at reflux for 72 hr. The reaction was concentrated; the addition of 100 ml of toluene (twice) followed by reconcentration insured the complete removal of any unreacted SOCl₂. The viscous syrup thus formed was dried in vacuo (0.01 mm) overnight prior to treatment with cold NaHCO₃ (sat). The ester was extracted with ether and dried (MgSO₄). Concentration gave 32.3 g (83%) of crude methyl fusarate which was redissolved in 100 ml of absolute methanol and cooled to 0° C. Under a nitrogen atmosphere, 5.5 ml (0.174 mol) of anhydrous hydrazine was slowly added by syringe. The reaction was allowed to slowly warm to ambient temperature and stir overnight. The methanol was removed and the yellow-brown residue was dried in vacuo (0.01 m) overnight where it solidified producing 31.7 g (98%) based on ester) of crude hydrazide. Recrystallization from ether/hexane gave colorless needles: mp 51-53° C. NMR (CDCl₃) δ 0.95 (t, J=7 Hz, 3H, CH₂CH₃); 1.30-1.45 (m, 2H, CH₂CH₃); 1.55-1.70 (m, 2H, CH₂CH₂CH₂); 2.67 (t, J=7 Hz, 2H, ArCH₂); 7.65 (d of d, J_{3,4}=7 Hz and J_{4,6}=2 Hz, 1H, ArH); 8.05 (d, J3,4=7 Hz, 1H, ArH); 8.37 (d, 1H, ArH, J_{4,6}=2 Hz); HRMS. Calcd for M+H: 194.1270. Found: 194.1293.

Step 2: Preparation of L-glutamic acid, 5-{[(5-butyl-2-pyridinyl)carbonyl]hydrazide}.

A solution of 7.27 g (24.0 mmol) of Boc-L-γglutamic acid-α-t-butyl ester (BACHEM) in 150 ml of anhydrous THF was cooled to 0° C. under static nitrogen and treated with 2.7 ml (2.46 g, 24.4 mmol) of anhydrous N-methyl morpholine. The mixture was then slowly treated with 3.1 ml (3.26 g, 23.9 mmol) of isobutyl chloroformate and allowed to stir for 1 hr prior to the dropwise addition of a solution of 3.86 g (20.0 mmol) of fusaric acid hydrazide from step 1 in 30 ml of anhydrous THF. The reaction mixture was stirred at 0° C. for 2 hr and then allowed to warm to ambient temperature and stir overnight. The N-methylmorpholine hydrochloride was removed by filtration and the filtrate concentrated in vacuo to give 11.5 g of crude product which was a colorless glass. This material was dissolved in 50 ml of CH₂Cl₂ and treated with 50 ml of CF₃CO₂H. After 4 hr at ambient temperataure, the volitiles were removed im vacuo. The addition of acetonitrile caused the product to precipitate producing 3.97 g (46%) of colorless material: mp 162-164° C. (dec.); NMR (DMSO-d₆) δ 1.90 (t, J=7 Hz, 3H, CH₂CH₃); 1.30-1.45 (m, 2H, CH₂CH₃); 1.50-1.65 (m, 2H, CH₂CH₂CH₂); 2.00-2.20 (m, 1H, CH₂CH); 2.30-2.50 (m, 1H, CH₂CH); 2.70 (t, J=7 Hz, 2H, ArCH₂); 3.60 (t, J=7 Hz, 2H, COCH₂); 3.95-4.05 (M, 1H, CH₂CH); 7.85 (d of d, J_{3,4}=7 Hz and J_{4,6}=2 Hz, 1H, ArH); 7.95 (d, J_{3,4=}7 Hz, 1H, ArH); 8.55 (d, J_{4,6=2} Hz, 1H, ArH).

### EXAMPLE 859

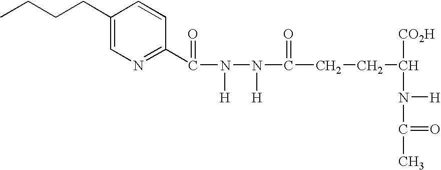

N-acetyl-L-alutamic acid, 5-[(5-butyl-2-pyridinyl)-carbonyl]hydrazide

A suspension of 2.85 g (6.54 mmol) of the compound of Example 858 in CH₃CN/H₂O (1:1) was treated with 2 equiv. of 1 M K₂CO₃ at 0° C. With efficient stirring, 1 ml (10.6 mmol) of acetic anhydride and 11 ml (11 mmol) of 1 M K₂CO₃ were added every 10 min for 1 hr; since the product is soluble, the mixture became homogenous as the reaction proceeded. The reaction mixture was stirred for 1 hr, filtered, and the filtrate cooled to 0° C. The pH was adjusted to pH 4 by the careful addition of cold dilute HCl. All volitiles were removed in vacuo and the product dissolved in ethanol. Recrystallization from ethanol/petroleum ether produced 2.16 g (69%) of colorless material: mp 191.5-192.0° C.; NMR (D₂O and NaOD) δ 0.85 (t, J=7 Hz, 3H, CH₂CH₃); 1.20-1.35 (m, 2H, CH₂CH₃); 1.55-1.70 (m, 2H, CH₂CH₂CH₂); 1.95-2.10 (m, 1H, CH₂CH); 2.05 (s, 3H, COCH₃); 2.20-2.35 (m, 1H, CH₂CH); 2.45 (t, J=7 Hz, 2H, COCH₂); 2.75 (t, 2H, ArCH₂); 3.45-3.55 (m, 1H, CH₂CH); 8.05 (s, 2H, ArH); 8.55 (s, 1H, ArH); HRMS. Calcd for M+H: 365.1825. Found 365.1860.

Anal. Calcd. for C₁₇H₂₄N₄O₅: C, 55.98; H, 6.58; N, 15.36. Found: C, 55.96; H, 6.64; N, 15.30.

### EXAMPLE 860

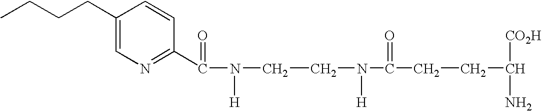

N-[2-[[(5-butyl-2-pyridinyl)carbonyl]amino]ethyl]-L-glutamine.

Step 1: Preparation of the ethylene diamine amide of fusaric acid.

A solution of 7.8 g (130 mmol) of ethylene diamine in 400 mL of anhydrous THF under nitrogen was treated with 27 mmol of n-butyllithium at 0° C. The solution was allowed to stir for 30 min and was treated with 5.0 g (26 mmol) of neat methyl fusarate (from step 1 of Example 690) by syringe. The reaction was kept at 0° C. for 2 hr and stirred at ambient temperature overnight. The reaction was quenched with water, filtered, and concentrated in vacuo. Purification by silica gel chromatography gave 3.8 g (66%) of pure amide: NMR (DMSO-d₆) δ 0.90 (t, J=8 Hz, 3H), 1.23-1.38 (m, 2H), 1.52-1.64 (m, 2H), 2.67 (t, J=8 Hz, 2H), 2.74 (t, J=8 Hz, 2H), 3.18-3.30 (br s, 2H), 3.34 (q, J=8 Hz, 2H), 7.82 d of d, J=9 Hz and 2 Hz, 1H), 7.96 (d, J=9 Hz, 1H), 8.47 (d, J=2 Hz, 1H), 8.75 (t, J=8 Hz, 1H).

Step 2: Preparation of N-[2-[[(5-butyl-2-pyridinyl)carbonyl]amino]ethyl]-L-glutamine.

Under nitrogen, a solution of 26.8 g (88.5 mmol) of N-Boc-L-γ-glutamic acid-α-t-butyl ester (BACHEM) in 125 mL of methylene chloride was treated with 9.14 g (44.3 mmol) of solid dicyclohexylcarbodiimide (DCC). The reaction was allowed to stir for 2 hr prior to filtration under a nitrogen atmosphere. The anhydride solution was slowly added to a solution of 8.5 g (38.5 mmol) of the ethylene diamine amide from step 1 in 100 mL of methylene chloride. The reaction was allowed to stir overnight and was concentrated in vacuo. The residue was dissolved in ethyl acetate, washed with 1 M K₂CO₃ followed by water, dried (MgSO₄) and reconcentrated in vacuo to give the protected coupled product; a solution of this material in 250 mL of methylene chloride was cooled to 0° C. and treated with 250 mL of trifluoroacetic acid (TFA). The reaction was allowed to warm to ambient temperature and stir overnight; the course of the reaction was monitored by analytical LC. Concentration in vacuo gave N-[2-[[(5-butyl-2-pyridinyl)carbonyl]amino]ethyl]-L-glutamine.

### EXAMPLE 861

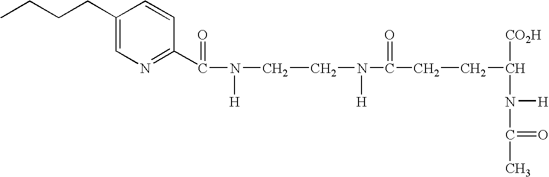

N²-acetyl-N-[2-[[(5-butyl-2-pyridinyl)carbonyl]amino]ethyl]-L-glutamine.

The compound of Example 860 was dissolved in 150 mL of acetonitrile/water (1:1) and the pH adjusted to 9 with 2 M K₂CO₃. The solution was cooled to 0° C. and 2.27 mL (24 mmol) of acetic anhydride and 12 mL (24 mmol) of 2 M K₂CO₃ was added every 30 min. for 5 h; the pH was maintained at 9 and the reaction temperature kept below 5° C. After the last addition, the reaction was allowed to warm to ambient temperature overnight. The pH was adjusted to 3 with 3 M HCl and concentrated to 300 mL. Purification by reverse phase chromatography (Waters Deltaprep-3000) using isocractic 30% acetonitrile/water (0.05% TFA) gave 7.8 g (52% overall yield from the amide of step 1) of colorless product; an analytical sample was recrystallized from acetonitrile and then water: mp 156-158° C.; Anal. Calcd for C₁₉H₂₈N₄O₅.0.83 TFA: C, 57.32; H, 7.00; N, 13,96; F, 1.14%. Found: C, 57.22; H, 7.07; N, 13.88; F, 1.07.

### EXAMPLE 862

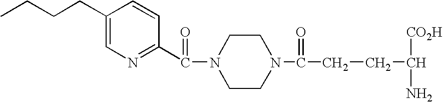

2-amino-5-[4-[(5-butyl-2-pyridinyl)carbonyl]-1-piperazinyl]-5-oxopentanoic acid.

Step 1: Preparation of the Piperizine Amide of Fusaric Acid.

A solution of 11.20 g (130 mmol) of piperazine in 400 mL of anhydrous THF under nitrogen was treated with 27.3 mmol of n-buytyllithium at 0° C. The solution was allowed to stir for 30 min and was treated with 5.0 g (26 mmol) of neat methyl fusarate (from step 1 of Example 690) by syringe. The reaction was kept at 0° C. for 2 hr and stirred at ambient temperature overnight. The reaction was quenched with water, filtered, and concentrated in vacuo. Purification by silica gel chromatography using chloroform/methanol (70:30) gave 5.82 g (90%) of pure amide: NMR (CDCl₃)δ 0.94 (t, J=8 Hz, 3H), 1.28-1.45 (m, 2H), 1.55-1.67 (m, 2H), 1.66-1.72 (br s, 1H), 2.64 (t, J=8 Hz, 2H), 2.86 (t, J=6 Hz, 2H), 2.97 (t, J=6 Hz, 2H), 3.58 (t, J=6 Hz, 2H) 3.77 (t, J=6 Hz, 2H), 7.54-7.63 (m, 2H), 8.37-8.43 (br s, 1H).

Step 2: Preparation of 2-amino-5-[4-[(5-butyl-2-pyridinyl)carbonyl]-1-piperazinyl]-5-oxopentanoic acid.

Under nitrogen, a solution of 17.4 g (57 mmol) of N-Boc-L-γ-glutamic acid-α-t-butyl ester (BACHEM) in 100 mL of anhydrous THF was treated with 5.57 g (27 mmol) of solid dicyclohexylcarbodiimide (DCC). The reaction was allowed to stir for 2 hr prior to filtration under a nitrogen atmosphere. The anhydride solution was slowly added to a solution of 5.82 g (23.5 mmol) of the piperazine amide from step 1 in 50 mL of anhydrous THF. The reaction was allowed to stir overnight and was concentrated in vacuo. The residue was dissolved in ethyl acetate, washed with 1 M K₂CO₃ followed by water, dried (MgSO₄), and reconcentrated in vacuo to give the protected coupled product; a solution of this material in 150 mL of methylene chloride was cooled to 0° C. and treated with 150 mL of trifluoroacetic acid (TFA) under nitrogen. The reaction was allowed to warm to ambient temperature and stir overnight; the course of the reaction was monitored by analytical LC. Concentration in gave 2-amino-5-[4-[(5-butyl-2-pyridinyl)carbonyl]-1-piperazinyl]-5-oxopentanoic acid.

### EXAMPLE 863

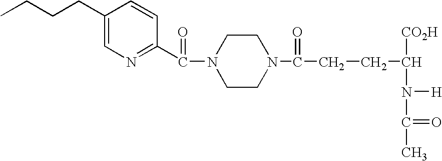

2-(acetylamino)-5-(4-[(5-butyl-2-pyridinyl)carbonyl]-1-piperazinyl]-5-oxopentanoic acid.

The compound of Example 862 was dissolved in 150 mL of acetonitrile/water (1:1) and the pH adjusted to 9 with 1 M K₂CO₃. The solution was cooled to 0° C. and 2.36 mL (25 mmol) of acetic anhydride and 25 mL (25 mmol) of 1 M K₂CO₃ was added every 30 min. for 5 h; the pH was maintained at 9 and the reaction temperature kept below 5° C. After the last addition, the reaction was allowed to warm to ambient temperature overnight. The pH was adjusted to 4 with 3 M HCl and concentrated to 300 mL. Purification by reverse phase chromatography (Waters Deltaprep-3000) using isocratic 25% acetonitrile/water (0.05% TFA) gave 8.13 g (78%) of colorless product: MS (FAB) m/e (rel intensity) 419 (100), 258 (10), 248 (37), 205 (28); HRMS. Calcd for M+H: 419.2294. Found: 419.2250.

### EXAMPLE 864

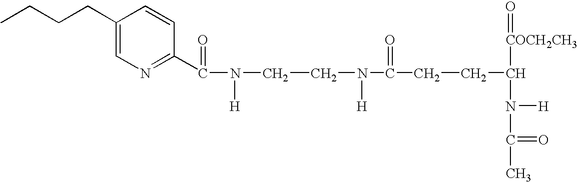

N²-acetyl-N-[2-[[5-butyl-2-pyridinyl)carbonyl]amino]ethyl]-L-glutamine, Ethyl Ester.

A suspension of 57.77 g (0.133 mol) of the compound of Example 858 in CH₃CN/H₂O (1:1) was treated with 2 equivalents of 1 M K₂CO₃ at 0° C. With efficient stirring, 133 mL (0.133 mol) of 1 M K₂CO₃ and 12.5 mL (0.133 mol) of acetic anhydride were added every thirty minutes for 5 h, until a total of 10 equivalents of 1 M K₂CO₃ and acetic anhydride had been added. The reaction was kept at 0° C. for

4 h then allowed to warm to room temperature overnight. The reaction mixture was filtered, the filtrate cooled to 0° C., and the pH adjusted to pH 4 by the careful addition of cold dilute HCl. All volatiles were removed in vacuo. The product was dissolved in absolute ethanol and allowed to stir at reflux for 30 min. Concentration provided 45.0 g of material of which 28.0 g was purified by reverse phase chromatography (Waters Deltaprep-3000) using isocratic 30% acetonitrile/water (0.05% TFA); 9.0 g of pale lavender material was collected which was redissolved in 150 mL of acetonitrile and precipitated with 500 mL of water. This material was collected by filtration and relyophilized in acetonitrile/water (1:1) to give 8.1 g (25%) of colorless ethyl ester: NMR (DMSO-d₆) d 0.86(t, J=7 Hz, 3H), 1.16(t, J=7H, 3H), 1.21-1.34(m, 2H), 1.49-1.61(m, 2H), 1.82(s, 3H), 2.22(t, J=8 Hz, 2H), 2.65(t, J=8 Hz, 2H), 4.02-4.11(m, 2H), 4.15-4.24(m, 1H), 7.78-7.83(m, 1H), 7.87-7.92(m, 1H), 8.21-8.27(m, 1H), 8.47(d, J=2H, 1H), 9.94(d, J=2H, 1H); HRMS. Calc'd for M+H: 393.2138. Found: 393.2097.

The following Examples #865-#1097 of Table XIII are highly preferred conjugates composed of dopamine-β-hydroxylase inhibitor compounds and glutamic acid derivatives. These dopamine-β-hydroxylase inhibitors utilized to make these conjugates are embraced by generic Formula XIV and XV, above.

TABLE XIII

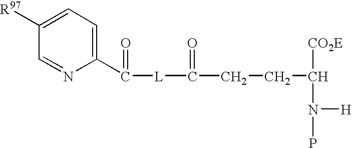

| EXAMPLE | | | | |
|---|---|---|---|---|
| NO. | L | R⁹⁷ | E | P |
| 865 | NHNH | C₂H₅ | CH₃ | H |
| 866 | NHNH | C₂H₅ | CH₃ | COCH₃ |
| 867 | NHNH | C₃H₇ | H | H |
| 868 | NHNH | C₃H₇ | H | COCH₃ |
| 869 | NHNH | C₃H₇ | CH₃ | H |
| 870 | NHNH | C₃H₇ | CH₃ | COCH₃ |
| 871 | NHNH | CH₃ | H | H |
| 872 | NHNH | CH₃ | H | COCH₃ |
| 873 | NHNH | C₄H₉ | CH₃ | H |
| 874 | NHNH | C₄H₉ | CH₃ | COCH₃ |
| 875 | NHNH | C₅H₁₁ | H | H |
| 876 | NHNH | C₅H₁₁ | H | COCH₃ |
| 877 | NHNH | C₅H₁₁ | CH₃ | H |
| 878 | NHNH | C₅H₁₁ | CH₃ | COCH₃ |
| 879 | NHNH | C₆H₁₃ | H | H |
| 880 | NHNH | C₆H₁₃ | H | COCH₃ |
| 881 | NHNH | C₆H₁₃ | CH₃ | COCH₃ |
| 882 | NHNH | OCH₃ | H | H |
| 883 | NHNH | OCH₃ | H | COCH₃ |
| 884 | NHNH | OCH₃ | CH₃ | H |
| 885 | NHNH | OCH₃ | CH₃ | COCH₃ |
| 886 | NHNH | OC₂H₅ | H | H |
| 887 | NHNH | OC₂H₅ | H | COCH₃ |
| 888 | NHNH | OC₂H₅ | CH₃ | H |
| 889 | NHNH | OC₂H₅ | CH₃ | COCH₃ |
| 890 | NHNH | OC₃H₇ | H | H |
| 891 | NHNH | OC₃H₇ | H | COCH₃ |
| 892 | NHNH | OC₃H₇ | CH₃ | H |
| 893 | NHNH | OC₃H₇ | CH₃ | COCH₃ |
| 894 | NHNH | OC₄H₉ | H | H |
| 895 | NHNH | OC₄H₉ | H | COCH₃ |
| 896 | NHNH | OC₄H₉ | CH₃ | H |
| 897 | NHNH | OC₄H₉ | CH₃ | COCH₃ |
| 898 | NHNH | SCH₃ | H | H |
| 899 | NHNH | SCH₃ | H | COCH₃ |
| 900 | NHNH | SCH₃ | CH₃ | H |
| 901 | NHNH | SCH₃ | CH₃ | COCH₃ |
| 902 | NHNH | SC₂H₅ | H | H |
| 903 | NHNH | SC₂H₅ | H | COCH₃ |
| 904 | NHNH | SC₂H₅ | CH₃ | H |
| 905 | NHNH | SC₂H₅ | CH₃ | COCH₃ |
| 906 | NHNH | SC₃H₇ | H | H |
| 907 | NHNH | SC₃H₇ | H | COCH₃ |
| 908 | NHNH | SC₃H₇ | CH₃ | H |
| 909 | NHNH | SC₃H₇ | CH₃ | COCH₃ |
| 910 | NHNH | F | H | H |
| 911 | NHNH | F | H | COCH₃ |
| 912 | NHNH | F | CH₃ | H |
| 913 | NHNH | F | CH₃ | COCH₃ |
| 914 | NHNH | Cl | H | H |
| 915 | NHNH | Cl | H | COCH₃ |
| 916 | NHNH | Cl | CH₃ | H |
| 917 | NHNH | Cl | CH₃ | COCH₃ |
| 918 | NHNH | Br | H | H |
| 919 | NHNH | Br | H | COCH₃ |
| 920 | NHNH | Br | CH₃ | H |
| 921 | NHNH | Br | CH₃ | COCH₃ |
| 922 | NHNH | I | H | H |
| 923 | NHNH | I | H | COCH₃ |
| 924 | NHNH | I | CH₃ | H |
| 925 | NHNH | I | CH₃ | COCH₃ |
| 926 | NHNH | CN | H | H |
| 927 | NHNH | CN | H | COCH₃ |
| 928 | NHNH | CN | CH₃ | H |
| 929 | NHNH | CN | CH₃ | COCH₃ |
| 930 | NHNH | NO₂ | H | H |
| 931 | NHNH | NO₂ | H | COCH₃ |
| 932 | NHNH | NO₂ | CH₃ | H |
| 933 | NHNH | NO₂ | CH₃ | COCH₃ |
| 934 | NHNH | OH | H | H |
| 935 | NHNH | OH | H | COCH₃ |
| 936 | NHNH | OH | CH₃ | H |
| 937 | NHNH | OH | CH₃ | COCH₃ |
| 938 | NHCH₂CH₂NH | CH₃ | H | H |
| 939 | NHCH₂CH₂NH | CH₃ | H | COCH₃ |
| 940 | NHCH₂CH₂NH | CH₃ | CH₃ | H |
| 941 | NHCH₂CH₂NH | CH₃ | CH₃ | COCH₃ |
| 942 | NHCH₂CH₂NH | C₂H₅ | H | H |
| 943 | NHCH₂CH₂NH | C₂H₅ | H | COCH₃ |
| 944 | NHCH₂CH₂NH | C₂H₅ | CH₃ | H |
| 945 | NHCH₂CH₂NH | C₂H₅ | CH₃ | COCH₃ |
| 946 | NHCH₂CH₂NH | C₃H₇ | H | H |
| 947 | NHCH₂CH₂NH | C₃H₇ | H | COCH₃ |
| 948 | NHCH₂CH₂NH | C₃H₇ | CH₃ | H |
| 949 | NHCH₂CH₂NH | C₃H₇ | CH₃ | COCH₃ |
| 950 | NHNH | CH₃ | CH₃ | H |
| 951 | NHNH | CH₃ | CH₃ | COCH₃ |
| 952 | NHCH₂CH₂NH | C₄H₉ | CH₃ | H |
| 953 | NHCH₂CH₂NH | C₄H₉ | CH₃ | COCH₃ |
| 954 | NHCH₂CH₂NH | C₅H₁₁ | H | H |
| 955 | NHCH₂CH₂NH | C₅H₁₁ | H | COCH₃ |
| 956 | NHCH₂CH₂NH | C₅H₁₁ | CH₃ | H |
| 957 | NHCH₂CH₂NH | C₅H₁₁ | CH₃ | COCH₃ |
| 958 | NHCH₂CH₂NH | C₆H₁₃ | H | H |
| 959 | NHCH₂CH₂NH | C₆H₁₃ | H | COCH₃ |
| 960 | NHCH₂CH₂NH | C₆H₁₃ | CH₃ | H |
| 961 | NHCH₂CH₂NH | C₆H₁₃ | CH₃ | COCH₃ |
| 962 | NHCH₂CH₂NH | OCH₃ | H | H |
| 963 | NHCH₂CH₂NH | OCH₃ | H | COCH₃ |
| 964 | NHCH₂CH₂NH | OCH₃ | CH₃ | H |
| 965 | NHCH₂CH₂NH | OCH₃ | CH₃ | COCH₃ |
| 966 | NHCH₂CH₂NH | OC₂H₅ | H | H |
| 967 | NHCH₂CH₂NH | OC₂H₅ | H | COCH₃ |
| 968 | NHCH₂CH₂NH | OC₂H₅ | CH₃ | H |
| 969 | NHCH₂CH₂NH | OC₂H₅ | CH₃ | COCH₃ |
| 970 | NHCH₂CH₂NH | OC₃H₇ | H | H |
| 971 | NHCH₂CH₂NH | OC₃H₇ | H | COCH₃ |
| 972 | NHCH₂CH₂NH | OC₃H₇ | CH₃ | H |
| 973 | NHCH₂CH₂NH | OC₃H₇ | CH₃ | COCH₃ |
| 974 | NHCH₂CH₂NH | OC₄H₉ | H | H |
| 975 | NHCH₂CH₂NH | OC₄H₉ | H | COCH₃ |
| 976 | NHCH₂CH₂NH | OC₄H₉ | CH₃ | H |
| 977 | NHCH₂CH₂NH | OC₄H₉ | CH₃ | COCH₃ |
| 978 | NHCH₂CH₂NH | SCH₃ | H | H |
| 979 | NHCH₂CH₂NH | SCH₃ | H | COCH₃ |
| 980 | NHCH₂CH₂NH | SCH₃ | CH₃ | H |
| 981 | NHCH₂CH₂NH | SCH₃ | CH₃ | COCH₃ |
| 982 | NHCH₂CH₂NH | SC₂H₅ | H | H |
| 983 | NHCH₂CH₂NH | SC₂H₅ | H | COCH₃ |
| 984 | NHCH₂CH₂NH | SC₂H₅ | CH₃ | H |
| 985 | NHCH₂CH₂NH | SC₂H₅ | CH₃ | COCH₃ |
| 986 | NHCH₂CH₂NH | SC₃H₇ | H | H |
| 987 | NHCH₂CH₂NH | SC₃H₇ | H | COCH₃ |
| 988 | NHCH₂CH₂NH | SC₃H₇ | CH₃ | H |
| 989 | NHCH₂CH₂NH | SC₃H₇ | CH₃ | COCH₃ |
| 990 | NHCH₂CH₂NH | F | H | H |
| 991 | NHCH₂CH₂NH | F | H | COCH₃ |
| 992 | NHCH₂CH₂NH | F | CH₃ | H |
| 993 | NHCH₂CH₂NH | F | CH₃ | COCH₃ |
| 994 | NHCH₂CH₂NH | Cl | H | H |
| 995 | NHCH₂CH₂NH | Cl | H | COCH₃ |
| 996 | NHCH₂CH₂NH | Cl | CH₃ | H |
| 997 | NHCH₂CH₂NH | Cl | CH₃ | COCH₃ |
| 998 | NHCH₂CH₂NH | Br | H | H |
| 999 | NHCH₂CH₂NH | Br | H | COCH₃ |
| 1000 | NHCH₂CH₂NH | Br | CH₃ | H |
| 1001 | NHCH₂CH₂NH | Br | CH₃ | COCH₃ |
| 1002 | NHCH₂CH₂NH | I | H | H |
| 1003 | NHCH₂CH₂NH | I | H | COCH₃ |
| 1004 | NHCH₂CH₂NH | I | CH₃ | H |
| 1005 | NHCH₂CH₂NH | I | CH₃ | COCH₃ |
| 1006 | NHCH₂CH₂NH | CN | H | H |
| 1007 | NHCH₂CH₂NH | CN | H | COCH₃ |
| 1008 | NHCH₂CH₂NH | CN | CH₃ | H |
| 1009 | NHCH₂CH₂NH | CN | CH₃ | COCH₃ |
| 1010 | NHCH₂CH₂NH | NO₂ | H | H |
| 1011 | NHCH₂CH₂NH | NO₂ | H | COCH₃ |
| 1012 | NHCH₂CH₂NH | NO₂ | CH₃ | H |
| 1013 | NHCH₂CH₂NH | NO₂ | CH₃ | COCH₃ |
| 1014 | NHCH₂CH₂NH | OH | H | H |
| 1015 | NHCH₂CH₂NH | OH | H | COCH₃ |
| 1016 | NHCH₂CH₂NH | OH | CH₃ | H |
| 1017 | NHCH₂CH₂NH | OH | CH₃ | COCH₃ |
| 1018 | piperzinyl | CH₃ | H | H |
| 1019 | piperzinyl | CH₃ | H | COCH₃ |
| 1020 | piperzinyl | CH₃ | CH₃ | H |
| 1021 | piperzinyl | CH₃ | CH₃ | COCH₃ |
| 1022 | piperzinyl | C₂H₅ | H | H |
| 1023 | piperzinyl | C₂H₅ | H | COCH₃ |
| 1024 | piperzinyl | C₂H₅ | CH₃ | H |
| 1025 | piperzinyl | C₂H₅ | CH₃ | COCH₃ |
| 1026 | piperzinyl | C₃H₇ | H | H |
| 1027 | piperzinyl | C₃H₇ | H | COCH₃ |
| 1028 | piperzinyl | C₃H₇ | CH₃ | H |
| 1029 | piperzinyl | C₃H₇ | CH₃ | COCH₃ |
| 1030 | NHNH | C₂H₅ | H | H |
| 1031 | NHNH | C₂H₅ | H | COCH₃ |
| 1032 | piperzinyl | C₄H₉ | CH₃ | H |
| 1033 | piperzinyl | C₄H₉ | CH₃ | COCH₃ |
| 1034 | piperzinyl | C₅H₁₁ | H | H |
| 1035 | piperzinyl | C₅H₁₁ | H | COCH₃ |
| 1036 | piperzinyl | C₅H₁₁ | CH₃ | H |
| 1037 | piperzinyl | C₅H₁₁ | CH₃ | COCH₃ |
| 1038 | piperzinyl | C₆H₁₃ | H | H |
| 1039 | piperzinyl | C₆H₁₃ | H | COCH₃ |
| 1040 | piperzinyl | C₆H₁₃ | CH₃ | H |
| 1041 | piperzinyl | C₆H₁₃ | CH₃ | COCH₃ |
| 1042 | piperzinyl | OCH₃ | H | H |
| 1043 | piperzinyl | OCH₃ | H | COCH₃ |
| 1044 | piperzinyl | OCH₃ | CH₃ | H |
| 1045 | piperzinyl | OCH₃ | CH₃ | COCH₃ |
| 1046 | piperzinyl | OC₂H₅ | H | H |
| 1047 | piperzinyl | OC₂H₅ | H | COCH₃ |
| 1048 | piperzinyl | OC₂H₅ | CH₃ | H |
| 1049 | piperzinyl | OC₂H₅ | CH₃ | COCH₃ |
| 1050 | piperzinyl | OC₃H₇ | H | H |
| 1051 | piperzinyl | OC₃H₇ | H | COCH₃ |
| 1052 | piperzinyl | OC₃H₇ | CH₃ | H |
| 1053 | piperzinyl | OC₃H₇ | CH₃ | COCH₃ |
| 1054 | piperzinyl | OC₄H₉ | H | H |
| 1055 | piperzinyl | OC₄H₉ | H | COCH₃ |
| 1056 | piperzinyl | OC₄H₉ | CH₃ | H |
| 1057 | piperzinyl | OC₄H₉ | CH₃ | COCH₃ |
| 1058 | piperzinyl | SCH₃ | H | H |
| 1059 | piperzinyl | SCH₃ | H | COCH₃ |
| 1060 | piperzinyl | SCH₃ | CH₃ | H |
| 1061 | piperzinyl | SCH₃ | CH₃ | COCH₃ |
| 1062 | piperzinyl | SC₂H₅ | H | H |
| 1063 | piperzinyl | SC₂H₅ | H | COCH₃ |
| 1064 | piperzinyl | SC₂H₅ | CH₃ | H |
| 1065 | piperzinyl | SC₂H₅ | CH₃ | COCH₃ |
| 1066 | piperzinyl | SC₃H₇ | H | H |
| 1067 | piperzinyl | SC₃H₇ | H | COCH₃ |
| 1068 | piperzinyl | SC₃H₇ | CH₃ | H |
| 1069 | piperzinyl | SC₃H₇ | CH₃ | COCH₃ |
| 1070 | piperzinyl | F | H | H |
| 1071 | piperzinyl | F | H | COCH₃ |
| 1072 | piperzinyl | F | CH₃ | H |
| 1073 | piperzinyl | F | CH₃ | COCH₃ |
| 1074 | piperzinyl | Cl | H | H |
| 1075 | piperzinyl | Cl | H | COCH₃ |
| 1076 | piperzinyl | Cl | CH₃ | H |
| 1077 | piperzinyl | Cl | CH₃ | COCH₃ |
| 1078 | piperzinyl | Br | H | H |
| 1079 | piperzinyl | Br | H | COCH₃ |
| 1080 | piperzinyl | Br | CH₃ | H |
| 1081 | piperzinyl | Br | CH₃ | COCH₃ |
| 1082 | piperzinyl | I | H | H |
| 1083 | piperzinyl | I | H | COCH₃ |
| 1084 | piperzinyl | I | CH₃ | H |
| 1085 | piperzinyl | I | CH₃ | COCH₃ |
| 1086 | piperzinyl | CN | H | H |
| 1087 | piperzinyl | CN | H | COCH₃ |
| 1088 | piperzinyl | CN | CH₃ | H |
| 1089 | piperzinyl | CN | CH₃ | COCH₃ |
| 1090 | piperzinyl | NO₂ | H | H |
| 1091 | piperzinyl | NO₂ | H | COCH₃ |
| 1092 | piperzinyl | NO₂ | CH₃ | H |
| 1093 | piperzinyl | NO₂ | CH₃ | COCH₃ |
| 1094 | piperzinyl | OH | H | H |
| 1095 | piperzinyl | OH | H | COCH₃ |
| 1096 | piperzinyl | OH | CH₃ | H |
| 1097 | piperzinyl | OH | CH₃ | COCH₃ |

The following Examples #1098-#1137 of Table XIV are highly preferred conjugates composed of dopamine-β-hydroxylase inhibitor compounds and glutamic acid derivatives. These dopamine-β-hydroxylase inhibitors utilized to make these conjugates are embraced by generic Formula XIV, above.

TABLE XIV

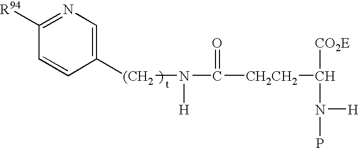

| EXAMPLE | | | | |
|---|---|---|---|---|
| NO. | R⁹⁴ | t | E | P |
| 1098 | CO₂H | 0 | H | H |
| 1099 | CO₂H | 0 | H | COCH₃ |
| 1100 | CO₂H | 0 | CH₃ | H |
| 1101 | CO₂H | 0 | CH₃ | COCH₃ |
| 1102 | CN₄H | 0 | H | H |
| 1103 | CN₄H | 0 | H | COCH₃ |
| 1104 | CN₄H | 0 | CH₃ | H |
| 1105 | CN₄H | 0 | CH₃ | COCH₃ |
| 1106 | CO₂H | 1 | H | H |
| 1107 | CO₂H | 1 | H | COCH₃ |
| 1108 | CO₂H | 1 | CH₃ | H |
| 1109 | CO₂H | 1 | CH₃ | COCH₃ |
| 1110 | CN₄H | 1 | H | H |
| 1111 | CN₄H | 1 | H | COCH₃ |
| 1112 | CN₄H | 1 | CH₃ | H |
| 1113 | CN₄H | 1 | CH₃ | COCH₃ |
| 1114 | CO₂H | 2 | H | H |
| 1115 | CO₂H | 2 | H | COCH₃ |
| 1116 | CO₂H | 2 | CH₃ | H |
| 1117 | CO₂H | 2 | CH₃ | COCH₃ |
| 1118 | CN₄H | 2 | H | H |
| 1119 | CN₄H | 2 | H | COCH₃ |
| 1120 | CN₄H | 2 | CH₃ | H |
| 1121 | CN₄H | 2 | CH₃ | COCH₃ |
| 1122 | CO₂H | 3 | H | H |
| 1123 | CO₂H | 3 | H | COCH₃ |
| 1124 | CO₂H | 3 | CH₃ | H |
| 1125 | CO₂H | 3 | CH₃ | COCH₃ |
| 1126 | CN₄H | 3 | H | H |
| 1127 | CN₄H | 3 | H | COCH₃ |
| 1128 | CN₄H | 3 | CH₃ | H |
| 1129 | CN₄H | 3 | CH₃ | COCH₃ |
| 1130 | CO₂H | 4 | H | H |
| 1131 | CO₂H | 4 | H | COCH₃ |
| 1132 | CO₂H | 4 | CH₃ | H |
| 1133 | CO₂H | 4 | CH₃ | COCH₃ |
| 1134 | CN₄H | 4 | H | H |
| 1135 | CN₄H | 4 | H | COCH₃ |
| 1136 | CN₄H | 4 | CH₃ | H |
| 1137 | CN₄H | 4 | CH₃ | COCH₃ |

The following Examples #1138-#1377 of Table XV are highly preferred conjugates composed of dopamine-pase hydroxylase inhibitor compounds and glutamic acid derivatives. These dopamine-β-hydroxylase inhibitors utilized to make these conjugates are embraced by generic Formula XVIII, above.

TABLE XV

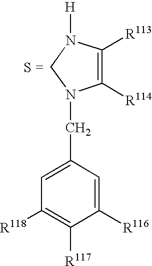

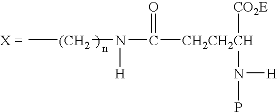

| EXAMPLE | | | | | | | | |
|---|---|---|---|---|---|---|---|---|
| NO. | n | R¹¹ | R¹¹⁴ | R¹¹⁶ | R¹¹⁷ | R¹¹⁸ | E | P |
| 1138 | 0 | X | H | H | OH | H | H | H |
| 1139 | 0 | X | H | H | OH | H | H | COCH₃ |
| 1140 | 0 | X | H | H | OH | H | CH₃ | H |
| 1141 | 0 | X | H | H | OH | H | CH₃ | COCH₃ |
| 1142 | 0 | X | H | H | F | H | H | H |
| 1143 | 0 | X | H | H | F | H | H | COCH₃ |
| 1144 | 0 | X | H | H | F | H | CH₃ | H |
| 1145 | 0 | X | H | H | F | H | CH₃ | COCH₃ |
| 1146 | 0 | X | H | H | CF₃ | H | H | H |
| 1147 | 0 | X | H | H | CF₃ | H | H | COCH₃ |
| 1148 | 0 | X | H | H | CF₃ | H | CH₃ | H |
| 1149 | 0 | X | H | H | CF₃ | H | CH₃ | COCH₃ |
| 1150 | 0 | X | H | OH | OH | H | H | H |
| 1151 | 0 | X | H | OH | OH | H | H | COCH₃ |
| 1152 | 0 | X | H | OH | OH | H | CH₃ | H |
| 1153 | 0 | X | H | OH | OH | H | CH₃ | COCH₃ |
| 1154 | 0 | X | H | F | H | F | H | H |
| 1155 | 0 | X | H | F | H | F | H | COCH₃ |
| 1156 | 0 | X | H | F | H | F | CH₃ | H |
| 1157 | 0 | X | H | F | H | F | CH₃ | COCH₃ |
| 1158 | 0 | X | H | CF₃ | H | CF₃ | H | H |
| 1159 | 0 | X | H | CF₃ | H | CF₃ | H | COCH₃ |
| 1160 | 0 | X | H | CF₃ | H | CF₃ | CH₃ | H |
| 1161 | 0 | X | H | CF₃ | H | CF₃ | CH₃ | COCH₃ |
| 1162 | 0 | H | X | H | OH | H | H | H |
| 1163 | 0 | H | X | H | OH | H | H | COCH₃ |
| 1164 | 0 | H | X | H | OH | H | CH₃ | H |
| 1165 | 0 | H | X | H | OH | H | CH₃ | COCH₃ |
| 1166 | 0 | H | X | H | F | H | H | H |
| 1167 | 0 | H | X | H | F | H | H | COCH₃ |
| 1168 | 0 | H | X | H | F | H | CH₃ | H |
| 1169 | 0 | H | X | H | F | H | CH₃ | COCH₃ |
| 1170 | 0 | H | X | H | CF₃ | H | H | H |
| 1171 | 0 | H | X | H | CF₃ | H | H | COCH₃ |
| 1172 | 0 | H | X | H | CF₃ | H | CH₃ | H |
| 1173 | 0 | H | X | H | CF₃ | H | CH₃ | COCH₃ |
| 1174 | 0 | H | X | OH | OH | H | H | H |
| 1175 | 0 | H | X | OH | OH | H | H | COCH₃ |
| 1176 | 0 | H | X | OH | OH | H | CH₃ | H |
| 1177 | 0 | H | X | OH | OH | H | CH₃ | COCH₃ |
| 1178 | 0 | H | X | F | H | F | H | H |
| 1179 | 0 | H | X | F | H | F | H | COCH₃ |
| 1180 | 0 | H | X | F | H | F | CH₃ | H |
| 1181 | 0 | H | X | F | H | F | CH₃ | COCH₃ |
| 1182 | 0 | H | X | CF₃ | H | CF₃ | H | H |
| 1183 | 0 | H | X | CF₃ | H | CF₃ | H | COCH₃ |
| 1184 | 0 | H | X | CF₃ | H | CF₃ | CH₃ | H |
| 1185 | 0 | H | X | CF₃ | H | CF₃ | CH₃ | COCH₃ |
| 1186 | 1 | X | H | H | OH | H | H | H |
| 1187 | 1 | X | H | H | OH | H | H | COCH₃ |
| 1188 | 1 | X | H | H | OH | H | CH₃ | H |
| 1189 | 1 | X | H | H | OH | H | CH₃ | COCH₃ |
| 1190 | 1 | X | H | H | F | H | H | H |
| 1191 | 1 | X | H | H | F | H | H | COCH₃ |
| 1192 | 1 | X | H | H | F | H | CH₃ | H |
| 1193 | 1 | X | H | H | F | H | CH₃ | COCH₃ |
| 1194 | 1 | X | H | H | CF₃ | H | H | H |
| 1195 | 1 | X | H | H | CF₃ | H | H | COCH₃ |
| 1196 | 1 | X | H | H | CF₃ | H | CH₃ | H |
| 1197 | 1 | X | H | H | CF₃ | H | CH₃ | COCH₃ |
| 1198 | 1 | X | H | OH | OH | H | H | H |
| 1199 | 1 | X | H | OH | OH | H | H | COCH₃ |
| 1200 | 1 | X | H | OH | OH | H | CH₃ | H |
| 1201 | 1 | X | H | OH | OH | H | CH₃ | COCH₃ |
| 1202 | 1 | X | H | F | H | F | H | H |
| 1203 | 1 | X | H | F | H | F | H | COCH₃ |
| 1204 | 1 | X | H | F | H | F | CH₃ | H |
| 1205 | 1 | X | H | F | H | F | CH₃ | COCH₃ |
| 1206 | 1 | X | H | CF₃ | H | CF₃ | H | H |
| 1207 | 1 | X | H | CF₃ | H | CF₃ | H | COCH₃ |
| 1208 | 1 | X | H | CF₃ | H | CF₃ | CH₃ | H |
| 1209 | 1 | X | H | CF₃ | H | CF₃ | CH₃ | COCH₃ |
| 1210 | 1 | H | X | H | OH | H | H | H |
| 1211 | 1 | H | X | H | OH | H | H | COCH₃ |
| 1212 | 1 | H | X | H | OH | H | CH₃ | H |
| 1213 | 1 | H | X | H | OH | H | CH₃ | COCH₃ |
| 1214 | 1 | H | X | H | F | H | H | H |
| 1215 | 1 | H | X | H | F | H | H | COCH₃ |
| 1216 | 1 | H | X | H | F | H | CH₃ | H |
| 1217 | 1 | H | X | H | F | H | CH₃ | COCH₃ |
| 1218 | 1 | H | X | H | CF₃ | H | H | H |
| 1219 | 1 | H | X | H | CF₃ | H | H | COCH₃ |
| 1220 | 1 | H | X | H | CF₃ | H | CH₃ | H |
| 1221 | 1 | H | X | H | CF₃ | H | CH₃ | COCH₃ |
| 1222 | 1 | H | X | 1H | OH | H | H | H |
| 1223 | 1 | H | X | 1H | OH | H | H | COCH₃ |
| 1224 | 1 | H | X | 1H | OH | H | CH₃ | H |
| 1225 | 1 | H | X | 1H | OH | H | CH₃ | COCH₃ |
| 1226 | 1 | H | X | F | H | F | H | H |
| 1227 | 1 | H | X | F | H | F | H | COCH₃ |
| 1228 | 1 | H | X | F | H | F | CH₃ | H |
| 1229 | 1 | H | X | F | H | F | CH₃ | COCH₃ |
| 1230 | 1 | H | X | CF₃ | H | CF₃ | H | H |
| 1231 | 1 | H | X | CF₃ | H | CF₃ | H | COCH₃ |
| 1232 | 1 | H | X | CF₃ | H | CF₃ | CH₃ | H |
| 1233 | 1 | H | X | CF₃ | H | CF₃ | CH₃ | COCH₃ |
| 1234 | 2 | X | H | H | OH | H | H | H |
| 1235 | 2 | X | H | H | OH | H | H | COCH₃ |
| 1236 | 2 | X | H | H | OH | H | CH₃ | H |
| 1237 | 2 | X | H | H | OH | H | CH₃ | COCH₃ |
| 1238 | 2 | X | H | H | F | H | H | H |
| 1239 | 2 | x | H | H | F | H | H | COCH₃ |
| 1240 | 2 | X | H | H | F | H | CH₃ | H |
| 1241 | 2 | X | H | H | F | H | CH₃ | COCH₃ |
| 1242 | 2 | X | H | H | CF₃ | H | H | H |
| 1243 | 2 | X | H | H | CF₃ | H | H | COCH₃ |
| 1244 | 2 | X | H | H | CF₃ | H | CH₃ | H |
| 1245 | 2 | X | H | H | CF₃ | H | CH₃ | COCH₃ |
| 1246 | 2 | X | H | OH | OH | H | H | H |
| 1247 | 2 | X | H | OH | OH | H | H | COCH₃ |
| 1248 | 2 | X | H | OH | OH | H | CH₃ | H |
| 1249 | 2 | X | H | OH | OH | H | CH₃ | COCH₃ |
| 1250 | 2 | X | H | F | H | F | H | H |
| 1251 | 2 | X | H | F | H | F | H | COCH₃ |
| 1252 | 2 | X | H | F | H | F | CH₃ | H |
| 1253 | 2 | X | H | F | H | F | CH₃ | COCH₃ |
| 1254 | 2 | X | H | CF₃ | H | CF₃ | H | H |
| 1255 | 2 | X | H | CF₃ | H | CF₃ | H | COCH₃ |
| 1256 | 2 | X | H | CF₃ | H | CF₃ | CH₃ | H |
| 1257 | 2 | X | H | CF₃ | H | CF₃ | CH₃ | COCH₃ |
| 1258 | 2 | H | X | H | OH | H | H | H |
| 1259 | 2 | H | X | H | OH | H | H | COCH₃ |
| 1260 | 2 | H | X | H | OH | H | CH₃ | H |
| 1261 | 2 | H | X | H | OH | H | CH₃ | COCH₃ |
| 1262 | 2 | H | X | H | F | H | H | H |
| 1263 | 2 | H | X | H | F | H | H | COCH₃ |
| 1264 | 2 | H | X | H | F | H | CH₃ | H |
| 1265 | 2 | H | X | H | F | H | CH₃ | COCH₃ |
| 1266 | 2 | H | X | H | CF₃ | H | H | H |
| 1267 | 2 | H | X | H | CF₃ | H | H | COCH₃ |
| 1268 | 2 | H | X | H | CF₃ | H | CH₃ | H |
| 1269 | 2 | H | X | H | CF₃ | H | CH₃ | COCH₃ |
| 1270 | 2 | H | X | OH | OH | H | H | H |
| 1271 | 2 | H | X | OH | OH | H | H | COCH₃ |
| 1272 | 2 | H | X | OH | OH | H | CH₃ | H |
| 1273 | 2 | H | X | OH | OH | H | CH₃ | COCH₃ |
| 1274 | 2 | H | X | F | H | F | H | H |
| 1275 | 2 | H | X | F | H | F | H | COCH₃ |
| 1276 | 2 | H | X | F | H | F | CH₃ | H |
| 1277 | 2 | H | X | F | H | F | CH₃ | COCH₃ |
| 1278 | 2 | H | X | CF₃ | H | CF₃ | H | H |
| 1279 | 2 | H | X | CF₃ | H | CF₃ | H | COCH₃ |
| 1280 | 2 | H | X | CF₃ | H | CF₃ | CH₃ | H |
| 1281 | 2 | H | X | CF₃ | H | CF₃ | CH₃ | COCH₃ |
| 1282 | 3 | X | H | H | OH | H | H | H |
| 1283 | 3 | X | H | H | OH | H | H | COCH₃ |
| 1284 | 3 | X | H | H | OH | H | CH₃ | H |
| 1285 | 3 | X | H | H | OH | H | CH₃ | COCH₃ |
| 1286 | 3 | X | H | H | F | H | H | H |
| 1287 | 3 | X | H | H | F | H | H | COCH₃ |
| 1288 | 3 | X | H | H | F | H | CH₃ | H |
| 1289 | 3 | X | H | H | F | H | CH₃ | COCH₃ |
| 1290 | 3 | X | H | H | CF₃ | H | H | H |
| 1291 | 3 | X | H | H | CF₃ | H | H | COCH₃ |
| 1292 | 3 | X | H | H | CF₃ | H | CH₃ | H |
| 1293 | 3 | X | H | H | CF₃ | H | CH₃ | COCH₃ |
| 1294 | 3 | X | H | OH | OH | H | H | H |
| 1295 | 3 | X | H | OH | OH | H | H | COCH₃ |
| 1296 | 3 | X | H | OH | OH | H | CH₃ | H |
| 1297 | 3 | X | H | OH | OH | H | CH₃ | COCH₃ |
| 1298 | 3 | x | H | F | H | F | H | H |
| 1299 | 3 | x | H | F | H | F | H | COCH₃ |
| 1300 | 3 | x | H | F | H | F | CH₃ | H |
| 1301 | 3 | X | H | F | H | F | CH₃ | COCH₃ |
| 1302 | 3 | X | H | CF₃ | H | CF₃ | H | H |
| 1303 | 3 | X | H | CF₃ | H | CF₃ | H | COCH₃ |
| 1304 | 3 | X | H | CF₃ | H | CF₃ | CH₃ | H |
| 1305 | 3 | X | H | CF₃ | H | CF₃ | CH₃ | COCH₃ |
| 1306 | 3 | H | X | H | OH | H | H | H |
| 1307 | 3 | H | X | H | OH | H | H | COCH₃ |
| 1308 | 3 | H | X | H | OH | H | CH₃ | H |
| 1309 | 3 | H | X | H | OH | H | CH₃ | COCH₃ |
| 1310 | 3 | H | X | H | F | H | H | H |
| 1311 | 3 | H | X | H | F | H | H | COCH₃ |
| 1312 | 3 | H | X | H | F | H | CH₃ | H |
| 1313 | 3 | H | X | H | F | H | CH₃ | COCH₃ |
| 1314 | 3 | H | X | H | CF₃ | H | H | H |
| 1315 | 3 | H | X | H | CF₃ | H | H | COCH₃ |
| 1316 | 3 | H | X | H | CF₃ | H | CH₃ | H |
| 1317 | 3 | H | X | H | CF₃ | H | CH₃ | COCH₃ |
| 1318 | 3 | H | X | OH | OH | H | H | H |
| 1319 | 3 | H | X | OH | OH | H | H | COCH₃ |
| 1320 | 3 | H | X | OH | OH | H | CH₃ | H |
| 1321 | 3 | H | X | OH | OH | H | CH₃ | COCH₃ |
| 1322 | 3 | H | X | F | H | F | H | H |
| 1323 | 3 | H | X | F | H | F | H | COCH₃ |
| 1324 | 3 | H | X | F | H | F | CH₃ | H |
| 1325 | 3 | H | X | F | H | F | CH₃ | COCH₃ |
| 1326 | 3 | H | X | CF₃ | H | CF₃ | H | H |
| 1327 | 3 | H | X | CF₃ | H | CF₃ | H | COCH₃ |
| 1328 | 3 | H | X | CF₃ | H | CF₃ | CH₃ | H |
| 1329 | 3 | H | X | CF₃ | H | CF₃ | CH₃ | COCH₃ |
| 1330 | 4 | X | H | H | OH | H | H | H |
| 1331 | 4 | X | H | H | OH | H | H | COCH₃ |
| 1332 | 4 | X | H | H | OH | H | CH₃ | H |
| 1333 | 4 | X | H | H | OH | H | CH₃ | COCH₃ |
| 1334 | 4 | X | H | H | F | H | H | H |
| 1335 | 4 | X | H | H | F | H | H | COCH₃ |
| 1336 | 4 | X | H | H | F | H | CH₃ | H |
| 1337 | 4 | X | H | H | F | H | CH₃ | COCH₃ |
| 1338 | 4 | X | H | H | CF₃ | H | H | H |
| 1339 | 4 | X | H | H | CF₃ | H | H | COCH₃ |
| 1340 | 4 | X | H | H | CF₃ | H | CH₃ | H |
| 1341 | 4 | X | H | H | CF₃ | H | CH₃ | COCH₃ |
| 1342 | 4 | X | H | OH | OH | H | H | H |
| 1343 | 4 | X | H | OH | OH | H | H | COCH₃ |
| 1344 | 4 | X | H | OH | OH | H | CH₃ | H |
| 1345 | 4 | X | H | OH | OH | H | CH₃ | COCH₃ |
| 1346 | 4 | X | H | F | H | F | H | H |
| 1347 | 4 | X | H | F | H | F | H | COCH₃ |
| 1348 | 4 | X | H | F | H | F | CH₃ | H |
| 1349 | 4 | X | H | F | H | F | CH₃ | COCH₃ |
| 1350 | 4 | X | H | CF₃ | H | CF₃ | H | H |
| 1351 | 4 | X | H | CF₃ | H | CF₃ | H | COCH₃ |
| 1352 | 4 | X | H | CF₃ | H | CF₃ | CH₃ | H |
| 1353 | 4 | X | H | CF₃ | H | CF₃ | CH₃ | COCH₃ |
| 1354 | 4 | H | X | H | OH | H | H | H |
| 1355 | 4 | H | X | H | OH | H | H | COCH₃ |
| 1356 | 4 | H | X | H | OH | H | CH₃ | H |
| 1357 | 4 | H | X | H | OH | H | CH₃ | COCH₃ |
| 1358 | 4 | H | X | H | F | H | H | H |
| 1359 | 4 | H | X | H | F | H | H | COCH₃ |
| 1360 | 4 | H | X | H | F | H | CH₃ | H |
| 1361 | 4 | H | X | H | F | H | CH₃ | COCH₃ |
| 1362 | 4 | H | X | H | CF₃ | H | H | H |
| 1363 | 4 | H | X | H | CF₃ | H | H | COCH₃ |
| 1364 | 4 | H | X | H | CF₃ | H | CH₃ | H |
| 1365 | 4 | H | X | H | CF₃ | H | CH₃ | COCH₃ |
| 1366 | 4 | H | X | OH | OH | H | H | H |
| 1367 | 4 | H | X | OH | OH | H | H | COCH₃ |
| 1368 | 4 | H | X | OH | OH | H | CH₃ | H |
| 1369 | 4 | H | X | OH | OH | H | CH₃ | COCH₃ |
| 1370 | 4 | H | X | F | H | F | H | H |
| 1371 | 4 | H | X | F | H | F | H | COCH₃ |
| 1372 | 4 | H | X | F | H | F | CH₃ | H |
| 1373 | 4 | H | X | F | H | F | CH₃ | COCH₃ |
| 1374 | 4 | H | X | CF₃ | H | CF₃ | H | H |
| 1375 | 4 | H | X | CF₃ | H | CF₃ | H | COCH₃ |
| 1376 | 4 | H | X | CF₃ | H | CF₃ | CH₃ | H |
| 1377 | 4 | H | X | CF₃ | H | CF₃ | CH₃ | COCH₃ |

The following Examples #1378-#1497 of Table XVI are highly preferred conjugates composed of dopamine-pxylase hydroxylase inhibitor compounds and glutamic acid derivatives. These dopamine-β-hydroxylase inhibitors utilized to make these conjugates are embraced by generic Formula XVIII, above.

TABLE XVI

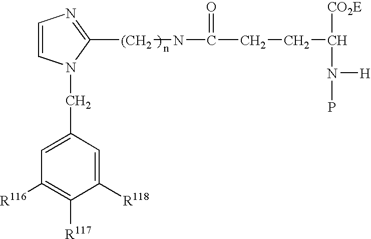

| EXAMPLE | | | | | | |
|---|---|---|---|---|---|---|
| NO. | n | R¹¹⁶ | R¹¹⁷ | R¹¹⁸ | E | P |
| 1378 | 0 | H | OH | H | H | H |
| 1379 | 0 | H | OH | H | H | COCH₃ |
| 1380 | 0 | H | OH | H | CH₃ | H |
| 1381 | 0 | H | OH | H | CH₃ | COCH₃ |
| 1382 | 0 | H | F | H | H | H |
| 1383 | 0 | H | F | H | H | COCH₃ |
| 1384 | 0 | H | F | H | CH₃ | H |
| 1385 | 0 | H | F | H | CH₃ | COCH₃ |
| 1386 | 0 | H | CF₃ | H | H | H |
| 1387 | 0 | H | CF₃ | H | H | COCH₃ |
| 1388 | 0 | H | CF₃ | H | CH₃ | H |
| 1389 | 0 | H | CF₃ | H | CH₃ | COCH₃ |
| 1390 | 0 | OH | OH | H | H | H |
| 1391 | 0 | OH | OH | H | H | COCH₃ |
| 1392 | 0 | OH | OH | H | CH₃ | H |
| 1393 | 0 | OH | OH | H | CH₃ | COCH₃ |
| 1394 | 0 | F | H | F | H | H |
| 1395 | 0 | F | H | F | H | COCH₃ |
| 1396 | 0 | F | H | F | CH₃ | H |
| 1397 | 0 | F | H | F | CH₃ | COCH₃ |
| 1398 | 0 | CF₃ | H | CF₃ | H | H |
| 1399 | 0 | CF₃ | H | CF₃ | H | COCH₃ |
| 1400 | 0 | CF₃ | H | CF₃ | CH₃ | H |
| 1401 | 0 | CF₃ | H | CF₃ | CH₃ | COCH₃ |
| 1402 | 1 | H | OH | H | H | H |
| 1403 | 1 | H | OH | H | H | COCH₃ |
| 1404 | 1 | H | OH | H | CH₃ | H |
| 1405 | 1 | H | OH | H | CH₃ | COCH₃ |
| 1406 | 1 | H | F | H | H | H |
| 1407 | 1 | H | F | H | H | COCH₃ |
| 1408 | 1 | H | F | H | CH₃ | H |
| 1409 | 1 | H | F | H | CH₃ | COCH₃ |
| 1410 | 1 | H | CF₃ | H | H | H |
| 1411 | 1 | H | CF₃ | H | H | COCH₃ |
| 1412 | 1 | H | CF₃ | H | CH₃ | H |
| 1413 | 1 | H | CF₃ | H | CH₃ | COCH₃ |
| 1414 | 1 | OH | OH | H | H | H |
| 1415 | 1 | OH | OH | H | H | COCH₃ |
| 1416 | 1 | OH | OH | H | CH₃ | H |
| 1417 | 1 | OH | OH | H | CH₃ | COCH₃ |
| 1418 | 1 | F | H | F | H | H |
| 1419 | 1 | F | H | F | H | COCH₃ |
| 1420 | 1 | F | H | F | CH₃ | H |
| 1421 | 1 | F | H | F | CH₃ | COCH₃ |
| 1422 | 1 | CF₃ | H | CF₃ | H | H |
| 1423 | 1 | CF₃ | H | CF₃ | H | COCH₃ |
| 1424 | 1 | CF₃ | H | CF₃ | CH₃ | H |
| 1425 | 1 | CF₃ | H | CF₃ | CH₃ | COCH₃ |
| 1426 | 2 | H | OH | H | H | H |
| 1427 | 2 | H | OH | H | H | COCH₃ |
| 1428 | 2 | H | OH | H | CH₃ | H |
| 1429 | 2 | H | OH | H | CH₃ | COCH₃ |
| 1430 | 2 | H | F | H | H | H |
| 1431 | 2 | H | F | H | H | COCH₃ |
| 1432 | 2 | H | F | H | CH₃ | H |
| 1433 | 2 | H | F | H | CH₃ | COCH₃ |
| 1434 | 2 | H | CF₃ | H | H | H |
| 1435 | 2 | H | CF₃ | H | H | COCH₃ |
| 1436 | 2 | H | CF₃ | H | CH₃ | H |
| 1437 | 2 | H | CF₃ | H | CH₃ | COCH₃ |
| 1438 | 2 | OH | OH | H | H | H |
| 1439 | 2 | OH | OH | H | H | COCH₃ |
| 1440 | 2 | OH | OH | H | CH₃ | H |
| 1441 | 2 | OH | OH | H | CH₃ | COCH₃ |
| 1442 | 2 | F | H | F | H | H |
| 1443 | 2 | F | H | F | H | COCH₃ |
| 1444 | 2 | F | H | F | CH₃ | H |
| 1445 | 2 | F | H | F | CH₃ | COCH₃ |
| 1446 | 2 | CF₃ | H | CF₃ | H | H |
| 1447 | 2 | CF₃ | H | CF₃ | H | COCH₃ |
| 1448 | 2 | CF₃ | H | CF₃ | CH₃ | H |
| 1449 | 2 | CF₃ | H | CF₃ | CH₃ | COCH₃ |
| 1450 | 3 | H | OH | H | H | H |
| 1451 | 3 | H | OH | H | H | COCH₃ |
| 1452 | 3 | H | OH | H | CH₃ | H |
| 1453 | 3 | H | OH | H | CH₃ | COCH₃ |
| 1454 | 3 | H | F | H | H | H |
| 1455 | 3 | H | F | H | H | COCH₃ |
| 1456 | 3 | H | F | H | CH₃ | H |
| 1457 | 3 | H | F | H | CH₃ | COCH₃ |
| 1458 | 3 | H | CF₃ | H | H | H |
| 1459 | 3 | H | CF₃ | H | H | COCH₃ |
| 1460 | 3 | H | CF₃ | H | CH₃ | H |
| 1461 | 3 | H | CF₃ | H | CH₃ | COCH₃ |
| 1462 | 3 | OH | OH | H | H | H |
| 1463 | 3 | OH | OH | H | H | COCH₃ |
| 1464 | 3 | OH | OH | H | CH₃ | H |
| 1465 | 3 | OH | OH | H | CH₃ | COCH₃ |
| 1466 | 3 | F | H | F | H | H |
| 1467 | 3 | F | H | F | H | COCH₃ |
| 1468 | 3 | F | H | F | CH₃ | H |
| 1469 | 3 | F | H | F | CH₃ | COCH₃ |
| 1470 | 3 | CF₃ | H | CF₃ | H | H |
| 1471 | 3 | CF₃ | H | CF₃ | H | COCH₃ |
| 1472 | 3 | CF₃ | H | CF₃ | CH₃ | H |
| 1473 | 3 | CF₃ | H | CF₃ | CH₃ | COCH₃ |
| 1474 | 4 | H | OH | H | H | H |
| 1475 | 4 | H | OH | H | H | COCH₃ |
| 1476 | 4 | H | OH | H | CH₃ | H |
| 1477 | 4 | H | OH | H | CH₃ | COCH₃ |
| 1478 | 4 | H | F | H | H | H |
| 1479 | 4 | H | F | H | H | COCH₃ |
| 1480 | 4 | H | F | H | CH₃ | H |
| 1481 | 4 | H | F | H | CH₃ | COCH₃ |
| 1482 | 4 | H | CF₃ | H | H | H |
| 1483 | 4 | H | CF₃ | H | H | COCH₃ |
| 1484 | 4 | H | CF₃ | H | CH₃ | H |
| 1485 | 4 | H | CF₃ | H | CH₃ | COCH₃ |
| 1486 | 4 | OH | OH | H | H | H |
| 1487 | 4 | OH | OH | H | H | COCH₃ |
| 1488 | 4 | OH | OH | H | CH₃ | H |
| 1489 | 4 | OH | OH | H | CH₃ | COCH₃ |
| 1490 | 4 | F | H | F | H | H |
| 1491 | 4 | F | H | F | H | COCH₃ |
| 1492 | 4 | F | H | F | CH₃ | H |
| 1493 | 4 | F | H | F | CH₃ | COCH₃ |
| 1494 | 4 | CF₃ | H | CF₃ | H | H |
| 1495 | 4 | CF₃ | H | CF₃ | H | COCH₃ |
| 1496 | 4 | CF₃ | H | CF₃ | CH₃ | H |
| 1497 | 4 | CF₃ | H | CF₃ | CH₃ | COCH₃ |

The following Examples #1498-#1857 of Table XVII are highly preferred conjugates composed of dopamine-pase hydroxylase inhibitor compounds and glutamic acid derivatives. These dopamine-β-hydroxylase inhibitors utilized to make these conjugates are embraced by generic Formula XVIII, above.

TABLE XVII

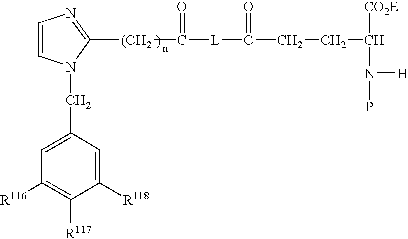

| EXAM- | | | | | | | |
|---|---|---|---|---|---|---|---|
| PLE | | | | | | | |
| NO. | n | L | R¹¹⁶ | R¹¹⁷ | R¹¹⁸ | E | P |
| 1498 | 0 | NHNH | H | OH | H | H | H |
| 1499 | 0 | NHNH | H | OH | H | H | COCH₃ |
| 1500 | 0 | NHNH | H | OH | H | CH₃ | H |
| 1501 | 0 | NHNH | H | OH | H | CH₃ | COCH₃ |
| 1502 | 0 | NHNH | H | F | H | H | H |
| 1503 | 0 | NHNH | H | F | H | H | COCH₃ |
| 1504 | 0 | NHNH | H | F | H | CH₃ | H |
| 1505 | 0 | NHNH | H | F | H | CH₃ | COCH₃ |
| 1506 | 0 | NHNH | H | CF₃ | H | H | H |
| 1507 | 0 | NHNH | H | CF₃ | H | H | COCH₃ |
| 1508 | 0 | NHNH | H | CF₃ | H | CH₃ | H |
| 1509 | 0 | NHNH | H | CF₃ | H | CH₃ | COCH₃ |
| 1510 | 0 | NHNH | OH | OH | H | H | H |
| 1511 | 0 | NHNH | OH | OH | H | H | COCH₃ |
| 1512 | 0 | NHNH | OH | OH | H | CH₃ | H |
| 1513 | 0 | NHNH | OH | OH | H | CH₃ | COCH₃ |
| 1514 | 0 | NHNH | F | H | F | H | H |
| 1515 | 0 | NHNH | F | H | F | H | COCH₃ |
| 1516 | 0 | NHNH | F | H | F | CH₃ | H |
| 1517 | 0 | NHNH | F | H | F | CH₃ | COCH₃ |
| 1518 | 0 | NHNH | CF₃ | H | CF₃ | H | H |
| 1519 | 0 | NHNH | CF₃ | H | CF₃ | H | COCH₃ |
| 1520 | 0 | NHNH | CF₃ | H | CF₃ | CH₃ | H |
| 1521 | 0 | NHNH | CF₃ | H | CF₃ | CH₃ | COCH₃ |
| 1522 | 0 | NHCH₂CH₂NH | H | OH | H | H | H |
| 1523 | 0 | NHCH₂CH₂NH | H | OH | H | H | COCH₃ |
| 1524 | 0 | NHCH₂CH₂NH | H | OH | H | CH₃ | H |
| 1525 | 0 | NHCH₂CH₂NH | H | OH | H | CH₃ | COCH₃ |
| 1526 | 0 | NHCH₂CH₂NH | H | F | H | H | H |
| 1527 | 0 | NHCH₂CH₂NH | H | F | H | H | COCH₃ |
| 1528 | 0 | NHCH₂CH₂NH | H | F | H | CH₃ | H |
| 1529 | 0 | NHCH₂CH₂NH | H | F | H | CH₃ | COCH₃ |
| 1530 | 0 | NHCH₂CH₂NH | H | CF₃ | H | H | H |
| 1531 | 0 | NHCH₂CH₂NH | H | CF₃ | H | H | COCH₃ |
| 1532 | 0 | NHCH₂CH₂NH | H | CF₃ | H | CH₃ | H |
| 1533 | 0 | NHCH₂CH₂NH | H | CF₃ | H | CH₃ | COCH₃ |
| 1534 | 0 | NHCH₂CH₂NH | OH | OH | H | H | H |
| 1535 | 0 | NHCH₂CH₂NH | OH | OH | H | H | COCH₃ |
| 1536 | 0 | NHCH₂CH₂NH | OH | OH | H | CH₃ | H |
| 1537 | 0 | NHCH₂CH₂NH | OH | OH | H | CH₃ | COCH₃ |
| 1538 | 0 | NHCH₂CH₂NH | F | H | F | H | H |
| 1539 | 0 | NHCH₂CH₂NH | F | H | F | H | COCH₃ |
| 1540 | 0 | NHCH₂CH₂NH | F | H | F | CH₃ | H |
| 1541 | 0 | NHCH₂CH₂NH | F | H | F | CH₃ | COCH₃ |
| 1542 | 0 | NHCH₂CH₂NH | CF₃ | H | CF₃ | H | H |
| 1543 | 0 | NHCH₂CH₂NH | CF₃ | H | CF₃ | H | COCH₃ |
| 1544 | 0 | NHCH₂CH₂NH | CF₃ | H | CF₃ | CH₃ | H |
| 1545 | 0 | NHCH₂CH₂NH | CF₃ | H | CF₃ | CH₃ | COCH₃ |
| 1546 | 0 | piperazinyl | H | OH | H | H | H |
| 1547 | 0 | piperazinyl | H | OH | H | H | COCH₃ |
| 1548 | 0 | piperazinyl | H | OH | H | CH₃ | H |
| 1549 | 0 | piperazinyl | H | OH | H | CH₃ | COCH₃ |
| 1550 | 0 | piperazinyl | H | F | H | H | H |
| 1551 | 0 | piperazinyl | H | F | H | H | COCH₃ |
| 1552 | 0 | piperazinyl | H | F | H | CH₃ | H |
| 1553 | 0 | piperazinyl | H | F | H | CH₃ | COCH₃ |
| 1554 | 0 | piperazinyl | H | CF₃ | H | H | H |
| 1555 | 0 | piperazinyl | H | CF₃ | H | H | COCH₃ |
| 1556 | 0 | piperazinyl | H | CF₃ | H | CH₃ | H |
| 1557 | 0 | piperazinyl | H | CF₃ | H | CH₃ | COCH₃ |
| 1558 | 0 | piperazinyl | OH | OH | H | H | H |
| 1559 | 0 | piperazinyl | OH | OH | H | H | COCH₃ |
| 1560 | 0 | piperazinyl | OH | OH | H | CH₃ | H |
| 1561 | 0 | piperazinyl | OH | OH | H | CH₃ | COCH₃ |
| 1562 | 0 | piperazinyl | F | H | F | H | H |
| 1563 | 0 | piperazinyl | F | H | F | H | COCH₃ |
| 1564 | 0 | piperazinyl | F | H | F | CH₃ | H |
| 1565 | 0 | piperazinyl | F | H | F | CH₃ | COCH₃ |
| 1566 | 0 | piperazinyl | CF₃ | H | CF₃ | H | H |
| 1567 | 0 | piperazinyl | CF₃ | H | CF₃ | H | COCH₃ |
| 1568 | 0 | piperazinyl | CF₃ | H | CF₃ | CH₃ | H |
| 1569 | 0 | piperazinyl | CF₃ | H | CF₃ | CH₃ | COCH₃ |
| 1570 | 1 | NHNH | H | OH | H | H | H |
| 1571 | 1 | NHNH | H | OH | H | H | COCH₃ |
| 1572 | 1 | NHNH | H | OH | H | CH₃ | H |
| 1573 | 1 | NHNH | H | OH | H | CH₃ | COCH₃ |
| 1574 | 1 | NHNH | H | F | H | H | H |
| 1575 | 1 | NHNH | H | F | H | H | COCH₃ |
| 1576 | 1 | NHNH | H | F | H | CH₃ | H |
| 1577 | 1 | NHNH | H | F | H | CH₃ | COCH₃ |
| 1578 | 1 | NHNH | H | CF₃ | H | H | H |
| 1579 | 1 | NHNH | H | CF₃ | H | H | COCH₃ |
| 1580 | 1 | NHNH | H | CF₃ | H | CH₃ | H |
| 1581 | 1 | NHNH | H | CF₃ | H | CH₃ | COCH₃ |
| 1582 | 1 | NHNH | OH | OH | H | H | H |
| 1583 | 1 | NHNH | OH | OH | H | H | COCH₃ |
| 1584 | 1 | NHNH | OH | OH | H | CH₃ | H |
| 1585 | 1 | NHNH | OH | OH | H | CH₃ | COCH₃ |
| 1586 | 1 | NHNH | F | H | F | H | H |
| 1587 | 1 | NHNH | F | H | F | H | COCH₃ |
| 1588 | 1 | NHNH | F | H | F | CH₃ | H |
| 1589 | 1 | NHNH | F | H | F | CH₃ | COCH₃ |
| 1590 | 1 | NHNH | CF₃ | H | CF₃ | H | H |
| 1591 | 1 | NHNH | CF₃ | H | CF₃ | H | COCH₃ |
| 1592 | 1 | NHNH | CF₃ | H | CF₃ | CH₃ | H |
| 1593 | 1 | NHNH | CF₃ | H | CF₃ | CH₃ | COCH₃ |
| 1594 | 1 | NHCH₂CH₂NH | H | OH | H | H | H |
| 1595 | 1 | NHCH₂CH₂NH | H | OH | H | H | COCH₃ |
| 1596 | 1 | NHCH₂CH₂NH | H | OH | H | CH₃ | H |
| 1597 | 1 | NHCH₂CH₂NH | H | OH | H | CH₃ | COCH₃ |
| 1598 | 1 | NHCH₂CH₂NH | H | F | H | H | H |
| 1599 | 1 | NHCH₂CH₂NH | H | F | H | H | COCH₃ |
| 1600 | 1 | NHCH₂CH₂NH | H | F | H | CH₃ | H |
| 1601 | 1 | NHCH₂CH₂NH | H | F | H | CH₃ | COCH₃ |
| 1602 | 1 | NHCH₂CH₂NH | H | CF₃ | H | H | H |
| 1603 | 1 | NHCH₂CH₂NH | H | CF₃ | H | H | COCH₃ |
| 1604 | 1 | NHCH₂CH₂NH | H | CF₃ | H | CH₃ | H |
| 1605 | 1 | NHCH₂CH₂NH | H | CF₃ | H | CH₃ | COCH₃ |
| 1606 | 1 | NHCH₂CH₂NH | OH | OH | H | H | H |
| 1607 | 1 | NHCH₂CH₂NH | OH | OH | H | H | COCH₃ |
| 1608 | 1 | NHCH₂CH₂NH | OH | OH | H | CH₃ | H |
| 1609 | 1 | NHCH₂CH₂NH | OH | OH | H | CH₃ | COCH₃ |
| 1610 | 1 | NHCH₂CH₂NH | F | H | F | H | H |
| 1611 | 1 | NHCH₂CH₂NH | F | H | F | H | COCH₃ |
| 1612 | 1 | NHCH₂CH₂NH | F | H | F | CH₃ | H |
| 1613 | 1 | NHCH₂CH₂NH | F | H | F | CH₃ | COCH₃ |
| 1614 | 1 | NHCH₂CH₂NH | CF₃ | H | CF₃ | H | H |
| 1615 | 1 | NHCH₂CH₂NH | CF₃ | H | CF₃ | H | COCH₃ |
| 1616 | 1 | NHCH₂CH₂NH | CF₃ | H | CF₃ | CH₃ | H |
| 1617 | 1 | NHCH₂CH₂NH | CF₃ | H | CF₃ | CH₃ | COCH₃ |
| 1618 | 1 | piperazinyl | H | OH | H | H | H |
| 1619 | 1 | piperazinyl | H | OH | H | H | COCH₃ |
| 1620 | 1 | piperazinyl | H | OH | H | CH₃ | H |
| 1621 | 1 | piperazinyl | H | OH | H | CH₃ | COCH₃ |
| 1622 | 1 | piperazinyl | H | F | H | H | H |
| 1623 | 1 | piperazinyl | H | F | H | H | COCH₃ |
| 1624 | 1 | piperazinyl | H | F | H | CH₃ | H |
| 1625 | 1 | piperazinyl | H | F | H | CH₃ | COCH₃ |
| 1626 | 1 | piperazinyl | H | CF₃ | H | H | H |
| 1627 | 1 | piperazinyl | H | CF₃ | H | H | COCH₃ |
| 1628 | 1 | piperazinyl | H | CF₃ | H | CH₃ | H |
| 1629 | 1 | piperazinyl | H | CF₃ | H | CH₃ | COCH₃ |
| 1630 | 1 | piperazinyl | OH | OH | H | H | H |
| 1631 | 1 | piperazinyl | OH | OH | H | H | COCH₃ |
| 1632 | 1 | piperazinyl | OH | OH | H | CH₃ | H |
| 1633 | 1 | piperazinyl | OH | OH | H | CH₃ | COCH₃ |
| 1634 | 1 | piperazinyl | F | H | F | H | H |
| 1635 | 1 | piperazinyl | F | H | F | H | COCH₃ |
| 1636 | 1 | piperazinyl | F | H | F | CH₃ | H |
| 1637 | 1 | piperazinyl | F | H | F | CH₃ | COCH₃ |
| 1638 | 1 | piperazinyl | CF₃ | H | CF₃ | H | H |
| 1639 | 1 | piperazinyl | CF₃ | H | CF₃ | H | COCH₃ |
| 1640 | 1 | piperazinyl | CF₃ | H | CF₃ | CH₃ | H |
| 1641 | 1 | piperazinyl | CF₃ | H | CF₃ | CH₃ | COCH₃ |
| 1642 | 2 | NHNH | H | OH | H | H | H |
| 1643 | 2 | NHNH | H | OH | H | H | COCH₃ |
| 1644 | 2 | NHNH | H | OH | H | CH₃ | H |
| 1645 | 2 | NHNH | H | OH | H | CH₃ | COCH₃ |
| 1646 | 2 | NHNH | H | F | H | H | H |
| 1647 | 2 | NHNH | H | F | H | H | COCH₃ |
| 1648 | 2 | NHNH | H | F | H | CH₃ | H |
| 1649 | 2 | NHNH | H | F | H | CH₃ | COCH₃ |
| 1650 | 2 | NHNH | H | CF₃ | H | H | H |
| 1651 | 2 | NHNH | H | CF₃ | H | H | COCH₃ |
| 1652 | 2 | NHNH | H | CF₃ | H | CH₃ | H |
| 1653 | 2 | NHNH | H | CF₃ | H | CH₃ | COCH₃ |
| 1654 | 2 | NHNH | OH | OH | H | H | H |
| 1655 | 2 | NHNH | OH | OH | H | H | COCH₃ |
| 1656 | 2 | NHNH | OH | OH | H | CH₃ | H |
| 1657 | 2 | NHNH | OH | OH | H | CH₃ | COCH₃ |
| 1658 | 2 | NHNH | F | H | F | H | H |
| 1659 | 2 | NHNH | F | H | F | H | COCH₃ |
| 1660 | 2 | NHNH | F | H | F | CH₃ | H |
| 1661 | 2 | NHNH | F | H | F | CH₃ | COCH₃ |
| 1662 | 2 | NHNH | CF₃ | H | CF₃ | H | H |
| 1663 | 2 | NHNH | CF₃ | H | CF₃ | H | COCH₃ |
| 1664 | 2 | NHNH | CF₃ | H | CF₃ | CH₃ | H |
| 1665 | 2 | NHNH | CF₃ | H | CF₃ | CH₃ | COCH₃ |
| 1666 | 2 | NHCH₂CH₂NH | H | OH | H | H | H |
| 1667 | 2 | NHCH₂CH₂NH | H | OH | H | H | COCH₃ |
| 1668 | 2 | NHCH₂CH₂NH | H | OH | H | CH₃ | H |
| 1669 | 2 | NHCH₂CH₂NH | H | OH | H | CH₃ | COCH₃ |
| 1670 | 2 | NHCH₂CH₂NH | H | F | H | H | H |
| 1671 | 2 | NHCH₂CH₂NH | H | F | H | H | COCH₃ |
| 1672 | 2 | NHCH₂CH₂NH | H | F | H | CH₃ | H |
| 1673 | 2 | NHCH₂CH₂NH | H | F | H | CH₃ | COCH₃ |
| 1674 | 2 | NHCH₂CH₂NH | H | CF₃ | H | H | H |
| 1675 | 2 | NHCH₂CH₂NH | H | CF₃ | H | H | COCH₃ |
| 1676 | 2 | NHCH₂CH₂NH | H | CF₃ | H | CH₃ | H |
| 1677 | 2 | NHCH₂CH₂NH | H | CF₃ | H | CH₃ | COCH₃ |
| 1678 | 2 | NHCH₂CH₂NH | OH | OH | H | H | H |
| 1679 | 2 | NHCH₂CH₂NH | OH | OH | H | H | COCH₃ |
| 1680 | 2 | NHCH₂CH₂NH | OH | OH | H | CH₃ | H |
| 1681 | 2 | NHCH₂CH₂NH | OH | OH | H | CH₃ | COCH₃ |
| 1682 | 2 | NHCH₂CH₂NH | F | H | F | H | H |
| 1683 | 2 | NHCH₂CH₂NH | F | H | F | H | COCH₃ |
| 1684 | 2 | NHCH₂CH₂NH | F | H | F | CH₃ | H |
| 1685 | 2 | NHCH₂CH₂NH | F | H | F | CH₃ | COCH₃ |
| 1686 | 2 | NHCH₂CH₂NH | CF₃ | H | CF₃ | H | H |
| 1687 | 2 | NHCH₂CH₂NH | CF₃ | H | CF₃ | H | COCH₃ |
| 1688 | 2 | NHCH₂CH₂NH | CF₃ | H | CF₃ | CH₃ | H |
| 1689 | 2 | NHCH₂CH₂NH | CF₃ | H | CF₃ | CH₃ | COCH₃ |
| 1690 | 2 | piperazinyl | H | OH | H | H | H |
| 1691 | 2 | piperazinyl | H | OH | H | H | COCH₃ |
| 1692 | 2 | piperazinyl | H | OH | H | CH₃ | H |
| 1693 | 2 | piperazinyl | H | OH | H | CH₃ | COCH₃ |
| 1694 | 2 | piperazinyl | H | F | H | H | H |
| 1695 | 2 | piperazinyl | H | F | H | H | COCH₃ |
| 1696 | 2 | piperazinyl | H | F | H | CH₃ | H |
| 1697 | 2 | piperazinyl | H | F | H | CH₃ | COCH₃ |
| 1698 | 2 | piperazinyl | H | CF₃ | H | H | H |
| 1699 | 2 | piperazinyl | H | CF₃ | H | H | COCH₃ |
| 1700 | 2 | piperazinyl | H | CF₃ | H | CH₃ | H |
| 1701 | 2 | piperazinyl | H | CF₃ | H | CH₃ | COCH₃ |
| 1702 | 2 | piperazinyl | OH | OH | H | H | H |
| 1703 | 2 | piperazinyl | OH | OH | H | H | COCH₃ |
| 1704 | 2 | piperazinyl | OH | OH | H | CH₃ | H |
| 1705 | 2 | piperazinyl | OH | OH | H | CH₃ | COCH₃ |
| 1706 | 2 | piperazinyl | F | H | F | H | H |
| 1707 | 2 | piperazinyl | F | H | F | H | COCH₃ |
| 1708 | 2 | piperazinyl | F | H | F | CH₃ | H |
| 1709 | 2 | piperazinyl | F | H | F | CH₃ | COCH₃ |
| 1710 | 2 | piperazinyl | CF₃ | H | CF₃ | H | H |
| 1711 | 2 | piperazinyl | CF₃ | H | CF₃ | H | COCH₃ |
| 1712 | 2 | piperazinyl | CF₃ | H | CF₃ | CH₃ | H |
| 1713 | 2 | piperazinyl | CF₃ | H | CF₃ | CH₃ | COCH₃ |
| 1714 | 3 | NHNH | H | OH | H | H | H |
| 1715 | 3 | NHNH | H | OH | H | H | COCH₃ |
| 1716 | 3 | NHNH | H | OH | H | CH₃ | H |
| 1717 | 3 | NHNH | H | OH | H | CH₃ | COCH₃ |
| 1718 | 3 | NHNH | H | F | H | H | H |
| 1719 | 3 | NHNH | H | F | H | H | COCH₃ |
| 1720 | 3 | NHNH | H | F | H | CH₃ | H |
| 1721 | 3 | NHNH | H | F | H | CH₃ | COCH₃ |
| 1722 | 3 | NHNH | H | CF₃ | H | H | H |
| 1723 | 3 | NHNH | H | CF₃ | H | H | COCH₃ |
| 1724 | 3 | NHNH | H | CF₃ | H | CH₃ | H |
| 1725 | 3 | NHNH | H | CF₃ | H | CH₃ | COCH₃ |
| 1726 | 3 | NHNH | OH | OH | H | H | H |
| 1727 | 3 | NHNH | OH | OH | H | H | COCH₃ |
| 1728 | 3 | NHNH | OH | OH | H | CH₃ | H |
| 1729 | 3 | NHNH | OH | OH | H | CH₃ | COCH₃ |
| 1730 | 3 | NHNH | F | H | F | H | H |
| 1731 | 3 | NHNH | F | H | F | H | COCH₃ |
| 1732 | 3 | NHNH | F | H | F | CH₃ | H |
| 1733 | 3 | NHNH | F | H | F | CH₃ | COCH₃ |
| 1734 | 3 | NHNH | CF₃ | H | CF₃ | H | H |
| 1735 | 3 | NHNH | CF₃ | H | CF₃ | H | COCH₃ |
| 1736 | 3 | NHNH | CF₃ | H | CF₃ | CH₃ | H |
| 1737 | 3 | NHNH | CF₃ | H | CF₃ | CH₃ | COCH₃ |
| 1738 | 3 | NHCH₂CH₂NH | H | OH | H | H | H |
| 1739 | 3 | NHCH₂CH₂NH | H | OH | H | H | COCH₃ |
| 1740 | 3 | NHCH₂CH₂NH | H | OH | H | CH₃ | H |
| 1741 | 3 | NHCH₂CH₂NH | H | OH | H | CH₃ | COCH₃ |
| 1742 | 3 | NHCH₂CH₂NH | H | F | H | H | H |
| 1743 | 3 | NHCH₂CH₂NH | H | F | H | H | COCH₃ |
| 1744 | 3 | NHCH₂CH₂NH | H | F | H | CH₃ | H |
| 1745 | 3 | NHCH₂CH₂NH | H | F | H | CH₃ | COCH₃ |
| 1746 | 3 | NHCH₂CH₂NH | H | CF₃ | H | H | H |
| 1747 | 3 | NHCH₂CH₂NH | H | CF₃ | H | H | COCH₃ |
| 1748 | 3 | NHCH₂CH₂NH | H | CF₃ | H | CH₃ | H |
| 1749 | 3 | NHCH₂CH₂NH | H | CF₃ | H | CH₃ | COCH₃ |
| 1750 | 3 | NHCH₂CH₂NH | OH | OH | H | H | H |
| 1751 | 3 | NHCH₂CH₂NH | OH | OH | H | H | COCH₃ |
| 1752 | 3 | NHCH₂CH₂NH | OH | OH | H | CH₃ | H |
| 1753 | 3 | NHCH₂CH₂NH | OH | OH | H | CH₃ | COCH₃ |
| 1754 | 3 | NHCH₂CH₂NH | F | H | F | H | H |
| 1755 | 3 | NHCH₂CH₂NH | F | H | F | H | COCH₃ |
| 1756 | 3 | NHCH₂CH₂NH | F | H | F | CH₃ | H |
| 1757 | 3 | NHCH₂CH₂NH | F | H | F | CH₃ | COCH₃ |
| 1758 | 3 | NHCH₂CH₂NH | CF₃ | H | CF₃ | H | H |
| 1759 | 3 | NHCH₂CH₂NH | CF₃ | H | CF₃ | H | COCH₃ |
| 1760 | 3 | NHCH₂CH₂NH | CF₃ | H | CF₃ | CH₃ | H |
| 1761 | 3 | NHCH₂CH₂NH | CF₃ | H | CF₃ | CH₃ | COCH₃ |
| 1762 | 3 | piperazinyl | H | OH | H | H | H |
| 1763 | 3 | piperazinyl | H | OH | H | H | COCH₃ |
| 1764 | 3 | piperazinyl | H | OH | H | CH₃ | H |
| 1765 | 3 | piperazinyl | H | OH | H | CH₃ | COCH₃ |
| 1766 | 3 | piperazinyl | H | F | H | H | H |
| 1767 | 3 | piperazinyl | H | F | H | H | COCH₃ |
| 1768 | 3 | piperazinyl | H | F | H | CH₃ | H |
| 1769 | 3 | piperazinyl | H | F | H | CH₃ | COCH₃ |
| 1770 | 3 | piperazinyl | H | CF₃ | H | H | H |
| 1771 | 3 | piperazinyl | H | CF₃ | H | H | COCH₃ |
| 1772 | 3 | piperazinyl | H | CF₃ | H | CH₃ | H |
| 1773 | 3 | piperazinyl | H | CF₃ | H | CH₃ | COCH₃ |
| 1774 | 3 | piperazinyl | OH | OH | H | H | H |
| 1775 | 3 | piperazinyl | OH | OH | H | H | COCH₃ |
| 1776 | 3 | piperazinyl | OH | OH | H | CH₃ | H |
| 1777 | 3 | piperazinyl | OH | OH | H | CH₃ | COCH₃ |
| 1778 | 3 | piperazinyl | F | H | F | H | H |
| 1779 | 3 | piperazinyl | F | H | F | H | COCH₃ |
| 1780 | 3 | piperazinyl | F | H | F | CH₃ | H |
| 1781 | 3 | piperazinyl | F | H | F | CH₃ | COCH₃ |
| 1782 | 3 | piperazinyl | CF₃ | H | CF₃ | H | H |
| 1783 | 3 | piperazinyl | CF₃ | H | CF₃ | H | COCH₃ |
| 1784 | 3 | piperazinyl | CF₃ | H | CF₃ | CH₃ | H |
| 1785 | 3 | piperazinyl | CF₃ | H | CF₃ | CH₃ | COCH₃ |
| 1786 | 4 | NHNH | H | OH | H | H | H |
| 1787 | 4 | NHNH | H | OH | H | H | COCH₃ |
| 1788 | 4 | NHNH | H | OH | H | CH₃ | H |
| 1789 | 4 | NHNH | H | OH | H | CH₃ | COCH₃ |
| 1790 | 4 | NHNH | H | F | H | H | H |
| 1791 | 4 | NHNH | H | F | H | H | COCH₃ |
| 1792 | 4 | NHNH | H | F | H | CH₃ | H |
| 1793 | 4 | NHNH | H | F | H | CH₃ | COCH₃ |
| 1794 | 4 | NHNH | H | CF₃ | H | H | H |
| 1795 | 4 | NHNH | H | CF₃ | H | H | COCH₃ |
| 1796 | 4 | NHNH | H | CF₃ | H | CH₃ | H |
| 1797 | 4 | NHNH | H | CF₃ | H | CH₃ | COCH₃ |
| 1798 | 4 | NHNH | OH | OH | H | H | H |
| 1799 | 4 | NHNH | OH | OH | H | H | COCH₃ |
| 1800 | 4 | NHNH | OH | OH | H | CH₃ | H |
| 1801 | 4 | NHNH | OH | OH | H | CH₃ | COCH₃ |
| 1802 | 4 | NHNH | F | H | F | H | H |
| 1803 | 4 | NHNH | F | H | F | H | COCH₃ |
| 1804 | 4 | NHNH | F | H | F | CH₃ | H |
| 1805 | 4 | NHNH | F | H | F | CH₃ | COCH₃ |
| 1806 | 4 | NHNH | CF₃ | H | CF₃ | H | H |
| 1807 | 4 | NHNH | CF₃ | H | CF₃ | H | COCH₃ |
| 1808 | 4 | NHNH | CF₃ | H | CF₃ | CH₃ | H |
| 1809 | 4 | NHNH | CF₃ | H | CF₃ | CH₃ | COCH₃ |
| 1810 | 4 | NHCH₂CH₂NH | H | OH | H | H | H |
| 1811 | 4 | NHCH₂CH₂NH | H | OH | H | H | COCH₃ |
| 1812 | 4 | NHCH₂CH₂NH | H | OH | H | CH₃ | H |
| 1813 | 4 | NHCH₂CH₂NH | H | OH | H | CH₃ | COCH₃ |
| 1814 | 4 | NHCH₂CH₂NH | H | F | H | H | H |
| 1815 | 4 | NHCH₂CH₂NH | H | F | H | H | COCH₃ |
| 1816 | 4 | NHCH₂CH₂NH | H | F | H | CH₃ | H |
| 1817 | 4 | NHCH₂CH₂NH | H | F | H | CH₃ | COCH₃ |
| 1818 | 4 | NHCH₂CH₂NH | H | CF₃ | H | H | H |
| 1819 | 4 | NHCH₂CH₂NH | H | CF₃ | H | H | COCH₃ |
| 1820 | 4 | NHCH₂CH₂NH | H | CF₃ | H | CH₃ | H |
| 1821 | 4 | NHCH₂CH₂NH | H | CF₃ | H | CH₃ | COCH₃ |
| 1822 | 4 | NHCH₂CH₂NH | OH | OH | H | H | H |
| 1823 | 4 | NHCH₂CH₂NH | OH | OH | H | H | COCH₃ |
| 1824 | 4 | NHCH₂CH₂NH | OH | OH | H | CH₃ | H |
| 1825 | 4 | NHCH₂CH₂NH | OH | OH | H | CH₃ | COCH₃ |
| 1826 | 4 | NHCH₂CH₂NH | F | H | F | H | H |
| 1827 | 4 | NHCH₂CH₂NH | F | H | F | H | COCH₃ |
| 1828 | 4 | NHCH₂CH₂NH | F | H | F | CH₃ | H |
| 1829 | 4 | NHCH₂CH₂NH | F | H | F | CH₃ | COCH₃ |
| 1830 | 4 | NHCH₂CH₂NH | CF₃ | H | CF₃ | H | H |
| 1831 | 4 | NHCH₂CH₂NH | CF₃ | H | CF₃ | H | COCH₃ |
| 1832 | 4 | NHCH₂CH₂NH | CF₃ | H | CF₃ | CH₃ | H |
| 1833 | 4 | NHCH₂CH₂NH | CF₃ | H | CF₃ | CH₃ | COCH₃ |
| 1834 | 4 | piperazinyl | H | OH | H | H | H |
| 1835 | 4 | piperazinyl | H | OH | H | H | COCH₃ |
| 1836 | 4 | piperazinyl | H | OH | H | CH₃ | H |
| 1837 | 4 | piperazinyl | H | OH | H | CH₃ | COCH₃ |
| 1838 | 4 | piperazinyl | H | F | H | H | H |
| 1839 | 4 | piperazinyl | H | F | H | H | COCH₃ |
| 1840 | 4 | piperazinyl | H | F | H | CH₃ | H |
| 1841 | 4 | piperazinyl | H | F | H | CH₃ | COCH₃ |
| 1842 | 4 | piperazinyl | H | CF₃ | H | H | H |
| 1843 | 4 | piperazinyl | H | CF₃ | H | H | COCH₃ |
| 1844 | 4 | piperazinyl | H | CF₃ | H | CH₃ | H |
| 1845 | 4 | piperazinyl | H | CF₃ | H | CH₃ | COCH₃ |
| 1846 | 4 | piperazinyl | OH | OH | H | H | H |
| 1847 | 4 | piperazinyl | OH | OH | H | H | COCH₃ |
| 1848 | 4 | piperazinyl | OH | OH | H | CH₃ | H |
| 1849 | 4 | piperazinyl | OH | OH | H | CH₃ | COCH₃ |
| 1850 | 4 | piperazinyl | F | H | F | H | H |
| 1851 | 4 | piperazinyl | F | H | F | H | COCH₃ |
| 1852 | 4 | piperazinyl | F | H | F | CH₃ | H |
| 1853 | 4 | piperazinyl | F | H | F | CH₃ | COCH₃ |
| 1854 | 4 | piperazinyl | CF₃ | H | CF₃ | H | H |
| 1855 | 4 | piperazinyl | CF₃ | H | CF₃ | H | COCH₃ |
| 1856 | 4 | piperazinyl | CF₃ | H | CF₃ | CH₃ | H |
| 1857 | 4 | piperazinyl | CF₃ | H | CF₃ | CH₃ | COCH₃ |

### Biological Evaluations

Conjugates of the invention were evaluated biologically by in vitro and in vivo assays to determine the ability of the conjugates to selectively inhibit renal sympathetic nerve activity and lower blood pressure. Three classes of conjugates of the invention were evaluated for their ability to inhibit the enzymes of the catecholamine cascade selectively within the kidney. These inhibitor conjugates variously inhibit tyrosine hydroxylase, dopa-decarboxylase and dopamine-β-hydroxylase in order to interfere ultimately with the synthesis of norepinephrine in the kidney.

Assays I and II evaluate in vivo the acute and chronic effects of Ex. #3 conjugate (a tyrosine hydroxylase inhibitor conjugated with N-acetyl-γ-glutamyl) in rats. Assay III evaluates the chronic effects of Ex. #464 conjugate (a dopa-decarboxylase inhibitor conjugated with N-acetyl-γ-glutamyl) in rats.

Assay IV and V describes in vitro experiments performed to determine if the Ex. #859 conjugate was capable of being specifically metabolized by enzymes known to be abundant in the kidney. In Assay IV, the Ex. #859 conjugate was incubated with either rat kidney homogenate or a solution containing purified kidney enzymes to characterize resulting metabolites. In Assay V, experiments were performed to determine the potency of the Ex. #858 and Ex. #859 conjugates and potential metabolites as inhibitors of purified dopamine-β-hydroxylase.

Assays VI through IX describe in vivo experiments performed to characterize and compare the effects of fusaric acid and various conjugates of fusaric acid (Ex. #859, Ex. #861 and Ex. #863) on spontaneously hypertensive rats (SHR) by acute administration i.v. and i.d. and by chronic administration i.v. Assay X describes analysis of catecholamine levels in tissue from rats used in the chronic administration experiment of Assay VIII. Assays XI and XII describe in vivo experiments in dogs to determine the renal and mean arterial pressure effects of fusaric acid and Ex. #859 conjugate. Assay XIII describes mechanisms of the antihypertensive response to Ex. #859 conjugate, Assay XIV describes the antihypertensive efficacy of Ex. #859 conjugate in a second species (DOCA hypertensive micropig).

Assay I: Acute In Vivo Effects of Ex #3 Conjugate

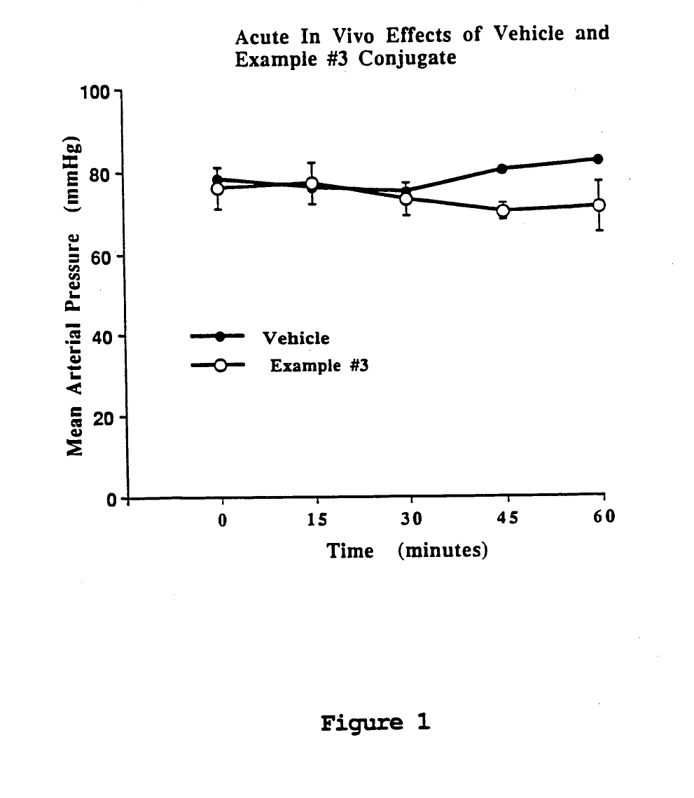

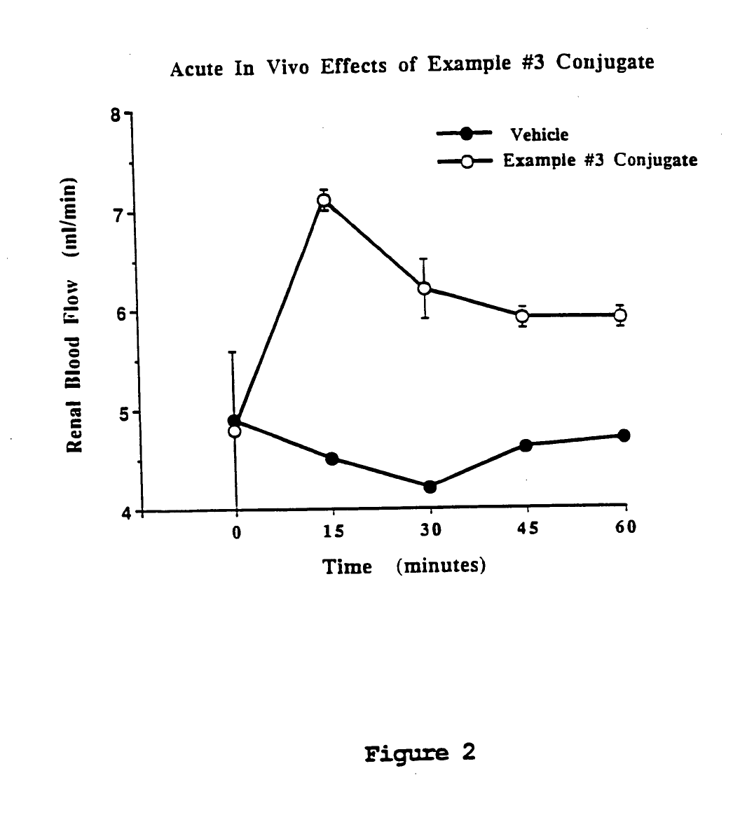

Sprague-Dawley rats were anesthetized with inactin (100 mg/kg, i.p.) and catheters were implanted into a carotid artery for measurement of mean arterial pressure (Gould model 3800 chart recorder; Statham pressure transducer model no. P23 DB) and into a jugular vein for compound administrations (i.v.). In addition, a flow probe was implanted around the left renal artery for measurement of renal blood flow using Carolina Medical Electronics flow probes. Rats were allowed 60 min to stabilize before 10 minutes of control recordings of mean arterial pressure and renal blood flow were obtained. Control measurements were followed by intravenous injection of Ex. #3 conjugate and saline vehicle. As shown in Table XVIII and in FIGS. 1 and 2, the Ex. #3 conjugate had no acute effects on mean arterial pressure (MAP), but increased renal blood flow (RBF).

TABLE XVIII
Acute In Vivo Effects of Ex. #3 Conjugate

| | Time After Injection (min) | | | | |
|---|---|---|---|---|---|
| | Zero | 15 | 30 | 45 | 60 |
| | Vehicle (0.5 ml 0.9% NaCl i.v.) | | | | |
| MAP | 78 | 76 | 75 | 80 | 82 |
| (mm Hg) | | | | | |
| RBF | 4.9 | 4.5 | 4.2 | 4.6 | 4.7 |
| (ml/min) | | | | | |
| | Ex. #3 Conjugate (100 mg/kg i.v.) | | | | |
| MAP | 76 ± 5 | 77 ± 5 | 73 ± 4 | 70 ± 2 | 71 ± 6 |
| (mm Hg) | | | | | |
| RBF | 4.8 ± 0.8 | 7.1 ± 0.1 | 6.2 ± 0.3 | 5.9 ± 0.1 | 5.9 ± 0.1 |
| (ml/min) | | | | | |

Assay II: Chronic In Vivo Effects of Ex. #3 Conjugate

The Ex. #3 conjugate and saline vehicle were infused continuously for four days in spontaneously hypertensive rats. Mean arterial pressure was measured (Gould Chart Recorder, model 3800; Statham P23 Db pressure transducer) via an indwelling femoral artery catheter between 10:00 a.m. and 2:00 p.m. each day. The Ex. #3 conjugate was infused at 5 mg/hr and the saline vehicle was infused at 300 μL/hr. via a jugular vein catheter with a Harvard infusion pump. Results are shown in Table XIX.

TABLE XIX
Chronic In Vivo Effects of Ex. #3 Conjugate

| | Time After Injection (days) | | | | |
|---|---|---|---|---|---|
| | Zero | 1 | 2 | 3 | 4 |
| | Vehicle (300 μL/hr) | | | | |
| MAP | 181 ± 8 | 172 ± 6 | 170 ± 7 | 174 ± 6 | 182 ± 3 |
| (mm Hg) | | | | | |
| | Ex. #3 Conjugate (5 mg/hr) | | | | |
| MAP | 164 ± 3 | 175 ± 5 | 174 ± 5 | 172 ± 2 | N.A. |
| (mm Hg) | | | | | |

Assay III: Chronic In Vivo Effects of Ex. #464 Conjugate

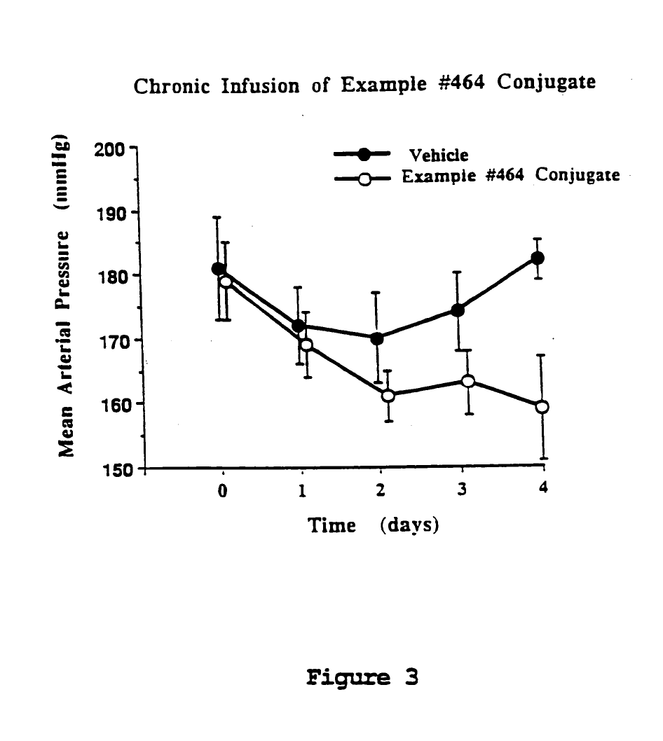

The Ex. #464 conjugate and saline vehicle were infused continuously for four days in spontaneously hypertensive rats. Mean arterial pressure was measured (Gould Chart Recorder, model 3800; Statham P23 Db pressure transducer) via an indwelling femoral artery catheter between 10:00 a.m. and 2:00 p.m. each day. The Ex. #464 conjugate was infused at 10 mg/hr and the saline vehicle was infused at 300 μL/hr. As shown in Table XX and in FIG. 3, mean arterial pressure was lowered significantly over the four-day period.

TABLE XX
Chronic In Vivo Effects of Ex. #464 Conjugate

| | Time After Injection (days) | | | | |
|---|---|---|---|---|---|
| | Zero | 1 | 2 | 3 | 4 |
| | Vehicle (300 μL/hr) | | | | |
| MAP | 181 ± 8 | 172 ± 6 | 170 ± 7 | 174 ± 6 | 182 ± 3 |
| (mm Hg) | | | | | |
| | Ex. #464 Conjugate (10 mg/hr) | | | | |
| MAP | 179 ± 6 | 169 ± 5 | 161 ± 4 | 163 ± 5 | 159 ± 8 |
| (mm Hg) | | | | | |

Assay IV: In Vitro Evaluation of Enzyme Metabolism Effects of Ex. #85% Conjugate

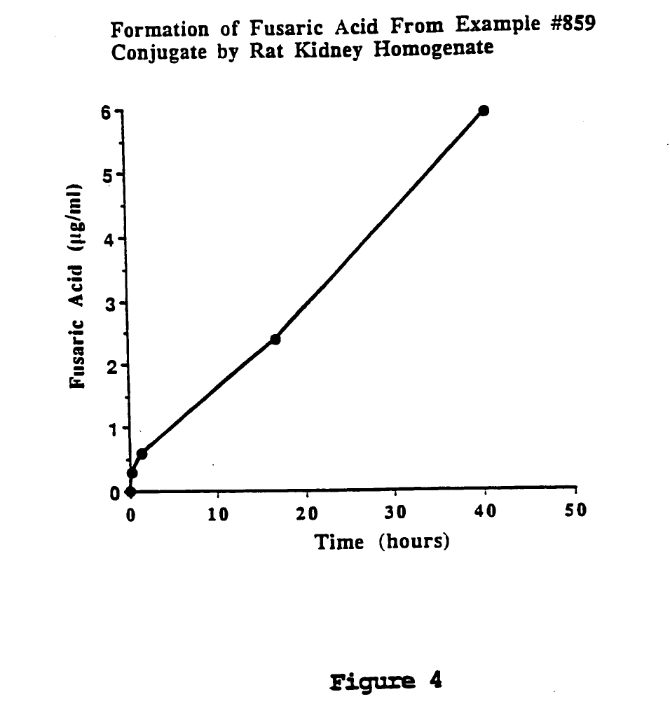

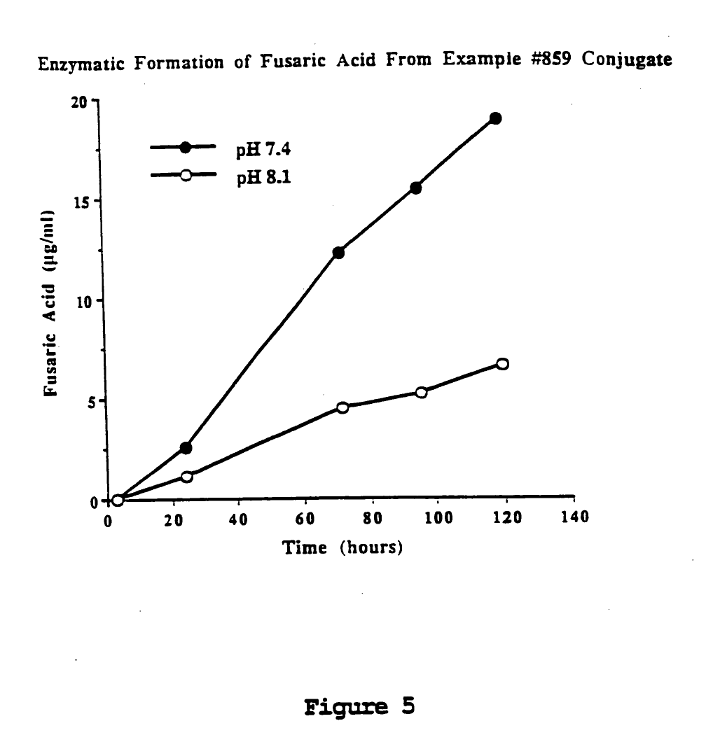

A freshly excised rat kidney was homogenized in 10 ml cold buffer (100 mM Tris, 15 mM glycylglycine, pH 7.4) with a Polytron Tissue Homogenizer (Brinkmann). The resulting suspension, diluted with buffer, was incubated in the presence of the Ex. #859 conjugate at 37° C. At various times aliquots were removed, deproteinized with an equal volume of cold trichloroacetic acid (25%) and centrifuged. The supernatant was injected onto a C-18 reverse-phase HPLC column and eluted isocratically with a mixture of acetonitrile and water (20:80 v/v) containing trifluoroacetic acid (0.05%). Eluted compounds were monitored by absorbance at 254 nm and compared to standards run under identical conditions. In the assay using pure kidney enzyme homogenate, the Ex. #859 conjugate was also incubated under the same conditions as described except that 5 mg of gamma-glutamyl transpeptidase (Sigma, 23 units/mg) and 10 mg of acylase I (Sigma, 4800 units/mg) were added in place of the homogenate. Analysis by HPLC was performed in a manner identical to that used for the kidney homogenate experiment. Following incubation of the Ex. #859 conjugate with kidney homogenate, there was a linear increase in the amount of fusaric acid liberated, as shown in FIG. 4. No fusaric acid hydrazide or gamma-glutamyl fusaric acid hydrazide was observed; nor was any metabolism observed in the buffer control incubations. These data (Table XXI, FIG. 4) show that renal tissue is able to metabolize the Ex. #859 conjugate to fusaric acid, which then remains stable under these conditions. Data from experiments using the purified enzymes show results similar to those seen for the kidney homogenate experiment, with only fusaric acid and the unreacted compound being present (see Table XXII, FIG. 5).

TABLE XXI
Formation of Fusaric Acid From the Ex. #859
Conjugate Incubated with Kidney Homogenate

| | Time (hrs.): | | | | |
|---|---|---|---|---|---|
| | 0.00 | 0.17 | 1.25 | 17.00 | 41.00 |
| Fusaric | 0.00 | 0.27 | 0.57 | 2.37 | 5.94 |
| Acid | | | | | |
| (μg/ml): | | | | | |

TABLE XXII
Formation of Fusaric Acid From Ex. #859 Conjugate
Incubated with Purified Transpeptidase and Acylase

| | Time (hrs.): | | | | |
|---|---|---|---|---|---|
| | 3 | 24 | 72 | 96 | 120 |
| Fusaric | 0.00 | 2.56 | 12.15 | 15.44 | 18.75 |
| Acid (μg/ml): | | | | | |
| @ pH 7.4 | | | | | |
| Fusaric | 0.00 | 1.12 | 4.46 | 5.22 | 6.55 |
| Acid (μg/ml): | | | | | |
| @ pH 8.1 | | | | | |

Assay V: In Vitro Evaluation of DBH Inhibition by Ex. #859 Conjugate

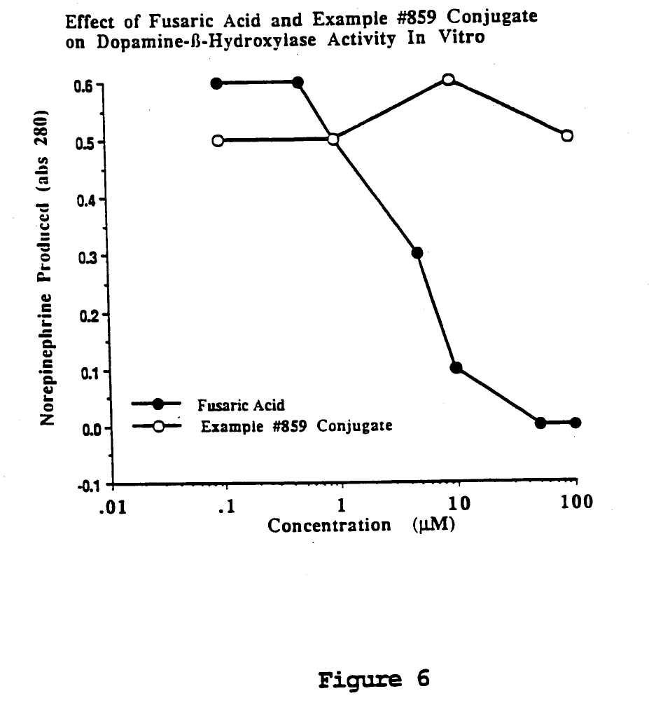

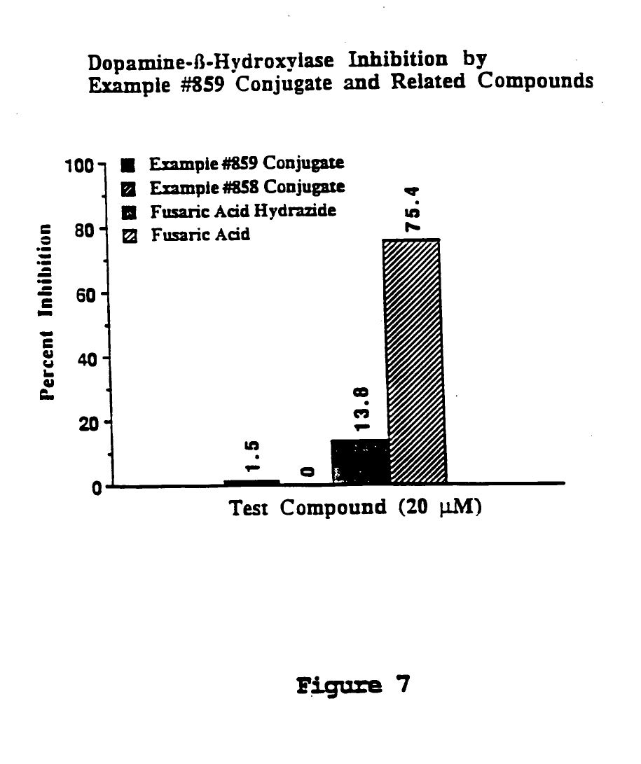

In order to characterize the relative potency of the Ex. #859 conjugate and its various potential metabolites as inhibitors of dopamine beta-hydroxylase (DBH; EC 1.14.17.1), the enzyme activity was determined in vitro in the presence of these compounds. DBH, purified from bovine adrenals (Sigma) was incubated at 37° C. in buffer containing 20 mM dopamine as substrate. The reaction was stopped by addition of 0.5 M perchloric acid. The precipitate was removed and the product of the enzyme activity (norepinephrine), contained in the clear supernatant, was analyzed by HPLC. The chromatographic separation used a reversed phase C-18 column run isocratically with 0.2 M ammonium acetate (pH 5.2) as the mobile phase. The amount of norepinephrine produced by the enzyme-substrate mixture was analyzed by measuring the peak intensity (absorbance) at 280 nm for norepinephrine as it was eluted at 4.5 minutes, using a photo-diode array detector. The result of adding either fusaric acid or the Ex. #859 conjugate to the incubate at various concentrations is shown in Table XXIII and FIG. 6. Above concentrations of 1 uM, fusaric acid inhibits the enzyme, while at concentrations up to 100 uM the Ex. #859 conjugate has no appreciable activity (Table XXIII and FIG. 6). Fusaric acid and Ex. #859 and two more possible metabolites (Ex #858 and fusaric acid hydrazide) were tested at 20 uM. Only fusaric acid had significant inhibitory effects on dopamine-β-hydroxylase activity (Table XXIV and FIG. 7).

TABLE XXIII
DBH inhibition by Fusaric Arid and the Ex. #859 Conjugate

| | Concentration (μM): | | | | | | | |
|---|---|---|---|---|---|---|---|---|
| | 0.01 | 0.10 | 0.50 | 1.00 | 5.00 | 10.00 | 50.00 | 100.00 |
| Nor- | 0.59 | 0.59 | 0.60 | 0.53 | 0.25 | 0.14 | 0.00 | 0.00 |
| epinephrine | | | | | | | | |
| Peak | | | | | | | | |
| Intensity | | | | | | | | |
| (Abs 280) | | | | | | | | |
| in the | | | | | | | | |
| presence of | | | | | | | | |
| Fusaric | | | | | | | | |
| Acid: | | | | | | | | |
| Nor- | | 0.51 | | 0.52 | | 0.61 | | 0.53 |
| epinephrine | | | | | | | | |
| Peak | | | | | | | | |
| Intensity | | | | | | | | |
| (Abs 280) | | | | | | | | |
| in the | | | | | | | | |
| presence of | | | | | | | | |
| Ex. #859 | | | | | | | | |
| Conjugate | | | | | | | | |

TABLE XXIV
DBH Inhibition by Fusaric Acid, Ex. #859
Conjugate and Various Potential Metabolites

| Test | Ex. | Ex. | Fusaric Acid | Fusaric |
|---|---|---|---|---|
| Compound (20 μM): | #859 | #858 | Hydrazide | Acid |
| % Inhibition: | 1.5 | 0.0 | 13.8 | 75.4 |

Assay VI: Acute In Vivo Effects of Ex. #859 and Ex. #863 Conjugates

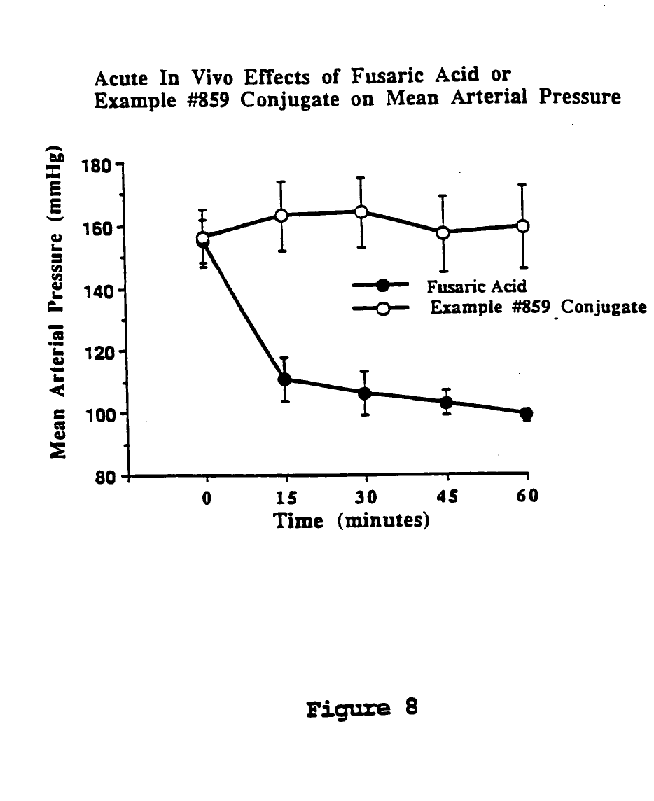

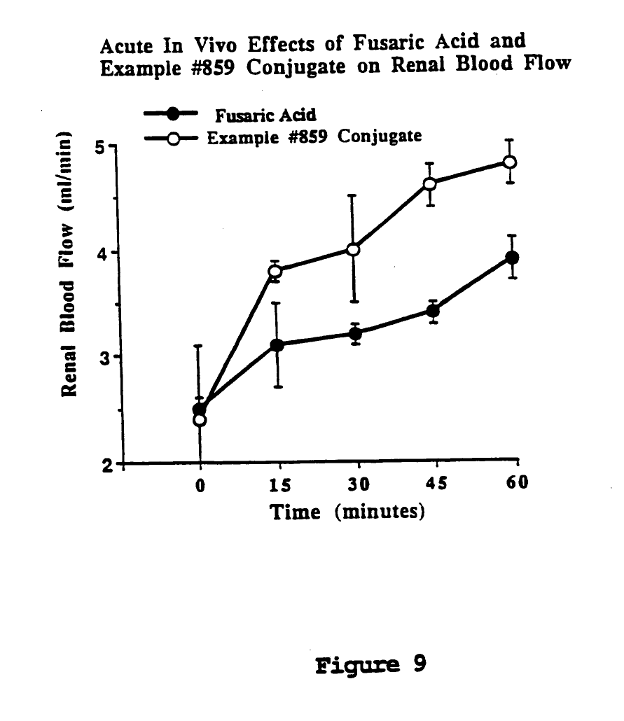

Spontaneously hypertensive rats were anesthetized with inactin (100 mg/kg, i.p.) and catheters were implanted into a carotid artery for measurement of mean arterial pressure (Gould model 3800 chart recorder; Statham pressure transducer model no. P23 DB) and into a jugular vein for compound administrations (i.v. or i.d.). In addition, a flow probe was implanted around the left renal artery for measurement of renal blood flow using pulsed Doppler flowmetry. Rats were allowed 60 min to stabilize before 10 minutes of control recordings of mean arterial pressure and renal blood flow were obtained. Control measurements were followed by intravenous injection of 50 mg/kg of fusaric acid or the Ex. #859 conjugate. As shown in FIGS. 8 and 9 and Table XXV, fusaric acid (a systemic dopamine-β-hydroxylase inhibitor) decreased mean arterial pressure and increased renal blood flow throughout the 60 minute post-injection observation period. In sharp contrast, the Ex. #859 conjugate had no acute effects on mean arterial pressure, but increased renal blood flow to a greater degree than fusaric acid (Table XXV and FIGS. 8 and 9). Similar results were found when these compounds were administered through a catheter implanted into the duodenum (i.d.). The Ex. #859 conjugate had no effect on mean arterial pressure at a dose of 100 mg/kg (n=4) during a 60 minute observation period. Renal blood flow (n=4) was unchanged 15 minutes after injection of the Ex. #859 conjugate but increased from 1.1 KHz (control period) to 3.5 KHz at 30 minutes postinjection. Renal blood flow remained at this level for the following 30 minute observation period. These data indicate that the Ex. #859 conjugate is active and displays renal selectivity whether administered i.d. or i.v. Results for Ex. #863 conjugate were similar to Ex. #859 and are shown in Table XXVI: Ex. #863 had no effect on mean arterial pressure, but increased renal blood flow, indicating renal selectivity.

TABLE XXV
Acute Effects of Fusaric Acid and Ex. #859
conjugate on Blood Pressure and Renal Blood Flow

| | Time (min) | | | | |
|---|---|---|---|---|---|
| | Zero | 15 | 30 | 45 | 60 |
| | Fusaric Acid (50 mg/kg i.v.) | | | | |
| MAP (mm Hg) | 155 | 111 | 106 | 103 | 99 |
| RBF (KHz) | 2.5 | 3.1 | 3.2 | 3.4 | 3.9 |
| | Ex. #859 Conjugate (50 mg/kg i.v.) | | | | |
| MAP (mm Hg) | 156 | 163 | 164 | 157 | 159 |
| RBF (KHz) | 2.4 | 3.8 | 4.0 | 4.6 | 4.8 |

TABLE XXVI
Acute Effects of Ex. #863 Conjugate

| | Time (min) | | | | |
|---|---|---|---|---|---|
| | Zero | 15 | 30 | 45 | 60 |
| | Ex. #863 (100 mg/kg i.v.) | | | | |
| MAP (mm Hg) | 149 ± 14 | N.A. | N.A. | N.A. | 147 ± 14 |
| RBF (KHz) | 1.6 ± 0.2 | N.A. | N.A. | N.A. | 4.3 ± 0.3 |

N.A. = Not Available

Assay VII: Comparison of Fusaric Acid, Fusaric Acid Hydrazide and Ex. #859 Conjugate on Arterial Pressure in Spontaneously Hypertensive Rats (SHR)

Mean arterial pressure effects of fusaric acid hydrazide (100 mg/kg, i.v.), fusaric acid (100 mg/kg, i.v.) and Ex. #859 conjugate (250 mg/kg, i.v.) are shown in Table XXVII during a vehicle control period and 60 min postinjection of compound in anesthetized SHR. Rats were prepared as described above, minus the renal artery flow probe.

TABLE XXVII
Acute Effects of Fusaric Acid, Fusaric Acid Hydrazide
and Ex. #859 Conjugate on Blood Pressure

| COMPOUND | ZERO | 60 MIN |
|---|---|---|
| Fusaric Acid (n = 4) | 164 ± 10 mmHg | 110 ± 21 mmHg |
| Fusaric Acid | 159 ± 8 mmHg | 104 ± 13 mmHg |
| Hydrazide (n = 4) | | |
| Ex. #859 Conjugate | 151 ± 9 mmHg | 146 ± 15 mmHg |
| (n = 4) | | |

The data show that the hypotensive effects of the fusaric acid hydrazide is similar to fusaric acid. The Ex. #859 conjugate had no effect on mean arterial pressure (Table XXV, XXVII and FIG. 8). The observation of no effect on mean arterial blood pressure confirms the expectation that the Ex. #859 conjugate does not act systemically.

Assay VIII Chronic In Vivo Effects of Ex. #859 Conjugate

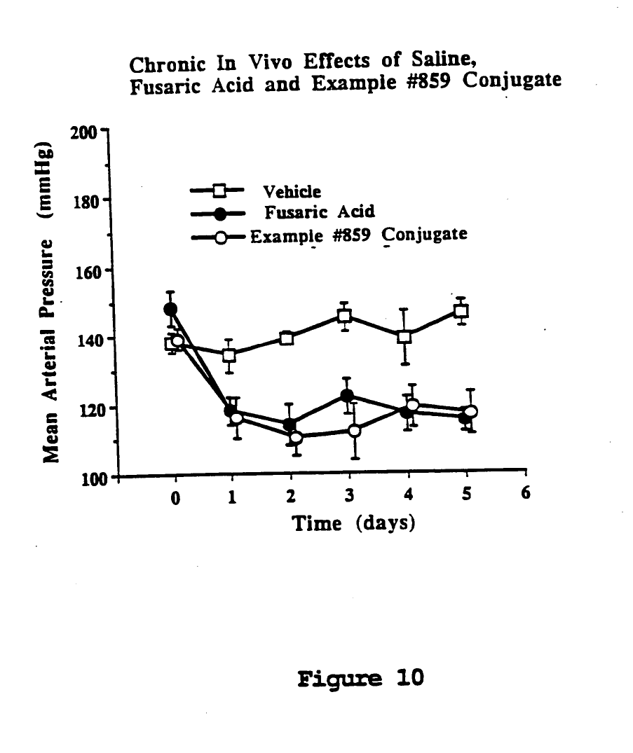

The Ex. #859 conjugate and saline vehicle were infused continuously for 5 days in SHR. Mean arterial pressure was measured (Gould Chart Recorder, model 3800; Statham P23 Db pressure transducer) via an indwelling femoral artery catheter between 10:00 a.m. and 2:00 p.m. each day. The Ex. #859 conjugate (5 mg/hr), fusaric acid (2.5 mg/hr), and saline (100 μl/hr) were infused via a jugular vein catheter with a Harvard infusion pump. Compared to the control vehicle fusaric acid and the Ex. #859 conjugate lowered mean arterial pressure similarly. Mean arterial pressure did not change in the saline vehicle group. Results are shown in Table XXVIII and FIG. 10.

TABLE XXVIII
Chronic Effects of Fusaric Acid and Ex.
#859 Conjugate on Blood Pressure

| | Time (days) | | | | | |
|---|---|---|---|---|---|---|
| | Zero | 1 | 2 | 3 | 4 | 5 |
| | Vehicle (25 μL/hr) | | | | | |
| MAP | 139 ± 2 | 139 ± 4 | 143 ± 4 | 146 ± 4 | 145 ± 7 | 146 ± 4 |
| (mm Hg) | | | | | | |
| (SE) | | | | | | |
| | Fusaric Acid (2.5 mg/hr) | | | | | |
| MAP | 148 ± 6 | 118 ± 5 | 114 ± 7 | 122 ± 5 | 114 ± 6 | 114 ± 3 |
| (mm Hg) | | | | | | |
| (SE) | | | | | | |
| | Ex. #859 Conjugate (5 mg/hr) | | | | | |
| MAP | 146 ± 5 | 122 ± 9 | 115 ± 9 | 119 ± 11 | 121 ± 7 | 115 ± 8 |
| (mm Hg) | | | | | | |
| (SE) | | | | | | |

Assay IX: Chronic In Vivo Effects of Ex. #861 and Ex. #863 Conjugates

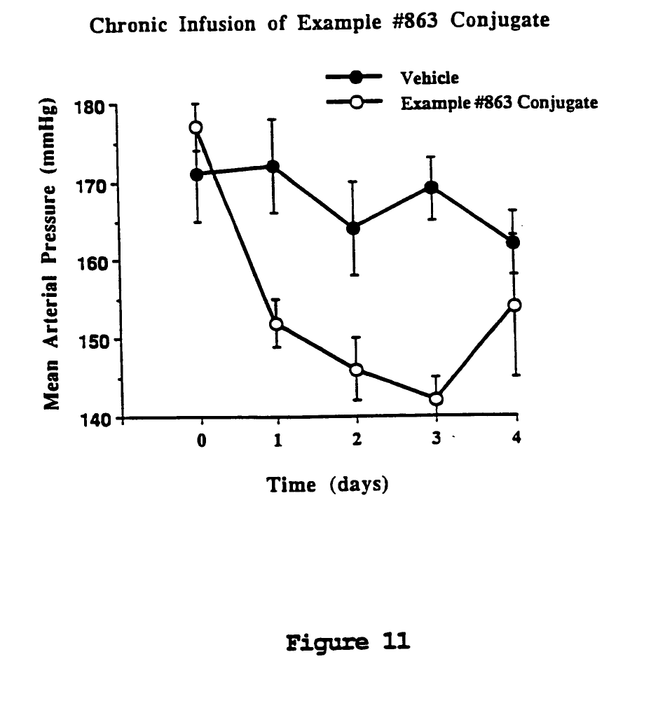

The conjugates of Ex. #861 and #863 and saline vehicle were infused continuously for 4 days in spontaneously hypertensive rats. Mean arterial pressure was measured (Gould Chart Recorder, model 3800; Statham P23 Db pressure transducer) via an indwelling femoral artery catheter between 10:00 a.m. and 2:00 p.m. each day. The Ex. #861 and Ex. #863 conjugates were infused at 5 mg/hr and the saline vehicle was infused at 100 μl/hr via a jugular vein catheter with a Harvard infusion pump. Results are shown in Table XXIX. The Ex. #863 conjugate lowered mean arterial pressure as shown in FIG. 11. Mean arterial pressure did not change for the Ex. #861 conjugate and the saline vehicle group (Table XXIX). It is believed that at a higher dose of the Ex. #861 conjugate, blood pressure lowering effects would be observed.

TABLE XXIX
Chronic Effects of Ex. #861 and Ex. #863 Conjugates
on Blood Pressure

| | Time (days) | | | | |
|---|---|---|---|---|---|
| | Zero | 1 | 2 | 3 | 4 |
| Vehicle | 171 ± 6 | 172 ± 6 | 164 ± 6 | 169 ± 4 | 162 ± 4 |
| Ex. #861 | 177 ± 3 | 173 ± 3 | 172 ± 4 | 172 ± 3 | 163 ± 9 |
| Ex. #863 | 177 ± 5 | 152 ± 6 | 146 ± 7 | 142 ± 7 | 154 ± 7 |

Assay X: Catecholamine Analysis of Tissue from Rats Treated with Ex. #859 Conjugate

In order to evaluate the renal selectivity of DBH inhibition by the Ex. #859 conjugate, the catecholamine levels of heart and kidneys, both of which have been shown to be highly sensitive to DBH inhibition [Racz, K. et al., Europ. J. Pharmacol., 109, 1 (1985)], were measured following chronic infusion of the Ex. #859 conjugate, fusaric acid and saline vehicle in rats. Following 5 days of infusion, the kidney was exposed through a small flank incision, made in the anesthetized rat, and the renal artery and vein were ligated. Following this the kidney was rapidly excised distal to the ligation and frozen in liquid nitrogen. Similarly, the heart was excised and frozen subsequent to the removal of both kidneys. The frozen tissues were stored in closed containers at −80° C. Tissue samples were thawed on ice and their weight recorded prior to being placed in a flat bottom tube. The cold extraction solvent (2 ml/g tissue) was then added and the sample was homogenized with a Polytron. Extraction Solvent: 0.1 M perchloric acid (3 ml of 70% PCA to 500 ml); 0.4 mM Na metabisulphite (38 mg/500 ml). The volume was then measured and 0.05 ml of a 1 uM/L solution of dihydroxybenzylamine (DHBA) in extraction solvent was added for every 0.95 ml of homogenate to yield a 50 nM/L internal standard concentration. The homogenate was then mixed and centrifuged at 4° C., 3000 rpm for 35 minutes. A 2 ml aliquot of the supernatant was then neutralized by adding 0.5 ml of 2 M Tris, pH 8.8 and mixing. The sample was then placed on an alumina column (40 mg, Spe-ed CAT cartridge; Applied Separations; Bethlehem, Pa.) and the catecholamines were bound, washed and eluted using a vacuum manifold system (Adsorbex SPU, EM Science, Cherry Hill, N.J.) operating at ca. 4 ml/min. until the column was dry. Washes of 1 ml H₂O— 0.5 ml MeOH— 1 ml H₂O were followed by elution with 1 ml of extraction solvent. A 200 μl sample of the eluant was injected onto a C-18 reversed phase analytical HPLC column, 5 um, 4.6 mm×250 mm (e.g., Beckman #235335, LKB 2134-630 Spherisorb ODS-2) and eluted with a recycled mobile phase run at ambient temperature and a flow rate of 0.5 ml/min (ca. 75 bar).

Mobile Phase: 0.02 M Na₂HPO₄ in 75/25(v/v) H₂O/MeOH 0.007% SDS pH 3.5 (conc. H₃PO₄). The separated catecholamines were detected with a LKB 2143 electrochemical detector at a potential setting of 500 mV using a teflon flow cell spacer of 2.2 μl and a time constant of 2 sec. Peak heights were measured and recorded along with the chromatogram tracing using a Spectra-Physics 4270 integrator. Sample runs were preceded by injection of a mixture of calibration standards (200 ul) containing 50 nM/L of epinephrine (Epi), norepinephrine (NE), dopamine (DA), and DHBA in extraction solvent. The peak heights for each sample run were corrected by dividing the peak height of the DHBA in the standard by the peak height of the DHBA in each sample. The resulting factor (calculated for each sample) was used to correct for losses due to dilution, non-specific binding to the tissue precipitate, incomplete elution, etc. Concentrations were calculated by multiplying the peak heights for Epi, Nebr. and DA by that samples correction factor and then dividing this value by the peak height of the respective standard. When this number is multiplied by the concentration of the standard (in this case 50 nM/L) the concentration of the catecholamine in the homogenate is obtained. This value is multiplied by the volume of the homogenate (determined previously) to get the total catecholamine content of the tissue expressed in moles/g tissue. The resolution and retention times for a mixture of standards run under the conditions described in the previous section are shown in Table XXX.

TABLE XXX

| Retention | |
|---|---|
| Time (min.) | Compound |
| 12.10 | 3,4-dihydroxylphenylacetic acid |
| | (DOPAC) |
| 18.24 | norepinephrine (NE) |
| 21.82 | epinephrine (Epi) |
| 23.19 | homovanillic acid (HVA) |
| 30.56 | dihydroxybenzylamine (DHBA) |
| 42.58 | dopamine (DA) |

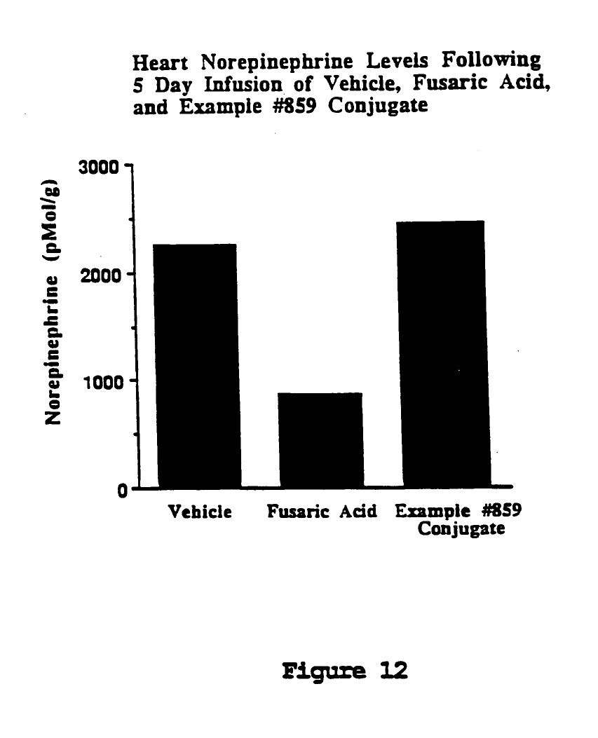

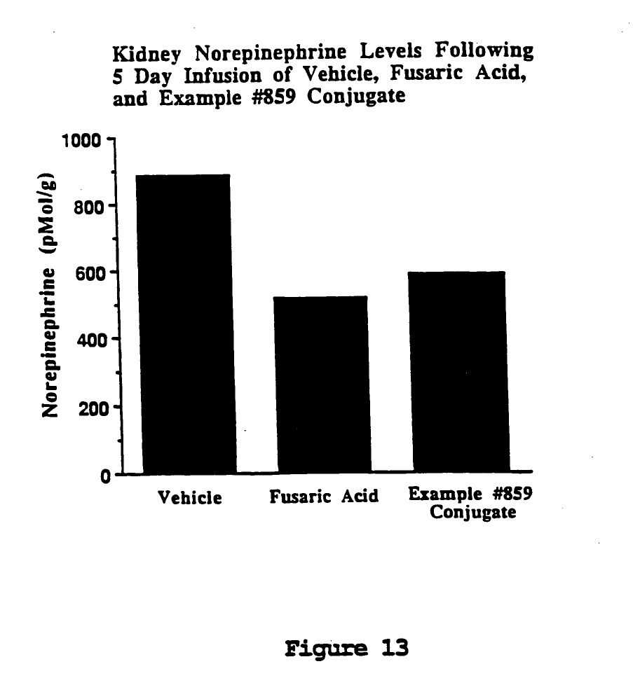

The linear response to various standards run over a 100 fold concentration range was excellent with values for both the correlation coefficient (r) and the coefficient of determination (r-squared) being >0.9999 for all standards, while the rank correlation (Spearman's rho) was 1.0. To confirm the precision and accuracy of the values, tissue analysis was performed on a control group of Sprague-Dawley rats. The cumulative results are within the range of values reported in the literature [(e.g. Racz, K. et al, J. Cardiovasc. Pharmacol. 8, 676 (1986)]. The precision in the efficiency of extraction measured by the addition of an internal standard (DHBA) was also excellent with a fractional efficiency of 0.779(SE=0.066) for the kidney extraction and 0.771(SE=0.083) for the heart extracts. Relative to vehicle administration, both the Ex. #859 conjugate and fusaric acid decreased kidney norepinephrine concentration; however, only fusaric acid decreased heart norepinephrine concentration (see Table XXXI and FIGS. 12 and 13). These data indicate that the Ex. #859 conjugate is renal selective with chronic infusion.

TABLE XXXI
Effect of Fusaric Acid and Ex. #859 conjugate on Tissue
Norepinephrine Concentration Following 5 Days of Infusion

Tissue:

| | Kidney | Heart |
|---|---|---|
| | Vehicle (25 μL/hr) | |
| Norepinephrine: | 889(72) | 2,248(164) |
| (pMol/g) (SD) | | |
| | Fusaric Acid (2.5 mg/hr) | |
| Norepinephrine: | 519(42) | 862(147) |
| (pMol/g) (SD) | | |
| | Ex. #859 Conjugate (5 mg/hr) | |
| Norepinephrine: | 589(54) | 2,444(534) |
| (pMol/g) (SD) | | |

Assay XI: Intrarenal Administration of Fusaric Acid in Anesthetized Dogs

In one anesthetized dog, bolus doses of fusaric acid (0.1-5.0 mg/kg) were administered into the renal artery. Mean arterial pressure (MAP), renal blood flow (RBF) and urinary sodium excretion (U_{Na}V) were measured. Bolus intrarenal injection of isotonic saline or 0.1 mg/kg of fusaric acid had no effect on any measure; however, 0.5, 1.0, and 5.0 mg/kg fusaric acid caused dose-related increases in renal blood flow, but had no significant effect on mean arterial pressure or urinary sodium excretion (see Table XXXII).

TABLE XXXII
Effect of Intrarenal Injection of Fusaric Acid
on Blood Pressure, Sodium Excretion and
Renal Blood Flow in the Dog

| Dose (mg/kg): | Saline | 0.1 | 0.5 | 1.0 | 5.0 |
|---|---|---|---|---|---|
| Δ RBF (ml/min): | 0 | 0 | +46 | +58 | +132 |
| U_{Na} V (μEq/min): | 42.8 | 21.2 | 23.8 | 21.1 | 34.8 |
| MAP (mm Hg): | 136 | 136 | 136 | 138 | 140 |

Similar results were also found in a second experiment where non-depressor doses of fusaric acid were infused into the renal arteries of two dogs (see Table XXXIII).

TABLE XXXIII
Effect of Intrarenal Infusion of Fusaric Acid on Blood Pressure,
Sodium Excretion and Renal Blood Flow in the Dog

| | Infusion: | | | |
|---|---|---|---|---|
| | Dog #1 | | Dog #2 | |
| | | Fusaric Acid | | Fusaric Acid |
| | | (1.25 | | (0.75 |
| | Saline | mg/kg/min) | Saline | mg/kg/min) |
| Δ RBF | 140 | 240 | 236 | 315 |
| (ml/min): | | | | |
| U_{Na} V | 95 | 82 | 44 | 13 |
| (μEqlmin): | | | | |
| MAP (mm Hg): | 136 | 136 | 140 | 148 |

These data indicate that intrarenal administration of fusaric acid increases renal blood flow in anesthetized dogs without altering systemic mean arterial pressure.

Assay XII: Acute In Vivo Effects of Ex. #859 Conjugate

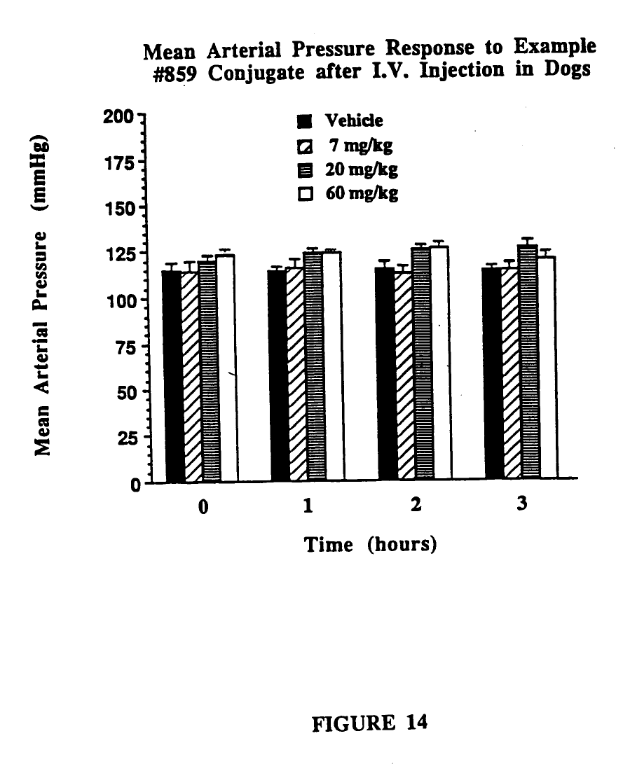

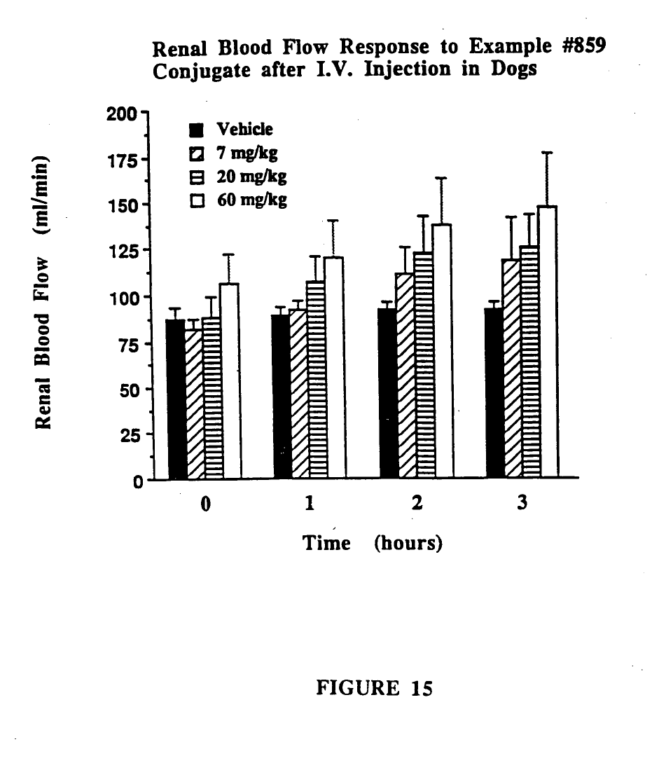

This experiment was run to determine the renal selectivity of conjugate of the invention in dogs. Male mongrel dogs (15-20 kg/n=8; Antech, Inc., Barnhard, Mo.) were anesthetized with sodium pentobarbital (30 mg/kg as i.v. bolus, and 4-6 mg/kg/hr infusion) and catheters were placed in the femoral veins for compound injection or pentobarbital infusion, and the femoral artery for arterial pressure recording. An electromagnetic flow probe (Carolina Medical Electronics, Inc., King, N.C.) was placed around the left renal artery for measurement of renal blood flow. Renal blood flow and arterial pressure were recorded on a Gould chart recorder. After surgery, 20-30 minutes were allowed for variables to stabilize. Then a 20 minute control measurement was followed by injection of Ex. #859 conjugate at doses of 20 and 60 mg/kg, i.v., to two different groups of dogs. Variables were monitored for the next three hours. Results are shown in Table XXXIV and FIGS. 14 and 15.

TABLE XXXIV
Renal Selectivity of Ex. #859 Conjugate in Dogs

Time After Injection of Ex. #859 Conjugate

| | | | |
|---|---|---|---|
| Zero | 1 Hour | 2 Hour | 3 Hour |

Mean Arterial Pressure (mmHg)

| | | | | |
|---|---|---|---|---|
| 7 mg/kg | 114 ± 6 | 116 ± 5 | 113 ± 4 | 114 ± 4 |
| 20 mg/kg | 120 ± 3 | 124 ± 2 | 125 ± 3 | 125 ± 4 |
| 60 mg/kg | 123 ± 3 | 124 ± 1 | 126 ± 3 | 120 ± 4 |
| Vehicle | 115 ± 4 | 114 ± 3 | 115 ± 4 | 114 ± 3 |

Renal Blood Flow (ml/min)

| | | | | |
|---|---|---|---|---|
| 7 mg/kg | 92 ± 5 | 92 ± 5 | 111 ± 14 | 118 ± 23 |
| 20 mg/kg | 88 ± 11 | 107 ± 14 | 122 ± 20 | 126 ± 24 |
| 60 mg/kg | 131 ± 21 | 145 ± 21 | 168 ± 28 | 176 ± 32 |
| Vehicle | 87 ± 7 | 89 ± 5 | 92 ± 4 | 92 ± 4 |

Assay XIII: Acute In Vivo Effects of Ex #859 Conjugate

This experiment was run to determine the roles of the renal sympathetic nerves and dopamine in the antihypertensive response to Ex. #859. For renal blood flow experiments, male SHR (11-13 weeks of age; Harlan Sprague-Dawley, Inc., Indianapolis, Ind.) were anesthetized (Inactin, 100 mg/kg, i.p.), catheters were implanted in a jugular vein and carotid artery, and an electromagnetic flow probe (Carolina Medical Electronics, Inc., King, N.C.) was placed on the left renal artery. Care was taken not to damage the renal nerves. A tracheal catheter maintained airway patency. The SHR were placed on a heated pad to maintain normal body temperature (Harvard Apparatus, South Natick, Mass.). In one group of SHR (n=6) surgical renal denervation was performed (prior to implanting the flow probe) through a left flank incision by surgically stripping the renal artery and vein of adventitia and cutting all visible renal nerve bundles under a dissection microscope (×25) and coating the vessels with a solution of 10% phenol in 95% ethanol, as previously described (9,10). In a second group of SHR (n=6) bulbocapnine (a dopamine receptor antagonist) was infused at 100 μg/kg/min starting 30 minutes prior to injection of Ex. #859 (50 mg/kg, i.v.) and continued for the duration of the study. In a third group of SHR (n=6) Ex. #859 (50 mg/kg, i.v.) was administered alone. In a final group of SHR (n=6) vehicle (0.9% NaCl) was administered. SHR were allowed 60 minutes for stabilization after surgery. After the stabilization period, 15 minutes of control mean arterial pressure and renal blood flow were obtained. Mean arterial pressure and renal blood flow were recorded for one hour.

For antihypertensive experiments, male SHR (11-13 weeks of age; Harlan Sprague-Dawley, Inc.; Indianapolis, Ind.) were habituated for 3-4 days in individual experimental cages, which became their home cages for the duration of the study. Five to seven days before experimentation, SHR were anesthetized with chloral hydrate (400 mg/kg; Sigma Chemical Co., St. Louis, Mo.) and catheters were implanted into a femoral artery and vein. The catheters were led to the back of the neck, exteriorized, and channeled through a tether and swivel system (Alice King Chatham, Los Angeles, Calif.). Surgical renal denervation was performed as above. SHR that did not resume normal food and water consumption were omitted from the study. Mean arterial pressure was measured via a pressure transducer (Model P23 Db, Statham, Oxnard, Calif.) and displayed on a chart recorder (Gould, model 3800, Cleveland, Ohio). In separate groups of conscious SHR, Ex. #859 (5 mg/kg/hr, n=6) was infused alone, Ex. #859 (5 mg/kg/hr, n=6) was coinfused with bulbocapnine (1001 g/kg/min), or Ex. #859 (10 mg/kg/hr, n=6) was infused 5-7 days after surgical renal denervation. Surgical renal denervation was performed as described above. After a one hour control measure of mean arterial pressure, compounds were infused for four hours and mean arterial pressure was measured continuously.

In anesthetized SHR, mean arterial pressure was not changed in any group (Table XXXV). Similarly, vehicle had no effect on renal blood flow in anesthetized SHR (Table XXXV). Renal blood flow was increased 60 minutes after injection of Ex. #859 alone, but renal blood flow was not changed by Ex. #859 during bulbocapnine infusion or after surgical renal denervation (Table XXXV).

In conscious SHR, continuous infusion of Ex. #859 was antihypertensive over a four hour period (Table XXXVI). Coinfusion of Ex. #859 with bulbocapnine lowered mean arterial pressure similar to Ex. #859 alone (Table XXXVI). Bulbocapnine alone had no effect on mean arterial pressure over the four hour period (Table XXXVI). In contrast, surgical denervation of the kidneys prevented the antihypertensive response to Ex. #859 (Table XXXVI). Renal denervation also lowered baseline mean arterial pressure relative to vehicle (Table XXXVI).

TABLE XXXV
Role of Dopamine and Renal Nerves on
Responses to Ex. #859 Conjugate

| | Mean Arterial | Renal Blood |
|---|---|---|
| | Pressure (mmHg) | Flow (ml/min) |
| Vehicle n = 6 | | |
| Time 0 minutes | 151 ± 8 | 8 ± 1 |
| Time 60 minutes | 151 ± 6 | 9 ± 1 |
| Ex. #859 n = 6 | | |
| Time 0 minutes | 149 ± 8 | 7 ± 2 |
| Time 60 minutes | 149 ± 7 | 12 ± 2 |
| Bulbocapnine + SC-47792 n = 6 | | |
| Time 0 minutes | 148 ± 7 | 7 ± 1 |
| Time 60 minutes | 146 ± 7 | 7 ± 1 |
| Renal Denervation + SC-47792 n = 6 | | |
| Time 0 minutes | 143 ± 6 | 6 ± 1 |
| Time 60 minutes | 139 ± 7 | 6 ± 1 |

TABLE XXXVI
Role of Dopamine and Renal Nerves on Antihypertensive
Response to Ex. #859 Conjugate

| | Time (hours) | | | | |
|---|---|---|---|---|---|
| | 0 | 1 | 2 | 3 | 4 |
| Vehicle (n = 6) | 186 ± 8 | 186 ± 8 | 184 ± 7 | 180 ± 8 | 179 ± 8 |
| Ex. #859 (n = 6) | 177 ± 6 | 172 ± 6 | 170 ± 7 | 164 ± 7 | 154 ± 6 |
| DNX (n = 6) | 157 ± 3 | 155 ± 4 | 53 ± 4 | 150 ± 4 | 147 ± 4 |
| BULBO (n = 6) | 168 ± 8 | 158 ± 6 | 148 ± 5 | 140 ± 7 | 140 ± 5 |
| BULBO (n = 6) | 160 ± 6 | 156 ± 7 | 161 ± 11 | 159 ± 6 | 157 ± 7 |
| alone | | | | | |

Assay XIV: Chronic In Vivo Effects of Ex. #859 Conjugate in DOCA Hypertensive Micropigs

This study examines the efficacy of Ex. #859 in deoxycorticosterone acetate (DOCA) hypertensive micropigs (Charles River; 6 months of age). Micropigs were made hypertensive by implanting subcutaneously DOCA strips (100 mg/kg) under isoflurane anesthesia. Hypertension stabilizes after one month. Mean arterial pressure was measured using a Gould chart recorder and Statham P23 dB transducers. After one month Ex. #859 conjugate was infused for three days at 5 mg/kg/hr).

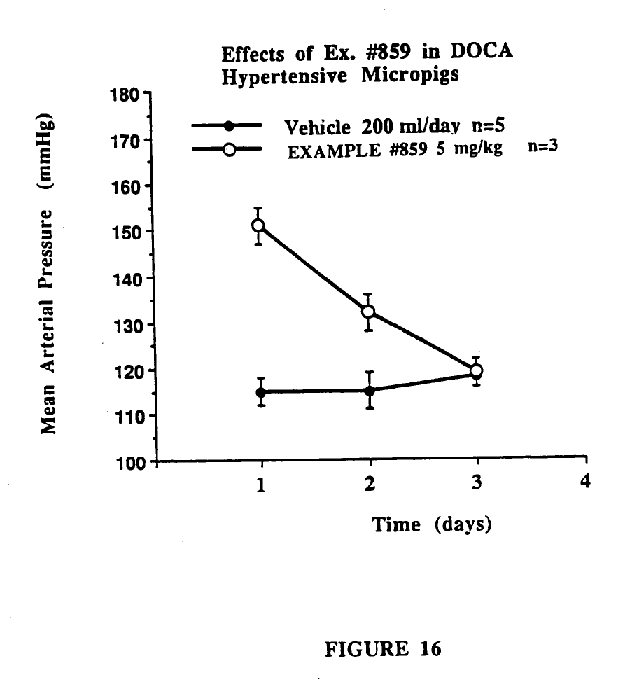

Vehicle infusion (200 ml/day) had no effect on mean arterial pressure over the three day study period Table XXXVI and FIG. 16). Example #859 normalized mean arterial pressure (Table XXXVI and FIG. 16).

TABLE XXXVI
Effects of Ex. #859 on Mean Arterial
Pressure in DOCA Hypertensive Micropigs

| Vehicle | Day 1 | Day 2 | Day 3 |
|---|---|---|---|
| | 115 ± 3 | 115 ± 4 | 118 ± 2 |
| Ex. #859 | 151 + 4 | 132 + 4 | 119 + 3 |

### Compositions of the Invention

Also embraced within this invention is a class of pharmaceutical compositions comprising one or more conjugates described above in association with one or more non-toxic, pharmaceutically acceptable carriers and/or diluents and/or adjuvants (collectively referred to herein as “carrier” materials) and, if desired, other active ingredients. The conjugates of the present invention may be administered by any suitable route, preferably in the form of a pharmaceutical composition adapted to such a route, and in a dose effective for the treatment intended. Therapeutically effective doses of the conjugates of the present invention required to prevent or arrest the progress of the medical condition are readily ascertained by one of ordinary skill in the art. The conjugates and composition may, for example, be administered intravascularly, intraperitoneally, subcutaneously, intramuscularly or topically.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. These may with advantage contain an amount of active ingredient from about 1 to 250 mg, preferably from about 25 to 150 mg. A suitable daily dose for a human may vary widely depending on the condition of the patient and other factors. However, a dose of from about 0.1 to 3000 mg/kg body weight, particularly from about 1 to 100 mg/kg body weight, may be appropriate.

The active ingredient may also be administered by injection as a composition wherein, for example, saline, dextrose solutions or water may be used as a suitable carrier. A suitable daily dose is from about 0.1 to 100 mg/kg body weight injected per day in multiple doses depending on the disease being treated.

A preferred daily dose would be from about 1 to 30 mg/kg body weight. Conjugates indicated for prophylactic therapy will preferably be administered in a daily dose generally in a range from about 0.1 mg to about 100 mg per kilogram of body weight per day. A more preferred dosage will be a range from about 1 mg to about 100 mg per kilogram of body weight. Most preferred is a dosage in a range from about 1 to about 50 mg per kilogram of body weight per day. A suitable dose can be administered, in multiple sub-doses per day. These sub-doses may be administered in unit dosage forms. Typically, a dose or sub-dose may contain from about 1 mg to about 100 mg of conjugate per unit dosage form. A more preferred dosage will contain from about 2 mg to about 50 mg of conjugate per unit dosage form. Most preferred is a dosage form containing from about 3 mg to about 25 mg of active compound per unit dose.

The dosage regimen for treating a disease condition with the conjugates and/or compositions of this invention is selected in accordance with a variety of factors, including the type, age, weight, sex and medical condition of the patient, the severity of the disease, the route of administration, and the particular compound employed, and thus may vary widely.

For therapeutic purposes, the conjugates of this invention are ordinarily combined with one or more adjuvants appropriate to the indicated route of administration. If administered per os, the conjugates may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of conjugate in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The conjugates may be dissolved in water, polyethylene glycol, propylene glycol, ethanol, corn oil, cottonseed oil, peanut oil, sesame oil, benzyl alcohol, sodium chloride solutions, and/or various buffer solutions. Other adjuvants and modes of administration are well and widely known in the pharmaceutical art. Appropriate dosages, in any given instance, of course depend upon the nature and severity of the condition treated, the route of administration, including the weight of the patient.

Representative carriers, diluents and adjuvants include for example, water, lactose, gelatin, starches, magnesium stearate, talc, vegetable oils, gums, polyalkylene glycols, petroleum jelly, etc. The pharmaceutical compositions may be made up in a solid form such as granules, powders or suppositories or in a liquid form such as solutions, suspensions or emulsions. The pharmaceutical compositions may be subjected to conventional pharmaceutical operations such as sterilization and/or may contain conventional pharmaceutical adjuvants such as preservatives, stabilizers, wetting agents, emulsifiers, buffers, etc.

Although this invention has been described with respect to specific embodiments, the details of these embodiments are not to be construed as limitations. Various equivalents, changes and modifications may be made without departing from the spirit and scope of this invention, and it is understood that such equivalent embodiments are part of this invention.

What is claim is:

1. A conjugate comprising a first residue and a second residue, said first and second residues connected together by a cleavable bond, wherein said first residue is provided by an inhibitor compound capable of inhibiting biosynthesis of an adrenergic neurotransmitter, and wherein said second residue is capable of being cleaved from said first residue by an enzyme located predominantly in the kidney.

2. Conjugate of claim 1 wherein said first and second residues are provided by precursor compounds, wherein the precursor compound of one of said first and second residues has a reactable carboxylic acid moiety and the precursor of the other of said first and second residues has a reactable amino moiety or a moiety convertible to a reactable amino moiety, whereby a cleavable bond may be formed between said carboxylic acid moiety and said amino moiety.

3. Conjugate of claim 2 wherein said inhibitor compound providing said first residue is selected from tyrosine hydroxylase inhibitor compounds, dopa-decarboxylase inhibitor compounds, dopamine-β-hydroxylase inhibitor compounds, and mimics of said inhibitor compounds.

4. Conjugate of claim 3 wherein said tyrosine hydroxylase inhibitor compound is of the formula 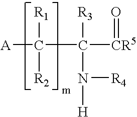
wherein each of R¹ through R³ is independently selected from hydrido, hydroxy, alkyl, cycloalkyl, cycloalkylalkyl, aralkyl, aryl, alkoxy, aryloxy, aralkoxy, alkoxyalkyl, haloalkyl, hydroxyalkyl, halo, cyano, amino, monoalkylamino, dialkylamino, carboxyl, carboxyalkyl, alkanoyl, alkenyl, cycloalkenyl and alkynyl; wherein R⁴ is selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein R⁵ is selected from —OR⁶ and 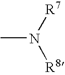
wherein R⁶ is selected from hydrido, alkyl, cycloalkyl, cycloalkylalkyl, aralkyl and aryl, and wherein each of R⁷ and R³ is independently selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; aralkyl; wherein m is a number selected from zero through six;
wherein A is a phenyl ring of the formula 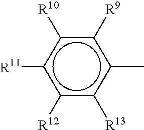
wherein each of R⁹ through R¹³ is independently selected from hydrido, hydroxy, alkyl, cycloalkyl, cycloalkylalkyl, aralkyl, aryl, alkoxy, aralkoxy, alkoxyalkyl, haloalkyl, hydroxyalkyl, halo, cyano, amino, monoalkylamino, dialkylamino, carboxyl, carboxyalkyl, alkanoyl, alkenyl, cycloalkenyl, alkynyl, cyanoamino, carboxyl, cyano, thiocarbamoyl, aminomethyl, alkylsulfanamido, nitro, alkylsulfonyloxy, carboxyalkoxy, formyl and a substituted or unsubstituted 5- or 6-membered heterocyclic ring selected from the group consisting of pyrrol-1-yl, 2-carboxypyrrol-1-yl, imidazol-2-ylamino, indol-1-yl, carbozol9-yl, 4,5-dihydro-4-hydroxy-4-trifluoro-methylthiazol-3-yl, 4-trifluoromethylthiazol-2-yl, imidazol-2-yl and 4,5-dihydroimidazol-2-yl; wherein any two of the R⁹ through R¹³ groups may be taken together to form a benzoheterocylic ring selected from the group consisting of indolin-5-yl, 1-(N-benzoylcarbamimidoyl) indolin-5-yl, 1-carbamimidoylindolin-5-yl, 1H-2-oxindol-5-yl, insol-5-yl, 2-mercaptobenzimidazol-5 (6)-yl, 2-aminobenzimidazol-5-(6)-yl, 2-methanesulfonamidobenzimidazol-5(6)-yl, 1H-benzoxanol-2 on-6-yl, 2-aminobenzothiazol-6-yl, 2-amino-4-mercaptobenzothiazol-6-yl, 2,1,3-benzothiadiazol-5-yl, 1,3-dihydro-2,2-dioxo2,1,3-benzothiadiazol-5-yl, 1,3-dihydro-1,3-dimethyl-2,2-dioxo-2,1,3-benzothiadiazol-5-yl, 4-methyl-2(H)oxoquinolin-6-yl, quinoxalin-6-yl, 2-hydroxyquinoxalin-6-yl, 2-hydroxquinoxalin-7-yl, 2,3-dihydroxyquinoxalin-6-yl and 2,3-didydro-3(4H)-oxo-1,4-benzoxazin-7-yl; 5-hydroxy-4H-pyran-4-on-2-yl, 2-hydroxypyrid-4-yl, 2-aminopyrid-4-yl, 2-carboxypyrid-4-yl or tetrazolo-[1,5-a]pyrid-7-yl; and wherein A may be selected from 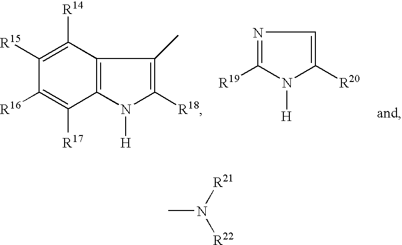
wherein each of R¹⁴ through R²⁰ is independently selected from hydrido, alkyl, hydroxy, hydroxyalkyl, alkoxy, cycloalkyl, cycloalkylalkyl, halo, haloalkyl, aryloxy, alkoxycarboxyl, aryl, aralkyl, cyano, cyanoalkyl, amino, monoalkylamino and dialkylamino, wherein each of R²¹ and R²² is independently selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; or a pharmaceutically-acceptable salt thereof.

5. Conjugate of claim 4 wherein said inhibitor compound is of the formula 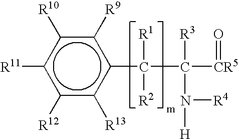
wherein each of R¹ and R² is hydrido; wherein m is one; wherein R³ is selected from alkyl, alkenyl and alkynyl; wherein R⁴ is selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein R⁵ is selected from OR⁶ and 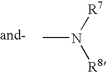
wherein R⁶ is selected from hydrido, alkyl, cycloalkyl, cycloalkylalkyl, phenalkyl and phenyl, and wherein each of R⁷ and R³ is independently selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein each of R⁹ through R¹³ is independently selected from hydrido, hydroxy, alkyl, cycloalkyl, cycloalkylalkyl, aralkyl, aryl, alkoxycarbonyl, alkoxycarbonyl, alkoxy, arykoxy, aralkoxy, alkoxyalkyl, haloalkyl, hydroxyalkyl, halo, cyano, amino, monoalkylamino, dialkylamino, dialkylamino, carboxyl, carboxyalkyl, alkanoyl, alkenyl, cycloalkenyl, alkynyl, pyrrol-1-yl 2-carboxypyrrol-1-yl, imidazol-2-ylamino, indol-1-yl, carbazol-9-yl, 4,5-dihydro-4-trifluoromethylthiazol-3-yl, 4-trifluoromethylthiazol-2-yl, imidazol-2-yl and 4,5-dihydroimidazol-2-yl, and wherein any two of the R⁹ through R¹³ groups may be taken together to form a benzoheterocyclic ring selected from the group consisting of indolin-5-yl, 1-(N-benzoylcarbamimidoyl)indolin-5-yl, 1-carbamimidoylindolin-5-yl, 1H-2-oxindol-5-yl, indol-5-yl, 2-mercaptobenzimidazol-5(6)yl, 2-aminobenzimidazol-5-(6)-yl, 2-methanesulfonamidobenzimidazol-5(6)-yl, 1H-benzoxanol-2-on-6-yl, 2-aminobenzothiazol-6-yl, 2-amino-4-mercaptobenzothiazol-6-yl, 2,1,3-benzothiadiazol-5-yl, 1,3-dihydro-2,2-dioxo-2,1, 3-benzothiadiazol-5-yl, 1,3-dihydro-1,3-dimethyl-2,2-dioxo-2,1,3-benzothiadiazol-5-yl, 4-methyl-2(H)oxoquinolin-6-yl, quinoxalin-6-yl, 2-hydroxyquinoxalin6-yl, 2-hydroxquinoxalin-7-yl, 2,3-dihydroxyquinoxalin-6-yl and 2,3-didydro-3 (4H)-oxo-1,4-benzoxazin-7-yl; wherein R³ is —CH═CH₂ or —C≡CH; wherein R⁵ is selected from OR⁶ and 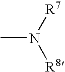
wherein R⁶ is selected from hydrido, alkyl, hydroxy, hydroxyalkyl, alkoxy, halo, haloalkyl, cycloalkyl, cycloalkylalkyl, aryl, aralkyl, amino, monoalkylamino, dialkylamino; and wherein each of R⁷ and R³ independently is selected from hydrido, alkyl, hydroxyalkyl, cycloalkyl, cycloalkylalkyl, aryl and aralkyl; or a pharmaceutically-acceptable salt thereof.

6. Conjugate of claim 5 wherein said inhibitor compound is selected from the group consisting of
4-cyanoamino-a-methylphenyalanine;
3-carboxy-a-methylphenylalanine;
3-cyano-a-methylphenylalanine methyl ester;
α-methyl-4-thiocarbamoylphenylalanine methyl ester;
4-(aminomethyl)-a-methylphenylalanine;
4-guanidino-a-methylphenylalanine;
3-hydroxy-4-methanesulfonamido-a-methylphenylalanine;
3-hydroxy-4-nitro-a-methylphenylalanine;
4-amino-3-methanesulfonyloxy-a-methylphenylalanine;
3-carboxymethoxy-4-nitro-a-methylphenylalanine;
α-methyl-4-amino-3-nitrophenylalanine;
3,4-diamino-a-methylphenylalanine;
α-methyl-4-(pyrrol-1-yl)phenylalanine;
4-(2-aminoimidazol-1-yl)-a-methylphenylalanine;
4-(imidazol-2-ylamino)-a-methylphenylalanine;
4-(4,5-dihydro-4-hydroxy-4-trifluoromethyl-thiazol-2-yl)a-methylphenylalanine methyl ester;
α-methyl-4-(4-trifluoromethylthiazol-2-yl)phenylalanine;
α-methyl-3-(4-trifluoromethylthiazol-2-yl)-phenylalanine;
4-(imidazol-2-yl)-a-methylphenylalanine;
4-(4,5-dihydroimidazol-2-yl)-a-methylphenylalanine;
3-(imidazol-2-yl)-a-methylphenylalanine;
3-(4,5-dihydroimidazol-2-yl)-a-methylphenylalanine;
4-(imidazol-2-yl)phenylalanine;
4,5-dihydroimidazol-2-yl)phenylalanine;
3-(imidazol-2-yl)phenylalanine;
3-(2,3-dihydro-1H-indol-4-yl)-a-methylalanine;
α-methyl-3-(1H-2-oxindol-5-yl)alanine;
3-[1-(N-benzoylcarbamimidoyl)-2,3-dihydro-1H-indol-5-yl)]-a-methylalanine;
3-1-carbamimidoyl-2,3-dihydro-1H-indol-5-yl-a-methylalanine;
3-(1H-indol-5-yl)-a-methylalanine;
3-(benzimidazol-2-thione-5-yl)-a-methylalanine;
3-(2-aminobenzimidazol-5-yl-2-methylalanine;
2-methyl-3-(benzoxazol-2-on-6-yl)alanine;
3-(2-aminobenzothiazol-6-yl)-2-methylalanine;
3-(2-amino-4-mercaptobenzothiazol-6-yl)-2-methylalanine;
3-(2-aminobenzothiazol-6-yl)alanine;
2-methyl-3-(2,1,3-benzothiadiazol-5-yl)alanine;
3-(1,3-dihydrobenzo-2,1,3-thiadiazol-5-yl)-2methylalanine2,2-dioxide;
3-(1,3-dihydrobenzo-2,1,3-thiadiazol-5-yl)-2-methylalanine-2,2-dioxide methyl ester;
3-(1,3-dihydrobenzo-2,1,3-thiadiaxol-5-yl)alanine 2,2-dioxide;
3-(1,3-dihydro-1,3-dimethylbenzo-2,1,3-thiadiazol-5-yl-)-2-methylalanine 2,2-dioxide;
α-methyl-3-[4-methyl-2(1H)-oxoquinolin-6-yl]alanine;
3-[4-methyl-2(1H)-oxoquinolin-6-yl]alanine;
2-methyl-3-(quinoxalin-6-yl)alanine;
2-methyl-3-(2-hydroxyquinoxalin-6-yl)alanine;
2-methyl-3-(2-hydroxyquinoxalin-7-yl)alanine;
3-(2,3-dihydroxyquinoxalin-6-yl)-2-methylalanine;
3-(quinoxalin-6-yl)alanine;
3-(2,3-dihydroxyquinoxalin-6-yl)alanine;
3-(1,4-benzoxazin-3-one-6-yl)-2-methylalanine;
3-(1,4-benzoxazin-3-one-7-yl)alanine;
3-(5-hydroxy-4H-pyran-4-on-2-yl)-2-methylalanine;
3-(2-hydroxy-4-pyridyl)-2-methylalanine;
3-(2-carboxy-4-pyridyl)-2-methylamine;
α-methyl-4-(pyrrol-1-yl)phenylalanine;
α-ethyl-4-(pyrrol-1-yl)phenylalanine;
α-propyl-4-(pyrrol-1-yl)phenylalanine;
4-[2-(carboxy)pyrrol-1-yl)phenylalanine;
α-methyl-4-(pyrrol-1-yl)phenylalanine;
3-hydroxy-α-methyl-4-(pyrrol-1-yl)phenylalanine;
3-methoxy-α-methyl-4-(pyrrol-1-yl) phenylalanine;
4-methoxy-α-methyl-3-(pyrrol-1-yl) phenylalanine;
4-(indol-1-yl)-a-methylphenylalanine;
4-(carbazol-9-yl)-a-methylphenylalanine;
2-methyl-3-(2-methanesulfonylamidobenzimidazol-5-yl)alanine;
2-methyl-3-(2-amino-4-pyridyl) alanine;
2-methyl-3[tetrazolo-(1,5)-α-pyrid-7-yl]alanine;
D,L-α-methyl-β-(4-hydroxy-3-methyl)phenylalanine;
D,L-α-methyl-β-(4-hydroxy-3-phenyl)phenylalanine;
D,L-α-methyl-β-(4-hydroxy-3-benzyl)phenylalanine;
D,L-α-methyl-β-(4-methoxy-3-cyclohexyl)phenylalanlne;
a, b, b trimethyl-β-(3,4-dihydroxyphenyl)alanine;
a, b, b trimethyl-β-(4-hydroxyphenyl)alanine;
N-methyl a, b, b, trimethyl-β-(3,4-dihydroxphenyl)alanine;
D,L a, b, b trimethyl-β-(3,4-dihyroxyphenyl)alanine;
a, b, b trimethyl-β-(3,4-dimethoxyphenyl)alanine;
L-α-methyl-β-3,4-dihydroxyphenylalanine;
L-α-ethyl-β-3,4-dihydroxyphenylalanine;
L-α-propyl-β-3,4-dihydroxyphenylalanine;
L-α-butyl-β-3,4-dihydroxyphenylalanine;
L-α-methyl-β-2,3-dihydroxphenylalanine;
L-α-ethyl-β-2,3-dihydroxphenylalanine;
L-α-propyl-β-2,3-dihydroxphenylalanine;
L-α-butyl-β-2,3-dihydroxphenylalanine;
L-α-methyl-4-chloro-2,3-dihydroxyphenylalanine;
L-α-ethyl-4-chloro-2,3-dihydroxyphenylalanine;
L-α-propyl-4-chloro-2,3-dihydroxyphenylalanine;
L-α-butyl-4-chloro-2,3-dihydroxyphenylalanine;
L-α-ethyl-β-4-methyl-2,3-dihydroxyphenylalanine;
L-α-methyl-β-4-methyl-2,3-dihydroxyphenylalanine;
L-α-propyl-β-4-methyl-2,3-dihydroxyphenylalanine;
L-α-butyl-β-4-methyl-2,3-dihydroxyphenylalanine;
L-α-methyl-β-4-fluoro-2,3-dihydroxyphenylalanine;
L-α-ethyl-β-4-fluoro-2,3-dihydroxyphenylalanine;
L-α-propyl-β-4-fluoro-2,3-dihydroxyphenylalanine; L-α-butyl-β-4-fluoro-2,3-dihydroxyphenylalanine;
L-α-methyl-β-4-trifluoromethyl-2,3-dihydroxyphenyl alanine
L-α-ethyl-β-4-trifluoromethyl-2,3-dihydroxyphenyl alanine
L-α-propyl-β-4-trifluoromethyl-2,3-dihydroxyphenyl alanine
L-α-butyl-β-4-trifluoromethyl-2,3-dihydroxyphenyl alanine
L-α-methyl-β-3,5-dihydroxyphenylalanine;
L-α-ethyl-β-3,5-dihydroxyphenylalanine;
L-α-propyl-β-3,5-dihydroxyphenylalanine;
L-α-butyl-β-3,5-dihydroxyphenylalanine;
L-α-methyl-β-4-chloro-3,5-dihydroxphenylalanine;
L-α-ethyl-β-4-chloro-3,5-dihydroxphenylalanine;
L-α-propyl-β-4-chloro-3,5-dihydroxphenylalanine;
L-α-butyl-β-4-chloro-3,5-dihydroxphenylalanine;
L-α-methyl-β-4-fluoro-3,5-dihydroxyphenylalanine;
L-α-ethyl-β-4-fluoro-3,5-dihydroxyphenylalanine;
L-α-propyl-β-4-fluoro-3,5-dihydroxyphenylalanine;
L-α-butyl-β-4-fluoro-3,5-dihydroxyphenylalanine;
L-α-methyl-β-4-trifluoromethyl-3,5-dihydroxyphenyl alanine;
L-α-ethyl-β-4-trifluoromethyl-3,5-dihydroxyphenyl alanine;
L-α-propyl-β-4-trifluoromethyl-3,5-dihydroxyphenylal anlne;
L-α-butyl-β-4-trifluoromethyl-3,5-dihydroxyphenylalanine;
L-α-methyl-2,5-dihydroxphenylalanine;
L-α-ethyl-2,5-dihydroxphenylalanine;
L-α-propyl-2,5-dihydroxphenylalanine;
L-α-butyl-2,5-dihydroxphenylalanine;
L-α-methyl-β-4-chloro-2,5-dihydroxyphenylalanine;
L-α-ethyl-β-4-chloro-2,5-dihydroxyphenylalanine;
L-α-propyl-β-4-chloro-2,5-dihydroxyphenylalanine;
L-α-butyl-β-4-chloro-2,5-dihydroxyphenylalanine;
L-α-methyl-β-4-chloro-2,5-dihydroxyphenylalanine;
L-α-ethyl-β-4-chloro-2,5-dihydroxyphenylalanine;
L-α-propyl-β-4-chloro-2,5-dihydroxyphenylalanine;
L-α-butyl-β-4-chloro-2,5-dihydroxyphenylalanine;
L-α-methyl-β-methyl-2,5-dihydroxyphenylalanine;
L-α-ethyl-β-methyl-2,5-dihydroxyphenylalanine;
L-α-propyl-β-methyl-2,5-dihydroxyphenylalanine;
L-α-butyl-β-methyl-2,5-dihydroxyphenylalanine;
L-α-methyl-β-4-trifluoromethyl-2,5-dihydroxyphenyl alanine;
L-α-ethyl-β-4-trifluoromethyl-2,5-dihydroxyphenyl alanine;
L-α-propyl-β-4-trifluoromethyl-2,5-dihydroxyphenyl alanlne;
L-α-butyl-β-4-trifluoromethyl-2,5-dihydroxyphenyl alanine;
L-α-methyl-β-3,4,5-trihydroxyphenylalanine;
L-α-ethyl-β-3,4,5-trihydroxyphenylalanine;
L-α-propyl-β-3,4,5-trihydroxyphenylalanine;
L-α-butyl-β-3,4,5-trihydroxyphenylalanine;
L-α-methyl-β-2,3,4-trihydroxyphenylalanine;
L-α-ethyl-β-2,3,4-trihydroxyphenylalanine;
L-α-propyl-β-2,3,4-trihydroxyphenylalanine;
L-α-butyl-β-2,3,4-trihydroxyphenylalanine;
L-α-methyl-β-2,4,5-trihydroxyphenylalanine;
L-α-ethyl-β-2,4,5-trihydroxyphenylalanine;
L-α-propyl-β-2,4,5-trihydroxyphenylalanine;
L-α-butyl-β-2,4,5-trihydroxyphenylalanine;
L-phenylalanine;
D,L-a-methylphenylalanine;
D,L-3-iodophenylalanine;
D, L-3-iodo-a-methylphenylalanine;
3-iodotyrosine;
3,5-diiodotyrosine;
L-a-methylphenylalanine;
D,L-α-methyl-β-(4-hydroxy-3-methylphenyl)alanine;
D, L-α-methyl-β-(4-methoxy-3-benzylphenyl)alanine;
D,L-α-methyl-β-(4-hydroxy-3-benzylphenyl)alanine;
D,L-α-methyl-β-(4-methoxy-3-cyclohexylphenyl)alanine;
D,L-α-methyl-β-(4-hydroxy-3-cyclohexylphenyl)alanine;
D,L-α-methyl-β-(4-methoxy-3-methylphenyl)alanine;
D,L-α-methyl-β-(4-hydroxy-3-methylphenyl)alanine;
N,O-dibenzyl oxycarbonyl-D,L-α-methyl-β-(4-hydroxy-3-methylphenyl)alanine;
N,O-dibenzyloxycarbonyl-D,L-α-methyl-β-(4-hydroxy-3-methylphenyl)alanine amide;
D,L-α-methyl-β-(4-hydroxy-3-methylphenyl)alanine amide;
N,O-diacetyl-D, L-α-methyl-β-(4-hydroxy-3-methyl-phenyl)alanine;
D,L-N-acetyl-α-methyl-β-(4-hydroxy-3-methylphenyl)alanine;
L-3,4-dihydroxy-a-methylphenylalanine;
L-4-hydroxy-3-methoxy-a-methylphenylalanine;
L-3,4-methylene-dioxy-a-methylphenylalanine;
2-vinyl-2-amino-3-(2-methoxyphenyl)propionic acid;
2-vinyl-2-amino-3-(2,5-dimethoxyphenyl)propionic acid;
2-vinyl-2-amino-3-(2-imidazolyl)propionic acid;
2-vinyl-2-amino-3-(2-methoxyphenyl)propionic acid ethyl ester;
α-methyl-β-(2,5-dimethoxyphenyl)alanine;
α-methyl-β-(2,5-dihydroxyphenyl)alanine;
α-ethyl-β-(2,5-dimethoxyphenyl)alanine;
α-ethyl-β-(2,5-dihydroxyphenyl)alanine;
α-methyl-β-(2,4-dimethoxyphenyl)alanine;
α-methyl-β-(2,4-dihydroxyphenyl)alanine;
α-ethyl-β-(2,4-dimethoxyphenyl)alanine;
α-ethyl-β-(2,4-dihydroxyphenyl)alanine;
α-methyl-β-(2,5-dimethoxyphenyl)alanine ethyl ester;
2-ethynyl-2-amino-3-(3-indolyl)propionic acid;
2-ethynyl-2,3-(2-methoxyphenyl)propionic acid;
2-ethynyl-2,3-(5-hydroxyindol-3-yl)propionic acid;
2-ethynyl-2-amino-3-(2,5-dimethoxyphenyl)propionic acid;
2-ethynyl-2-amino-3-(2-imidazolyl)propionic acid;
2-ethynyl-2-amino-3-(2-methoxyphenyl)propionic acid ethyl ester;
3-carbomethoxy-3-(4-benzyloxybenzyl)-3-aminoprop-1-yne;
α-ethynyltyrosine hydrochloride;
α-ethynyltyrosine;
α-ethynyl-m-tyrosine;
α-ethynyl-β-(2-methoxyphenyl)alanine;
α-ethynyl-β-(2,5-dimethoxyphenyl)alanine; and
α-ethynylhistidine.

7. Conjugate of claim 5 wherein at least one of R¹⁰, R¹¹ and R¹² is selected from hydroxy, alkoxy, aryloxy, aralkoxy and alkoxycarbonyl; or a pharmaceutically-acceptable salt thereof.

8. Conjugate of claim 7 wherein said inhibitor compound is selected from the group consisting of
α-methyl-3-(pyrrol-1-yl)tyrosine;
α-methyl-3-(4-trifluoromethylthiazol-2-yl)tyrosine;
3-(imidazol-2-yl)-b-methyltyrosine;
L-α-methyl-m-tyrosine;
L-α-ethyl-m-tyrosine;
L-α-propyl-m-tyrosine;
L-α-butyl-m-tyrosine;
L-α-methyl-p-chloro-m-tyrosine;
L-α-ethyl-p-chloro-m-tyrosine;
L-α-butyl-p-chloro-m-tyrosine;
L-α-methyl-p-bromo-m-tyrosine;
L-α-ethyl-p-bromo-m-tyrosine;
L-α-butyl-p-bromo-m-tyrosine;
L-α-methyl-p-fluoro-m-tyrosine;
L-α-methyl-p-iodo-m-tyrosine;
L-α-ethyl-p-iodo-m-tyrosine;
L-α-methyl-p-methyl-m-tyrosine;
L-α-methyl-p-ethyl-m-tyrosine;
L-α-ethyl-p-ethyl-m-tyrosine;
L-α-ethyl-p-methyl-m-tyrosine;
L-α-methyl-p-butyl-m-tyrosine;
L-α-methyl-p-trifluoromethyl-m-tyrosine;
L-3-iodotyrosine;
L-3-chlorotyrosine;
L-3,5-diiodotyrosine;
L-a-methyltyrosine;
D,L-a-methyltyrosine;
D,L-3-iodo-a-methyltyrosine;
L-3-bromo-a-methyltyrosine;
D,L-3-bromo-a-methyltyrosine;
L-3-chloro-a-methyltyrosine;
D,L-3-chloro-a-methyltyrosine; and
2-vinyl-2-amino-3-(4-hydroxyphenyl)propionic acid.

9. Conjugate of claim 4 wherein said inhibitor compound is of the formula 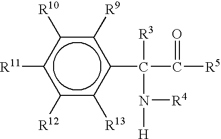
wherein R³ is selected from alkyl, alkenyl and alkynyl;
wherein R⁴ is selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein m is a number selected from zero through five, inclusive;
wherein R⁵ is selected from OR⁶ and 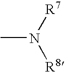
wherein R⁶ is selected from
hydrido, alkyl, cycloalkyl, cycloalkylalkyl, phenalkyl and phenyl, and wherein each of R⁷ and R³ is independently selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein each of R⁹ through R¹³ is independently selected from hydrido, hydroxy, alkyl, cycloalkyl, cycloalkylalkyl, aralkyl, aryl, alkoxycarbonyl, alkoxy, aryloxy, aralkoxy, alkoxyalkyl, haloalkyl, alkoxycarbonyl, hydroxyalkyl, halo, cyano, amino, monoalkylamino, dialkylamino, carboxyl, carboxyalkyl, alkanoyl, alkenyl, cycloalkenyl and alkynyl; or a pharmaceutically-acceptable salt thereof.

10. Conjugate of claim 9 wherein at least one of R¹⁰, R¹¹ and R¹² is selected from hydroxy, alkoxy, aryloxy, aralkoxy and alkoxycarbonyl; or a pharmaceutically-acceptable salt thereof.

11. Conjugate of claim 10 wherein said inhibitor compound is selected from the group consisting of methyl(+)-2-(4-hydroxyphenyl)glycinate; isopropyl and 3-methyl butyl esters of (+)-2-(4-hydroxyphenyl)glycine; (+)-(2-(4-hydroxyphenyl)glycine; 2-(4-hydroxyphenyl)glycine; (+)-2-(4-methoxyphenylglycine; and (+)-2-(4-hydroxyphenyl)glycinamide.

12. Conjugate of claim 4 wherein said inhibitor compound is of the formula 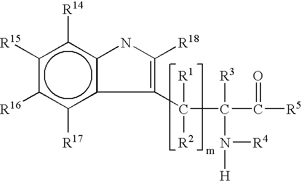
wherein each of R¹ and R² is hydrido; wherein R³ is selected from alkyl, alkenyl and alkynyl; wherein R⁴ is selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein m is a number selected from zero through five, inclusive; wherein each of R¹⁴ through R¹⁷ is independently selected from hydrido, hydroxy, alkyl, cycloalkyl, cyclo-alkylalkyl, aralkyl, aryl, alkoxy, aralkoxy, alkoxyalkyl, haloalkyl, hydroxyalkyl, halo, cyano, amino, monoalkylamino, dialkylamino, carboxyl, carboxyalkyl, alkanoyl, alkenyl, cycloalkenyl, alkynyl, cyanoamino, carboxyl, cyano, thiocarbamoyl, aminomethyl, alkylsulfanamido, nitro, alkylsulfonyloxy, carboxyalkoxy and formyl; or a pharmaceutically-acceptable salt thereof.

13. Conjugate of claim 12 wherein said inhibitor compound is selected from the group consisting of
L-a-methyltryptophan;
D,L-5-methyltryptophan;
D,L-5-chlorotryptophan;
D,L-5-bromotryptophan;
D,L-5-iodotryptophan;
L-5-hydroxytryptophan;
D,L-5-hydroxy-α-methyltryptophan;
α-ethynyltryptophan;
5-Methoxymethoxy-α-ethynyltryptophan; and
5-Hydroxy-α-ethynyltryptophan.

14. Conjugate of claim 4 wherein A is 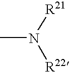
and m is a number selected from zero to three, inclusive; or a pharmaceutically-acceptable salt thereof.

15. Conjugate of claim 14 wherein said inhibitor compound is selected from the group consisting of 2-vinyl-2-amino-5-aminopentanoic acid and 2-ethynyl-2-amino-5-aminopentanoic acid.

16. Conjugate of claim 4 wherein said inhibitor compound is of the formula 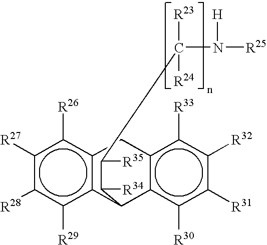
wherein each of R²³ and R²⁴ is independently selected from hydrido, hydroxy, alkyl, cycloakyl, cycloalkylalkyl, aralkyl, aryl, alkoxy, aralkoxy, aryloxy, alkoxyalkyl, haloalkyl, hydroxyalkyl, halo, cyano, amino, monoalkylamino, dialkylamino, carboxy, carboxyalkyl, alkanoyl, alkenyl, cycloalkenyl and alkynyl; wherein R²⁵ is selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein each of R²⁶ through R³⁵ is independently selected from hydrido, hydroxy, alkyl, cycloalkyl, cycloalkylalkyl, aralkyl, aryl, alkoxy, aralkoxy, alkoxyalkyl, haloalkyl, hydroxyalkyl, halo, cyano, amino, monoalkylamino, dialkylamino, carboxyl, carboxyalkyl, alkanoyl, alkenyl, cycloalkenyl, alkynyl, cyanoamino, carboxyl, cyano, thiocarbamoyl, aminomethyl, alkylsulfanamido, nitro, alkylsulfonyloxy, alkoxy and formyl; wherein n is a number selected from zero to five, inclusive; or a pharmaceutically-acceptable salt thereof.

17. Conjugate of claim 16 wherein said inhibitor compound is benzoctamine.

18. Conjugate of claim 3 wherein said inhibitor compound is a dopa-decarboxylase inhibitor of the formula 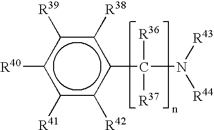
Wherein each of R³⁶ through R⁴² is independently selected from hydrido, hydroxy, alkyl, cycloalkyl, cycloalkylalkyl, aralkyl, aryl, alkoxy, aralkoxy, alkoxyalkyl, haloalkyl, hydroxyalkyl, halo, cyano, amino, monoalkylamino, dialkylamino, carboxyl, carboxyalkyl, alkanoyl, alkenyl, cycloalkenyl, alkynyl, cyanoamino, cyano, thiocarbamoyl, aminomethyl, alkylsulfanamido, nitro, alkylsulfonyloxy, carboxyalkoxy and formyl; wherein n is a whole number from zero through four; wherein each of R⁴³ and R⁴⁴ is independently selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl, arylsulfonyl, alkenyl, cycloalkenyl and alkynyl; and wherein any R⁴³ and R⁴⁴ substituent having a substitutable position may be further substituted with one or more groups selected from hydroxyalkyl, halo, haloalkyl, carboxyl, alkoxyalkyl, alkoxycarbonyl; with the proviso that R⁴³ and R⁴⁴ cannot both be carboxyl at the same time, with the further proviso that when R³⁶ is hydrido then R³⁷ cannot be carboxyl, and with the further proviso that at least one of R⁴³ through R⁴⁴ must be a primary or secondary amino group; or a pharmaceutically-acceptable salt thereof.

19. Conjugate of claim 18 wherein each of R³⁶ through R⁴² is independently selected from hydrido, hydroxy, alkyl, cycloalkyl, cycloalkylalkyl, aralkyl, aryl, alkoxy, aralkoxy, alkoxyalkyl, haloalkyl, hydroxyalkyl, halo, amino, monoalkylamino, dialkylamino, carboxyl, carboxyalkyl, alkanoyl, alkenyl, cycloalkenyl, alkynyl, cyanoamino, cyano, aminomethyl, carboxyalkoxy and formyl; wherein n is a whole number from one through three; wherein each of R⁴³ and R⁴⁴ is independently selected from hydrido, alkyl, cycloalkyl, cycloalkylalkyl, aralkyl, aryl, alkoxyalkyl, haloalkyl, hydroxyalkyl, amino, monoalkylamino, dialkylamino, carboxyl, carboxyalkyl and alkanoyl; and wherein any R⁴³ and R⁴⁴ substituent having a substitutable position may be further substituted with one or more groups selected from hydroxyalkyl, halo, haloalkyl, carboxyl, alkoxyalkyl, alkoxycarbonyl; or a pharmaceutically-acceptable salt thereof.

20. Conjugate of claim 19 wherein each of R³⁶ through R⁴² is independently selected from hydrido, hydroxy, alkyl, benzyl, phenyl, alkoxy, benzyloxy, alkoxyalkyl, haloalkyl, hydroxyalkyl, amino, monoalkylamino, dialkylamino, carboxyl, carboxyalkyl, alkanoyl, cyanoamino, cyano, minomethyl, carboxyl, carboxyalkoxy and formyl; wherein n is one or two; wherein each of R⁴³ and R⁴⁴ is independently selected from hydrido, alkyl, benzyl, phenyl, alkoxyalkyl, haloalkyl, hydroxyalkyl, cyano, amino, monoalkylamino, dialkylamino, carboxyl, carboxyalkyl and alkanoyl; and wherein any R⁴³ and R⁴⁴ substituent having a substitutable position may be further substituted with one or more groups selected from hydroxyalkyl, halo, haloalkyl, carboxyl, alkoxyalkyl, alkoxycarbonyl; or a pharmaceutically-acceptable salt thereof.

21. Conjugate of claim 20 wherein each of R³⁶ through R⁴² is independently selected from hydrido, hydroxy, alkyl, alkoxy, haloalkyl, hydroxyalkyl, amino, monoalkylamino, carboxyl, carboxyalkyl, aminomethyl, carboxyalkoxy and formyl; wherein n is on or two; wherein each of R⁴³ and R⁴⁴ is independently selected from hydrido, alkyl, haloalkyl, hydroxyalkyl, amino, monoalkylamino, carboxyl and carboxyalkyl; and wherein any R⁴³ and R⁴⁴ substituent having a substitutable position may be further substituted with one or more groups selected from hydroxyalkyl, halo, haloalkyl, carboxyl, alkoxyalkyl, alkoxycarbonyl; or a pharmaceutically-acceptable salt thereof.

22. Conjugate of claim 21 wherein each of R³⁶ and R⁴² is hydrido and n is one; wherein each of R³³ through R⁴² is independently selected from hydroxy, alkyl, alkoxy, haloalkyl, hydroxyalkyl, amino, monoalkylamino, carboxyl, carboxyalkyl, aminomethyl, carboxyalkoxy and formyl; wherein each of R⁴³ and R⁴⁴ is independently selected from hydrido, alkyl, haloalkyl, hydroxyalkyl, amino, monoalkylamino, carboxyl and carboxyalkyl; and wherein any R⁴³ and R⁴⁴ substituent having a substitutable position may be further substituted with one or more groups selected from hydroxyalkyl, halo, haloalkyl, carboxyl, alkoxyalkyl, alkoxycarbonyl; or a pharmaceutically-acceptable salt thereof.

23. Conjugate of claim 22 wherein said inhibitor compound is selected from (2,3,4-trihydroxy)benzylhydrazine; 1-(D,L-seryl-2-(2,3,4-trihydroxybenzyl)hydrazine; and 1-(3-hydroxyl-benzyl)-l-methylhydrazine.

24. Conjugate of claim 21 wherein each of R³⁶ and R³⁷ is independently selected from hydrido, alkyl and amino and n is two; wherein each of R³⁸ through R⁴² is independently selected from hydroxy, alkyl, alkoxy, haloalkyl, hydroxyalkyl, amino, monoalkylamino, carboxyl, carboxyalkyl, aminomethyl, carboxyalkoxy and formyl; wherein each of R⁴³ and R⁴⁴ is independently selected from hydrido, alkyl, haloalkyl, hydroxyalkyl, amino, monoalkylamino, carboxyl and carboxyalkyl; or a pharmaceutically-acceptable salt thereof.

25. Conjugate of claim 24 wherein said inhibitor compound is selected from 2-hydrazino-2-methyl-3-(3,4-dihydroxyphenyl)propionic acid;
α-(monofluoromethyl)dopa; α-(difluoromethyl)dopa; and α-methyldopa.

26. Conjugate of claim 3 wherein said inhibitor compound is a dopa-decarboxylase inhibitor of the formula 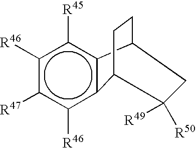
wherein each of R⁴⁵ through R⁴³ is independently selected from hydrido, hydroxy, alkyl, cycloalkyl, cycloalkylalkyl, aralkyl, aryl, alkoxy, aralkoxy, alkoxyalkyl, haloalkyl, hydroxyalkyl, halo, amino, monoalkylamino, dialkylamino, carboxyl, carboxyalkyl, alkanoyl, alkenyl, cycloalkenyl, alkynyl, cyanoamino, cyano, thiocarbamoyl, aminomethyl, alkylsulfanamido, nitro, alkylsulfonyloxy, carboxyalkoxy and formyl; wherein each of R⁴⁹ and R⁵⁰ is independently selected from hydrido, alkyl, cycloalkyl, cycloalkylalkyl, aralkyl, aryl, alkoxyalkyl, haloalkyl, hydroxyalkyl, cyano, amino, monoalkylamino, dialkylamino, carboxyalkyl, alkanoyl, alkenyl, cycloalkenyl, alkynyl and 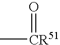
wherein R⁵¹ is selected from hydroxy, alkoxy, aryloxy, aralkoxy, amino, monoalkylamino and dialkylamino; with the proviso that R⁴⁹ and R⁵⁰ cannot both be carboxyl at the same time, and with the further proviso that at least one of R⁴⁵ through R⁴³ is a primary or secondary amino group or a carboxyl group; or a pharmaceutically-acceptable salt thereof.

27. Conjugate of claim 26 wherein each of R⁴⁵ through R⁴³ is independently selected from hydrido, hydroxy, alkyl, cycloalkyl, cycloalkylalkyl, aralkyl, aryl, alkoxy, aralkoxy, alkoxyalkyl, haloalkyl, hydroxyalkyl, halo, cyano, amino, monoalkylamino, dialkylamino, carboxyl, carboxyalkyl, alkanoyl, alkenyl, cycloalkenyl, alkynyl, cyanoamino, cyano, aminomethyl, carboxyalkoxy and formyl; wherein each of R⁴⁹ and R⁵⁰ is independently selected from hydrido, alkyl, cycloalkyl, cycloalkylalkyl, aralkyl, aryl, alkoxyalkyl, haloalkyl, hydroxyalkyl, cyano, amino, monoalkylamino, dialkylamino, carboxyalkyl and alkanoyl and 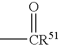
wherein R⁵¹ is selected from hydroxy, alkoxy, phenoxy, benzyloxy, amino, monoalkylamino and dialkylamino; or a pharmaceutically-acceptable salt thereof.

28. Conjugate of claim 27 wherein each of R⁴⁵ through R⁴⁸ is independently selected from hydrido, hydroxy, alkyl, benzyl, phenyl, alkoxy, benzyloxy, alkoxyalkyl, haloalkyl, hydroxyalkyl, cyano, amino, monoalkylamino, dialkylamino, carboxyl, carboxyalkyl, alkanoyl, cyanoamino, cyano, aminomethyl, carboxyalkoxy and formyl; wherein each of R⁴⁹ and R⁵⁰ is independently selected from hydrido, alkyl, benzyl, phenyl, alkoxyalkyl, haloalkyl, hydroxyalkyl, cyano, amino, monoalkylamino, dialkylamino, carboxyalkyl and alkanoyl and 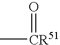
wherein R⁵¹ is selected from hydroxy, alkoxy, amino and monoalkylamino; or a pharmaceutically-acceptable salt thereof.

29. Conjugate of claim 28 wherein each of R⁴⁵ through R⁴⁸ is independently selected from hydrido, hydroxy, alkyl, alkoxy, haloalkyl, hydroxyalkyl, amino, monoalkylamino, carboxyl, carboxyalkyl aminomethyl, carboxyalkoxy and formyl; wherein each of R⁴⁹ and R⁵⁰ is independently selected from hydrido alkyl, amino, monoalkylamino, carboxyalkyl and 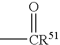
wherein R⁵¹ is selected from hydroxy, alkoxy, amino and monoalkylamino; or a pharmaceutically-acceptable salt thereof.

30. Conjugate of claim 29 wherein each of R⁴⁵ through R⁴⁸ is independently selected from hydrido, hydroxy, alkyl, alkoxy and hydroxyalkyl; wherein each of R⁴⁹ and R⁵⁰ is independently selected from alkyl, amino, monoalkylamino, and 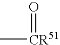
wherein R⁵¹ is selected from hydroxy, methoxy, ethoxy, propoxy, butoxy, amino, methylamino and ethylamino; or a pharmaceutically-acceptable salt thereof.

31. Conjugate of claim 30 wherein said inhibitor compound is selected from endo-2-amino-1,2,3,4-tetrahydro-1,4-ethanonaphthalene2-carboxylic acid; ethyl-endo-2-amino-1,2,3,4-tetrahydro-1,4-ethanonaphthalene-2-carboxylate hydrochloride; exo-2-amino-1,2,3,4-tetrahydro-1,4-ethanonaphthalene2-carboxylic acid; and ethyl-exo-2-amino-1,2,3,4-tetrahydro-1,4-ethanonaphthalene-2-carboxylate hydrochloride.

32. Conjugate of claim 3 wherein said inhibitor compound is a dopa-decarboxylase inhibitor selected from
2,3-dibromo-4,4-bis(4-ethylphenyl)-2-butenoic acid;
3-bromo-4-(4-methoxyphenyl)-4-oxo-2-butenoic acid;
N-(5′-phosphopyridoxyl)-L-3,4-dihydroxyphenylalanine;
N-(5′-phosphopyridoxyl)-L-m-aminotyrosine;
D,L-b-(3,4-dihydroxyphenyl)lactate;
D,L-b-(5-hydroxyindolyl-3)lactate;
2,4-dihydroxy-5-(1-oxo-2-propenyl)benzoic acid;
2,4-dimethoxy-5-[1-oxo-3-(2,3,4-trimethoxyphenyl-2 propenyl]benzoic acid;
2,4-dihydroxy-5-[1-oxo-3-(2-thienyl)-2-propenyl]benzoic acid;
2,4-dihydroxy-5-[3-(4-hydroxyphenyl)-1-oxo-2-propenyl]benzoic acid;
5-[3-(4-chlorophenyl)-1-oxo-2-propenyl]-2,4-dihydroxy benzoic acid;
2,4-dihydroxy-5-(1-oxo-3-phenyl-2-propenyl)benzoic acid;
2,4-dimethoxy-5-[1-oxo-3-(4-pyridinyl)-2-propenyl]benzoic acid;
5-[3-(3,4-dimethoxyphenyl)-1-oxo-2-propenyl]-2,4 dimethoxy benzoic acid;
2,4-dimethoxy-5-(1-oxo-3-phenyl-2-propenyl)benzoic acid;
5-[3-(2-furanyl)-1-oxo-2-propenyl]-2,4-dimethoxy benzoic acid;
2,4-dimethoxy-5-[1-oxo-3-(2-thienyl)-2-propenyl]benzoic acid;
2,4-dimethoxy-5-[3-(4-methoxyphenyl)-1-oxo-2-propenyl]benzoic acid;
5-[3-(4-chlorophenyl)-1-oxo-2-propenyl]-2,4-dimethoxy benzoic acid; and
5-[3-[4-(dimethylamino)phenyl]-1-oxo-2-propenyl]-2,4 dimethoxy benzoic acid.

33. Conjugate of claim 3 wherein said inhibitor compound is a dopa-decarboxylase inhibitor of the formula: 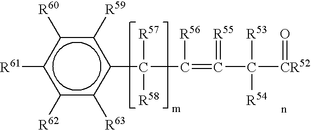
wherein R⁵² is selected from hydrido, OR⁶⁴ and 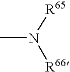
wherein R⁶⁴ is selected from hydrido, alkyl, cycloalkyl, cycloalkylalkyl, phenalkyl and phenyl, and wherein each of R⁶⁵ and R⁶⁶ is independently selected from hydrido, alkyl, alkanoyl, amino, monoalkylamino, dialkylamino, phenyl and phenalkyl; wherein each of R⁵³, R⁵⁴ and R⁵⁷ through R⁶³ is independently selected from hydrido, hydroxy, alkyl, cycloalkyl, cycloalkylalkyl, aralkyl, aryl, alkoxycarbonyl, hydroxyalkyl, halo, cyano, amino, monoalkylamino, dialkylamino, carboxyl, carboxyalkyl, alkanoyl, alkenyl, cycloalkenyl and alkynyl; wherein each of R⁵⁵ and R⁵⁶ is independently selected from hydrido, alkyl, cycloalkyl, cycloalkylalkyl, aralkyl, aryl, alkoxyalkyl, haloalkyl, hydroxyalkyl and carboxyalkyl; wherein each of m and n is a number independently selected from zero through six, inclusive; or a pharmaceutically-acceptable salt thereof.

34. Conjugate of claim 33 wherein R⁵² is OR⁶⁴ wherein R⁶⁴ is selected from hydrido, alkyl, cycloalkyl, cycloalkylalkyl, benzyl and phenyl; wherein each of R⁵³, R⁵⁴ and R⁵⁷ through R⁶³ is independently selected from hydrido, alkyl, cycloalkyl, hydroxy, alkoxy, benzyl and phenyl; wherein each of R⁵⁵ and R⁵⁶ is independently selected from hydrido, alkyl, cycloalkyl, benzyl and phenyl; wherein each of m and n is a number independently selected from zero through three, inclusive; or a pharmaceutically-acceptable salt thereof.

35. Conjugate of claim 34 wherein R⁵² is OR⁶⁴ wherein R⁶⁴ is selected from hydrido and lower alkyl; wherein each of R⁵³ through R⁵⁸ is hydrido; wherein each of R⁵⁹ through R⁶³ is independently selected from hydrido, alkyl, hydroxy and alkoxy, with the proviso that two of the R⁵⁹ through R⁶³ substituents are hydroxy; wherein each of m and n is a number independently selected from zero through two, inclusive; or a pharmaceutically-acceptable salt thereof.

36. Conjugate of claim 35 which is 3-(3,4-dihydroxyphenyl)-2-propenoic acid.

37. Conjugate of claim 26 wherein said dopa-decarboxylase inhibitor is a compound selected from aminohaloalkyl-hydroxyphenyl propionic acids; alpha-halomethylphenylalanine derivatives; and indole-substituted halomethylamino acids.

38. Conjugate of claim 26 wherein said dopa-decarboxylase inhibitor is a compound selected from isoflavone extracts from fungi and streptomyces; sulfinyl substituted dopa and tyrosine derivatives; hydroxycoumarin derivatives; 1-benzylcyclobutenyl alkyl carbamate derivatives; aryl/thienyl-hydroxylamine derivatives; and b-2-substituted-cyclohepta-pyrrol-8lH-on-7-yl alanine derivatives.

39. Conjugate of claim 3 wherein said dopamineβ-hydroxylase inhibitor compound is of the formula 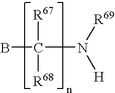
wherein B is selected from an ethylenic moiety, an acetylenic moiety and an ethylenic or acetylenic moiety substituted with one or more radicals selected from substituted or unsubstituted alkyl, aryl and heteroaryl; wherein each of R⁶⁷ and R⁶⁸ is independently selected from hydrido and alkyl; wherein R⁶⁹ is selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; and wherein n is a number selected from one through five; or a pharmaceutically-acceptable salt thereof.

40. Conjugate of claim 39 wherein B is an ethylenic or an acetylenic moiety substituted with an aryl or heteroaryl radical; and wherein n is a number from one through three; or a pharmaceutically-acceptable salt thereof.

41. Conjugate of claim 39 wherein B is an ethylenic or acetylenic moiety incorporating carbon atoms in the beta- and gamma-positions relative to the nitrogen atom; and wherein n is one; or a pharmaceutically-acceptable salt thereof.

42. Conjugate of claim 41 wherein said ethylenic or acetylenic moiety is substituted at the gamma carbon with an aryl or heteroaryl radical; or a pharmaceutically-acceptable salt thereof.

43. Conjugate of claim 42 wherein said aryl radical is selected from phenyl, 2-thiophene, 3-thiophene, 2-furanyl, 3-furanyl, oxazolyl, thiazolyl and isoxazolyl, any one of which radicals may be substituted with one or more groups selected from halo, hydroxyl, alkyl, haloalkyl, cyano, alkoxy, alkoxyalkyl and cycloalkyl; or a pharmaceutically-acceptable salt thereof.

44. Conjugate of claim 43 wherein said aryl radical is selected from phenyl, hydroxyphenyl, 2-thiophene and 2-furanyl; and wherein each of R⁶⁷, R⁶⁸ and R⁶⁹ is hydrido; or a pharmaceutically-acceptable salt thereof.

45. Conjugate of claim 44 wherein said inhibitor compound is selected from the group consisting of
3-amino-2-(2′-thienyl)propene;
3-amino-2-(2′-thienyl)butene;
3-(N-methylamino)-2-(2′-thienyl)propene;
3-amino-2-(3′-thienyl)propene;
3-amino-2-(2′-furanyl)propene;
3-amino-2-(3′-furanyl)propene;
1-phenyl-3-aminopropyne; and
3-amino-2-phenylpropene.

46. Conjugate of claim 44 wherein said inhibitor compound is selected from the group consisting of (±)4-amino-3-phenyl-1-butyne;
(±)4-amino-3-(3′-hydroxyphenyl)-1-butyne;
(±)4-amino-3-(4′-hydroxyphenyl)-1-butyne;
(±)4-amino-3-phenyl-1-butene;
(±)4-amino-3-(3′-hydroxyphenyl)-1-butene; and
(±)4-amino-3-(4′-hydroxyphenyl)-1-butene.

47. Conjugate of claim 3 wherein said inhibitor compound is of the formula 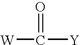
wherein W is selected from alkyl, cycloalkyl, alkenyl, alkynyl, cycloalkenyl, cycloalkynyl, cycloalkylalkyl, aryl, aralkyl, heterocycloalkyl and heteroaryl; wherein Y is selected from 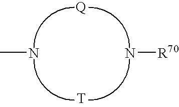
wherein R⁷⁰ is selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein each of Q and T is one or more groups independently selected from 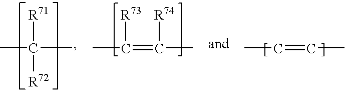
wherein each of R⁷¹ through R⁷⁴ is independently selected from hydrido, hydroxy, alkyl, cycloalkyl, cycloalkylalkyl, aralkyl, aryl, alkoxy, aralkoxy, aryloxy, alkoxyalkyl, haloalkyl, hydroxyalkyl, halo, cyano, amino, monoalkylamino, dialkylamino, carboxy, carboxyalkyl, alkanoyl, alkenyl, cycloalkenyl and alkynyl; or a pharmaceutically-acceptable salt thereof.

48. Conjugate of claim 47 wherein W is heteroaryl and Y is 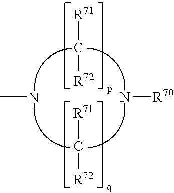
wherein R⁷⁰ is selected from hydrido, alkyl, amino, monoalkylamino, dialkylamino, phenyl and phenalkyli wherein each of R⁷¹ and R⁷² is independently selected from hydrido, hydroxy, alkyl, phenalkyl, phenyl, alkoxy, benzyloxy, phenoxy, alkoxyalkyl, hydroxyalkyl, halo, amino, monoalkylamino, dialkylamino, carboxy, carboxyalkyl and alkanoyl; and wherein each of p and q is a number independently selected from one through six, inclusive; or a pharmaceutically-acceptable salt thereof.

49. Conjugate of claim 48 wherein R⁷⁰ is selected from hydrido, alkyl, amino and monoalkylamino; wherein each of R⁷¹ and R⁷² is independently selected from hydrido, hydroxy, alkyl, alkoxy, amino, monoalkylamino, carboxy, carboxyalkyl and alkanoyl; and wherein each of p and q is a number indpendently selected from two through four, inclusive; or a pharmaceutically-acceptable salt thereof.

50. Conjugate of claim 49 wherein R⁷⁰ is selected from hydrido, alkyl and amino; wherein each of R⁷¹ and R⁷² is independently selected from hydrido, amino, monoalkylamino and carboxyl; and wherein each of p and q is independently selected from the numbers two and three; or a pharmaceutically-acceptable salt thereof.

51. Conjugate of claim 50 wherein R⁷⁰ is hydrido; wherein each of R⁷¹ and R⁷² is hydrido; and wherein each of p and q is two; or a pharmaceutically-acceptable salt thereof.

52. Conjugate of claim 3 wherein said inhibitor compound is of the formula 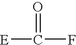
wherein E is selected from alkyl, cycloalkyl, alkenyl, alkynyl, cycloalkenyl, cycloalkynyl, cycloalkylalkyl, aryl, aralkyl, heterocycloalkyl and heteroaryl; wherein F is selected from 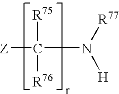
wherein Z is selected from O, S and N—R⁷⁸; wherein each of R⁷⁵ and R⁷⁶ is independently selected from hydrido, hydroxy, alkyl, cycloalkyl, cycloalkylalkyl, aralkyl, aryl, alkoxy, aralkoxy, aryloxy, alkoxyalkyl, haloalkyl, hydroxyalkyl, halo, cyano, amino, minoalkylamino, dialkylamino, carboxy, carboxyalkyl, alkanoyl, alkenyl, cycloalkenyl and alkynyl; wherein R⁷⁵ and R⁷⁶ may form oxo or thio; wherein r is a number selected from zero through six, inclusive; wherein each of R⁷⁷ and R⁷⁸ is independently selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyli or a pharmaceutically-acceptable salt thereof.

53. Conjugate of claim 3 wherein said dopamine-β-hydroxylase inhibitor compound is of the formula 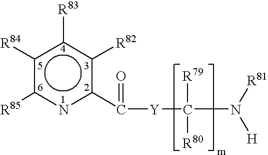
wherein each of R⁸² through R⁸⁵ is independently selected from hydrido, alkyl, haloalkyl, mercapto, alkylthio, cyano, alkoxy, alkoxyalkyl and cycloalkyli wherein Y is selected from oxygen atom and sulfur atom; wherein each of R⁷⁹ and R⁸⁰ is independently selected from hydrido and alkyl; wherein R⁵⁹ is selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; and wherein m is a number from one through six; or a pharmaceutically-acceptable salt thereof.

54. Conjugate of claim 53 wherein each of R⁸² through R⁸⁵ is independently selected from hydrido, alkyl and haloalkyl; wherein Y is selected from oxygen atom or nitrogen atom; wherein each of R⁷⁹, R⁸⁰ and R⁸¹ is independently hydrido and alkyl; and wherein m is a number selected from one through four, inclusive; or a pharmaceutically-acceptable salt thereof.

55. Conjugate of claim 54 wherein said inhibitor compound is selected from
aminomethyl-5-n-butylthiopicolinate;
aminomethyl-5-n-butylpicolinate;
2′-aminoethyl-5-n-butylthiopicolinate;
2′-aminoethyl-5-n-butylpicolinate;
(2′-amino-1′,1′-dimethyl)ethyl-5-n-butylthiopicolinate;
(2′-amino-1′,1′-dimethyl)ethyl-5-n-butylpicolinate;
(2′-amino-1′-methyl) ethyl-5-n-butylthiopicolinate;
(2′-amino-1′-methyl) ethyl-5-n-butylpicolinate;
3′-aminopropyl-5-n-butylthiopicolinate;
3′-aminopropyl-5-n-butylpicolinate;
(2′-amino-2′-methyl) propyl-5-n-butylthiopicolinate;
(2′-amino-2′-methyl) propyl-5-n-butylpicolinate;
(3′-amino-1′,1′-dimethyl)propyl-5-n-butylthiopicolinate;
(3′-amino-1′,1′-dimethyl)propyl-5-n-butylpicolinate;
(3′-amino-2′,2′-dimethyl) propyl-5-n-butylthiopicolinate;
(3′-amino-2′,2′-dimethyl)propyl-5-n-butylpicolinate;
2′-aminopropyl-5-n-butylthiopicolinate;
2′-aminopropyl-5-n-butylpicolinate;
4′-aminobutyl-5-n-butylthiopicolinate;
4′-amino-3′-methyl)butyl-5-n-butylthiopicolinate;
(3′-amino-3′-methyl)butyl-5-n-butylthiopicolinate; and
(3′-amino-3′-methyl)butyl-5-n-butylpicolinate.

56. Conjugate of claim 47 wherein said inhibitor compound is of the formula 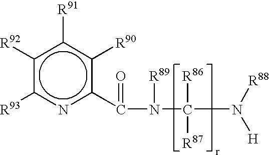
wherein each of R⁸⁶, R⁸⁷ and R⁹⁰ through R⁹³ is independently selected from hydrido, hydroxy, alkyl, cycloalkyl, cycloalkylalkyl, aralkyl, aryl, alkoxy, aralkoxy, aryloxy, alkoxyalkyl, haloalkyl, hydroxyalkyl, halo, cyano, amino, monoalkylamino, dialkylamino, carboxy, carboxyalkyl, alkanoyl, alkenyl, cycloalkenyl and alkynyl; wherein R⁸⁶ and R⁸⁷ together may form oxo or thio; wherein r is a number selected from zero through six, inclusive; wherein each of R⁸⁸ and R⁸⁹ is independently selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; or a pharmaceutically-acceptable salt thereof.

57. Conjugate of claim 56 wherein each of R⁸⁶, R⁸⁷ and R⁹⁰ through R⁹³ is independently selected from hydrido, hydroxy, alkyl, phenalkyl, phenyl, alkoxy, benzyloxy, phenoxy, alkoxyalkyl, hydroxyalkyl, halo, amino, monoalkylamino, dialkylamino, carboxy, carboxyalkyl and alkanoyl; wherein r is a number selected from zero through four, inclusive; wherein each of R⁸⁸ and R⁸⁹ is independently selected from hydrido, alkyl, amino, monoalkylamino, dialkylamino, phenyl and phenalkyl; or a pharmaceutically-acceptable salt thereof.

58. Conjugate of claim 57 wherein each of R⁸⁶, R⁸⁷ and R⁹⁰ through R⁹³ is independently selected from hydrido, hydroxy, alkyl, alkoxy, amino, monoalkylamino, carboxy, carboxyalkyl and alkanoyl; and wherein r is a number selected from zero through three, inclusive; and wherein each of R⁸⁸ and R⁸⁹ is selected from hydrido, alkyl, amino and monoalkylamino; or a pharmaceutically-acceptable salt thereof.

59. Conjugate of claim 58 wherein each of R⁹⁰ through R⁹³ is independently selected from hydrido and alkyl; wherein each of R⁸⁶ and R⁸⁷ is hydrido; wherein r is selected from zero, one and two; wherein R⁸⁸ is selected from hydrido, alkyl and amino; and wherein R⁸⁹ is selected from hydrido and alkyl; or a pharmaceutically-acceptable salt thereof.

60. Conjugate of claim 59 wherein said inhibitor compound is 5-n-butylpicolinic acid hydrazide.

61. Conjugate of claim 3 wherein said dopamine-β-hydroxylase inhibitor compound is of the formula 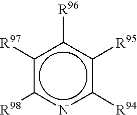
wherein each of R⁹⁴ through R⁹⁸ is independently selected from hydrido, hydroxy, alkyl, cycloalkyl, cycloalkylalkyl, aralkyl, aryl, aryloxy, alkoxy, alkylthio, aralkoxy, alkoxyalkyl, haloalkyl, hydroxyalkyl, halo, cyano, amino, monoalkylamino, dialkylamino, amido, alkylamido, hydroxyamino, carboxyl, carboxyalkyl, alkanoyl, alkenyl, cycloalkenyl, alkynyl, cyanoamino, carboxyl, thiocarbamoyl, aminomethyl, alkylsulfanamido, nitro, alkylsulfonyloxy, formoyl and alkoxycarbonyl; with the proviso that at least one of R⁹⁴ through R⁹⁸ is 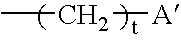
wherein A′ is 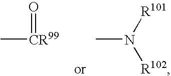
wherein R⁹⁹ is selected from hydrido, alkyl, hydroxy, alkoxy, alkylthio, phenyl, phenoxy, benzyl, benzyloxy, —OR¹⁰⁰ and 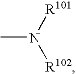
wherein R¹⁰⁰ is selected from hydrido, alkyl, cycloalkyl, cycloalkylalkyl, phenyl and benzyl; wherein each of R¹⁰¹ and R¹⁰² is independently selected from hydrido, alkyl, cycloalkyl, hydroxyalkyl, haloalkyl, cycloalkylalkyl, alkoxyalkyl, aralkyl, aryl, alkanoyl, alkoxycarbonyl, carboxyl, amino, cyanoamino, monoalkylamino, dialkylamino, alkylsulfinyl, alkylsulfonyl, arylsulfinyl and arylsulfonyl; wherein t is a number selected from zero through four, inclusive; or a pharmaceutically-acceptable salt thereof.

62. Conjugate of claim 61 wherein said inhibitor compound is of the formula 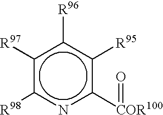
wherein each of R⁹⁵ through R⁹⁸ is independently selected from hydrido, hydroxy, alkyl, cycloalkyl, phenyl, benzyl, alkoxy, phenoxy, benzyloxy, alkoxyalkyl, hydroxyalkyl, halo, cyano, amino, monoalkylamino, dialkylamino, amido, alkylamido, hydroxyamino, carboxyl, carboxyalkyl, alkanoyl, cyanoamino, carboxyl, thiocarbamoyl, aminomethyl, nitro, formoyl, formyl and alkoxycarbonyl; and wherein R¹⁰⁰ is selected from hydrido, alkyl, phenyl and benzyl; or a pharmaceutically-acceptable salt thereof.

63. Conjugate of claim 62 wherein said inhibitor compound is selected from
5-n-butylpicolinic acid;
5-ethylpicolinic acid;
lcollnlc acId;
5-nitropicolinic acid;
5-aminopicolinic acid;
5-N-acetylaminopicolinic acid;
5-N-propionylaminopicolinic acid;
5-N-hydroxyaminopicolinic acid;
5-iodopicolinic acid;
5-bromopicolinic acid;
5-chloropicolinic acid;
5-hydroxypicolinic acid
5-methoxypicolinic acid;
5-N-propoxypicolinic acid;
5-N-butoxypicolinic acid;
5-cyanopicolinic acid;
5-carboxylpicolinic acid;
5-n-butyl-4-nitropicolinic acid;
5-n-butyl-4-methoxypicolinic acid;
5-n-butyl-4-ethoxypicolinic acid;
5-n-butyl-4-aminopicolinic acid;
5-n-butyl-4-hydroxyaminopicolinic acid; and
5-n-butyl-4-methylpicolinic acid.

64. Conjugate of claim 63 wherein said inhibitor compound is 5-n-butylpicolinic acid.

65. Conjugate of claim 3 wherein said dopamine-β-hydroxylase inhibitor compound is of the formula 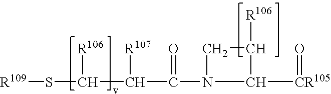
wherein R¹⁰⁵ is hydrido, hydroxy, alkyl, amino and alkoxy; wherein R¹⁰⁶ is selected from hydrido, hydroxy and alkyl; wherein each of R¹⁰⁷ and R¹⁰⁸ is independently selected from hydrido, alkyl and phenalkyl; wherein R¹⁰⁹ is selected from hydrido and 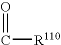
with R¹¹⁰ selected from alkyl, phenyl and phenalkyl;
wherein u is a number from one to three, inclusive; and
wherein v is a number from zero to two, inclusive; or a pharmaceutically-acceptable salt thereof.

66. Conjugate of claim 65 wherein R¹⁰⁵ is selected from hydroxy and lower alkoxy; wherein R¹⁰⁶ is hydrido; wherein R¹⁰⁷ is selected from hydrido and lower alkyl; wherein R¹⁰⁸ is hydrido; wherein R¹⁰⁹ is selected from hydrido and 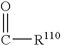
with R¹¹⁰ selected from lower alkyl and phenyl; wherein u is two; and wherein v is a number from zero to two, inclusive; or a pharmaceutically-acceptable salt thereof.

67. Conjugate of claim 66 wherein said inhibitor compound is of the formula 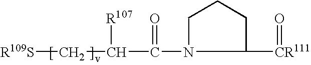
wherein R¹¹¹ is selected from hydroxy and lower alkyl;
wherein R¹⁰⁷ is selected from hydrido and lower alkyl;
wherein R¹⁰⁹ is selected from hydrido and 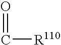
with R¹¹⁰ selected from lower alkyl and phenyl and v is a number from zero to two, inclusive; or a pharmaceutically-acceptable salt thereof.

68. Conjugate of claim 67 wherein R¹¹¹ is hydroxy; wherein R¹⁰⁷ is hydrido or methyl; wherein R¹⁰⁹ is hydrido or acetyl; and wherein n is a number from zero to two, inclusive; or a pharmaceutically-acceptable salt thereof.

69. Conjugate of claim 68 wherein said inhibitor compound is 1-(3-mercapto-2-methyl-loxopropyl)-L-proline.

70. Conjugate of claim 3 wherein said dopamine-β-hydroxylase inhibitor compound is of the formula 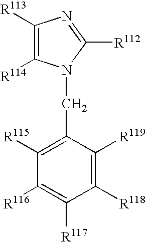
wherein each of R¹¹² through R¹¹⁹ is independently selected from hydrido, hydroxy, alkyl, cycloalkyl, cycloalkylalkyl, alkoxy, alkoxyalkyl, aralkyl, aryl, alkoxycarbonyl, hydroxyalkyl, halo, haloalkyl, cyano, amino, aminoalkyl, monoalkylamino, dialkylamino, carboxyl, carboxyalkyl, alkanoyl, alkenyl, cycloalkenyl, alkynyl, mercapto and alkylthio; or a pharmaceutically-acceptable salt thereof.

71. Conjugate of claim 70 wherein R¹¹² is selected from mercapto and alkylthio; wherein each of R¹¹³ and R¹¹⁴ is independently selected from hydrido, amino, aminoalkyl, monoalkylamino, monoalkylaminoalkyl, carboxyl and carboxyalkyl; wherein each of R¹¹⁵ and R¹¹⁹ is hydrido; and wherein each of R¹¹⁶, R¹¹⁷ and R¹¹⁸ is independently selected from hydrido, hydroxy, alkyl, halo and haloalkyl; or a pharmaceutically-acceptable salt thereof.

72. Conjugate of claim 71 wherein R¹¹² is selected from amino, aminoalkyl, monoalkylamino, monoalkylaminoalkyl, carboxy and carboxyalkyl; wherein each of R¹¹³, R¹¹⁴, R¹¹⁵ and R¹¹⁹ is hydrido; and wherein each of R¹¹⁶, R¹¹⁷ and R¹¹⁸ is independently selected from hydrido, hydroxy, alkyl, halo and haloalkyl; or a pharmaceutically-acceptable salt thereof.

73. Conjugate of claim 2 wherein said precursor compound providing the second residue has a reactable acid moiety.

74. Conjugate of claim 73 wherein said second residue precursor compound of said conjugate is selected from a class of glutamic acid derivatives of the formula 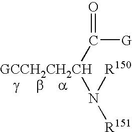
wherein each of R¹⁵⁰ and R¹⁵¹ may be independently selected from hydrido, alkylcarbonyl, alkoxycarbonyl, alkoxyalkyl, hydroxyalkyl and haloalkyl; and wherein G is selected from hydroxyl, halo, mercapto, —OR¹⁵², —SR¹⁵³ and 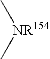
with each R¹⁵², R¹⁵³ and R¹⁵⁴ is independently selected from hydrido and alkyl; with the proviso that said glutamic acid derivative is selected such that formation of the cleavable bond occurs at the carbonyl moiety attached at the gamma-position carbon of said gamma-glutamic acid derivative.

75. Conjugate of claim 74 wherein R¹¹⁰ wherein each G is hydroxy; wherein R¹⁵⁰ is hydrido; and wherein R¹⁵¹ is selected from 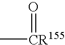
wherein R¹⁵⁵ is selected from methyl, ethyl, n-propyl, isopropyl, n-butyl, sec-butyl, iso-butyl, tert-butyl, n-pentyl, neopentyl, n-hexyl and chloromethyl.

76. Conjugate of claim 2 wherein said first and second residues are connected through a cleavable bond provided by a linker group between said first and second residues.

77. Conjugate of claim 76 wherein said linker group is selected from a class of diamino-terminated linker groups of the formula 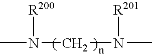
wherein each of R²⁰⁰ and R²⁰¹ may be independently selected from hydrido, alkyl, cycloalkyl, cycloalkylalkyl, alkoxyalkyl, hydroxyalkyl, aralkyl, aryl, haloalkyl, amino, monoalkylamino, dialkylamino, cyanoamino, carboxyalkyl, alkylsulfino, alkylsulfonyl, arylsulfinyl and arylsulfonyl;
and wherein n is zero or a number selected from three through seven, inclusive.

78. Conjugate of claim 77 wherein each of R²⁰⁰ and R²⁰¹ is hydrido; and wherein n is zero.

79. Conjugate of claim 76 wherein said linker group is selected from diamino terminal linker groups of the formula 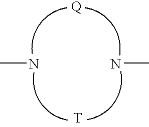
wherein each of Q and T is one or more groups independently selected from 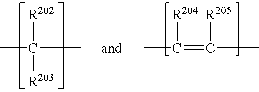
wherein each of R²⁰² through R²⁰⁵ is independently selected from hydrido, hydroxy, alkyl, cycloalkyl, cycloalkylalkyl, aralkyl, aryl, alkoxy, aralkoxy, aryloxy, alkoxyalkyl, haloalkyl, hydroxyalkyl, halo, cyano, amino, monoalkylamino, dialkylamino, carboxy, carboxyalkyl, alkanoyl, alkenyl, cycloalkenyl and alkynyl.

80. Conjugate of claim 79 wherein said linker group is of the formula 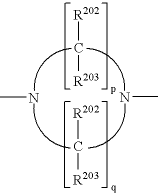
wherein each of R²⁰² and R²⁰³ is independently selected from hydrido, hydroxy, alkyl, phenalkyl, phenyl, alkoxy, benzyloxy, phenoxy, alkoxyalkyl, hydroxyalkyl, halo, amino, monoalkylamino, dialkylamino, carboxy, carboxyalkyl and alkanoyl; and wherein each of p and q is a number independently selected from one through six, inclusive; with the proviso that when each of R²⁰² and R²⁰³ is selected from halo, hydroxy, amino, monoalkylamino and dialkylamino, then the carbon to which R²⁰² or R²⁰³ is attached not adjacent to a nitrogen atom.

81. Conjugate of claim 80 wherein said linker group is selected from divalent radicals wherein each of R²⁰² and R²⁰³ is independently selected from hydrido, hydroxy, alkyl, alkoxy, amino, monoalkylamino, carboxy, carboxyalkyl and alkanoyl; and wherein each of p and q is a number independently selected from two through four, inclusive.

82. Conjugate of claim 81 wherein each of R²⁰² and R²⁰³ is independently selected from hydrido, amino, monoalkylamino and carboxyl; and wherein each of p and q is independently selected from the numbers two and three.

83. Conjugate of claim 82 wherein each of R²⁰² and R²⁰³ is hydrido; and wherein each of p and q is two.

84. Conjugate of claim 76 wherein said linker group is selected from diamino terminal linker groups of the formula 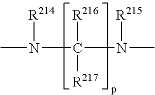
wherein each of R²¹⁴ through R²¹⁷ is independently selected from hydrido, alkyl, cycloalkyl, cycloalkylalkyl, hydroxyalkyl, alkoxyalkyl, aralkyl, aryl, haloalkyl, amino, monoalkylamino, dialkylamino, cyanoamino, carboxyalkyl, alkylsulfino, alkylsulfonyl, arylsulfinyl and arylsulfonyl; and wherein p is a number selected from one through six, inclusive.

85. Conjugate of claim 84 wherein each of R²¹⁴ and R²¹⁵ is hydrido; wherein each of R²¹⁶ and R²¹⁷ is independently selected from hydrido, alkyl, phenalkyl, phenyl, alkoxyalkyl, hydroxyalkyl, haloalkyl and carboxyalkyl; and wherein p is two or three.

86. Conjugate of claim 86 wherein each of R²¹⁴ and R²¹⁵ is hydrido; wherein each of R²¹⁶ and R²¹⁷ is independently selected from hydrido and alkyl; and wherein p is two.

87. Conjugate of claim 86 wherein each of R²¹⁴ through R²¹⁷ is hydrido; and wherein p is two.

88. Conjugate of claim 3 selected from the group consisting of
4-amino-4-carboxy-1-oxobutyl-α-methyl-L-tyrosine, methyl ester;
N-[4-(acetylamino)-4-carboxy-1-oxobutyl]-α-methyl-L-tyrosine, methyl ester;
N-[4-(acetylamino)-4-carboxy-1-oxobutyl]-α-methyl-L-tyrosine;
4-amino-4-carboxy-1-oxobutyl-3-hydroxy-α-methyl-L-tyrosine, methyl ester;
N-[4-(acetylamino)-4-carboxy-1-oxobutyl]-3-hydroxy-α-methyl-L-tyrosine, methyl ester;
N-[4-(acetylamino)-4-carboxy-1-oxobutyl]-3-hydroxy-α-methyl-L-tyrosine;
L-glutamic acid, 5-{[(5-butyl-2-pyridinyl)carbonyl]hydrazide};
N-acetyl-L-glutamic acid, 5-[(5-butyl-2-pyridinyl)carbonyl]hydrazide;
N-[2-[[(5-butyl-2-pyridinyl)carbonyl]amino]ethyl]-L-glutamine;
N²-acetyl-N-[2-[[(5-butyl-2-pyridinyl)carbonyl]amino]ethyl]-L-glutamine;
2-amino-5-[4-[(5-butyl-2-pyridinyl)carbonyl]-1-piperazinyl]-5-oxopentanoic acid;
2-(acetylamino)-5-(4-[(5-butyl-2-pyridinyl)carbonyl]-1-piperazinyl]-5-oxopentanoic acid; and
N²-acetyl-N-[2-[[5-butyl-2-pyridinyl)carbonyl]amino]ethyl]-L-glutamine, ethyl ester.

89. Conjugate of claim 8 which comprises a first residue provided by a tyrosine hydroxylase inhibitor compound and a second residue provided by a gamma glutamic acid derivative.

90. Conjugate of claim 89 which is 4-amino-4-carboxy-1-oxobutyl-α-methyl-L-tyrosine, methyl ester.

91. Conjugate of claim 89 which is N-[4-(acetylamino) -4-carboxy-1-oxobutyl]-α-methyl-L-tyrosine, methyl ester.

92. Conjugate of claim 89 which is N-[4-(acetylamino)-4-carboxy-1-oxobutyl]-α-methyl-L-tyrosine; 4-amino-4-carboxy-1-oxobutyl-3-hydroxy-α-methyl-L-tyrosine, methyl ester.

93. Conjugate of claim 25 which comprises a first residue provided by a dopa-decarboxylase inhibitor compound and a second residue provided by a gamma glutamic acid derivative.

94. Conjugate of claim 93 which is 4-amino-4-carboxy-1-oxobutyl-3-hydroxy-α-methyl-L-tyrosine, methyl ester.

95. Conjugate of claim 93 which is N-[4-(acetylamino)-4-carboxy-1-oxobutyl]-3-hydroxy-α-methyl-L-tyrosine, methyl ester.

96. Conjugate of claim 93 which is N-[4-(acetylamino)-4-carboxy-1-oxobutyl]-3-hydroxy-α-methyl-L-tyrosine.

97. Conjugate of claim 64 which comprises a first residue provided by a dopamine-β-hydroxylase inhibitor compound and a second residue provided by a gamma glutamic acid derivative.

98. Conjugate of claim 97 which is L-glutamic acid, 5-{[(5-butyl-2-pyridinyl)carbonyl]hydrazide}.

99. Conjugate of claim 97 which is N-acetyl-L-glutamic acid, 5-[(5-butyl-2-pyridinyl)-carbonyl]hydrazide.

100. Conjugate of claim 97 which is N-[2-[[(5-butyl-2-pyridinyl)carbonyl]amino]ethyl]-L-glutamine.

101. Conjugate of claim 97 which is N²-acetyl-N-[2-[[(5-butyl-2-pyridinyl)carbonyl]amino]ethyl]-L-glutamine.

102. Conjugate of claim 97 which is 2-amino-5-[4-[(5-butyl-2-pyridinyl)carbonyl]-1-piperazinyl]-5-oxopentanoic acid.

103. Conjugate of claim 97 which is 2-(acetylamino)-5-(4-[(5-butyl-2-pyridinyl)carbonyl]-1-piperazinyl)-5-oxopentanoic acid.

104. Conjugate of claim 97 which is N²-acetyl-N-[2-[[5-butyl-2-pyridinyl)carbonyl]amino]ethyl]-L-glutamine, ethyl ester.

105. A pharmaceutical composition comprising one or more pharmaceutically-acceptable carriers or diluents and a therapeutically-effective amount of a conjugate of claim 1.

106. A method for treating a hypertensive-related disorder or a sodium-retaining disorder, said method comprising administering to a patient afflicted with or susceptible to said disorder a therapeutically-effective amount of a conjugate of claim 1.

107. The method of claim 106 wherein said hypertensive-related disorder is chronic hypertension.

108. The method of claim 106 wherein said sodium-retaining disorder is congestive heart failure.

109. The method of claim 106 wherein said sodium-retaining disorder is cirrhosis.

110. The method of claim 106 wherein said sodium-retaining disorder is nephrosis.
